SEQUENCE LISTING (77 sequences)

SEQ ID NO: 1 - DNA, 20 bp, Homo sapiens
  source 1..20: mol_type = genomic DNA
ggagtcaaatctgtaggcca

SEQ ID NO: 2 - DNA, 20 bp, Homo sapiens
  source 1..20: mol_type = genomic DNA
gtaagcaacagattcagctg

SEQ ID NO: 3 - DNA, 20 bp, Homo sapiens
  source 1..20: mol_type = genomic DNA
gagacccagctaagcaacca

SEQ ID NO: 4 - DNA, 20 bp, Homo sapiens
  source 1..20: mol_type = genomic DNA
tcccaatctccatatcgggt

SEQ ID NO: 5 - DNA, 20 bp, Homo sapiens
  source 1..20: mol_type = genomic DNA
tgcagccgtggaagagacag

SEQ ID NO: 6 - DNA, 20 bp, Homo sapiens
  source 1..20: mol_type = genomic DNA
ggcacgtaccaaagccaatg

SEQ ID NO: 7 - DNA, 20 bp, Homo sapiens
  source 1..20: mol_type = genomic DNA
gaggccagaaggaggaaccg

SEQ ID NO: 8 - DNA, 20 bp, Homo sapiens
  source 1..20: mol_type = genomic DNA
gagacggggtctgaatggag

SEQ ID NO: 9 - DNA, 20 bp, Homo sapiens
  source 1..20: mol_type = genomic DNA
ggggaaggttcaggaagcga

SEQ ID NO: 10 - DNA, 20 bp, Homo sapiens
  source 1..20: mol_type = genomic DNA
gatgctggtgagccaggagc

SEQ ID NO: 11 - DNA, 20 bp, Homo sapiens
  source 1..20: mol_type = genomic DNA
ggagccgggcccattcgcac

SEQ ID NO: 12 - DNA, 20 bp, Homo sapiens
  source 1..20: mol_type = genomic DNA
gccactacccaggagaccga

SEQ ID NO: 13 - DNA, 20 bp, Homo sapiens
  source 1..20: mol_type = genomic DNA
gaacaagatggccgacgcgg

SEQ ID NO: 14 - DNA, 20 bp, Homo sapiens
  source 1..20: mol_type = genomic DNA
gagtgccgagaaagcatccg

SEQ ID NO: 15 - DNA, 20 bp, Homo sapiens
  source 1..20: mol_type = genomic DNA
gtgcaggagccacacacaaa

SEQ ID NO: 16 - DNA, 20 bp, Homo sapiens
  source 1..20: mol_type = genomic DNA
tgatgatgtagaggaaatcg

SEQ ID NO: 17 - DNA, 20 bp, Homo sapiens
  source 1..20: mol_type = genomic DNA
tacctggatagaaaggtcgt

SEQ ID NO: 18 - DNA, 20 bp, Homo sapiens
  source 1..20: mol_type = genomic DNA
ccttaccaagagaaaccagg

SEQ ID NO: 19 - DNA, 20 bp, Homo sapiens
  source 1..20: mol_type = genomic DNA
gtggtgaacggatcgagctg

SEQ ID NO: 20 - DNA, 20 bp, Homo sapiens
  source 1..20: mol_type = genomic DNA
tctggatgaaaatggagccg

SEQ ID NO: 21 - DNA, 3160 bp, Homo sapiens
  source 1..3160: mol_type = genomic DNA
gtgcggctgcatcggcggctccgggaagatggcggcccgggcgggtttccagtctgtggctccaagcggcggcgccggagcctcaggaggggcgggcgcggctgctgccttgggcccgggcggaactccggggcctcctgtgcgaatgggcccggctccgggtcaagggctgtaccgctccccgatgcccggagcggcctatccgagaccaggtatgttgccaggcagccgaatgacacctcagggaccttccatgggaccccctggctatggggggaacccttcagtccgacctggcctggcccagtcagggatggatcagtcccgcaagagacctgcccctcagcagatccagcaggtccagcagcaggcggtccaaaatcgaaaccacaatgcaaagaaaaagaagatggctgacaaaattctacctcaaaggattcgtgaactggtaccagaatcccaggcctatatggatctcttggcttttgaaaggaaactggaccagactatcatgaggaaacggctagatatccaagaggccttgaaacgtcccatcaagcaaaaacggaagctgcgaattttcatttctaacactttcaatccggctaagtcagatgccgaggatggggaagggacggtggcttcctgggagcttcgggtagaaggacggctcctggaggattcagccttgtccaaatatgatgccactaaacaaaagaggaagttctcttccttttttaagtccttggtgattgaactggacaaagacctgtatgggccagacaaccatctggtagaatggcacaggaccgccactacccaggagaccgatggctttcaggtgaagcggccgggagacgtgaatgtacggtgtactgtcctactgatgctggattaccagcctccccagtttaaattagacccccgcctagctcgactcctgggcatccatacccagactcgtccagtgatcatccaagcactgtggcaatatattaagacacataagctccaggaccctcacgagcgggagtttgtcatctgtgacaagtacctgcagcagatctttgagtctcaacgtatgaagttttcagagatccctcagcggctccatgccttgcttatgccaccagaacctatcatcattaatcatgtcatcagtgttgacccgaatgatcagaaaaagacagcttgttatgacattgatgttgaagtggatgacaccttgaagacccagatgaattcttttctgctgtccactgccagccaacaggagattgctactctagacaacaagacaatgactgatgtggtgggtaacccagaggaggagcgccgagctgagttctacttccagccctgggctcaggaggctgtgtgccgatacttctactccaaggtgcagcagagacgacaagaattagagcaagccctgggaatccggaatacatagggcctctcccacagccctgattcgactgcaccaattcttgatttgggccctgtgctgcctgcctcatagtatctgccttggtcttgcttggggcgttccaggggatgctgttggttcaaggacaacaccagaatgaagagggtctcacaagacacctgttatcctcttctttcaccctatctcttcccacccccagcttccctttgccccacaaagttcccatgtgcctgtaccctcccctggtctacataggacctctagatagtgttagagagagaacatgtagtggtaatgagtgcttggaatggattgggcctcaggccaggtggtcttcaaggggaccagctaactgatcctgcccttcagagacccaggagttgggagctttcgctccttctccaagactcaggcctgtgggcactctataagctagttgatcttggctctcctgataacagaatccaatttccttccttccctccacaggtttggaacaaactctcccttcacttgttgccctgtagcactacagaaaccctggttcttgggctccactgagccccaggtcagtccccagccctctgggttggcctgctgtcagtgcttctctcactccttagttggggtccacatcagtattggagttttgttctttattgctccctcccagacactccctgtggctgccctttgtgattccctcagatctgccctaatcccgggcatttgggtgggggaatcttgcctttccctttcagagccccagggatctcatctggggaactgtcattgccagcagaggctgttccttcctgctgtttggagatgtgactcattcattcactcactccaccctgcctctgcatcccttaatggagaaacgggcctaaaaccaaacgggtaaaaagccctgggccatccctgtcttcctgtcccttgtctgcccagttgacacctactggtgacttctagggcactgaggagtgaaagcgcctagggctggagaatagcgctgagttgggtttgtgactcttccctctccctgcctcacaggattgtgactccccagcccctgccctcaaagcttcagacccctcaggtagcagcaggaccttgtgatcttggccccttggatctgagatggtttttgcatctttccaggagagcctcacattcttcttccaggttgtatcacccccgagttagcatatcccaggctcgcagactcaacacagcaagggtgggagacagctgggcacaaagggggaattccgttcagcatgggctctaaacccacagaactgacaaagcccctgcttccccaccccctcctcaggctcctgcgagcacacccccacccccaaatccctccctgttctacactggggacagcagaattttctccccgtcttccccttcctgccattttccctcccttgaaaggttgacactggacaaccttggggcagctgagccctggccgcctcctggctggaaccatgagaaggaagctcagtacttcccacagtgtccctgttgataactgtttttattaactgaattgtttttttcatggaccaaacttttttttgtactgtccccttattgatgttacccagttttaataaaagaatcttctgaaggatgggtcctcctacctactgtgagagagctcttccctgagctcttcttccttcaataccatta

SEQ ID NO: 22 - DNA, 3283 bp, Homo sapiens
  source 1..3283: mol_type = genomic DNA
gtgcggctgcatcggcggctccgggaagatggcggcccgggcgggtttccagtctgtggctccaagcggcggcgccggagcctcaggaggggcgggcgcggctgctgccttgggcccgggcggaactccggggcctcctgtgcgaatgggcccggctccgggtcaagggctgtaccgctccccgatgcccggagcggcctatccgagaccaggtatgttgccaggcagccgaatgacacctcagggaccttccatgggaccccctggctatggggggaacccttcagtccgacctggcctggcccagtcagggatggatcagtcccgcaagagacctgcccctcagcagatccagcaggtccagcagcaggcggtccaaaatcgaaaccacaatgcaaagaaaaagaagatggctgacaaaattctacctcaaaggattcgtgaactggtaccagaatcccaggcctatatggatctcttggcttttgaaaggaaactggaccagactatcatgaggaaacggctagatatccaagaggccttgaaacgtcccatcaagcaaaaacggaagctgcgaattttcatttctaacactttcaatccggctaagtcagatgccgaggatggggaagggacggtggcttcctgggagcttcgggtagaaggacggctcctggaggattcagccttgtccaaatatgatgccactaaacaaaagaggaagttctcttccttttttaagtccttggtgattgaactggacaaagacctgtatgggccagacaaccatctggtagaatggcacaggaccgccactacccaggagaccgatggctttcaggtgaagcggccgggagacgtgaatgtacggtgtactgtcctactgatgctggattaccagcctccccagtttaaattagacccccgcctagctcgactcctgggcatccatacccagactcgtccagtgatcatccaagcactgtggcaatatattaagacacataagctccaggaccctcacgagcgggagtttgtcatctgtgacaagtacctgcagcagatctttgagtctcaacgtatgaagttttcagagatccctcagcggctccatgccttgcttatgccaccagaacctatcatcattaatcatgtcatcagtgttgacccgaatgatcagaaaaagacagcttgttatgacattgatgttgaagtggatgacaccttgaagacccagatgaattcttttctgctgtccactgccagccaacaggagattgctactctagacaacaagatccatgagacaatagaaaccatcaaccagctgaagactcagcgggagttcatgctgagctttgccagagaccctcagggtttcatcaatgactggcttcagtcccagtgcagggacctcaagacaatgactgatgtggtgggtaacccagaggaggagcgccgagctgagttctacttccagccctgggctcaggaggctgtgtgccgatacttctactccaaggtgcagcagagacgacaagaattagagcaagccctgggaatccggaatacatagggcctctcccacagccctgattcgactgcaccaattcttgatttgggccctgtgctgcctgcctcatagtatctgccttggtcttgcttggggcgttccaggggatgctgttggttcaaggacaacaccagaatgaagagggtctcacaagacacctgttatcctcttctttcaccctatctcttcccacccccagcttccctttgccccacaaagttcccatgtgcctgtaccctcccctggtctacataggacctctagatagtgttagagagagaacatgtagtggtaatgagtgcttggaatggattgggcctcaggccaggtggtcttcaaggggaccagctaactgatcctgcccttcagagacccaggagttgggagctttcgctccttctccaagactcaggcctgtgggcactctataagctagttgatcttggctctcctgataacagaatccaatttccttccttccctccacaggtttggaacaaactctcccttcacttgttgccctgtagcactacagaaaccctggttcttgggctccactgagccccaggtcagtccccagccctctgggttggcctgctgtcagtgcttctctcactccttagttggggtccacatcagtattggagttttgttctttattgctccctcccagacactccctgtggctgccctttgtgattccctcagatctgccctaatcccgggcatttgggtgggggaatcttgcctttccctttcagagccccagggatctcatctggggaactgtcattgccagcagaggctgttccttcctgctgtttggagatgtgactcattcattcactcactccaccctgcctctgcatcccttaatggagaaacgggcctaaaaccaaacgggtaaaaagccctgggccatccctgtcttcctgtcccttgtctgcccagttgacacctactggtgacttctagggcactgaggagtgaaagcgcctagggctggagaatagcgctgagttgggtttgtgactcttccctctccctgcctcacaggattgtgactccccagcccctgccctcaaagcttcagacccctcaggtagcagcaggaccttgtgatcttggccccttggatctgagatggtttttgcatctttccaggagagcctcacattcttcttccaggttgtatcacccccgagttagcatatcccaggctcgcagactcaacacagcaagggtgggagacagctgggcacaaagggggaattccgttcagcatgggctctaaacccacagaactgacaaagcccctgcttccccaccccctcctcaggctcctgcgagcacacccccacccccaaatccctccctgttctacactggggacagcagaattttctccccgtcttccccttcctgccattttccctcccttgaaaggttgacactggacaaccttggggcagctgagccctggccgcctcctggctggaaccatgagaaggaagctcagtacttcccacagtgtccctgttgataactgtttttattaactgaattgtttttttcatggaccaaacttttttttgtactgtccccttattgatgttacccagttttaataaaagaatcttctgaaggatgggtcctcctacctactgtgagagagctcttccctgagctcttcttccttcaataccatta

SEQ ID NO: 23 - DNA, 3257 bp, Homo sapiens
  source 1..3257: mol_type = genomic DNA
attggctgtgagggttgaatgtaagatggcgcccagggagctgtgaggagaaaaccctgtcggtcttggagcgacgacggcagaaccagggtccctggcggtgcggcggggccggcgggtgcagcggaggcggcggcggcggcggcagtgacgtcgccgggaaccctaaggactctgcaatatgaataattccctagagaacaccatctcctttgaagagtacatccgagtaaaggcacggtctgtcccgcaacacaggatgaaggaatttctggactcactggcctctaaggggccagaagcccttcaggagttccagcagacagccaccactaccatggtgtaccaacagggtgggaactgcatatacacagacagcactgaagtggctgggtctttgcttgaacttgcctgtccagtcaccaccagtgttcagccacaaacccagcaagaacagcagatccaggttcagcagccgcagcaggttcaggtccaggtgcaggtacagcagtctccgcaacaggtctcggctcagctctccccacaactcaccgttcaccagcctactgagcaacccatccaggtccaggtgcagatccaaggccaggcaccacagtcagcagccccctccattcagaccccgtctctgcagagtcccagtccctcgcagctgcaagcagctcagatccaggtgcagcacgtgcaagcagcccagcagatccaggctgcagaaatcccggaggagcacatcccacatcagcaaatccaggctcagctggtggctggccagtctcttgctggtggtcagcagatccaaatccagaccgtgggtgccctttccccaccaccatcccagcagggctcaccccgggaaggggagcggcgggttggcacggccagtgtcctccaaccagtgaagaagcgcaaagtggacatgcccatcactgtgtcctacgccatctcagggcagccggtggccaccgtgctggccattccacagggccagcagcagagttatgtgtctttgaggccagacttactgacagtagacagtgcccacctgtacagtgccactgggaccattactagccctacaggagaaacctggaccatccctgtttattctgcccagccccggggggaccctcagcagcagagcattacccacattgccattccccaggaagcctacaacgcagttcacgtcagtggctcacccacagccctggcagctgttaagctggaggatgacaaggagaagatggtgggcaccacatctgtagtgaaaaactcccatgaagaggtagtgcagacccttgcaaactctctctttccagcacagttcatgaatggcaacatccacattccagtggctgtgcaggctgtggcaggcacgtaccagaatacggctcaaactgtccatatatgggacccccaacagcagccgcagcagcaaactccccaggaacagacaccaccaccacagcagcagcagcagcaactccaagttacttgttcagctcaaactgtccaggttgctgaagttgaaccacagtcacagccacagccttccccagaacttctgcttccaaattctttgaagccagaagaagggcttgaagtatggaaaaactgggcccagaccaagaatgctgaactagagaaggatgctcagaacagattggcacccattgggaggcgccaactgctgcgattccaggaagatctcatctcctctgctgtggcagagttgaattatgggctctgtctaatgacacgggaagctcgaaatggagaaggtgaaccctatgacccagatgtgctctactatattttcctgtgtattcaaaagtatctttttgaaaatggaagggtagatgacattttctccgatctttattatgttcggttcacggagtggctacatgaagttctgaaggatgttcagccccgggtcactccacttggctatgtcttgcccagccacgtgactgaggagatgctatgggagtgcaagcagcttggggctcactccccctccaccttgctgaccaccctcatgttctttaataccaagtacttcctattgaagacagtggaccagcacatgaagctggccttctccaaggtcttgcgacagacaaagaagaacccctctaatcccaaggataaaagcacgagtatccggtacttgaaggcccttggaatacaccagactggccagaaagttacagatgacatgtatgcagaacagacggaaaatccagagaatccattgagatgtcccatcaagctctatgatttctacctcttcaaatgcccccagagtgtgaaaggccggaatgacaccttttacctgacacctgagccagtggtggcccccaacagcccaatctggtactcagtccagcctatcagcagagagcagatgggacaaatgctgacgcggatcctggtgataagagaaattcaggaggccatcgcagtggccaatgcaagcactatgcactgagatgccttggccatggcacaagagaaaccagccaggaaaaaccagacagactttcacactaaagaagaggcctccatttttttttttcttttttttattggtgtagttacgaagcctttcaggctgcttctgtttaaaatataaaagaaaactttgccccctttgcatcttcataaacctgctgcggcagactcctcagccgatggtggctctgggtttccttgagtgtcatatgtcctagaaagttgctggctgactcttttttgtctggggcctggggaaagggcttggactgtgaaaagaaatgtggcccctttccatcttcaagagagatggaattaatgatggatggaccctggagggaatctccccagccgacttccactgggctgacagactttgctgaccacaggggaacgatgttcttttctttcttcatgatcagacataaacttagcatcttaatggaagaaaaatgaggggaacttcaattatgatttattaaagacaatttctattacaccctcctttatgacaagtgacattttagatgtaaaagtaaaaactttaccatgccttttttttttttgttggcctaacattgaggccttaaaacctgaggctcctgtgcctgatggaattcttgtaacatacacttgtgtatcatataaagataccactctgtttctcttatgtattcttactctagttgtttattaagaatgacaagcacgtcttttcaaca

SEQ ID NO: 24 - DNA, 1865 bp, Homo sapiens
  source 1..1865: mol_type = genomic DNA
agcttgcggcctgtgtctatggtcgggccctctgcgtccagctgctccggaccgagctcgggtgtatggggccgtaggaaccggctccggggccccgataacgggccgcccccacagcaccccgggctggcgtgagggtctcccttgatctgagaatggctacctctcgatatgagccagtggctgaaattggtgtcggtgcctatgggacagtgtacaaggcccgtgatccccacagtggccactttgtggccctcaagagtgtgagagtccccaatggaggaggaggtggaggaggccttcccatcagcacagttcgtgaggtggctttactgaggcgactggaggcttttgagcatcccaatgttgtccggctgatggacgtctgtgccacatcccgaactgaccgggagatcaaggtaaccctggtgtttgagcatgtagaccaggacctaaggacatatctggacaaggcacccccaccaggcttgccagccgaaacgatcaaggatctgatgcgccagtttctaagaggcctagatttccttcatgccaattgcatcgttcaccgagatctgaagccagagaacattctggtgacaagtggtggaacagtcaagctggctgactttggcctggccagaatctacagctaccagatggcacttacacccgtggttgttacactctggtaccgagctcccgaagttcttctgcagtccacatatgcaacacctgtggacatgtggagtgttggctgtatctttgcagagatgtttcgtcgaaagcctctcttctgtggaaactctgaagccgaccagttgggcaaaatctttgacctgattgggctgcctccagaggatgactggcctcgagatgtatccctgccccgtggagcctttccccccagagggccccgcccagtgcagtcggtggtacctgagatggaggagtcgggagcacagctgctgctggaaatgctgacttttaacccacacaagcgaatctctgcctttcgagctctgcagcactcttatctacataaggatgaaggtaatccggagtgagcaatggagtggctgccatggaaggaagaaaagctgccatttcccttctggacactgagagggcaatctttgcctttatctctgaggctatggagggtcctcctccatctttctacagagattactttgctgccttaatgacattcccctcccacctctccttttgaggcttctccttctccttcccatttctctacactaaggggtatgttccctcttgtccctttccctacctttatatttggggtccttttttatacaggaaaaacaaaacaaagaaataatggtcttttttttttttttaatgtttcttcctctgtttggctttgccattgtgcgatttggaaaaaccacttggaagaagggactttcctgcaaaaccttaaagactggttaaattacagggcctaggaagtcagtggagccccttgactgacaaagcttagaaaggaactgaaattgcttctttgaatatggattttaggcggggcgtggtggctcacgcctataatcccagcacgttgggaggccaacgcgggtggatcacctgaggtcaggagttcgagaccagcctgactaacatggtgaaaccctgtctctactaaaaatacaaaattagtcaggcgtggtggtgcacacctgtaatcccagctacttgggagactgaggcaggaggatcgcttgaacccgggaggcagaggttgcggtgagccgagatcatgccattgcactccagcctgggcaacagagcaagactctgtgtcaaaaaaaaaaaaagaatatagatttttaaatggc

SEQ ID NO: 25 - DNA, 6678 bp, Homo sapiens
  source 1..6678: mol_type = genomic DNA
acactacgccagaacaagatggccgacgcggcggccacagctggggccggtggctccggaacgagatcgggaagtaaacagtccactaaccctgccgataactatcatctggcccggaggagaaccctgcaggtggttgtgagctccttgctgacagaggcagggtttgagagtgccgagaaagcatccgtggaaacgctgacagagatgctgcagagctacatttcagaaattgggagaagtgccaagtcttactgtgagcacacagccaggacccagcccacactgtccgatatcgtggtcacacttgttgagatgggtttcaatgtggacactctccctgcttatgcaaaacggtctcagaggatggtcatcactgctcctccggtgaccaatcagccagtgacccccaaggccctcactgcagggcagaaccgaccccacccgccgcacatccccagccattttcctgagttccctgatccccacacctacatcaaaactccgacgtaccgtgagcccgtgtcagactaccaggtcctgcgggagaaggctgcatcccagaggcgcgatgtggagcgggcacttacccgtttcatggccaagacaggcgagactcagagtcttttcaaagatgacgtcagcacatttccattgattgctgccagacctttcaccatcccctacctgacagctcttcttccgtctgaactggagatgcaacaaatggaagagacagattcctcggagcaggatgaacagacagacacagagaaccttgctcttcatatcagcatggaggattctggagccgagaaggagaacacctctgtcctgcagcagaacccctccttgtcgggtagccggaatggggaggagaacatcatcgataacccttatctgcggccggtgaagaagcccaagatccgcaggaagaagtccctctcctgagctgagaaggaaacctggcttgtacaggggcgcagattccaccctcccggggagttaaagccactcaagggaagaagagggtgacctcctcatggccaagccgaggctgcagggtgtgatcggacatgattttcatggcaaaccgtcttattaagatgacctattttcactggatgtttgggttgaggaagatatgaacagaataatgagaatttttttttttattttgagatggagtcttactctatcacccaggctggaatgcagtggtgtgatctcagctcactgcagtctcagcaaccctgggtccaagtgattctcctgccttggcctcccgagtagctgggactagaggcaagcgccaccatgctggctaatttttgtattttgagtagagatagggtttcgccatgttggccaggctggtcttgaactcctgacctcaagtgatccacccgcattggcctcccaaagagttgcgattacaggtgtgagccaccacgcccagctaatttttgtatttttaatagagataaggtttcgccatgttggccaggctggtcttgaactcctgacctcaagtgataacccgcctcggcctcccaaagtgctgagattacaggcgtgagccaccgcaccccaccagagactttcttaatcaatcaggctatgtggtggggcatagcagcagccagtgacttcgttcattatcacaggatttgattcctttgaaactcaagagaccagggaaagtgacaggaaggaaagggctctttgctgctgtgcttattacaaggaagactgtttgtccagcgtgtatttcaggatatctggatcccttttattgactcacaatttgtggcaccactttctcatcccagaacttcattcttatttctctcctcatctggcctcccaagtgctccgttgagctgatgaaaagttctttgtactccctcaacgtgtcggaaacaggaggccacacagcacagctttgtttggggtgggcaggagtcaggagtcttgagcagatgcatcactgtgaagaagaacgacatgtcggggctgcacctgtcctcccgtcggcatttgacgaaagctccctgaagcggggcagcactctcctcctgagagatttaccatttattgcccctgtgaggaatgtgtgcttgggaactgccaagtcttaccccttctggaagaagaggttttctctgacaagagcctagagcgtcggctctattatgctgggacttgacagaggagccatggggtttaaacagtaggaaagaggggctacgcgcagtggctcacgcctgtaatcccagcacttcgggaggcgaaggcaggtggatcacttgaggtcaggagttcgagaccagcctggccaacatagtgaaaccccgtctctactaaaaatacaaaaattagccgggtgtggttactcatgcctgtaatcccagctactcgggaggctgaggcaggagaatcacttgaacctgggaggcggaggttgcagtgagccgagatcgtgccactgcactccagcctgggtgacagagcgagactccatttcaaaaaaataaaaaaaggataaagtaaacaataggaaaggatttggtggggtgagggtggggtgttgagggctgggcctcatcttgtattctgaaagctgagaaacggctggtcagctggaaggctggtggatgggcaggagtgagccaagctgaggcgttctgcaagaagcaggtctgagtctgtccaaccaatttgtatcctgtggggagaacaacttcattcttaacataccctacaaaccagaaaacaagtttttaaggaagatgctgggcatgctgatagcttttctggtctcctaaggaagagtttttaaaactaaatccaaggaagaaaaatgtaacaacacgtggaagtgaacactggatgaataagcttgtttcccaaattagaatcctaacgtccatatttagcttcatgtattcccctttggcttccacagttcaactccttttattttgagacagggtctcgctgtgtcgccccagctggagtatagtggcactacctcggctcactgcaacctccactccccgggttcaagcaattctcagagtagctgggattacagatgcccgccaccacatctggctaattttttttttttttttttagtagacacgggattttgctatgttgcccaggctggtcttgaactcctggcctcaagtgatccacccgccttggccttccaaagtgttgagattacaggcgtgagccacggtgcccagcccatctcttagtcttccattcccttggtgaatttggaaacttgaaaaacttggggacttgacagcaggctccatgttcttctggcctcactgtactgtgtaaggaaagagctgcttgtcaggaacggaagaggggacgctagtaaaaaaaaaaaaattggattccccaactggactaaataggagtttttcaggtttgtagtaacgtgaaactctcctagatcgaagcagttagactggcctatgtggccttctcagggtttgtgattcagttcttagcgattcttggcctgataagtttatgaacacagccccacattcaagccgtggccaatcccagctgttctcactgctcttgggaggtgttgtcccagtcagtgtttctgcagcttccatttgttgttgttatccagtgtaagcacctgttgaatgcagatgtgctgagttagaggtgggatttggaaaagggctgctatttctgctgttgagatttcagaggctatactctttttttttcttttcttttttttttttttttgagacggcatctcactcttatcgcccaggctggagtgcaatggcgtgatctcggctcactgcaacctccacctcctgggttcaagggattctcctgcctcagcctcctgagtagctgggattacaggcacctaccacaattctcggctaattttttttgtatttttggtagagacggggtttcaccaggttggccagggtggtctcgaacacctgacctcagatgatccacccacctcggcctcccagtgtgggtgggattacaggcatgagccaccgcgcctggcctcagaggctatactcttataattttgttctgaggggagacagggaaagggttagacaacttgaaacattgaccctgtataaaaatgcaaaattctcaatgtgtatttataaatttctattttttataactttatgggacttcttagaggccaaaagtagtaaacatgcagattaaaaatgtttatgaaaatctcaataaaaattcataataaaaactttgtaaataaatagaactgtaagctttgaacttaatttgtgttcaaaagttaacagcaatgtggacaaataagtcaggcttagcccttgtgttggcctaagcacacctgggaacttagtaaaacttttattttggaaattttcaaatacaaaattagagagcaaaatatatgaacccctgtatatgcctcatcaagcttccacacttgtcagtagcttgttgattctgtttcatctgtttctcccactttttgtcctttattttctttcctcctggagtgtgtgtgtgtgtgtgtgtgcgtgtgtgtgcgtgtgagacagagtttcgcgctggagtgcaatggtgcaatcttggcccactgcaacttctgcctcccgggttcaaatgattctactgcctcagcttcctgagtagctgggattataggtatctaccaccaccaccacgcctggctaattttttcttttttttttggagacggagtctcgctctgtcgcccaggctggattgcagtggattgtcaagattgcagtggtgcaatctcagctcaccgcaagctccgccacccaggttcacgccattctcctgcctcagcctcccaagtagctgggactacaggtgcctgctaccacgcccagctaattttttgtatttttcagtagagatggagtttcactgcattagccaggatggtctcgatctcctgacctcatgatccgcccgcctcggcctcccaaagtgctgggattacaggcatgagccaccgcacctggcctaatttttttgtaggtttagtagagatggggtttcaccatgttggccagactggtctccaactcctgacctcaggtgatcacctgcctcagcctcctaaagtgctgaggttacaggcgtgagcccccgcgcctggctcttctggagtatttttaaatgcaaactccagacctgtgtcatttaatctaaatgtgtctatacgtatctctgattgatctttctttaaattttaattttggaataattatagattcacaagatgttgcaaaaaatgttgagagagatcctatgtacccatcacccactttctcccagtggtagcatcttgcataactgtggtgcaatatcaaaacccttgcattcagtttcagtgtacaatgtagatggtatattagtacaatgcatagagcttatttagttttcaccatgtgcacgtgtgtgttatgtgtgtgttgttccgtgcagtttcatcgtgtagatttgtgtaaccaccagcacacaaatggtgaagacacagaaccattgcatcagtacagttctccctcatgctgtctctttgcaatcacatccacctctctgctcttccacttctctcctggcagccactactctgttctctatctctaatgttattgaaagaatgttataaattgaatcataaagtatgtgacttttttctttttgagacagagtcttgctctatcgcctaggctggatggagtgcagtggagcgatcccagctcactgcaactcctccacctcccgggttcaagagattctcctgcctcagcctcccaagtagctgggattacaggcatgtgccaccacacccagctaacttttgtatttttagtagagatggggtttcaccatgttggccaggttggtctccaactcctgacctcaagtgatccgcccacctcagcctcccaaagtgctaggattataggtatgacccaccgcacctggccgagtatgggactttttgacgttggctttttcagttctgcataattctcttgaggtccatctaggtggttgtgtgtatcagtagttcctttctttttattgctgagtagcatggaggcaccatagtttaacacagtttctttaaccattcacgcattgagacaaaaaaagttcttaattctgcgaacatcaacttcctgtgcccaggtgcaagtgcgggggcagctggcccagggtagagctttaccatggaccctggcgctcctggaaaaaaattcaaggtatttttcctcctcttggagcgtgtggcctacctcatctccatttgattactagcttttctttttctcctttgttaaacagttatttaattttgaagagataatatatgtatacagtataaaatttaaaagacagaaaaagatataagtgataacaaaatatcttcctcctcccctgtttcccagaggctcagacctcatccctggcagctgctattatgttactgtcaatatgaccttatgtattcttccagaagtattctgtgcatcaaatgcaaatacttatattttctacttctcagtgtattttttggtgatgttccatgtcagtaataaagagcttctctgtttcgtg

SEQ ID NO: 26 - DNA, 4558 bp, Homo sapiens
  source 1..4558: mol_type = genomic DNA
acttccagttctgccccaccgctgcggccattgtccgaccccggtgcggctgaggcccctttgggcagcccctccgcagatcagaattggagataaccgaggcttcggcgggggcgggaggagctgcccgaggctctgggtgggccggaggtcgcgaaatccggagccccccagaggcgctcctgccccggagccgggccctggcgaggcaggaatggccccgaggcctccgaccgccgcgccccaggaatcagtgacattcaaagatgtgtctgtggacttcacccaggaagaatggtaccatgtcgaccctgctcagaggagcttatacagggatgtgatgctggagaactatagccacctggtttctcttggatatcaagtttccaagccagaggtgatcttcaaattggagcaaggagaagagccatggatatcagagggagaaatccaacgacctttctatccagactggaagaccaggcctgaagtcaaatcatcacatttgcagcaggatgtatcagaagtatcccactgcacacatgatctcttacatgctacattagaagactcctgggatgttagcagccagttagacaggcaacaggaaaactggaagagacatctgggatcagaggcatccacccagaagaaaataattacaccacaagaaaattttgagcaaaataaatttggtgaaaattctagattgaacaccaatttggttacacaactgaacattcctgcaagaataaggcctagtgaatgtgagacccttggaagcaatttgggacataatgcagacttacttaatgagaataatattcttgcaaaaaagaaaccctataagtgtgataaatgtagaaaagcctttattcatagatcatcgcttactaaacatgagaaaacacataaaggagagggagctttccctaatggaacagatcaaggaatttatcctggaaagaaacaccatgaatgtaccgactgtgggaaaacctttctctggaagacacagcttactgagcatcagagaattcacactggggagaaaccctttgaatgcaatgtatgtggaaaggccttcaggcatagctcatctcttggtcagcatgagaatgctcataccggagagaaaccctatcagtgtagtctctgtgggaaagccttccagcgcagctcctcccttgttcaacaccagcgaattcacactggagagaaaccctatcgatgtaatctatgtgggaggtcctttaggcatggcacatccctcactcaacacgaggtcacacacagtggagagaagcccttccagtgtaaggaatgtgggaaagcctttagtcgatgttcttcccttgtccaacatgagaggactcatactggagagaaaccttttgaatgtagcatatgtgggagggcttttggtcagagcccatccctttataaacatatgaggattcataagagaggcaaaccttaccaaagcagtaactacagcatagatttcaagcacagcacatctctcactcaagatgaaagcactcttaccgaagtgaaatcctaccattgtaatgactgtggggaagactttagtcacattacagactttactgaccatcagaggatccatactgcagagaacccctatgattgtgagcaggcttttagtcagcaagctatttctcatcctggagagaaaccctatcaatgtaatgtatgtgggaaagctttcaaaaggagtacaagtttcatagagcatcacagaattcatactggagagaaaccctatgaatgtaatgagtgtggagaagcctttagtcgacgctcatcgcttactcaacatgagagaacccacactggagagaaaccctatgaatgtattgactgtgggaaagcctttagtcaaagttcatctctcattcagcatgagagaactcatactggagagaagccctatgaatgtaatgaatgtgggagagccttccgaaaaaaaaccaacctgcatgatcatcagagaattcatactggagaaaaaccctattcttgtaaggaatgtgggaaaaacttcagccgaagttcagctcttactaaacaccagagaattcatactcgaaataaactctaggaaccgtgaaattaaggaatttgcagaatgctttagctaaaatgttctgattcaggatcagaggattcttagagagcttgggaatgtaatgaattacgtgtgtgtttatacgttgtgtgtggagaaaactgccagtagacagattttttttttttaacataaagacacattctcagatctgattacagactagtgtaaaaacagctacatgtatgtagctggttggggatgatatgcctgtatgttggactttgcttttgaatatatgtatgcaggatatcatcaagtttcaacatcttgacttgtgacccccaatgtcaacagcttttttaaaaagcaaattcctgcagtaatgaccaaaacccattttaaaaattgcttgacaactgcactcaactgcagctcttacattaacttcaccatggaaaccagttccaactccaggaagtcaccattcaaagaattagatcaactagcccaaccacttcattgtacagatgaagactgaaagccaaagatgtgaagtggtttccacagtatgatacagcctataagggtaaagctgggttaaaaatgcaggtttcctggatttggggccccatggccttgccagtgaaaaggttatttttggactcagagggctttaaaataaattttaagatgtatcagatacacaaacatttaatgggcacctatgggttggacactttgagaattcttaaaagtataagtgggagcaaaatgtatgcaaatttatcacaaactatttaaagcaacttcttggaggcttacaaaccacaatttaacagaaactgtagatggttgaactactagtgacttttttccccttttcccagttacaattatactttcagctaacatatgccagtttcacagaactattaagtccccttattgtactttttatggcatgcccatgaaaaagcactttcttaagcctacagtatcagatcaatgggaaaacaacagaaaactaagaggagaattttcccgttaattttcttgcagaaaagttaagtctaattgcccattgccataaattttgccttgtactcagagaagcaacatgcactggctcattttatgtgcaaagaaaagatttcaccattaaaaaaattaacttggctaggtatggtgtctcacacctgtaatcccagcactttgggtggctaaggcagatagactgcttgaacccaggagttcaagaccagcctggacaacatggtgaaaccccatctctttaaaaaaaaaaaaaaatccaaaaattagctgggcatggtggcatgcagtggtagtcccagctactcaggaggctgaggtgggaggatcactggaacccgggagcagagactgcagtgagctgagatcacactactgcattccagcctgagcaacagagcaagacacacacacatcaatttattttagttgtataatgcttttctattagtaaagcatcagctaagcttcagtggcctgctccatcccctaatgactcccatgggctatcctaaaggaacttccagaacctttgttggtgtgttgacattgaccatgcagaccaatttgggcacaactggacattgattccttttacacaagagctgcctcccaaagatagataaattttcccagccctaaatatgaatcatggggcaagatattggtcgtattgatggtgaacctttcctactggattctttgcatgccacatagcaggattcattgcctttctctcatcatggatggcatgcagcagcacccaagtattcttcattctttgcagggaaaaaattgtgcatgggggctgaaatgtaatatgtgtagctcaattagtctctcctctgtgatgcaaaatggaatattcaatggcagatctgcccttctgagatgctgaccatccaaaacaccttgtttatggtgcaccatgattagctcacacacaatgccaaggctgtgcttctattatctgatacatagtttgacaatgggtaattctactcagaccctccctactgattggctaggatgcctgtcaggaactcattatgctactggttgtttggggatccccatagtggactactttcaggaatggcatgaattgtaaccaactgagtgctgcccccactgttacggaagtttataaaaccttagttccagaagacccaaaggagagtactggtttgtgtttggtgcttggcctagatccagccaccactctgaaactcagcacatcttcattgacagggagggagcccaggacatatgtgtggctcattgaccagaaggctttcttagtcccaacagccatgaaccatgcacttatggatacccagccttttagggctacgtgaaatgcatccttgtaacatcattgtattctttcaataaatagccttctgagttgaa

SEQ ID NO: 27 - DNA, 6588 bp, Homo sapiens
  source 1..6588: mol_type = genomic DNA
ctttctcggccgccattttggctagggcaggttcggggactcggtccgaggcgcttgtattgtctgctgaggggagacgcgggatcagccccctcccccgcccgttgccgccgccgcctccgccggcccggtctcccctccgccgcccgccgggatccatgagctgaactcccgcccccccccccccggccgccgccatcttgtgccccctccccctcccgcaggcagcgctaagggggattttggcgtcttctcagacacagctccctctctcggtccccgctgctgaggagcgagaggagcgcggccgccgccgcccgcgccggtgaagccgcctctcccacaccctccgtctcctccctccgcccctcctttgtctgcaccgctcgacgacgccgccgccgcccgccgctctgctgcgcccgccgcgccccccggcagcacccgcggccgcggcgcgagccggagtccgccgagccggagcgcgacgaggccccgggcgcgccctccccgctgccgccaccgccgtgccgccgccatccgcccgccgccgccgccgctgtccggcccccgagcacgccggccccgcgcgcgcctcgaggccgagtcaaggtgtgagatgcacaatgcgaaacctaggccccagcttttacaccatgatgcgcagggttgtactttttgtactgaactgataggtggcctagtggttatgccctgtactaccattttgaggatctggactccgtttcctgccttgctctttggaccacattgtcaattcacaccgaaactatgttccaacttcctgtcaacaatcttggcagtttaagaaaagcccggaaaactgtgaaaaaaatacttagtgacattgggttggaatactgtaaagaacatatagaagattttaaacaatttgaacctaatgacttttatttgaaaaacactacatgggaggatgtaggactgtgggacccatcacttacgaaaaaccaggactatcggacaaaacctttctgctgcagcgcttgtccattttcctcaaaattcttctctgcctacaaaagtcatttccgcaatgtccatagtgaagactttgaaaataggattctccttaattgcccctactgtaccttcaatgcagacaaaaagactttggaaacacacattaaaatatttcatgctccgaacgccagcgcaccaagtagcagcctcagcactttcaaagataaaaacaaaaatgatggccttaaacctaagcaggctgacagtgtagagcaagctgtttattactgtaagaagtgcacttaccgagatcctctttatgaaatagttaggaagcacatttacagggaacattttcagcatgtggcagcaccttacatagcaaaggcaggagaaaaatcactcaatggggcagtccccttaggctcgaatgcccgagaagagagtagtattcactgcaagcgatgccttttcatgccaaagtcctatgaagctttggtacagcatgtcatcgaagaccatgaacgtataggctatcaggtcactgccatgattgggcacacaaatgtagtggttccccgatccaaacccttgatgctaattgctcccaaacctcaagacaagaagagcatgggactcccaccaaggatcggttcccttgcttctggaaatgtccggtctttaccatcacagcagatggtgaatcgactctcaataccaaagcctaacttaaattctacaggagtcaacatgatgtccagtgttcatctgcagcagaacaactatggagtcaaatctgtaggccagggttacagtgttggtcagtcaatgagactgggtctaggtggcaacgcaccagtttccattcctcaacaatctcagtctgtaaagcagttacttccaagtggaaacggaaggtcttatgggcttgggtcagagcagaggtcccaggcaccagcaagatactccctgcagtctgctaatgcctcttctctctcatcgggccagttaaagtctccttccctctctcagtcacaggcatccagagtgttaggtcagtccagttccaaacctgctgcagctgccacaggccctcccccaggtaacacttcctcaactcaaaagtggaaaatatgtacaatctgtaatgagctttttcctgaaaatgtctatagtgtgcacttcgaaaaagaacataaagctgagaaagtcccagcagtagccaactacattatgaaaatacacaattttactagcaaatgcctctactgtaatcgctatttacccacagatactctgctcaaccatatgttaattcatggtctgtcttgtccatattgccgttcaactttcaatgatgtggaaaagatggccgcacacatgcggatggttcacattgatgaagagatgggacctaaaacagattctactttgagttttgatttgacattgcagcagggtagtcacactaacatccatctcctggtaactacatacaatctgagggatgccccagctgaatctgttgcttaccatgcccaaaataatcctccagttcctccaaagccacagccaaaggttcaggaaaaggcagatatccctgtaaaaagttcacctcaagctgcagtgccctataaaaaagatgttgggaaaaccctttgtcctctttgcttttcaatcctaaaaggacccatatctgatgcacttgcacatcacttacgagagaggcaccaagttattcagacggttcatccagttgagaaaaagctcacctacaaatgtatccattgccttggtgtgtataccagcaacatgaccgcctcaactatcactctgcatctagttcactgcaggggcgttggaaagacccaaaatggccaggataagacaaatgcaccctctcggcttaatcagtctccaagtctggcacctgtgaagcgcacttacgagcaaatggaatttcccttactgaaaaaacgaaagttagatgatgatagtgattcacccagcttctttgaagagaagcctgaagagcctgttgttttagctttagaccccaagggtcatgaagatgattcctatgaagccaggaaaagctttctaacaaagtatttcaacaaacagccctatcccaccaggagagaaattgagaagctagcagccagtttatggttatggaagagtgacatcgcttcccattttagtaacaaaaggaagaagtgtgtccgtgattgtgaaaagtacaagcctggcgtgttgctggggtttaacatgaaagaattaaataaagtcaagcatgagatggattttgatgctgagtggctatttgaaaatcatgatgagaaggattccagagtcaatgctagtaagactgctgacaaaaagctcaaccttgggaaggaagatgacagttcctcagacagttttgaaaatttggaagaagaatccaatgaaagtggtagcccttttgaccctgtttttgaagttgaacctaaaatctctaacgataacccagaggaacatgtactgaaggtaattcctgaggatgcttcagaatctgaggagaagctagaccaaaaagaggatggttcaaaatacgaaactattcatttgactgaggaaccaaccaaactaatgcacaatgcatctgatagtgaggttgaccaagacgatgttgttgagtggaaagacggtgcttctccatctgagagtgggcctggatcccaacaagtgtcagactttgaggacaatacctgcgaaatgaaaccaggaacctggtctgacgagtcttcccaaagcgaagatgcaaggagcagtaagccagctgccaaaaaaaaggctaccatgcaaggtgacagagagcagttgaaatggaagaatagttcctatggaaaagttgaagggttttggtctaaggaccagtcacagtggaagaatgcatctgagaatgatgagcgcttatctaacccccagattgagtggcagaatagcacaattgacagtgaggatggggaacagtttgacaacatgactgatggagtagctgagcccatgcatggcagcttagccggagttaaactgagcagccaacaggcctaagtgccaggttccctggcgttggtgacatgctgcagcctggaactctgatatccagtgtgactgcaaagctgtcttctcactggtactgccttgtgagtactggttggactgtggggcatgtggccgctgcagttccagtggttatttctaagtctatgacaggacaggctgttcttgcttcagaaccttctctgacagacacggtaactaaatgtgaaaaaccaataagctggtgactcatgaatacacacgaggaaaagcagaggtttattttatctgccttttcaacatttctttccctctgtgaaatgattggtcagatgtctttgagaagtgttaaactaattcacatggtagtgtagggccaacatacaagctaccagtctaatgtgtatagtagactttgggaaaagcgattttttttcatgtattcattctgaatagttgaaatgtatatttgtacagtcttttagacctattcaagtgatgctcatgatcctgttactgtgtgcccatcatagatttctttttttagtgttgcccttgctgtgtaataaacgctctatctagtttacctagcaaaagctcaaaactgcgctagtatggactttttggacagacttagtttttgcacataaccttgtacaatcttgcaacagaggccagccacgtaagatatatatctggactctcttgtattataggatttttcttgttctgaatatccttgacattacagctgtcaaaaacaaaaactggtatttcagatctgttttctgaaatcttttaagctaaaatcacatgcaagaattgactttgcagctactaattttgacaccttttagatctgtataaaagtgtgttgtgttgaagcagcaaaccaatgagtgctgcattttggatatttagttttatctttagttcaacaccatcatggtggattcatttataccatctaatatatgacacactgttgtagtatgtataattttgtgatctttattttccctttgtattcattttaagcatctaaataaattgctgtattgtgcttaatgtaaatatttgctttattacacatgacagtcctgtgtgtccattatatcttttgccatttaatgccataactctttcatcagagatggcagccacagtcacagaggaaacttgtggaaaacccaagatctgtcatctgcaaatagaatgctttctcttgaggcacctggtacaaaatctgtttttcttttttttttttttttttttttttttttttaactgaaaaacaaacacccttgagtttttcagttcaccttttgttaaggagcagagcctgcatatatatgtaaatgtctcaatgataaaaaaaaaaatgtaaacatggattggcattaatttgcaagcagaatcttaatagcctgatgggttttttgtttgttttttcttctagatgaagttaaacgttcgttttcaccttacgatgggaacaactgtagtgcaggcggctctgcctctatttgccactcttggccctcttgctattaagggaaaggctttgaagagcacagcactgacagggctgtaaagatggaaggagctggtatttttaccatgcgtagattcccaagagaacttgtactaaaaagcagaaagttacaaggaagcagaattaggccaacatttccagcaattaaaacaatgatgggaaactccaatccatgaggaggctggtcagtcacctcaaggatagtggcctaaagaagtctgaagtggctgatgtccataacttctgagtctgtttccaaaagaaagactcttgttaatgctgttaccagagagtagagatttgaccagcctctccctaaaatgtaagcttctcaacaacagtacaggaagaagtgcaagagtagactagaacctttaaggaatttggggctggaacggagctatgttgcatctgttgttatacaggtagattataagttctacatgcaccagccaaaacttaggacaggatgtccccaaattaagatactacagattaccaaatcagaaataaccagctggatttaggccagctcacatttatttggaaattaccctccctgattaacaggcttctgagttgtacgtggaggactaatgagacttgtacaagacaccacggggcttggggtccaggtcccagttggatatcggaacagacacagataagtgtgtttcctcattaaggtggtgagagtgtgccttttggcattcagaaaagggtttagccaccctaagagttggaaataaagattcccaactggaaatgttgcagaatccctacaagtgtctcaataaaagccattttaaaagaatattgttgctactgtttgcttttcaaataatctggagatcgttggctatggcaacgtgctgccacctaattagagaatgcctgtaaatgtaggctgaacacttctaacatgagaaaacctaagtatccta

SEQ ID NO: 28 - DNA, 3105 bp, Homo sapiens
  source 1..3105: mol_type = genomic DNA
ggggaggaggccgcgcggggtggggtctggcggtacgcgctggctgcgtcgacgtgctgacgccatgacgccccggctggtgtgtgtcggtgtgtatgtgtgtgtgtgagtgtgcgcgctccgagtgtgtgtgtatttgtgtatcggcggtcccgcaggtcccggatgttgcggacagtatgaggcaagcgcagggggacggggaccagcagctgtcgccgccgctctcagatcgagtcttgctctgtcacccaggctggagtgcagtggcgcgatctcagctcactgccacctttgcctcctgggttcaagcgattcttctgcctcagcctcccgagtagctgggattacaggctctgggaaccacccttctactttctgtctctaggaatttcactactctagggtgaagagggaacagaaatctttgccccctgactttggaaatctcgtttaaccttcaaactggcgatgtcaagggttccaagtcctccacctccggcagaaatgtcgagtggccccgtagctgagagttggtgctacacacagatcaaggtagtgaaattctcctacatgtggaccatcaataactttagcttttgccgggaggaaatgggtgaagtcattaaaagttctacattttcatcaggagcaaatgataaactgaaatggtgtttgcgagtaaaccccaaagggttagatgaagaaagcaaagattacctgtcactttacctgttactggtcagctgtccaaagagtgaagttcgggcaaaattcaaattctccatcctgaatgccaagggagaagaaaccaaagctatggagagtcaacgggcatataggtttgtgcaaggcaaagactggggattcaagaaattcatccgtagagattttcttttggatgaggccaacgggcttctccctgatgacaagcttaccctcttctgcgaggtgagtgttgtgcaagattctgtcaacatttctggccagaataccatgaacatggtaaaggttcctgagtgccggctggcagatgagttaggaggactgtgggagaattcccggttcacagactgctgcttgtgtgttgccggccaggaattccaggctcacaaggctatcttagcagctcgttctccggtttttagtgccatgtttgaacatgaaatggaggagagcaaaaagaatcgagttgaaatcaatgatgtggagcctgaagtttttaaggaaatgatgtgcttcatttacacggggaaggctccaaacctcgacaaaatggctgatgatttgctggcagctgctgacaagtatgccctggagcgcttaaaggtcatgtgtgaggatgccctctgcagtaacctgtccgtggagaacgctgcagaaattctcatcctggccgacctccacagtgcagatcagttgaaaactcaggcagtggatttcatcaactatcatgcttcggatgtcttggagacctctgggtggaagtcaatggtggtgtcacatccccacttggtggctgaggcataccgctctctggcttcagcacagtgcccttttctgggacccccacgcaaacgcctgaagcaatcctaagatcctgcttgttgtaagactccgtttaatttccagaagcagcagccactgttgctgccactgaccaccaggtagacagcgcaatctgtggagcttttactctgttgtgaggggaagagactgcattgtggccccagacttttaaaacagcactaaataacttgggggaaacggggggagggaaaatgaaatgaaaaccctgttgctgcgtcactgtgttccctttggcctggctgagtttgatactgtggggattcagtttaggcgctggcccgaggatatcccagcggtggtacttcggagacacctgtctgcatctgactgagcagaacaaatcgtcaggtgcctggagcaaaaaggaaaaaaaaaaaagaaaggacattgagttttaacagaagggaaaaggaaagaagaaaagatttttgcagaatttctcaaaaatcagtttgtggattccagtagtatttatattgagagaaacaaattttagtccttctaactgtgctaaaacttggatatttgtgaaaactccttaccaccatacaagcatcagaagagctctcttgttgttagcacttattgtttgcaagaacagaatacatccttttatccttttatgaaaaatgacaagtgaaggcaaaaggggaaggttatttgatctggaagatgagtgttctgatgtggtggcttttgcaaaaatctttattggtgttgaaaactggaaaaaataactcatccagaattcatattgtcttgacaagaactatggttctctgtttttagatattgtggaaaatgtttttgggcatttttctctgattttatttcttctcccccacccctttttctaaaaaacaaacaaaaaaaaaaacacacaaaacaaaaacagaacaaaagaagagagaaggaaattttatcaattaaaaatgctgtgtgataaaatcccagcccagattgctcagctgtttgtacctgacttgccgcctgcataggagccagttctgttccttctgactagcccctcttcctccaggggagaacttccaaatgttaatttttttttttttgaaaatataaataattactattttgtactgtgtggtatctctggtcttttgtttcactcacctgccttgtctcttgggtctgagtcccttgcttaagggattttgaagtcctagttttcagcttgcagagattatgtctgaaatgcctaatgagtcgcagggatttgttgagactccgtaatctcaagttctctttgtgagctatcagcatctgccagtctcttgtcctccctgagtatctcacagtccatatcctgatgagggatcaggcccctacctctgccaaggcaagtaatggtagtgggcttttaaactgccccccgtatgttttaagacctaatccccacctcccttcttctaactaaatataaaaagatccaggggacataaatgtggagattaaataaagggaaattattgtctctaaaaaaaaaaaaaaaaa

SEQ ID NO: 29 - DNA, 5657 bp, Homo sapiens
  source 1..5657: mol_type = genomic DNA
attcaaacccggccggcccgagtggtgtcagctcagtcccggccgccgccgcgcgaggaaatggccgaggagccggagccgcagggtttggaaatcccttgtctccaggttgctgggattgacttcttgctcaattgaaacactcattcaatggagacaaagagaactaatgctttgtgctgattcatatttgaatcgaggcattgggaaccctgtatgccttgtttgtggaaagaaccagtgacaccatcactgagcttcctaaaagttcgaagaagttagaggactatacactttcttttgaacttttataataaatatttgctctggtttttggaacccagggctgttagaggggtgagtgacaagtcttacaagtggccttattccaactccagaaattgcccaacggaactttgagattatatgcaatcgaaagtgacaggaaacatgccaactcaatccctcttaatgtacatggatgggccagaagtgattggcagctctcttggcagtccgatggagatggaggatgccttgtcaatgaaagggaccgctgttgttccattccgagctacacaagaaaaaaatgtcatccaaatcgaggggtatatgcccttggattgcatgttctgcagccagaccttcacacattcagaagaccttaataaacatgtcttaatgcaacaccggcctaccctctgtgaaccagcagttcttcgggttgaagcagagtatctcagtccgcttgataaaagtcaagtgcgaacagaacctcccaaggaaaagaattgcaaggaaaatgaatttagctgtgaggtatgtgggcagacatttagagtcgcttttgatgttgagatccacatgagaacacacaaagattctttcacttacgggtgtaacatgtgcggaagaagattcaaggagccttggtttcttaaaaatcacatgcggacacataatggcaaatcgggggccagaagcaaactgcagcaaggcttggagagtagtccagcaacgatcaacgaggtcgtccaggtgcacgcggccgagagcatctcctctccttacaaaatctgcatggtttgtggcttcctatttccaaataaagaaagtctaattgagcaccgcaaggtgcacaccaaaaaaactgctttcggtaccagcagcgcgcagacagactctccacaaggaggaatgccgtcctcgagggaggacttcctgcagttgttcaacttgagaccaaaatctcaccctgaaacggggaagaagcctgtcagatgcatccctcagctcgatccgttcaccaccttccaggcttggcagctggctaccaaaggaaaagttgccatttgccaagaagtgaaggaatcggggcaagaagggagcaccgacaacgacgattcgagttccgagaaggagcttggagaaacaaataagggcagttgtgcaggcctctcgcaagagaaagagaagtgcaaacactcccacggcgaagcgccctccgtggacgcggatcccaagttacccagtagcaaggagaagcccactcactgctccgagtgcggcaaagctttcagaacctaccaccagctggtcttgcactccagggtccacaagaaggaccggagggccggcgcggagtcgcccaccatgtctgtggacgggaggcagccggggacgtgttctcctgacctcgccgcccctctggatgaaaatggagccgtggatcgaggggaaggtggttctgaagacggatctgaggatgggcttcccgaaggaatccatctggataaaaatgatgatggaggaaaaataaaacatcttacatcttcaagagagtgtagttattgtggaaagtttttccgttcaaattattacctcaatattcatctcagaacgcatacaggtgaaaaaccatacaaatgtgaattttgtgaatatgctgcagcccagaagacatctctgaggtatcacttggagagacatcacaaggaaaaacaaaccgatgttgctgctgaagtcaagaacgatggtaaaaatcaggacactgaagatgcactattaaccgctgacagtgcgcaaaccaaaaatttgaaaagattttttgatggtgccaaagatgttacaggcagtccacctgcaaagcagcttaaggagatgccttctgtttttcagaatgttctgggcagcgctgtcctctcaccagcacacaaagatactcaggatttccataaaaatgcagctgatgacagtgctgataaagtgaataaaaaccctacccctgcttacctggacctgttaaaaaagagatcagcagttgaaactcaggcaaataacctcatctgtagaaccaaggcggatgttactcctcctccggatggcagtaccacccataaccttgaagttagccccaaagagaagcaaacggagaccgcagctgactgcagatacaggccaagtgtggattgtcacgaaaaacctttaaatttatccgtgggggctcttcacaattgcccggcaatttctttgagtaaaagtttgattccaagtatcacctgtccattttgtaccttcaagacattttatccagaagttttaatgatgcaccagagactggagcataaatacaatcctgacgttcataaaaactgtcgaaacaagtccttgcttagaagtcgacgtaccggatgcccgccagcgttgctgggaaaagatgtgcctcccctctctagtttctgtaaacccaagcccaagtctgctttcccggcgcagtccaaatccctgccatctgcgaaggggaagcagagccctcctgggccaggcaaggcccctctgacttcagggatagactctagcactttagccccaagtaacctgaagtcccacagaccacagcagaatgtgggggtccaaggggccgccaccaggcaacagcaatctgagatgtttcctaaaaccagtgtttcccctgcaccggataagacaaaaagacccgagacaaaattgaaacctcttccagtagctccttctcagcccaccctcggcagcagtaacatcaatggttccatcgactaccccgccaagaacgacagcccgtgggcacctccgggaagagactatttctgtaatcggagtgccagcaatactgcagcagaatttggtgagccccttccaaaaagactgaagtccagcgtggttgcccttgacgttgaccagcccggggccaattacagaagaggctatgaccttcccaagtaccatatggtcagaggcatcacatcactgttaccgcaggactgtgtgtatccgtcgcaggcgctgcctcccaaaccaaggttcctgagctccagcgaggtcgattctccaaatgtgctgactgttcagaagccctatggtggctccgggccactttacacttgtgtgcctgctggtagtccagcatccagctcgacgttagaaggtcttggtggatgtcagtgcttactccccatgaaattaaattttacttcatcctttgagaagcgaatggtgaaagctactgaaataagctgtgattgtactgtacataaaacatatgaggaatctgcaaggaacactacagttgtgtaaagttgttctgttaacttttgtaccaaatagcaatacaaactagttggaacagttggaacttacatacatggggactggaaatctctattttgtccctgaataatatttttcttagaattgaccaaataagaagtggaatttttgcatacttgagcgctgctgaaaagaaatcatttgggttgggtggggcgggatgggggaaaagtatataatgcttgcacctcaggtaaaaatctgtaaatatctaagttgtaaacctgcttgttcaaatactgtgtgtattccttttctctaatgcagctcatcacttggagcagtttctgctattgtgctctttcatttaaaatgtatgtttttttttttttaaactgtcaatgattttgtattatgttgaatccacccaaatctattgttgtcttaaaattgttaatggaagtattgaccctctatgatatgtgctgcagatatcgaggtcagccattcggaagctggcagcattttatcgcaactttgagcatctcagatggggaaggcaccttcttcctcgcctctccagattgtcctggaacctccaggatccttgactgagggcgttgggatggcttgtaggatttttaagagagtgtgtctacagacaagcattttctctgtagagcagccacacgttgtataaacataaactgtatgtgcagttatttaaatttgtttctgtcaaattaatcatttttgttggacgattcagtggcgggggggttttgcctaaattagtatataaaaacaaaaatgctaaattatatctgtgaattgcaggtattggggaacagttttaagggaaattttggggggaacattttaggtttgtatttggtagtcttaatgtatctggcatttgggtgaactgtggacatactagagttgattatagacacattgattttgaataaggaactgctggccgagcccgctgggagtctagaaagagaaaatctgtttctagacctcagttattttcccatttttggttgttttgaagcagtaacatttttctcagtgcacatgcaatttgggttttagagaagatggccaccagctggcttcctagatattttaaacttttgttctttaatatgctgtccatggctgagtttattagtacatgggcttagtgaccacaaaatattttattaagaaactgtttcaaaaataaatttgcactgttcatttttctggcctcgctgttctccatagagcaagggtaatcctagaaaaaattttttttttttaaattatgcaacgtaagatgtcctccttgatagaagtcttagctcctgtgttacaagggagaactcatttgagatcagtctgttggcattgcaatgaagtgctttgtatcaggaaagtgtacactattgaccttttttcctgttcacaagctgagccatatgtacataatctagattttgttttcatagttttgcacttttatagcctatttttgaagattaacacatttgcaagatgattgactcaatctttgcctaatccaatgagtgttacagagagcttgctgtgactagaaccataaatcttaaagggggtatgtgataatagagggctggaatttaaacctgtatttaaaaaaaagaatcaccaaatctatttgaaaacaagtcgatttgtattatgctggaattttttgggctttcagatttctctttttaaccacatttctgaatgtataaaaataccaattattttcctacagccctttgtacttcaaaatatgtttttgtgtccatcagtattaactattggtatactactggttttatatttttttttctttgagacaacagtacatataatagaggtacaattcgttggatttttgtttatgtatttatttcattccagtttgatttattttaattgttgatacttaagttgtcgaacagtagacattacttgttttatttatgatatatttcagcttaaagttatgttattatatgtggaagtgtaaatatagatttggtgttttgcaa

SEQ ID NO: 30 - DNA, 5790 bp, Homo sapiens
  source 1..5790: mol_type = genomic DNA
attcaaacccggccggcccgagtggtgtcagctcagtcccggccgccgccgcgcgaggaaatggccgaggagccggagccgcagggtttggaaatcccttgtctccaggttgctgggattgacttcttgctcaattgaaacactcattcaatggagacaaagagaactaatgctttgtgctgattcatatttgaatcgaggcattgggaaccctgtatgccttgtttgtggaaagaaccagtgacaccatcactgagcttcctaaaagttcgaagaagttagaggactatacactttcttttgaacttttataataaatatttgctctggtttttggaacccagggctgttagaggggtgagtgacaagtcttacaagtggccttattccaactccagaaattgcccaacggaactttgagattatatgcaatcgaaagtgacaggaaacatgccaactcaatccctcttaatgtacatggatgggccagaagtgattggcagctctcttggcagtccgatggagatggaggatgccttgtcaatgaaagggaccgctgttgttccattccgagctacacaagaaaaaaatgtcatccaaatcgaggggtatatgcccttggattgcatgttctgcagccagaccttcacacattcagaagaccttaataaacatgtcttaatgcaacaccggcctaccctctgtgaaccagcagttcttcgggttgaagcagagtatctcagtccgcttgataaaagtcaagtgcgaacagaacctcccaaggaaaagaattgcaaggaaaatgaatttagctgtgaggtatgtgggcagacatttagagtcgcttttgatgttgagatccacatgagaacacacaaagattctttcacttacgggtgtaacatgtgcggaagaagattcaaggagccttggtttcttaaaaatcacatgcggacacataatggcaaatcgggggccagaagcaaactgcagcaaggcttggagagtagtccagcaacgatcaacgaggtcgtccaggtgcacgcggccgagagcatctcctctccttacaaaatctgcatggtttgtggcttcctatttccaaataaagaaagtctaattgagcaccgcaaggtgcacaccaaaaaaactgctttcggtaccagcagcgcgcagacagactctccacaaggaggaatgccgtcctcgagggaggacttcctgcagttgttcaacttgagaccaaaatctcaccctgaaacggggaagaagcctgtcagatgcatccctcagctcgatccgttcaccaccttccaggcttggcagctggctaccaaaggaaaagttgccatttgccaagaagtgaaggaatcggggcaagaagggagcaccgacaacgacgattcgagttccgagaaggagcttggagaaacaaataagggcagttgtgcaggcctctcgcaagagaaagagaagtgcaaacactcccacggcgaagcgccctccgtggacgcggatcccaagttacccagtagcaaggagaagcccactcactgctccgagtgcggcaaagctttcagaacctaccaccagctggtcttgcactccagggtccacaagaaggaccggagggccggcgcggagtcgcccaccatgtctgtggacgggaggcagccggggacgtgttctcctgacctcgccgcccctctggatgaaaatggagccgtggatcgaggggaaggtggttctgaagacggatctgaggatgggcttcccgaaggaatccatctggataaaaatgatgatggaggaaaaataaaacatcttacatcttcaagagagtgtagttattgtggaaagtttttccgttcaaattattacctcaatattcatctcagaacgcatacaggtgaaaaaccatacaaatgtgaattttgtgaatatgctgcagcccagaagacatctctgaggtatcacttggagagacatcacaaggaaaaacaaaccgatgttgctgctgaagtcaagaacgatggtaaaaatcaggacactgaagatgcactattaaccgctgacagtgcgcaaaccaaaaatttgaaaagattttttgatggtgccaaagatgttacaggcagtccacctgcaaagcagcttaaggagatgccttctgtttttcagaatgttctgggcagcgctgtcctctcaccagcacacaaagatactcaggatttccataaaaatgcagctgatgacagtgctgataaagtgaataaaaaccctacccctgcttacctggacctgttaaaaaagagatcagcagttgaaactcaggcaaataacctcatctgtagaaccaaggcggatgttactcctcctccggatggcagtaccacccataaccttgaagttagccccaaagagaagcaaacggagaccgcagctgactgcagatacaggccaagtgtggattgtcacgaaaaacctttaaatttatccgtgggggctcttcacaattgcccggcaatttctttgagtaaaagtttgattccaagtatcacctgtccattttgtaccttcaagacattttatccagaagttttaatgatgcaccagagactggagcataaatacaatcctgacgttcataaaaactgtcgaaacaagtccttgcttagaagtcgacgtaccggatgcccgccagcgttgctgggaaaagatgtgcctcccctctctagtttctgtaaacccaagcccaagtctgctttcccggcgcagtccaaatccctgccatctgcgaaggggaagcagagccctcctgggccaggcaaggcccctctgacttcagggatagactctagcactttagccccaagtaacctgaagtcccacagaccacagcagaatgtgggggtccaaggggccgccaccaggcaacagcaatctgagatgtttcctaaaaccagtgtttcccctgcaccggataagacaaaaagacccgagacaaaattgaaacctcttccagtagctccttctcagcccaccctcggcagcagtaacatcaatggttccatcgactaccccgccaagaacgacagcccgtgggcacctccgggaagagactatttctgtaatcggagtgccagcaatactgcagcagaatttggtgagccccttccaaaaagactgaagtccagcgtggttgcccttgacgttgaccagcccggggccaattacagaagaggctatgaccttcccaagtaccatatggtcagaggcatcacatcactgttaccgcaggactgtgtgtatccgtcgcaggcgctgcctcccaaaccaaggttcctgagctccagcgaggtcgattctccaaatgtgctgactgttcagaagccctatggtggctccgggccactttacacttgtgtgcctgctggtagtccagcatccagctcgacgttagaaggaaaaaggcctgtgtcatatcaacacttatctaacagcatggcacaaaagagaaactatgagaattttattgggaatgcacattatcgaccaaatgacaaaaaaacttgattcactaattagggggaaaaaaggtcttggtggatgtcagtgcttactccccatgaaattaaattttacttcatcctttgagaagcgaatggtgaaagctactgaaataagctgtgattgtactgtacataaaacatatgaggaatctgcaaggaacactacagttgtgtaaagttgttctgttaacttttgtaccaaatagcaatacaaactagttggaacagttggaacttacatacatggggactggaaatctctattttgtccctgaataatatttttcttagaattgaccaaataagaagtggaatttttgcatacttgagcgctgctgaaaagaaatcatttgggttgggtggggcgggatgggggaaaagtatataatgcttgcacctcaggtaaaaatctgtaaatatctaagttgtaaacctgcttgttcaaatactgtgtgtattccttttctctaatgcagctcatcacttggagcagtttctgctattgtgctctttcatttaaaatgtatgtttttttttttttaaactgtcaatgattttgtattatgttgaatccacccaaatctattgttgtcttaaaattgttaatggaagtattgaccctctatgatatgtgctgcagatatcgaggtcagccattcggaagctggcagcattttatcgcaactttgagcatctcagatggggaaggcaccttcttcctcgcctctccagattgtcctggaacctccaggatccttgactgagggcgttgggatggcttgtaggatttttaagagagtgtgtctacagacaagcattttctctgtagagcagccacacgttgtataaacataaactgtatgtgcagttatttaaatttgtttctgtcaaattaatcatttttgttggacgattcagtggcgggggggttttgcctaaattagtatataaaaacaaaaatgctaaattatatctgtgaattgcaggtattggggaacagttttaagggaaattttggggggaacattttaggtttgtatttggtagtcttaatgtatctggcatttgggtgaactgtggacatactagagttgattatagacacattgattttgaataaggaactgctggccgagcccgctgggagtctagaaagagaaaatctgtttctagacctcagttattttcccatttttggttgttttgaagcagtaacatttttctcagtgcacatgcaatttgggttttagagaagatggccaccagctggcttcctagatattttaaacttttgttctttaatatgctgtccatggctgagtttattagtacatgggcttagtgaccacaaaatattttattaagaaactgtttcaaaaataaatttgcactgttcatttttctggcctcgctgttctccatagagcaagggtaatcctagaaaaaattttttttttttaaattatgcaacgtaagatgtcctccttgatagaagtcttagctcctgtgttacaagggagaactcatttgagatcagtctgttggcattgcaatgaagtgctttgtatcaggaaagtgtacactattgaccttttttcctgttcacaagctgagccatatgtacataatctagattttgttttcatagttttgcacttttatagcctatttttgaagattaacacatttgcaagatgattgactcaatctttgcctaatccaatgagtgttacagagagcttgctgtgactagaaccataaatcttaaagggggtatgtgataatagagggctggaatttaaacctgtatttaaaaaaaagaatcaccaaatctatttgaaaacaagtcgatttgtattatgctggaattttttgggctttcagatttctctttttaaccacatttctgaatgtataaaaataccaattattttcctacagccctttgtacttcaaaatatgtttttgtgtccatcagtattaactattggtatactactggttttatatttttttttctttgagacaacagtacatataatagaggtacaattcgttggatttttgtttatgtatttatttcattccagtttgatttattttaattgttgatacttaagttgtcgaacagtagacattacttgttttatttatgatatatttcagcttaaagttatgttattatatgtggaagtgtaaatatagatttggtgttttgcaa

SEQ ID NO: 31 - DNA, 6989 bp, Homo sapiens
  source 1..6989: mol_type = genomic DNA
aagtttttgtgtgtgtgtccctgtgtgcgtgtgtcattttaaggtggctcgggagcggcccggaggagccggcggcggcccgaggagcgagggaccgaaccgggagccatgccgcgctgagggggggccgcacagccgccgccaccgccaccgccgccgggtggggtgggaggggcgggagacgccgccgccgccgcctccagggtgggcgcctttcgccgtggacgccgaccgtccgggacgagggtttcatcaccttaaatggttttgaaccaatgaaggtgtattcccttaaaaagacggacagcccatcgtgtgaactatagagtttgtggacagatttatattgggttcatagtggcgtcatgcacgcagactcctgcaagttcccctaagttcttagaggactgctttgccttttgatctgagagttgcaaagttccataaagaatggcccttgtggataagcacaaagtcaagagacagcgattggacagaatttgtgaaggtatccgcccccagatcatgaacggccccctgcacccccgccccctggtggcgctgctggacggccgcgactgcactgtggagatgcccatcctgaaggacctggccactgtggccttctgtgacgcgcagtcgacgcaggaaatccacgagaaggttctaaacgaagccgtgggcgccatgatgtaccacaccatcaccctcaccagggaggacctggagaagttcaaggccctgagagtgatcgtgcggataggcagtggctatgacaacgtggacatcaaggctgccggcgagctcggaattgccgtgtgcaacatcccgtctgcagccgtggaagagacagcggactctaccatctgccacatcctcaacctgtaccggaggaacacgtggctgtaccaggcactgcgggaaggcacgcgggttcagagcgtggagcagatccgcgaggtggcctcgggagcggcccgcatccgtggggagacgctgggcctcattggctttggtcgcacggggcaggcggttgcagttcgagccaaggcctttggattcagcgtcatattttatgacccctacttgcaggatgggatcgagcggtccctgggcgtgcagagggtctacaccctgcaggatttgctgtatcagagcgactgcgtctccttgcactgcaatctcaacgaacataaccaccacctcatcaatgactttaccataaagcagatgaggcagggagcattccttgtgaacgcagcccgtggcggcctggtggacgagaaagccttagcacaagccctcaaggagggcaggatacgaggggcagccctcgacgtgcatgagtcagagcccttcagctttgctcagggtccgttgaaagatgccccgaatctcatctgcactcctcacactgcctggtacagtgagcaggcgtcactggagatgagggaggcagctgccaccgagatccgccgagccatcacaggtcgcatcccagaaagcttaagaaattgtgtgaacaaggaattctttgtcacatcagcgccttggtcagtaatagaccagcaagcaattcatcctgagctcaatggtgccacatacagatatccgccaggcatcgtgggtgtggctccaggaggacttcctgcagccatggaagggatcatccctggaggcatcccagtgactcacaacctcccgacagtggcacatccttcccaagcgccctctcccaaccagcccacaaaacacggggacaatcgagagcaccccaacgagcaatagcagagaatgccagaaggtaatcactcagatacacttgggaccaagagacagtgaaaaatagatgaactaagagaaaaagaatcggatggtctttgtaactgattctggacatatgcatcattgatgttgcagtgttgaaactacaagagctagaaaactgaagatgtcgtctgcttacggaagcgctgaaagactaggatgtgatttattaacgaccaacttctgttattgtgtgttaagtttttcatctgtgcatcaaatcacaaaaagaataaatagagctttttcctttatcagtcccttgggcacagcaggtcctgaacaccctgctctacaatgttgcatcaagagttcaaacaacaaaataaaaaatattaagaggaaatccccatcctgtgacttgagtcccttaagtctacaggggctggtgacctctttttgctaataggaaaatcacattactacaaaatggggagaaaactgtttgcctgtggtagacacctgcacgcataggattgaagacagtacaggctgctgtacagagaagcgcctctcacatctgaactgcatactgagcgggcaagtcggttgtaagttcagtaaaaccctctgatgatgcaaaaaaaaaaaaaaaagtattaagtttcacaagctgtttgtactcaaatatattttctcagtttcagatcctctgctattttattgagtggaaagtcttgagctaaaagggttcaagaagaataatgttgcatttccttatgtctcaggaaacactttttatggtaacttgtcagattgtctatgaacaaacccacttttttagacattgataaagtcttcttttcttcacgtgatattttatacaagaacacttcagatgtattagatgtgactgattttaacaaatcctattagatttgtatcaactagttacatgttctattcatagtcttttgtgaatcattgcctttttgtttaaaaagatggcctattttgagcctttgtataggtacattcctgtttttgtgacaaaagaaaaactttaaaattgtcccaaacagaaaaataatggctatcagaagtatgttttgttttagtgtgagttaccgttactgtatttgtttattgtaaaggtggacatttagcgttcagtgcagttttcaataaaaagtaattaaaatttgttaagttctgaaattcaagtacatctcactaatgtaaatgttctctacttgagatgtttaaggcaattgcattgtcaattagccaatttccagctcttgttactacagggttccataaccagactcaagaccgctgacaattaattacctgtgataacaaaaagtttaattgaaaaatcaaaacctcacacaagtccatcattatcacgtcatgccatccttaagatgcaatggtgggttagtgctaaatcaattcaaaaaaaaacaaagttgctcaacttttagagttctgactttaatctaccccaaagcaaaatgacctggacctggttcaagggagggaagtgaaccttgaaactgttttgccaataacctaacaaacaaaatgatatttacaaagaagtgttgcaaatagtcccatgagttaagagcttgatttaatggatcttctttttaaatagaattaaacctttatactaaaagtatttgcaagtgtcaattaagtccaacaattccaggtatgaaactccctctgagctcttccttatacttcccttcccaattaaaacaaaacaagaaaatcatggtgtcttaaagcctttggcttgcctggccttgtctgctcactcattttaaggtggtggccccatcccaactctaccataaaagtgtctattaacacaagctcacatggagagagacggcgctcatagttactgacctattaccccagggaacaaaaaggtagtttaacgtcttcgtaaccactcatcaaagaggcaatgaaatatgcgtgaaaaggaggccaagcgcacacagaatatcttaccttcacgaatatgtgaagaagtctgggacacgatgaacctaagagtcaaaaacataaaaaggcaggtctgaatccatggtccacctgggttttaattattagatcttggtcgcccaaacaaaaatttatatatatatatataaatttgaccccaaggaagaacaagtcaaaacaaaatgccacgctctgtttgctctagaaaaagaaccatcaagtccacagagtgcaggtatgacttactctccagagtcagtcattaatgtctgtcacctagccacgtataatcatctttgaaaaaggactatattctatcttgggacaataattatggctgttttaatagttcaagagcataatatagcattgatttaccaaaatttttggcctttaacagccagtctatgaaaaccatttagagccccattcctgtctgtagcctaggaaatggaaccatctttaattctttcccacagggaagaatgctgcacctatcacactatgtggtcagggtcactgattgataataaataaataaataatagagcgctcaggggaaacaacactcaccagaccacaacacacagtagaagctaagtgctgagcgtgtactctggacaacgctggctgcaatgcagactgcaatggctacatgcttttccctctaacaattctctttggctttttcaattgggagggcagattttgcttaacaggcaactagaacaggatctctgcccactaaaaaattatccctatacatagagaaattactacagatgctaagtagtcagaatgctaacattttctacaaacttgattacttattggccaaaatgaaagcaaacagccaatttttaacaacacattcaccacttgaaacaaacagcagcatttacattatatcaaagattcatttctacagaatcgtactaaaaaatcatttttcctgcaagatttagcatgcagcaaaaaatgatcttttcatcctgggaacacctttttactgagttttcggggggaaagaactaaagacatgacagttttgtgggtatttttctatttttagtattctggcagcaaatttgaggttgtacacatttcattatcaactaaacaaaacaagaagcaagcaaatactttaaggttttcacagcctttttcatcacagggaaggaaaaaactgtgtgtgtgtgtgtgtgtgtgtgtgtgtgtgcgcgcgcgcgcgtgcgtgtgtcgtctttccaaattcctggccacatcaatagtctagaaagtgcttatcaggacaccaaatatttctgtgttcctgagataggtctgaaatttttacccagtattgaatgtatacctgaagtggtcaaataatacttcttataaaaaagtttttctacctaagattatagttttaactttaaattaaagaatggccacaattaaccaacatgcaaaaattctcagactaaacactgagaaattcttcatacaatgcatttgccaccttattgcatttttaaaatctttattctgtagtgaattggtattcccaatctgcctaagcaaaggcatgcccttctaacaagatttgcttagagcagaggtgatagaaggaagaatccgaagaccctctggcatggcaatctgggagcagcacattgttgatggagtccaagtgagcacatttcacacaattcatttagtgacaagtgggcttgctcccttttcatccaggaaaaaaactactcacagaccactgcccagaatctggaataagaaccctcattttaaggtattcttcccaacaaataaatatctaaatattgaaagggggcatatcagaaaacttaaagacacaataaccaaaaccaaaaccctcttcaaaacaagtaagcaatgtctgtatttagttcactctaaaacattcttagcttttcttgcagtttgttcctaaaagatttgattgggcacaagaggaacgaaattattaataaaataaaagcttatttttgttttgctgtggataatcggtacaaaacgtttccagatctgagacttagatggatcttttaaggtgaaagtagaatgccaggttctactgaaataatatctgaaaggaacaaagagccatttgccatgtgctttttctaagtgcccatgctggaaaaaattttctcaataatatgaagatgctggtacaacttttcctattaaacaatgtcttacacaaccacatgcgttacatttatagacagatttacaatttggaaggaaaaaaagttctgtcttaataaaattagttttttttacaaactgtagtccaactaaagttctctgtctcctcacgcagcagaggcattatctccataaggtgtgtgtggtccaggaaaacacttccccgagcagatcctgccagatttggttcattccatgatgtcgactctgcttggagcaccacactaactttagggtcattacagatctgatgccttggtcttctgctgtttgattttttttttcattcatcttcatcatcctcatcttcctctccatcagacagggatgtacacggccggatctgtcggtagcggtcaagccatttttctaccatctgagtgaccagggggtgatttcgccgccgagaactcttctgcatggcaaccagaactcccccctccgtcacacagcagtccagccactccctgagcagtttttcttgctgttcctcattacctagcttttggaggaatatgaagacaaagggaaaatcatacaacagatcagagacttggtaggaatgtgacatggaaacctacagctggtatgctaactcatgaaaaggccaaagcaatcaacttttcacatggttttgacctcttggaagttttgtgattaaagtccagtttgctctggttaaatttaaaaagacataaatgggaaatttctcagaaaaaaactaaagaggagcacactagagggtaatttgttagataaaataaaaaacaaatgtcctaataaactgatcctggcaagccacagcttcaaacacatgacaaggtaagtttacttagtcaattatgtacaagctgaaattatacaagttacggtctatgaactttttttcttcatgttgtgattataaacgttttataattactagtatattaaataaaagaccaatacttttataagaa

SEQ ID NO: 32 - DNA, 3733 bp, Homo sapiens
  source 1..3733: mol_type = genomic DNA
agtactttgtgagcatcgaggcaacccaacgtcactgtgctcagctgagttggcttgtatttcagagagagagagagagggagagagagtgagagagactgactcttacctcgaatccgggaactttaatcctgaaagctgcgctcagaaaggacttcgaccattcactgggcttccaactttccctccctgggggtgtaaaggaggagcggggcactgagattatatggttgccggtgctcttggaggctattttgtgttctttggcgcttgccaactgggaagtatttagggagagcaagcgcacagcagaggaggtgtgtgttggaggtgggcagtcgccgcggaggctccagcggtaggtgcgccctagtaggcagcagtagccgctattctgggtaagcagtaaaccccgcataaaccccggagccaccatgcctgctcccccgcctcaccgccggcttccctgctaggagcagcagaggatgtggtgaatgcaccggcttcaccgaacgagagcagaaccttgcgcgggcacagggccctgggcgcaccatggccgacgcagacgagggctttggcctggcgcacacgcctctggagcctgacgcaaaagacctgccctgcgattcgaaacccgagagcgcgctcggggcccccagcaagtccccgtcgtccccgcaggccgccttcacccagcagggcatggagggaatcaaagtgtttctccatgaaagagaactgtggctaaaattccacgaagtgggcacggaaatgatcataaccaaggctggaaggcggatgtttcccagttacaaagtgaaggtgacgggccttaatcccaaaacgaagtacattcttctcatggacattgtacctgccgacgatcacagatacaaattcgcagataataaatggtctgtgacgggcaaagctgagcccgccatgcctggccgcctgtacgtgcacccagactcccccgccaccggggcgcattggatgaggcagctcgtctccttccagaaactcaagctcaccaacaaccacctggacccatttgggcatattattctaaattccatgcacaaataccagcctagattacacatcgtgaaagcggatgaaaataatggatttggctcaaaaaatacagcgttctgcactcacgtctttcctgagactgcgtttatagcagtgacttcctaccagaaccacaagatcacgcaattaaagattgagaataatccctttgccaaaggatttcggggcagtgatgacatggagctgcacagaatgtcaagaatgcaaagtaaagaatatcccgtggtccccaggagcaccgtgaggcaaaaagtggcctccaaccacagtcctttcagcagcgagtctcgagctctctccacctcatccaatttggggtcccaataccagtgtgagaatggtgtttccggcccctcccaggacctcctgcctccacccaacccatacccactgccccaggagcatagccaaatttaccattgtaccaagaggaaagaggaagaatgttccaccacagaccatccctataagaagccctacatggagacatcacccagtgaagaagattccttctaccgctctagctatccacagcagcagggcctgggtgcctcctacaggacagagtcggcacagcggcaagcttgcatgtatgccagctctgcgccccccagcgagcctgtgcccagcctagaggacatcagctgcaacacgtggccaagcatgccttcctacagcagctgcaccgtcaccaccgtgcagcccatggacaggctaccctaccagcacttctccgctcacttcacctcggggcccctggtccctcggctggctggcatggccaaccatggctccccacagctgggagagggaatgttccagcaccagacctccgtggcccaccagcctgtggtcaggcagtgtgggcctcagactggcctgcagtcccctggcacccttcagccccctgagttcctctactctcatggcgtgccaaggactctatcccctcatcagtaccactctgtgcacggagttggcatggtgccagagtggagcgacaatagctaaagtgaggcctgcttcacaacagacatttcctagagaaagagagagagagaggagaaagagagagaaggagagagacagtagccaagagaaccccacggacaagatttttcatttcacccaatgttcacatctgcactcaaggtcgctggatgctgatctaatcagtagcttgaaaccacaattttaaaaatgtgactttcttgttttgtctcaaaacttaaaaaaacaaacacaaaaagatgagtcccaccccccactaccaccacacccatcaaccagccacattcacgctactccccagatctcttcccccattccttcttttgggctctagaaagtcttgcctcattgagtgtttttccctagtgcgtagttggagtctgtccctgtcttggtgttaatgttgacattgttatataataaatgataatatatttttttctttcaattttcttaatgggacccagtcccttatttggggggaggtctgaggcaagtatatttcaaaatatgtacttgcgggattcccttcaagtaaaccatccctgaaacctaaattcacgtttccccttgactaagaaaagcacctacctctgccatgtgatgtttctgaaaagcctctgtatgtccccatttgctttggttttgtcctgccttctccaatatcacgtgctcagttttgcctctacttacccatggagtcaggataacactgacgctccctggcatcctatcttattcagccctaccatcttgccagctctgtctttccagctgtctgtcgctaaaacgtggcctatagcttcccttccggaaagcttgctttgaaaaacttaaaaagcccccgtttacatgtaggcaggactgtgataacagtgcaagctctgtgttgacaagagttgtggacaaaaagccaaaataaatattcttcctgattaaaaaaattttttttgaaaaaaacaaggccagccccaaccttccaaacctccatcaccaacaacccaaactggatgtcaagcaaaatgcacaattcctacagaagaggcaagacacagtcaccaatgatatctcgccaaagaaaccacgcccacaccaatgccaacacaaaactgtgtttactgaaagccgaaaacagtattaaaaaaagtgtgtaagtaaagtgttatggtagggttcttcagatgtaatattttactggtactatttatttataaataggaattctaattaagtaataacatgaaatgaaacccagcataggagctggccaagagcttttaattttattgatactcaaaaccaagtttgtgtttttttgtttttttttgtttttttcctctttcgaatgtgctttgctttttttgattaaaaagaattttttttttccttttttataaacagaccctaataaagagaacagggtaagatgtgaggctgagtgtgtttaagtacgtgagagagtgtgagtgtgtttgtaagtgagtgtccctatgcgattatgtctctttacgttgctaaggggggagggtgaggattaagtactcgtgccttatatttgtgtgccaattaatgcctaataaataccatgtgcttaaacaagtacaaggaagttgaacaccca

SEQ ID NO: 33 - DNA, 2512 bp, Homo sapiens
  source 1..2512: mol_type = genomic DNA
aattggtaccaggagagcccagaagccacacacgcatctctgcctctttgctctggctctcccaccctcttcccgtctttctctgcttccttgtcactgttactccaggagacgttcacttttcaccaacctttctccaagcatctccaagccactgactatatatattttgctacaagagacctagaagaggaaaaaaggcacgagacttttcaacacatcgtttaaaaaaatcatcaacgaatttcttgcaatgtttgagaacaattaaaaggaaaaagcgttttttaaaaaatctatcaactaagtgaacaatcattcatatcgtctacatcctgagatattattgtccctcgctgcggacaccagcgagatgacagctgctttttcatttttgaatgtaataaatatttgctgcttttaatattttccggaatctcccctcgctattccttggaattttaactctaaagattggagaacagattatcaaaaaggcttttgcaagcctccaaaatgatgctgagtccggaccaagcagcagattcggaccatcccagctcggcgcactcggatccggagtccctgggcggcacggacaccaaggtgctcggcagcgtgtcggacctggagccggtggaggaggccgagggcgacggcaagggcggcagccgagccgcgctctacccgcacccgcagcagctgagccgcgaggagaagcgccgccgccggcgcgccacggccaagtaccgctcggcccacgccacccgcgagcgcatccgcgtggaagccttcaacttggccttcgccgagctccgcaaattgctgcccacgctgcccccggacaagaagctctccaagatcgagatcctgcgcctggccatctgctacatctcctatctcaaccacgtcctggacgtgtagggcggggtgcgccgcggggggccgcctgtcccggcgtccgcgaaacgctacggatccggacgggcgccgtcgctcgggtggcggaggctgcggtggcccagcgagacggccgcctcgagagacggccggggaagaagctcgctacctgttctctctggtcagcagggcgacgagggaaggtttccgacgcaggcaggtggggttgtctggcgagcccgggggctcgcaggggggattttccaggccaggagaacctcgtggagacacccgagatgagccacttctggggctccccaagggttgtggttttaacccactggagactttgagttctcctacattcatccgccacaaatcttaatgtgttgaaagaatgagggcagaagttcagagtatgcaggctcataactttaggctatctggaatttttctaatggacaaatcaccaaacactgggaaggaggagagaggagggatctctcccatagagatggaaggcacagaaaggtgcgactggaaactttcctttctgttgtgaaaataaagattatattagaatccaaataaaccctgaatatttagtaagtttaaaaagacaaattgagaacaagtagacacactgtgggaggatctgagcttggtgatattttatccccaagatatgacaccaaggtagacaggaaaatccatatttaaatggaaactttaatatctgattacttttgcaggcgaagtgcccctttatatatccaaaaacatctatttgtgaccttaaacatgtggacccataggtgcagttagaaaaagacaacctatttttatttatgttagaaggagtagagtatttttttcaagacatttatttttcagagtggtgatacttttactttggatactctgtgccaatttatttatagtcaagtgtttacactttttcctgtggaataattatgtctaactttttacgtgtttgttgagattatactgtggtctttctttctgctctaattatattgcacttgtataacaaatttcccacttctccctgtttctaaacatattttatatattaagatgtttgttcttgaaaggttcttttgttgtgagatcagcaacactagcacttcactattatagttttttaaaaagtgtattattcttatctgtagttatgtctgagaagtcctttacaagctgtttataaggagagtagcttctttgtgtgtgtgatgtttgtgcgtgtgatgaatttagcaaattaagttattttcctaaaaaaaaaagaaaaaaagaattcagaactactttcctctgtgacaccaaaaagaaaagtctataacctgaaaatgtttcatgttctctgctgtgaaactcttaaaacaaggagtgtattttagagacaagggaaaaaaaccacatctgctatccaaagattttagtctttttcagggtttttttgtgtctctgttgctttatataatgcaatattttgtagtgaaaatagatataatctggtttctatctgacgagtttttcatatctcatgaatggtgcgctgttttgcatataaataaaggtatcaaataaa

SEQ ID NO: 34 - DNA, 1368 bp, Homo sapiens
  source 1..1368: mol_type = genomic DNA
gcagctgcgagttcatggcgtcccggtgctccgagccctacccggaggtctccagaatccctaccgtcaggggatgcaacggctccctgtctggtgccctctcctgctgcgaggactcggcccggggctcgggcccgcccaaggcccctacggtggccgagggtcccagctcctgccttcggcggaacgtgatcagcgagagggagcgcaggaagcggatgtcgttgagctgtgagcgtctgcgggccctgctgccccagttcgatggccggcgggaggacatggcctcggtcctggagatgtctgtgcagttcctgcggcttgccagcgccctggggcccagtcaggagcagcacgctattcttgcttcctccaaggaaatgtggcactcgttgcaggaggatgttttacagttgacgttgtcgagtcagattcaagcaggtgtgccagaccctgggacgggagcgtccagcgggactcgaaccccagatgtgaaggcgtttctggaaagtccttggtccctggatccagcgtcggccagcccagagcccgtgccgcacatccttgcgtcctccaggcagtgggaccccgcgagctgcacgtccctgggcacggacaagtgtgaggcactgttggggctgtgccaggtgcggggtgggctgccccctttctcagaaccttccagcctggtgccgtggcccccaggccggagtcttcctaaggctgtgaggccacccctgtcctggcctccgttctcgcagcagcagaccttgcccgtgatgagcggggaggcccttggctggctgggccaggctgggcccctggccatgggggctgcacctctgggggagccagccaaggaggaccccatgctggcgcaggaggccgggtctgcgttggggtctgatgtggacgatgggacgtccttcctgctgactgctggtcccagctcgtggccggggtctctggagggtcgaggaggcagtggccctgcatgggccccagctgagagcagtccactggatgttggagagccaggcttcctaggggaccctgagcttggctcccaggagctccaggacagccctctggagccgtggggcctggatgtggactgtgcaggcctggccctgaaggacgaggtggagagcatcttccctgacttctttgcctgctagcagccaggctgtggagaggaggcgggcggggccggtggatctggagccgtgtgtgtccctgtcgcgtgcttgggcattttattttgggtttgggttgcagctgcaccctaatttgagcgaggcttacaggctgtgagaagaggatagtgaatattaaagagggaaagcagttttcctggaggaagcaaaa

SEQ ID NO: 35 - DNA, 10895 bp, Homo sapiens
  source 1..10895: mol_type = genomic DNA
agaagtgaagatggcggcggcagcttctcgctagtgacgcgcggtgcgggctcatctccttcccctccttcctcacctcccctagtccggaccccgtccgccgccgggaccagaccgtccccgtagcccgcagccagcagccgcccctctcctcccccggccgcccctctcccaacgcccgtctgccgctggccggcggcgcgcccctggtcccggccgcctgcctcgcactccgcgctgtcgccgggggagcgggtggcgtgtggattcgccctcctgcccgcccctccgggccgcggacacacgcccggcagggaggcggcggcggcccctgcgtatttgcttccctccatttggggactgggaggcggcggcggctgtgggttttggggaggggtagagggctggggggcgggcgagagcgggagggggccgccctcgagtggaggacgagaaggaaagcaccatgacgtccatccatttcgtggttcacccgctgccgggcaccgaggaccagctcaatgacaggttacgagaagtttctgagaagctgaacaaatataatttaaacagccacccccctttgaatgtattggaacaggctactattaaacagtgtgtggtgggaccaaatcatgctgcctttcttcttgaggatggtagagtttgcaggattggtttttcagtacagccagacagattggaattgggtaaacctgataataatgatgggtcaaagttgaacagcaactcgggggcagggaggacgtcaaggcctggtaggacaagcgactctccatggtttctctcaggttctgagactctaggcaggctggcaggcaacaccttaggaagccgctggagttctggagtgggtggaagtggtggaggatcctctggtaggtcatcagctggagctcgagattcccgccggcagactcgagttattcggacaggacgggatcgagggtctgggcttttgggcagtcagccccagccagttattccagcatctgtcattccagaggagctgatttcacaggcccaagttgttttacaaggcaaatccagaagtgtcattattcgagaacttcagagaacaaatcttgatgtgaaccttgctgtaaataatttacttagccgggatgatgaagatggagatgatggggatgatacagccagcgaatcttatttgcctggagaggatcttatgtctctccttgatgccgacattcattctgcccacccaagtgtcattattgatgcagatgccatgttttctgaagacattagctattttggttacccttcttttcgtcgttcatcactttccaggctaggctcatctcgagttctccttcttcccttagagagagactctgagctgttgcgtgaacgtgaatccgttttacgtttacgtgaacgaaggtggcttgatggagcctcatttgataatgaaaggggttctaccagcaaggaaggagagccaaacttggataagaagaatacacctgttcaaagtccagtatctctaggagaagatttgcagtggtggcctgataaggatggaacaaaattcatctgtattggggctctgtattctgaacttctggctgtcagcagtaaaggagaactttatcagtggaaatggagtgaatctgagccttacagaaatgcccagaatccttcattacatcatccacgagcaacatttttggggttaaccaatgaaaagatagtcctcctgtctgcaaatagcataagagcaactgtagctacagaaaataacaaggttgctacatgggtggatgaaactttaagttctgtggcttctaaattagagcacactgctcagacttactctgaacttcaaggagagcggatagtttctttacattgctgtgccctttacacctgcgctcagctggaaaacagtttatattggtggggtgtagttccttttagtcaaaggaagaaaatgttagagaaagctagagcaaaaaataaaaagcctaaatccagtgctggtatttcttcaatgccgaacatcactgttggtacccaggtatgcttgagaaataatcctctttatcatgctggagcagttgcattttcaattagtgctgggattcctaaagttggtgtcttaatggagtcagtttggaatatgaatgacagctgtagatttcaacttagatctcctgaaagcttgaaaaacatggaaaaagctagcaaaactactgaagctaagcctgaaagtaagcaggagccagtgaaaacagaaatgggtcctccaccatctccagcatccacgtgtagtgatgcatcctcaattgccagcagtgcatcaatgccatacaaacgacgacggtcaacccctgcaccaaaagaagaggaaaaggtgaatgaagagcagtggtctcttcgggaagtggtttttgtggaagatgtcaagaatgttcctgttggcaaggtgctaaaagtagatggtgcctatgttgctgtaaaatttccaggaacctccagtaatactaactgtcagaacagctctggtccagatgctgacccttcttctctcctgcaggattgtaggttacttagaattgatgaattgcaggttgtcaaaactggtggaacaccgaaggttcccgactgtttccaaaggactcctaaaaagctttgtatacctgaaaaaacagaaatattagcagtgaatgtagattccaaaggtgttcatgctgttctgaagactggaaattgggtacgatactgtatctttgatcttgctacaggaaaagcagaacaggaaaataattttcctacaagcagcattgctttccttggtcagaatgagaggaatgtagccattttcactgctggacaggaatctcccattattcttcgagatggaaatggtaccatctacccaatggccaaagattgcatgggaggaataagggatcccgattggctggatcttccacctattagtagtcttggaatgggtgtgcattctttaataaatcttcctgccaattcaacaatcaaaaagaaagctgctgttatcatcatggctgtagagaaacaaaccttaatgcaacacattctgcgctgtgactatgaggcctgtcgacaatatctaatgaatcttgagcaagcggttgttttagagcagaatctacagatgctgcagacattcatcagccacagatgtgatggaaatcgaaatattttgcatgcttgtgtatcagtttgctttccaaccagcaataaagaaactaaagaagaagaggaagcggagcgttctgaaagaaatacatttgcagaaaggctttctgctgttgaggccattgcaaatgcaatatcagttgtttcaagtaatggcccaggtaatcgggctggatcatcaagtagccgaagtttgagattacgggaaatgatgagacgttcgttgagagcagctggtttgggtagacatgaagctggagcttcatccagtgaccaccaggatccagtttcaccccccatagctccccctagttgggttcctgaccctcctgcgatggatcctgatggtgacattgattttatcctggcccccgctgtgggatctcttaccacagcagcaaccggtactggtcaaggaccaagcacctccactattccaggtccttccacagagccatctgtagtagaatccaaggatcgaaaggcgaatgctcattttatattgaaattgttatgtgacagtgtggttctccagccctatctacgagaacttctttctgccaaggatgcaagagggatgaccccatttatgtcagctgtaagtggccgagcttatcctgctgcaattaccatcttagaaactgctcagaaaattgcaaaagctgaaatatcctcaagtgaaaaagaggaagatgtattcatgggaatggtttgcccatcaggtaccaaccctgatgactctcctttatatgttttatgttgtaatgacacttgcagttttacatggactggagcagagcacattaaccaggatatttttgagtgtcgaacttgtggcttgctggagtcactgtgttgttgtacggaatgtgcaagggtttgtcataaaggtcatgattgcaaactcaaacggacatcaccaacagcctactgtgattgttgggagaaatgtaaatgtaaaactcttattgctggacagaaatctgctcgtcttgatctactttatcgcctgctcactgctactaatctggttactctgccaaacagcaggggagagcacctcttactattcttagtacagacagtcgcaaggcagacggtggagcattgtcaatacaggccacctcgaatcagggaagatcgtaaccgaaaaacagccagtcctgaagattcagatatgccagatcatgatttagagcctccaagatttgcccagcttgcattggagcgtgttctacaggactggaatgccttgaaatctatgattatgtttgggtcgcaggagaataaagaccctcttagtgccagcagtagaataggccatcttttgccagaagagcaagtatacctcaatcagcaaagtggcacaattcggctggactgtttcactcattgccttatagttaagtgtacagcagatattttgcttttagatactctactaggtacactagtgaaagaactccaaaacaaatatacacctggacgtagagaagaagctattgctgtgacaatgaggtttctacgttcagtggcaagagtttttgttattctgagtgtggaaatggcttcatccaaaaagaaaaacaactttattccacagccaattggaaaatgcaagcgtgtattccaagcattgctaccttacgctgtggaagaattgtgcaacgtagcagagtcactgattgttcctgtcagaatggggattgctcgtccaactgcaccatttaccctggctagtactagcatagatgccatgcagggcagtgaagaattattttcagtggaaccactaccaccacgaccatcatctgatcagtctagcagctccagtcagtctcagtcatcctacatcatcaggaatccacagcagaggcgcatcagccagtcacagcccgttcggggcagagatgaagaacaggatgatattgtttcagcagatgtggaagaggttgaggtggtggagggtgtggctggagaagaggatcatcatgatgaacaggaagaacacggggaagaaaatgctgaggcagagggacaacatgatgagcatgatgaagacgggagtgatatggagctggacttgttagcagcagctgaaacagaaagtgatagtgaaagtaaccacagcaaccaagataatgctagtgggcgcagaagcgttgtcactgcagcaactgctggttcagaagcaggagcaagcagtgttcctgccttcttttctgaagatgattctcaatcgaatgactcaagtgattctgatagcagtagtagtcagagtgacgacatagaacaggagacctttatgcttgatgagccattagaaagaaccacaaatagctcccatgccaatggtgctgcccaagctccccgttcaatgcagtgggctgtccgcaacacccagcatcagcgagcagccagtacagccccttccagtacatctacaccagcagcaagttcagcgggtttgatttatattgatccttcaaacttacgccggagtggtaccatcagtacaagtgctgcagctgcagcagctgctttggaagctagcaacgccagcagttacctaacatctgcaagcagtttagccagggcttacagcattgtcattagacaaatctcggacttgatgggccttattcctaagtataatcacctagtatactctcagattccagcagctgtgaaattgacttaccaagatgcagtaaacttacagaactatgtagaagaaaagcttattcccacttggaactggatggtcagtattatggattctactgaagctcaattacgttatggttctgcattagcatctgctggtgatcctggacatccaaatcatcctcttcacgcttctcagaattcagcgagaagagagaggatgactgcgcgagaagaagctagcttacgaacacttgaaggcagacgacgtgccaccttgcttagcgcccgtcaaggaatgatgtctgcacgaggagacttcctaaattatgctctgtctctaatgcggtctcataatgatgagcattctgatgttcttccagttttggatgtttgctcattgaagcatgtggcatatgtttttcaagcacttatatactggattaaggcaatgaatcagcagacaacattggatacacctcaactagaacgcaaaaggacgcgagaactcttggaactgggtattgataatgaagattcagaacatgaaaatgatgatgacaccaatcaaagtgctactttgaatgataaggatgatgactctcttcctgcagaaactggccaaaaccatccatttttccgacgttcagactccatgacattccttgggtgtatacccccaaatccatttgaagtgcctctggctgaagccatccccttggctgatcagccacatctgttgcagccaaatgctagaaaggaggatctttttggccgtccaagtcagggtctttattcttcatctgccagtagtgggaaatgtttaatggaggttacagtggatagaaactgcctagaggttcttccaacaaaaatgtcttatgctgccaatctgaaaaatgtaatgaacatgcaaaaccggcaaaaaaaagaaggggaagaacagcccgtgctgccagaagaaactgagagttcaaaaccagggccatctgctcatgatcttgctgcacaattaaaaagtagcttactagcagaaataggacttactgaaagtgaagggccacctctcacatctttcaggccacagtgtagctttatgggaatggttatttcccatgatatgctgctaggacgttggcgcctttctttagaactgttcggcagggtattcatggaagatgttggagcagaacctggatcaatcctaactgaattgggtggttttgaggtaaaagaatcaaaattccgcagagaaatggaaaaactgagaaaccagcagtcaagagatttgtcactagaggttgatcgggatcgagatcttctcattcagcagactatgaggcagcttaacaatcactttggtcgaagatgtgctactacaccaatggctgtacacagagtaaaagtcacatttaaggatgagccaggagagggcagtggtgtagcacgaagtttttatacagccattgcacaagcatttttatcaaatgaaaaattgccaaatctagagtgtatccaaaatgccaacaaaggcacccacacaagtttaatgcagagattaaggaaccgaggagagagagaccgggaaagggagagagaaagggaaatgaggaggagtagtggtttgcgagcaggttctcggagggaccgggatagagactttagaagacagctttccatcgacactaggccctttagaccagcctctgaagggaatcctagcgatgatcctgagcctttgccagcacatcggcaggcacttggagagaggctttatcctcgtgtacaagcaatgcaaccagcatttgcaagtaaaatcactggcatgttgttggaattatccccagctcagctgcttctccttctagcaagtgaggattctctgagagcaagagtggatgaggccatggaactcattattgcacatggacgggaaaatggagctgatagtatcctggatcttggattagtagactcctcagaaaaggtacagcaggaaaaccgaaagcgccatggctctagtcgaagtgtagtagatatggatttagatgatacagatgatggtgatgacaatgcccctttgttttaccaacctgggaaaagaggattttatactccaaggcctggcaagaacacagaagcaaggttgaattgtttcagaaacattggcaggattcttggactatgtctgttacagaatgaactatgtcctatcacattgaatagacatgtaattaaagtattgcttggtagaaaagtcaattggcatgattttgctttttttgatcctgtaatgtatgagagtttgcggcaactaatcctcgcgtctcagagttcagatgctgatgctgttttctcagcaatggatttggcatttgcaattgacctgtgtaaagaagaaggtggaggacaggttgaactcattcctaatggtgtaaatataccagtcactccacagaatgtatatgagtatgtgcggaaatacgcagaacacagaatgttggtagttgcagaacagcccttacatgcaatgaggaaaggtctactagatgtgcttccaaaaaattcattagaagatttaacggcagaagattttaggcttttggtaaatggctgcggtgaagtcaatgtgcaaatgctgatcagttttacctctttcaatgatgaatcaggagaaaatgctgagaagcttctgcagttcaagcgttggttctggtcaatagtagagaagatgagcatgacagaacgacaagatcttgtttacttttggacatcaagcccatcactgccagccagtgaagaaggattccagcctatgccctcaatcacaataagaccaccagatgaccaacatcttcctactgcaaatacttgcatttctcgactttacgtcccactctattcctctaaacagattctcaaacagaaattgttactcgccattaagaccaagaattttggttttgtgtagagtataaaaagtgtgtattgctgtgtaatattactagcaaattttgtagatttttttccatttgtctataaaagtttatggaagttaatgctgtcatacccccctggtggtaccttaaagagataaaatgcagacattccttgctgagtttatagcttaaaggcctaaggagcactagcaacatttggctatattggtttgctagtcaccaacttctgggtctaaccccagccaaagatgacagcagaacaacataatttacactgtgatttatctttttgctgagggggaaaaaatgtaaatgttctgaaaattcactgctgcctttgtggaaactgtttcagcaaaggttcttgtatagagggaatagggaatttcaaaataaaaaattaagtatgttctgtgttttcattttaactttttttatggtgtttaatttgtggttggctgcaactgtgtatcatgtatatggaacttgtaaaaaagttctcgacattcagatcttaagagatgaaatcacttttacctataaaaaccacttttattgcggtttgactgcattgagctctaggatattaaatgatatcactaatattttgcatgtaatttgctcatttgagtgagggcactttttttgtacatatgatggggccaatgcacaatacttttatcacaatcaactttttctttgtatccctatttcaatgagcagtcagtctcaagaggttactgcacttcagttctaactagacatttgtactaaggtatttcagttatgtaaactcagcctgggcactttctgataactgtaaaatgttttataagatcatgattattgaagatacattttggaaaattttaaatgttcgtgagcagcttaactacttttgtatctagccttttttaagtatcttgttacatttacttttttaaataaagaaattacagaagaaatgtcaagtaatattgaagaaacaatagtttttatttatgtagttgtacatttttaaactaagggcaatacactgacatggttatgtgcataaaaattttgacttaaagaactggaagtttatatacacctggactataagaaacagaagaaaatcagtccacattttacagttagcagagaatcctaaatggcactggcctggccaccttttcattttacaaatgggggaagtgaagtgtgaccccttacttggcataggaagttaacttacacctaataactgacaggtttttgttttgatgacctattaattatgtagcctaggattaatatcccaaaattactctggtttaagtagctttattcagtggcataataacactgttttcttccttaagtcttcaatgaagtgacttaaaacagtcactttacatattaaaaatgaggagagcaattctctggaatctctcctttcagttcctttgtaggatttctggccttgaggatagtcttcatgttcaaaggcactatgcttttattatataacttccttcagaagactgaaccacatgatattctcagccctgttaacactaaaaatatttaaaactgaatgatagtagtgactcattgtattacttaaaacttatataacacgctgtattagatgtgtgtaaattagccaaaggttattttacaaagtgagacattggtttttatgtctaaatgctatttctgaataaatgaaatagtaattagatcaagagctgattagcatcaatgtgtttgaaagatataaaatttatacatcaccttaacctctgtatgcacatgatgggattgataaaatattaaatgagaacaaactagatatgattaggacatttgaaaccctaattgtgaatttatttttaatagttactgaaatgaaaatatttaaaataatgcacaatgtcttaagtcttcctaaatcaagattttggttaaaaaatacttctaataatagtaaaagattttttttttaagtaaatcataaaacggttctaaatgtaaaataaagacatgtaaaataaa

SEQ ID NO: 36 - DNA, 10520 bp, Homo sapiens
  source 1..10520: mol_type = genomic DNA
ggcaattccgcagcctcgcccgaactgaggggagccatcttgtccctctctgcctccgagtttgtatcgttgcctccatctgtgtctgtgtgtctgtattatcgttgtttggcgtgtttttttttttttttgttttttgtcactgcctgcctgggtcctgcccgaggtctccatcctcggtttccctgtccttgccccgggccctgggagtgctctggaaggctgcgcagtattggaggggacagaatgaccttccggccttgagtccctggggagcagatggaccctactggaagtcagttggattcagatttctctcagcaagatactccttgcctgataattgaagattctcagcctgaaagccaggttctagaggatgattctggttctcacttcagtatgctatctcgacaccttcctaatctccagacgcacaaagaaaatcctgtgttggatgttgtgtccaatcctgaacaaacagctggagaagaacgaggagacggtaatagtgggttcaatgaacatttgaaagaaaacaaggttgcagaccctgtggattcttctaacttggacacatgtggttccatcagtcaggtcattgagcagttacctcagccaaacaggacaagcagtgttctgggaatgtcagtggaatctgctcctgctgtggaggaagagaagggagaagagttggaacagaaggagaaagagaaggaagaagatacttcaggcaatactacacattcccttggtgctgaagatactgcctcatcacagttgggttttggggttctggaactctcccagagccaggatgttgaggaaaatactgtgccatatgaagtggacaaagagcagctacaatcagtaaccaccaactctggttataccaggctgtctgatgtggatgctaatactgcaattaagcatgaagaacagtccaacgaagatatccccatagcagaacagtccagcaaggacatccctgtgacagcacagcccagtaaggatgtacatgttgtaaaagagcaaaatccaccacctgcaaggtcagaggacatgccttttagccccaaagcatctgttgctgctatggaagcaaaagaacagttgtctgcacaagaacttatggaaagtggactgcagattcagaagtcaccagagcctgaggttttgtcaactcaggaagacttgtttgaccagagcaataaaacagtatcttctgatggttgctctactccttcaagggaggaaggtgggtgttctttggcttccactcctgccaccactctgcatctcctgcagctctctggtcagaggtcccttgttcaggacagtctttccacgaattcttcagatcttgttgctccttctcctgatgctttccgatctactccttttatcgttcctagcagtcccacagagcaagaagggagacaagataagccaatggacacgtcagtgttatctgaagaaggaggagagccttttcagaagaaacttcaaagtggtgaaccagtggagttagaaaacccccctctcctgcctgagtccactgtatcaccacaagcctcaacaccaatatctcagagcacaccagtcttccctcctgggtcacttcctatcccatcccagcctcagttttctcatgacatttttattccttccccaagtctggaagaacaatcaaatgatgggaagaaagatggagatatgcatagttcatctttgacagttgagtgttctaaaacttcagagattgaaccaaagaattcccctgaggatcttgggctatctttgacaggggattcttgcaagttgatgctttctacaagtgaatatagtcagtccccaaagatggagagcttgagttctcacagaattgatgaagatggagaaaacacacagattgaggatacggaacccatgtctccagttctcaattctaaatttgttcctgctgaaaatgatagtatcctgatgaatccagcacaggatggtgaagtacaactgagtcagaatgatgacaaaacaaagggagatgatacagacaccagggatgacattagtattttagccactggttgcaagggcagagaagaaacggtagcagaagatgtttgtattgatctcacttgtgattcggggagtcaggcagttccgtcaccagctactcgatctgaggcactttctagtgtgttagatcaggaggaagctatggaaattaaagaacaccatccagaggaggggtcttcagggtctgaggtggaagaaatccctgagacaccttgtgaaagtcaaggagaggaactcaaagaagaaaatatggagagtgttccgttgcacctttctctgactgaaactcagtcccaagggttgtgtcttcaaaaggaaatgccaaaaaaagaatgctcagaagctatggaagttgaaaccagtgtgattagtattgattcccctcaaaagttggcaatacttgaccaagaattggaacataaggaacaggaagcttgggaagaagctacttcagaggactccagtgttgtcattgtagatgtgaaagagccatctcccagagttgatgtttcttgtgaacctttggagggagtggagaagtgctcagattcccagtcatgggaggatattgctccagaaatagaaccatgtgctgagaatagattagacaccaaggaagaaaagagtgtagaatatgaaggagatctgaaatcagggactgcagaaacagaacctgtagagcaagattcttcacagccttccttacctttagtgagagcagatgatcctttaagacttgaccaggagttgcagcagccccaaactcaggagaaaacaagtaattcattaacagaagactcaaaaatggctaatgcaaagcagctaagctcagatgcagaggcccagaagctggggaagccctctgcccatgcctcacaaagcttctgtgaaagttctagtgaaaccccatttcatttcactttgcctaaagaaggtgatatcatcccaccattgactggtgcaaccccacctcttattgggcacctaaaattggagcccaagagacacagtactcctattggtattagcaactatccagaaagcaccatagcaaccagtgatgtcatgtctgaaagcatggtggagacccatgatcccatacttgggagtggaaaaggggattctggggctgccccagacgtggatgataaattatgtctaagaatgaaactggttagtcctgagactgaggcgagtgaagagtctttgcagttcaacctggaaaagcctgcaactggtgaaagaaaaaatggatctactgctgttgctgagtctgttgccagtccccagaagaccatgtctgtgttgagctgtatctgtgaagccaggcaagagaatgaggctcgaagtgaggatccccccaccacacccatcagggggaacttgctccactttccaagttctcaaggagaagaggagaaagaaaaattggagggtgaccatacaatcaggcagagtcaacagcctatgaagcccattagtcctgtcaaggaccctgtttctcctgcttcccagaagatggtcatacaagggccatccagtcctcaaggagaggcaatggtgacagatgtgctagaagaccagaaagaaggacggagtactaataaggaaaatcctagtaaggccttgattgaaaggcccagccaaaataacataggaatccaaaccatggagtgttccttgagggtcccagaaactgtttcagcagcaacccagactataaagaatgtgtgtgagcaggggaccagtacagtggaccagaactttggaaagcaagatgccacagttcagactgagagggggagtggtgagaaaccagtcagtgctcctggggatgatacagagtcgctccatagccagggagaagaagagtttgatatgcctcagcctccacatggccatgtcttacatcgtcacatgagaacaatccgggaagtacgcacacttgtcactcgtgtcattacagatgtgtattatgtggatggaacagaagtagaaagaaaagtaactgaggagactgaagagccaattgtagagtgtcaggagtgtgaaactgaagtttccccttcacagactgggggctcctcaggtgacctgggggatatcagctccttctcctccaaggcatccagcttacaccgcacatcaagtgggacaagtctctcagctatgcacagcagtggaagctcagggaaaggagccggaccactcagagggaaaaccagcgggacagaacccgcagattttgccttacccagctcccgaggaggcccaggaaaactgagtcctagaaaaggggtcagtcagacagggacgccagtgtgtgaggaggatggtgatgcaggccttggcatcagacagggagggaaggctccagtcacgcctcgtgggcgtgggcgaaggggccgcccaccttctcggaccactggaaccagagaaacagctgtgcctggccccttgggcatagaggacatttcacctaacttgtcaccagatgataaatccttcagccgtgtcgtgccccgagtgccagactccaccagacgaacagatgtgggtgctggtgctttgcgtcgtagtgactctccagaaattcctttccaggctgctgctggcccttctgatggcttagatgcctcctctccaggaaatagctttgtagggctccgtgttgtagccaagtggtcatccaatggctacttttactctgggaaaatcacacgagatgtcggagctgggaagtataaattgctctttgatgatgggtacgaatgtgatgtgttgggcaaagacattctgttatgtgaccccatcccgctggacactgaagtgacggccctctcggaggatgagtatttcagtgcaggagtggtgaaaggacataggaaggagtctggggaactgtactacagcattgaaaaagaaggccaaagaaagtggtataagcgaatggctgtcatcctgtccttggagcaaggaaacagactgagagagcagtatgggcttggcccctatgaagcagtaacacctcttacaaaggcagcagatatcagcttagacaatttggtggaagggaagcggaaacggcgcagtaacgtcagctccccagccacccctactgcctccagtagcagcagcacaacccctacccgaaagatcacagaaagtcctcgtgcctccatgggagttctctcaggcaaaagaaaacttatcacttctgaagaggaacggtcccctgccaagcgaggtcgcaagtctgccacagtaaaacctggtgcagtaggggcaggagagtttgtgagcccctgtgagagtggagacaacaccggtgaaccctctgccctggaagagcagagagggcctttgcctctcaacaagaccttgtttctgggctacgcatttctccttaccatggccacaaccagtgacaagttggccagccgctccaaactgccagatggtcctacaggaagcagtgaagaagaggaggaatttttggaaattcctcctttcaacaagcagtatacagaatcccagcttcgagcaggagctggctatatccttgaagatttcaatgaagcccagtgtaacacagcttaccagtgtcttctaattgcggatcagcattgtcgaacccggaagtacttcctgtgccttgccagtgggattccttgtgtgtctcatgtctgggtccatgatagttgccatgccaaccagctccagaactaccgtaattatctgttgccagctgggtacagccttgaggagcaaagaattctggactggcaaccccgtgaaaatcctttccagaatctgaaggtactcttggtatcagaccaacagcagaacttcctggagctctggtctgagatcctcatgactggtggtgcagcctctgtgaagcagcaccattcaagtgcccataacaaagatattgctttaggggtatttgatgtggtggtgacggacccctcatgcccagcctcggtgctgaagtgtgctgaagcattgcagctgcctgtggtgtcacaagagtgggtgatccagtgcctcattgttggggagagaattggattcaagcagcatccaaaatataaacacgattatgtttctcactaaagatacttggtcttactggttttattccctgctatcgtggagattgtgttttaaccaggttttaaatgtgtcttgtgtgtaactggattccttgcatggatcttgtatatagttttatttgctgaacttttatgataaaataaatgttgaatctctttggttgtagtaactgggatttcttcatctgtttttttgagcttaatctcagaacaaatgacaagacatagtactttctctgagtctttcaacaggcttattcacttacggaggacagctggccaaggaaattgaaaagttaagagtgaactttattctgtggcatcattcccaaaaggttattccagggtgtctaaaatgctatgcttgcagaaactcagtttaaggtaggtgaaggcccagattaacagttgtgccaaaagttgagtggaattgggcacagctctgtttcctgacagttaaaaaagacctcatgctctctctctgagctgagatcacagctcacctgtgggtactccccaactcttagagctaaagggagaacgaaaggaccaactgccatgaagggacagtgaccataagcttgatggaatgaccttccgtaagataaacatgggaagcacaagtgagaacacctggaaatgttacacgttctagtcaaagacccaatattattattattattattgtcacaatagctggaagcagttccttcccttcctctggcatcactgatccctgcatggcttctcattctctaaagcaggggtcaacaaggtttttttctgtaaagggtcaaagagtaaatatttcaggctttgtgggccatttgatccatcacaactactcgcctttgctgtgagggcatgaaagcaaccatagacaatgagtaaacaaatgggcatggctgtgtttcagtaaaactgtacaaaaacagacagcaggccatagtttgccagctcctgctccagagacagcagtagaaagggtgatctttagttgataatagcagggaataagttgtcagagcttcccagtgtgtgtagaatatgtagtgatgaaaaccagatgcagtgactataacctgatgccagaacactgcattctttttcagtttggagggcgttgttcagtgaatatttctttttacttacactgatatgaatattgattaccagtgatggctgggccatattaagataacttcaacccctatggtttgtgtaagatgggtaattgggcctgcaatcttcagtatttaaaaatctaacaacttgatctcaattttttcttaaggacctttttcttggagaataatactttttttttttttttttttttttttgagacggaatttcgctcttgttgcccgggctggaatgcaatggcacaatctcagctcactgcagcgtctgcttcccaggttcaagcaattctcctgtctcagcctcctgagtagctgggattacaggcacatgccaccacacctggctaatttttgtatttttagtagaatcgaggtttcatcatgttggtcaggctggtctcaaactcctgacttcaggtgatccgcccgcctcggcctcccaaagtgctgggattacaggtgtgagccaccatgcccggcctaagaaatacttttaagtatattttcattagctagaattgcccaatctgtgtaggtataaattacttggtatagggagagagaaagcctatcttacctgttgctttcttacttggtggtaacatccagcagttagtctatttataaacataattactttttcacatatgaaccataaaatatttaactttctgctctatattgtttgtttaccgctgtatctcccacagcttgaacagtaccaaggtacgtagtaggtgctcaataaatgactattgaataaatgaacatatccaacaaatgttctcaatgtaaaggatcagagatgccacatgttctccttgatgggagagacccttccacatgggaatgatgggaaggagttgtactcctggatgttcagtaactgcttctaggagaaaaggtagagtcctatcactaagccgcagatatttatttgtgtgtggctagaatgggatgttttgaatcttctgttacaaccttgggaacgtggctgttatttcaatttatgagccagaaattttcacatcccgaaactacaaaagagaaaaagagccttattaagtgtcatgctttcccaagactaccttcaaagaaatatgaatcaggataacctgtgatctaaataatgtcatcttaaaactgaagagtttcttttgactcttctgctacaatagcttagaaaaaaatctgcttgcagacattttagagagaaaggacaatgaagtgattttctgaatgggaatgacagacctctgggaagccagctaccactgaatctcggtatcagttttttttaaagtttagagttagaaggggtggtcgcctcctttcacagatgcggaagctagggaccagcaaggcggggtgcccacggctgcacagctagttcatatcagaattgggagtggaaggcccactgcctcccagcatagcaatacataacctagcaaaggacttaacacctatctcactgtcaggttttttagtattttatgatgatgatgacttctactagaaaataacctccattaaaattattaaagatggtcacacctctatctctaagccttacttataaaatgagggtatttggactaaagtcttcttccagttctagaattctacaactcattaaaaagccaccttaaaaagtctactgagttacccaagggttgctcctacctgcccagagttccaccagcctgggtatagtatttgttataatctagtcgtaacagtagttgagccaaatctgagttgatctgatgattccgaacactggagagaatcttgaacaggagtgaagactggcggctaaagccctgcagagagaaggactcagctgtcattccacttcagctcaccaactctccatatggaggatgggggcggagggaggagtttcttggaaaagccttgttcaaaattctacagaaccacctggccttcccacattcctatttctattagttcctaaaatgacttgtaccaaatccatacatgcatgacttcctatgaaagtactctttcatcagtaggaatttagtagctggtttccagttaatgtattttgtcaagtactggggttggggagaacccgttttgattacaagcagataattatctcagtgagatgggggttagttcaaggaagtaaggaggggggaggatgtgaggaagttagaacaacccaatgcttatttgatgggctgaataaactattcaggactgaactatttttgagcactgtgaggtggcacagtattacctgcttcaaaatcaactgataccaacatttttatctttgtatcttatctctgtacgtgtgtgtattgaggaaatgctttactgactcagaggaaagatcatgaattctccattggcaaaaccacctctgtcctttcggcaaggctgcatacttccaggcagacgcaccttcacgagaatgctcagctgggcggctccacgctcatccagtgggcctaggttctgactgaccagcgaacaaaaactgtgacagagatctaggatttcattcaggcagtgaaacacctacccgggaaacagagttggcattaggaaaggaaggaaggtacatccatgaagttaaagtgttaggagaacagtctgattaatagctgatctaattaatagctgacctcccaaatctgacaggatagacactgccacgtgcaaggcctgccagcccctcagacgcacaaaatgcgtaaaacaaatgcatcctttcctggctaagcgagtattactctcttagccctgcaccaaacctccaatctagccacatttaactcttcatttcttagacccgcagagtgtcttcctgcctctgagctgtgagtgttgttccctttgcccgggatgctcttgtttttaataccagttcaagtcccactctctcagtgaagcactcccttccccactatagcctttagtgaaccctcgtttcttgcttctttattatctgtactgttgtccacttggcaattgttcaggcctctgtgttgttactgatttttgtatgtatatatatatatatgtcttgtttttccaactagattgtgagctccttaagggcagagccatgaattatacctctttgtatccccagtgccttgcatacagtaagcactcaataaatatttaatgatgatga

SEQ ID NO: 37 - DNA, 2330 bp, Homo sapiens
  source 1..2330: mol_type = genomic DNA
acttccgggccctgcgtggcagttgaaagagtggcgggagaagttgcagggaattatttgatagcacatactgatggctctaaagaagtcatcaccctcactggattctggtgatagtgactctgaggagttgccaacatttgcctttctgaagaaggaaccatcttcaacaaagaggagacagcctgaaagggaagagaagattgtagtggttgacatctcagattgtgaagcctcctgtcctccagcaccagagttattttcaccacctgtcccagaaatagctgaaactgtcacacaaacacagccagtcaggttgctaagcagtgaaagtgaagatgaagaagaatttattcctctggctcaaaggcttacatgtaagtttctgacccacaagcaactgagccctgaggactctagctccccagttaaaagtgttttggatcatcaaaataatgaaggtgcatcatgtgactggaaaaagccctttccaaagatccctgaagttcccctccatgataccccagagaggagtgcagcagataacaaggacctgatcttagatccatgctgtcagcttccagcctacctgtctacctgccctggccagagcagcagcttggcagtaaccaaaacaaattctgacatccttccaccccagaagaaaaccaagccgagtcagaaggtccagggaagaggctcacacggatgccggcagcagagacaagcaaggcagaaggaaagcaccctgagaagacaggaaagaaagaatgcagcactggttaccaggatgaaagcccagaggccagaggaatgcttaaaacacatcattgtagtgctggatccagtgctcttacagatggaaggtgggggccagctcctaggagcactgcagaccatggagtgccgctgtgtgattgaggcgcaggctgtgccttgcagtgtcacttggaggagaagggctgggccgtctgaggacagagaggactgggtggaggagccaacagtactggtgttgctccgggcagaggcatttgtgtccatgatcgacaatggaaagcagggaagcctggacagcactatgaaagggaaggaaacgcttcagggctttgtaactgacatcacagcaaagacagcagggaaagctctgtcactggtgattgtggatcaggagaaatgcttcagtgctcagaatcctccaagaagagggaaacagggagcaaataaacagaccaagaagcagcagcagagacaaccagaggccagcatagggtccatggtatccagggtagacgctgaagaggcattggtggatctgcagctacacacagaagcccaggctcaaattgtgcagagctggaaagagctggccgacttcacatgcgcattcacaaaggctgtggctgaggcgcccttcaagaagctccgagatgaaactaccttctccttctgtctggagagtgactgggctggaggggtgaaggtggaccttgctggcaggggactcgcactagtctggaggagacagattcagcagctgaaccgagtcagcctggaaatggccagtgcagttgtgaatgcctatccctccccacagctcctggtacaggcttatcagcagtgtttttcggataaagaacgccagaatttgctcgcagacatacaggtgcgccgtggggaaggtgtgacatccacttctcgccgcattggaccagaactatccaggcgtatctaccttcagatgaccactttacagccacatctctctttagatagtgctgactgattctagccctcagggatgaggatgaaaagctggaaacttccacttccccaacctcagagcctgactgtaatgaagagactggcagcacctcctggaacacaagcctaggtgaggcccagtctttcttgggtcttattatttgtgaaggtctctctgcctgtcggctggggcagagactgaaatactgccacctacctttggcatttaatgttcctctcctggcaaaaattcactgccacagacaaaccacccccactcctacccagccagccctcaaaacacaaaggaacaaagacagtccactcagacacttatttaataactgtagaaatccaaaagaattagcatcaaatcttgaagtcgtgagtgaagctgcgggttggcttgactgggctcagccactgagctgcctcaaccggccaaggaacgggattatgatgactatgcggacttctatattgtcttcatctcattgtgtgtattatgtatttagtttcaataaagcatttgtaccaatggctctggagcttggaggaagactaaa

SEQ ID NO: 38 - DNA, 1053 bp, Homo sapiens
  source 1..1053: mol_type = genomic DNA
agttcccgaagcttccggtggccggcttagttaggagctatggctaaacatcatcctgatttgatcttttgccgcaagcaggctggtgttgccatcggaagactgtgtgaaaaatgtgatggcaagtgtgtgatttgtgactcctatgtgcgtccctgcactctggtgcgcatatgtgatgagtgtaactatggatcttaccaggggcgctgtgtgatctgtggaggacctggggtctctgatgcctattattgtaaggagtgcaccatccaggagaaggacagagatggctgcccaaagattgtcaatctggggagctctaagacagacctcttctatgaacgcaaaaaatacggcttcaagaagaggtgattggtgggtggccccttcctccccccaacatcagtctgctgcagctgccagaaaacatgcctactactaccagcagaaagggagcagagcccagagcatcaccaggagtgcctgctagtgtactggcagcttgccaccccctcctctcccttcacccagacacgtggtagggatggaaaaggattcttcacagagcactctggcacaccatatcggagaaaacttgatagattagttaatggtttttcttgaattcgagaagcaaagatctgttctccatattggtatgttctccctcaaccaagatcttctaaaaagaaataatattttagtcttctgcttgaggaactgactgtgaagcgacgcccagtgaaaaacatgttcttgcagcagctctggtggcagctgtccttgaggaacctttggtgtgtggtgggaagctatcagaacaagaaatgtaggcatttcccgttttttttggggggggggtgggggggcagggctctgccctcttgaaaggcatttacttgtttaacacttgtccagctacagtggggtacagtagctggctattcacaggcatcatcatagcccactagtctcatattattttccttttgagaaattggaaactctttctgttgctattatattaataaagttggtgtttattttctggta

SEQ ID NO: 39 - DNA, 3475 bp, Homo sapiens
  source 1..3475: mol_type = genomic DNA
attctgtgctcgtgcaggaggagggctgctaccatcagggacgtgcacgatttcccccaccccgccccctccccaaactccaaaaaaaaatccaaaacacactgccacccaccgtgcccccgcgccccgagcccctccggtcccggcctgcgtttgctagcgagcgcggggaacccacggcagtgtctgtgcctgtgccaggctgcaggtctggcgtgctcctgatcagagttactctgatctgaagattttggcgttcaaggcattaagataatagcctgagttgttcatggagtttttcatcagtatgtctgaaaccattaaatataatgacgatgatcataaaactctgtttctgaaaacactaaatgaacaacgcctggaaggagaattttgtgatattgctattgtggttgaggatgtgaaattcagagcacacagatgtgttcttgctgcctgcagcacttactttaaaaagcttttcaagaagcttgaggttgatagttcttcggtcatagaaatagattttcttcgttctgatatatttgaagaggtcctgaactacatgtacacagcaaagatttccgtgaaaaaagaagatgttaacttaatgatgtcatcgggtcagattcttggtatccgatttttggataaactgtgttctcagaagcgtgatgtgtccagtcccgatgaaaacaatggtcagtccaaaagtaagtattgccttaaaataaatcgccccattggagatgctgctgacacccaggatgatgatgtagaggaaatcggggatcaggatgacagtccttctgatgacacagtagaaggcacacccccgagtcaggaggacggcaagtcgcccaccacaacgctcagggttcaggaagcgatcctgaaagagctggggagtgaggaagttcggaaggtcaattgctacggccaggaagtagaatccatggagaccccagaatcaaaagacttggggtcccagacccctcaagccttaacatttaatgatgggatgagtgaagtgaaagatgaacagacaccaggctggacaacagccgccagtgacatgaagtttgagtatttgctttatggtcaccatcgggagcagattgcctgccaggcgtgtgggaagacgttttctgatgaaggcagattgaggaagcatgagaaactccacacggcggacaggccatttgtttgtgaaatgtgcacaaaaggtttcaccacacaggcccacctgaaagaacacctaaaaatccacacaggatataagccctatagctgtgaggtgtgtggaaaatcatttatccgtgccccagacttaaagaagcatgagagagttcacagtaatgaaagaccgtttgcgtgccacatgtgtgacaaagccttcaaacacaagtctcacctcaaggatcatgaaagaagacacagaggggaaaagccttttgtgtgtggctcctgcaccaaggcatttgccaaggcatctgatctgaaaaggcacgagaacaatatgcacagtgaaaggaagcaggttacccccagtgccatccagagcgagacagaacagttgcaggcggcagcgatggctgcggaagcagaacagcagctggagacgatagcctgtagctagaggcggtgggacagggacactttgcctggaaagtggagactgagatgacgtggatcataatgagtgaatgccagttacaatatttttgtggaaacgtatggaacattgtactcactggacttaaggcagtgcttggttagctatttttaagacttttcaaggaaatggtgttcctcagttctgaccaaaccgtttcactgtcttgtctggtgtctagtattaatgttgcccagtaagcacctctctcccttttttttttttattattttaatttgagaactcctgtgtccagtttagaagtgagagacttccatttttagttcctttacactcaccaccctagcaagtgccctgcacagagtaataagtaaattgatttcctaatcacaattctatgtgacttatggtcaaaagagcagttttaataactttaaaagtacttcagatagacgcagaaaattggtgagtggttgaccaagaacactgcacaaatataaaaaaagttctggaaatgcagaatggcgttagatttatatttggtttgttaattttatatcactgtttttcactgtttttgtggacaaataatggttgctttgctgaagtgttcttcctcaatcttgattgccctgtacctacccaaaagctgtagtcacacatcctaaaggccaagcaaacccaccgggatggtggggggtcttggagccaagctcttaggttcctcttatttggggcagtaccagtccataccagctgcgatttgtgagtggacctgtggtaagaagaatagaaaaggctctcagagataaggttttttacatgtgtaacaatcccaagatttcctagattaaaatcttaattgattttgaaattggatttttatttagaatcaaaattaggacaagaacagataacttcttcagatacatttgtgtaactttacagaatgtcatcaagctttggggctctgtggggcacatgatttatccataaaggagatgcagtatgcttacttaaattaataaatttaaaatcttttaagtgtgtaaatagtagtgttggtcttacgtatttcaagtaaaaagtagacagctgcactttttttgcacattggattaaaataacttccatcagcaacaaacatcagactgtttttaacaaatattaaagattgtcagaccaaatgtttatgttttcgaaatatatttcatcactggttacagttttaaatagaagttgattgccttttcatagccgtaaatgagaattataaactctattccagttttggtatactaaatgttcttttaaccatctttaggaatatattgaaatgccaacaatagtttgaattgtgttctgtaaaaaagtattagtcaattatttttcaaaatgtagaattgtagaaaatgtcaatttttcaaactcatttttcattgctaggatttcttttaaaaaaattaaagtaatttcacttcatagcttgttgcattaacacctgtaattctattgtgaaagaaatttgattttttaattggtgtgtgttatgcccaagatgttaggatgaagatgctacctttttaaaaattttagttattggtaaaatgtttaattttacttaaaattaatttcttttcttgaaagttgtttattctgttctaaaagtagtcctgtcactgtgtttttatgctacttatagaaggggaactgattgccgttcctaggctgttttaaccaaagcaaaggaaaaataattttaacatcttgtcaatagctacttcaacaaaccatattttgggacttcattttttgtatataaaagaaaatatacttcta

SEQ ID NO: 40 - DNA, 2880 bp, Homo sapiens
  source 1..2880: mol_type = genomic DNA
gttctcctcaactggcggcggcgcgaacgaatagtcgccggcgacctcttggctctctggcagatttcctctagtaagaggtggctctggaggccccgcgaaacgagtgtggtgtgtggttgcaaggcatgatggctgcaaaagtggttcctatgcccccaaagccaaagcagtcctttatactgagagttccgccagactccaagctgggccaagacctacttcgagatgccactaacgggcccaagaccatccaccagctagtgctggagcacttcctcaccttcttgcccaagccaagcctggtccagcccagtcagaaagtcaaggagaccttggttattatgaaagatgtgagctcaagccttcagaacagagtgcatcctcgtcccttggtgaagcttctgcccaaaggagtccaaaaggaacaagagacagtgtctctgtatttgaaagctaaccctgagctggtggtctttgaggatttgaatgtatttcactgccaggaagaatgtgtgagcttggatcctactcaacaactcacgtcagagaaggaagatgacagcagtgtcggggaaatgatgttactggcagtcaatggcagtaatcctgaaggtgaagatcctgagagggaacctgtagaaaatgaagattatagagaaaagtcttcagatgatgatgaaatggattcttccttggtctctcagcagcctcccgataaccaggaaaaggaacgactaaatacatccattccacaaaaaaggaaaatgagaaatctgttagttaccattgagaatgatactcctctagaggaactctcaaaatatgtagacatcagtattattgcccttactcgaaatcggaggacaaggagatggtacacttgtccactgtgtgggaaacagtttaatgaaagttcttacctcatttcccaccagaggacccacactggagaaaaaccctatgactgtaatcactgtgggaaaagcttcaatcataaaacaaacctcaataaacatgagcgaattcatacaggagagaaaccttattcctgttctcagtgtggaaaaaacttccgtcagaattctcatcggagtcgtcatgaaggaatccatataagggagaagatatttaagtgtccagaatgtgggaaaaccttcccaaagaatgaggagtttgtgcttcatctgcagagtcatgaggctgagagaccatatggttgcaaaaaatgtgggagaagatttggtcggctgtcaaactgtacccggcatgagaaaacccactcagcctgtaagacccgaaagcagaagtaatactgggaaccctttctgggtctgatggtgctgcctcaacctgagagctttcataagtagttctgaattcccaagctgcctaaaaaggtataaatgtgtaaaaatctcattattgccaaaattggataaatgcccatcttagctaaaacctcaaattgctagaaaattcacagggaagaaaacatttcaagggctatacctcagcatctaggctttttggactaaggagctttcctttttgaagttatatgataatgtacaggtcacagatcccctttcccaacactttgaagatgaatctggagtctgcttacttggaaggcaaagagtgacttgtgtctattgaaagtatatccgttttccccccacatggggattcatacttgagaaatagtgcaaagatgcttatctggaactgtgttctggtgaaagaaccaaattactggcttgttagccaacagcttctgatagcaattcatataaccctctaagaatacctgtttaagtcttgagtgttgaaaggaattgtttactttggaatataggaaaacagttgaatgtcagactctcatttgtatgtgatctaaatttgcaatcaatttcaataatatttacaatttgtgataaaactgacttttacagattccttttcacaacataatttaggtgtctactgttcttattgtattttgttctgctgttgatctctccagcagccgtctcatgcttctcccttgctaaaagaagtttggattactcaggcagggccatccagccccaccactagaaaagctcttcagaatcttgtccctctgttgagcccagatctcatgtgctacgaaggaaaccccaagacccagagaggaagggtcaacctggaggcaggaaaaagttggcttggatccatgtctcatcaataaccttaccatatgcttaggtcccctctatgctgtcatcagacctttggcaatggggtggtcactacctcacaaggcaaagtgttgtatgattagaaattacgtctccagtggttagctcacattgcctctcaagagacaggtttccaggtgtcttcattgtagtgggtattaattgtcttcagcctcttgatatccataccttcctgtcctctgcctagaagcaaggccagcggtgcctttacggactgatcgtgtggtgcgatttagggattcttcagttttgcttgctttaggtttccaaaagttatacattggtgttttgattggaataaagaaatcctataagctatttgggaaaaattatagtgtatgtttcccatccagaaacatgcctttctatttattagagtattatattcctgtgaaaatttttctaattttcttcacttgttttacacaattttgttattgtagttttttccattatatttttatagttgattattgcttttacatgggaaagttatttttaattatatatttgtatagtcatctcactgttgttaattttcaatagtttgttggtttagttctgttaacttttggtaaaatgacaccatctacaaagaatagtttttgtttcttgc

SEQ ID NO: 41 - DNA, 5301 bp, Homo sapiens
  source 1..5301: mol_type = genomic DNA
agaggcgcttaagttaccaagggattagggctgatctcaggagaggtaaacgaccatccttggaacacggagcccttcttccctgcccggtatctgcgcgtgccttgggtagtccgcacaaccctccccagctccggatgccctgggatacccggacccaggagagagcgcgtcagcggggcgcagctactttgcactcgccgattctgacacaacagatagttaattggggccttcgaaatcaaggactaagaagtactgaactcctaatatcaccaattcttctaagttcctggacattgatccggaggaggattcgcagttcaacatcaaggtccctgtgcgttttattgcgacctgccggtgggaactttgtctccgagtcggagcagcatggagcggcggagcgagagcccgtgtctgcgggacagccccgaccggcggagcggcagcccggacgtcaaggggcctcccccagtgaaggtggcccggctggagcagaacggcagccccatgggagcccgcgggaggcccaacggcgccgtggccaaggccgtgggaggtttgatgattcctgtcttttgtgtcgtggagcagttggacggctctcttgaatatgacaacagagaagaacacgccgagtttgtcctggtgcggaaagatgtgctttttagccagctggtggagactgcgctcctggccctggggtattctcacagctctgcggcccaggcccaaggaataatcaagctgggaaggtggaaccctctccccctcagttatgtgacagatgcacccgacgcgacagtggccgacatgctacaagatgtctatcatgttgtgacgttgaaaatccaattacaaagttgttcaaagttggaagacttgcctgcggagcagtggaaccatgccacagtccgcaatgccttaaaggaactgctcaaagagatgaaccagagcacattagccaaagaatgccctctctcccagagtatgatttcatccattgtaaatagcacatattatgccaatgtgtcagcaaccaagtgccaggagtttgggagatggtataaaaagtacaagaagattaaagtggaaagagtggaacgagaaaacctttcagactattgtgttctgggccagcgtccaatgcatttaccaaatatgaaccagctggcatccctggggaaaaccaacgaacagtctcctcacagccaaattcaccacagtactccaatccgaaaccaagtgcccgcattacagcccatcatgagccctggtcttctttctccccagcttagtccacaacttgtaaggcaacaaatagccatggcccatctgataaaccaacagattgccgttagccggctcctggctcaccagcatcctcaagccatcaaccagcagttcctgaaccatccacccatccccagagcagttaagccagagccaaccaactcttccgtggaagtctctccagatatctaccagcaagtcagagatgagctgaagagggccagtgtgtcccaagctgtctttgcaagagtggcattcaaccgcacacagggattgttgtctgagattctgcgtaaggaagaagaccctcggacagcctctcagtctcttctagtaaacctgagggccatgcagaatttcctcaatctgccagaagtggagcgagatcgcatctaccaggatgagagggagcggagcatgaatcccaatgtgagcatggtctcctcggcctccagcagtcccagctcctcccgaacccctcaggccaaaacctcgacaccgacaacagacctccctattaaggtggacggcgccaacatcaacatcacagctgccatttatgacgagatccaacaggagatgaaaagggccaaggtgtctcaagccctgtttgccaaagtggctgcaaataaaagtcagggctggctgtgtgaactgctccgctggaaggagaacccaagcccagaaaaccgcaccctctgggaaaacctctgtaccatccgtcgcttcctgaaccttccccagcatgagagggatgtcatctatgaggaggagtcaaggcatcaccacagcgaacgcatgcaacacgtggtccagcttccccctgagccggtgcaggtacttcatagacagcagtctcagccagccaaggagagttcccctcccagagaagaagcgcctcccccacctcctccgactgaagacagttgtgccaaaaagccccggtctcgcacaaagatctccttagaagccctggggatcctccaaagctttattcatgatgtaggcctgtacccagaccaggaagccatccacactctttcggctcagctggatctccccaaacacaccatcatcaagttcttccagaaccagcggtaccacgtgaagcaccacgggaagctgaaagagcacctgggctccgcggtggacgtggctgaatataaggacgaggagctgctgaccgagtcagaggagaacgacagcgaggaaggctccgaggagatgtacaaagtggaggctgaggaggaaaatgctgacaaaagcaaggcagcacctgccgaaattgaccagagataatgtgaacttctactaggcaaagcaatacatcggtccaaggattttctgctttcatttctttaaaagttttttgttagtttgttttttgtttttgtttttgggtttttttggctttatttttgtctttttatgtctgttttgtttttcttacccttttggacatttctttgttgcacaggatacacctatagactgaataagttcagtatttccgaatcagacatcgccttggcaaagacactaaagcgttacactttatcccgtctctatgactggatcatagtcattataatcacaggagactctgccttcattatccttgcacttaacggaagttacatcaggcaagtaccaggatgaaaagaactatgaaataaatgaaggaagctacaagtgtgtgtgtatatgtatatgtatatatctctatatttacatatatatattaaaattgcatgggacagagactttgcaatccgaaagaatagactgtgaaatgagttcttaaagaaaagacttgtttatgtattaaaaaaaccacttcacagtgagtcgctttggctttttgataaactgcggcctgctctcagggtggggtgactatttttgaattcctatttattttttgtgtttgtccctgattttttttttttaattctatggcttcctatctggcagcttaatgggtaatttttgaggtatgtatttaacaaaataaacgacactgccgaaaaaaaaaaaagtgaagtgaaaacaatcagggcacattaaaatgatacaagtcaaataaatcttaaagacacaatgcacacttaaaatgactcaataaaatgacttgctacgttccgttattcaatttgtcattactgtagtgaacagatgcatttctgtggaattccaaataagtaaaactgaaattcagtgcagagaaaactttgtccactagtgcaagtcttgatcaaatgacattttgacattggacatatggaattcatagtatgagccacattttgttgtgaaatttatttacctgcttgtggcttcaaatctgaaaattaataagcctgctcgtttaaaagttgtttgttgttgctgtttttttgtctttttgttttttactagaaaatagttcagtgtaatattaagttagaaaagaagttgctgcccagttaaaggggctccctctcaaataaatctccatccttccctctcccaaaagacatttctgatttctgcttcactttgggcttcctcttcttcgtacacattccatctacctaatcaaacattttcagtccctgatctctcctgtcccttttcctgggatgacagccctaacaagaactgtttttgaatcgttgtgcagctccaggcaatagagtatgtgaagcgatttcagtagaatcacttactcatcctaaaagaaaacattatcccagttacctacatcgcaattaccttatgtaaagcagaactaatgctgactggatgtttaatgggatgagcattaaagctgcaatctactatagtactccagatctctttcggcttcctatgagaaacaccagaagcattactttccacttctacttacagtaattgcaagaggagacctcacattcaggactggcctagtgaacgtaatccatgctttaaactggccattaaacagtcccacatggttggattttttttttttttttgagttgtgctttcacaaaaccttgtcaaagacctcatgcaatatcactttgaaagttattttctgtttactacacaaacattgtaatataactgttaatactatttatatatttgaaaggtataaaaggtaggagttaaaaaaaaaacctctatgtgtagatattaactcagaacttacaatatacagggagaagacatgttgcaatacaagctaattctagctgctcagtaacctctggagtttttaaagggacattttcctgtactttttcaaataatgatgtttaaaaattatcttgacataagcgtcatatacctttgcaaaaggatggttgtttgcagttagccctggccccatccttcctatttctgtagtatgctgcagctttaatcagaaagtccatggttgctgcttcctgatctccgagttactctttccaaattgtcttcttacactgttgctgaaggtcactctgtacacgtaatggaaactgattttgccaagctcttacaaggtggttcatctatcgatggcatccgcatttggtatcttttacacttcaaccaaaaatttattaggtatttttcaatgctaagtcttgccttttattttttaatttcactgccaagtttgcagtggttctaagtgaatctgtgggcattttagcctgtggtcttgccagatctttgcgaattacaatgcatatatgtctatttattcaatatctgtcatataatatctatttggaagaagaaactttctcttgtagtgcctcttgacaaagcacaatttcccgcctttttttttttttgtgaaatgaaaaaaacaaattgtgttttattgcggtatcaacaatgtgaataaggattaacatattgtaaatgttcttttttccatgtaaatcaactatctttgttatcactaagtgataattaatttttaacttatgtgcattgttaggctgttagaattttttggttgttaaaataaacgcattcaataaa

SEQ ID NO: 42 - DNA, 9790 bp, Homo sapiens
  source 1..9790: mol_type = genomic DNA
gacgaaggcggctgtggtagcggcggcggcggcggcggagccctgggtcggtgtctgcgcgctggtgtctgaggcccaggctgaggcctccgctattgctggagcgcaggcggcggagaggatgactgccgctgccattctctcttgagctagcgagccgccgccaccctccaccctcccccggcagggcggagaggagcggccggagtcagcgatggtgcccggcgaggagaaccaactggtcccgaaagaggcaccactggatcataccagtgacaagtcacttctcgacgctaattttgagccaggaaagaagaactttctgcatttgacagataaagatggtgaacaacctcaaatactgctggaggattccagtgctggggaagacagtgttcatgacaggtttataggtccgcttccaagagaaggttctgggggttctaccagtgattatgtcagccaaagctactcctactcatctattttgaataaatcagaaactggatatgtgggactagtaaaccaagcaatgacttgctatttgaatagccttttgcaaacactttttatgactcctgaatttaggaatgcattatataagtgggaatttgaagaatctgaagaagatccagtgacaagtattccataccaacttcaaaggctttttgttttgttacaaaccagcaaaaagagagcaattgaaaccacagatgttacaaggagctttggatgggatagtagtgaggcttggcagcagcatgatgtacaagaactatgcagagtcatgtttgatgctttggaacagaaatggaagcaaacagaacaggctgatcttataaatgagctatatcaaggcaagctgaaggactacgtgagatgtctggaatgtggttatgagggctggcgaatcgacacatatcttgatattccattggtcatccgaccttatgggtccagccaagcatttgctagtgtggaagaagcattgcatgcatttattcagccagagattctggatggcccaaatcagtatttttgtgaacgttgtaagaagaagtgtgatgcacggaagggccttcggtttttgcattttccttatctgctgaccttacagctgaaaagattcgattttgattatacaaccatgcataggattaaactgaatgatcgaatgacatttcccgaggaactagatatgagtacttttattgatgttgaagatgagaaatctcctcagactgaaagttgcactgacagtggagcagaaaatgaaggtagttgtcacagtgatcagatgagcaacgatttctccaatgatgatggtgttgatgaaggaatctgtcttgaaaccaatagtggaactgaaaagatctcaaaatctggacttgaaaagaattccttgatctatgaacttttctctgttatggttcattctgggagcgctgctggtggtcattattatgcatgtataaagtcattcagtgatgagcagtggtacagcttcaatgatcaacatgtcagcaggataacacaagaggacattaagaaaacacatggtggatcttcaggaagcagaggatattattctagtgctttcgcaagttccacaaatgcatatatgctgatctatagactgaaggatccagccagaaatgcaaaatttctagaagtggatgaatacccagaacatattaaaaacttggtgcagaaagagagagagttggaagaacaagaaaagagacaacgagaaattgagcgcaatacatgcaagataaaattattctgtttgcatcctacaaaacaagtaatgatggaaaataaattggaggttcataaggataagacattaaaggaagcagtagaaatggcttataagatgatggatttagaagaggtaatacccctggattgctgtcgccttgttaaatatgatgagtttcatgattatctagaacggtcatatgaaggagaagaagatacaccaatggggcttctactaggtggcgtcaagtcaacatatatgtttgatctgctgttggagacgagaaagcctgatcaggttttccaatcttataaacctggagaagtgatggtgaaagttcatgttgttgatctaaaggcagaatctgtagctgctcctataactgttcgtgcttacttaaatcagacagttacagaattcaaacaactgatttcaaaggccatccatttacctgctgaaacaatgagaatagtgctggaacgctgctacaatgatttgcgtcttctcagtgtctccagtaaaaccctgaaagctgaaggattttttagaagtaacaaggtgtttgttgaaagctccgagactttggattaccagatggcctttgcagactctcatttatggaaactcctggatcggcatgcaaatacaatcagattatttgttttgctacctgaacaatccccagtatcttattccaaaaggacagcataccagaaagctggaggcgattctggtaatgtggatgatgactgtgaaagagtcaaaggacctgtaggaagcctaaagtctgtggaagctattctagaagaaagcactgaaaaactcaaaagcttgtcactgcagcaacagcaggatggagataatggggacagcagcaaaagtactgagacaagtgactttgaaaacatcgaatcacctctcaatgagagggactcttcagcatcagtggataatagagaacttgaacagcatattcagacttctgatccagaaaattttcagtctgaagaacgatcagactcagatgtgaataatgacaggagtacaagttcagtggacagtgatattcttagctccagtcatagcagtgatactttgtgcaatgcagacaatgctcagatccctttggctaatggacttgactctcacagtatcacaagtagtagaagaacgaaagcaaatgaagggaaaaaagaaacatgggatacagcagaagaagactctggaactgatagtgaatatgatgagagtggcaagagtaggggagaaatgcagtacatgtatttcaaagctgaaccttatgctgcagatgaaggttctggggaaggacataaatggttgatggtgcatgttgataaaagaattactctggcagctttcaaacaacatttagagccctttgttggagttttgtcctctcacttcaaggtctttcgagtgtatgccagcaatcaagagtttgagagcgtccggctgaatgagacactttcatcattttctgatgacaataagattacaattagactggggagagcacttaaaaaaggagaatacagagttaaagtataccagcttttggtcaatgaacaagagccatgcaagtttctgctagatgctgtgtttgctaaaggaatgactgtacggcaatcaaaagaggaattaattcctcagctcagggagcaatgtggtttagagctcagtattgacaggtttcgtctaaggaaaaaaacatggaagaatcctggcactgtctttttggattatcatatttatgaagaagatattaatatttccagcaactgggaggttttccttgaagttcttgatggggtagagaagatgaagtccatgtcacagcttgcagttttgtcaagacggtggaagccttcagagatgaagttggatcccttccaggaggttgtattggaaagcagtagtgtggacgaattgcgagagaagcttagtgaaatcagtgggattcctttggatgatattgaatttgctaagggtagaggaacatttccctgtgatatttctgtccttgatattcatcaggatttagactggaatcctaaagtttctaccctgaatgtctggcctctttatatctgtgatgatggtgcggtcatattttatagggataaaacagaagaattaatggaattgacagatgagcaaagaaatgaactgatgaaaaaagaaagcagtcgactccagaagactggacatcgtgtaacatactcacctcgtaaagagaaagcactaaaaatatatctggatggagcaccaaataaagatctgactcaagactgactctgatagtgtagcattttccctgggggagttttggttttaattagatggttcactaccactgggtagtgccattttggccggacatggttggggtaacccagtgacaccagcactgattggactgccctacaccaatcagaagctcagtgcccaatgggccactgttttgactcggaatcatgttgtgcactatagtcaaatgtactgtaaagtgaaaagggatgtgcaaaaaaataaaaaaaaacaacaaaaaaagctaaccttctattagaaaaggggacaggggaatgagtaaacttcttttattgcggacaaatgtgcacatagccgctagtaaaactagcctcaaacaggatgctcatagcttaataataaaagctgtgcaaaggccatgaatgaatgaattttctgtttatttcactgatgcacacattacctcattgacaattcagaagtaaatccaacgtgtgttgactcttggaaagcagcaaaaacaggagctgaagaaaagaaattcttggaaccagccgtaacccagtaaggaattgtgaagttgtgtttttattttgtttcattttttgcagagtattaagaacattattctggaacatcagaacgtttcccttagaccgatcccagcaggtggcagctcagattgctgcagtgttgtaattataactgattgtacttaagttatggatgtagagaatatgtttcattcatttattcagcatgtaaataaaattgatcctgttgagttatcataattgcagttcaactatctgccatgattattcttttcacgtatcattcattctgtacatttgtgtacattgagaagtatagcaatctatgtaaatgtaatcctcagtgaggttcctcagtgctaggtcccataggattgtcgttgcccttgttaatgaggtttctctgttcagcggcttcaatttttttctctttgtacatctagttttgaagatttacttcaagtttgaatcttctagaatgcttgtaagtccagttttaatttttagagtcaatttgtagttacatgtagtttaacttttgggaaacgtcttaacattgttctgaataaacttgctaatgaggtcaggtcatggtacagactgatgcagtcaacatgatttcattgcagagtttattagtatcagcaagtttttgctttgctaaataaaagtactcaatgaacacaattctacataaattttgacataccatctaatttataaaaatcaataaaaaaggttttggtaaaactttttcatgccagatgctgtttacaacaatgaacatgccaataaaacatttgttcattctgttgtgttattttagtcattaaacttctgtggatgaagaatctgggttaagaatagatttgtcatctttaaatatgacattttgtaatgtgtattggatatctcatttctatgataaaggtatatttacagtaaagttctcataagagaaatgaaaagctgtgttaatatctaactttggggaaccctgtcagtatttcagatccgatttttaccctttttttcttataagaaagataaaattagaaaatactgttagcaaatgtggctctgccatttgaatataatcaccgagaattccatgtcttaaaagtctcctggaatccacaatgaaaaaaaaaatcttttctaaggtatttttctggctaatttttatttgaagaaagctatagcatttagcgaaatttgactgaagtaatgttctgagtttgcattagtgggattggtgatgttctcagaagaaaattggaaacacttgtgatgaattgtctttcagatcacttagattttctgatgtaagaggacagctgtttggttctgatacaggcctgcttacttgggatgtagggttagtaaatggggtttctgctttaaaggactgacttgctatcacacaaaagaggcagacttgtaaacacaatgggctttggagtttggtctgattgggtttggtttagtattcctatgagcgtaaatggtaaaattcttctgatacccactctttagactgtgccttctgctctgttctttgttttatgtttaactgctgtttctaattgcaggtgtattacagatacaaataagagtaaagaaaatatatttcattatagaaaagaaaaaattaaaagcttcttgcttttcagtgcctgatagagtgaaaacacaaagttgcactttaataatttcaataaaagctaatctgtgtcagcctccctctgcttcagagagtcaggtgagcatccataacctaacaggcagagccctagcgatgtggatcaagtttcctgagcccgggggcggtggagcctcatgatctcttatcttttgaggctgaggcaggtcacatgcaacaaattgtgaccctgctccccacaagtcatgcaaaggttttgaagagcttttaccgtggggcagatgaacttgtgtcaaccatgcacaccctgtgagaaccaagtacctgtgtttctaaggcgggcactcaaggtgaggggtgcattctggccaaagaaacaaaagctgtggtttcaggaccatgccgtgtgtagctgatctgtacgggacgtgtatgtaaggaagagcaatcatgatagataagaacagtgtgtgaagcagccttcacactagagtgtttggtcatctcttataatgtaagggaaggtactttaaaattctgggaagatgcgatgaactcatgtcccagtcagaaaataatccaatgaaataagcattggttgccaggccacagttaggaattgtattgtgatacatctagaggccaagagagcaggagagagctaccaacttacactgtggtttaagctaaatgaccgcacagcatcatagcattgcagtgttgttactaaatctggaagtgacctgtgaatgtatggaatacaataaagtcttttattctggttcatttgctagtacttcctttttgattggatactgtagttcttcctctggattttattttgttcagcgtcaaggccctaattttgcaaatgtagtctaaaccacattacgtggactagaggatactctgaattagcaagttttttgtttgctgaataaaactattccatcttaacaattctgtgtaatttttgaaaaacatcaacattttggtcactagtagtcaaaatcttataaggccatatgtcctgaaaggcaaaactctgtgtatgtgcgatttaccagggagatagtaggacagtttttatgagggtttcaaaggagttcatggcccaaatgaagcttgtggccacctaggtttagtgtacatgaaattggaagatgcttcttgattgtagttcccatggggaagagggaggctgtgatcatttggttagatacagcattaattgtcacaacttgactttcagaaacaagaatactgcacaaactcaagcatgcattggagcatgtgttactatttagaaaagagtgcagccatcagtaggtcaagctaggagggcaaaagagagttgagaggaatgctctgccccttccccatcacagcactgcccctttccacctcctcaccagctcacctgtacaagagaattgatctcaactgagtgtcagctcaccaggtcagaaattgttattggaccctaaaaccttgcctcacaggtgggcaccaagctccccttccctctgctttccatgtttcccacctgccctctccccacctgttttcagcctcttttataatgcttaagtaacctgcttgaagagtataatttatggagataaaaatactcaatgcttacatttttttcataactgtgccaattgttttgtcaaatgcagttacccactgggcagtgggcttatcaagtgctcagtgcaatcctgtgacttttaagtcattttgcattattaagacaatcttatcttaaagttcagtggtttcattcaaagctcccctgaacctccatagtgcctccaagatgtgctgtccctgccaactcctgcaccagcttgactggtggtggaagaggccaggaagagcggggggaagacatgtgctaaccacttcttagcaatgaggctggtaaggttactgtccccttgtgcctggtcactagtaacttttttgtagacatgaaggtagatgccctgcaggtcctatcagcaaaggacccaactcctaacagcattggtgctccctcatgaaatttgacagtgtcttgccagcctcctgctggcagacacttctctgcagtgaataccactatcccatgctgcactagtccctaccaagtcttcaaccctcaatttgatcttccatgagtgtctcaaaaagttcccctcagaagtttcagccaagggaataaaatctaacctctcctccctcaacggagtgtcagggaggggaagaatctgttttccaaacaccacaatctcccaaaaggggatttcaccctcccttggtcatctttatacagggcaattgtgctgctgctgctgaggtggccactttcagcaagccctgtgtgcaggggcctagctgcatttagaaaccaagcatagctatcaaaaagtaatctccaaatagttgaattgcatttcttgagtatctggagaggatgtgcagaccacaggaatacctaatgccttttttctcttcctgtctttgtccctcacactacagcaggcccctcccttccctcttcaacctcatcctccctccccacaggcccagagaaccagttgggctttgttctcctgcaggctatggttcatcatgcaaatagctcctgtgtcagaaatgctttttggcttcaaataacagaaaagctaacaccagctttatcaataataatatcggtggtttacttaaggtgtccagagatggtggagaacaggattggtttcctcctcaatgtcaaggactcaaagactctttctgtggtagggccacatcctaaaccctgtatcctgtgattatttacctgacagggcaaaagagattttgcagatgcaattaaggttaaggaccttgacgtgggaagattgtgattatttacctgacagggcaaaagagattttgcagatgcaattaaggttaaggaccttgacgtgggaagattattctggattatctaggtgggcgcaatttgatcacatgggtccccagaagtggagaacctttcccacctgtagaaagccagagagctggcacctgagaaggacagaactgtcattgcaggatttgaagatgaaggggcccatgagccaaggaatgccagtgacctatagaggctaaaaaacagcaaggaaatggactctccccagagcctccagaggaatgcagccctgttgatcacatgatcaccagatggctgccccagagccaaatgtcgcttcctgagcaccatactcaaaggcaggggaagtggatggagggcaggagctccattcttgtttgccactctccttttgtcaattgggaaaaaattccagaaactctgggagccctccccttacatttcctgggtcatggggccagccctagctgctggagggactgagaacagctgttgagcagtttacctgacggcatctgccatggcttggcaggaactctggctttgggagagagcagcagcaaggtattcaagcaccacctccacccagcccctcc

SEQ ID NO: 43 - DNA, 4055 bp, Homo sapiens
  source 1..4055: mol_type = genomic DNA
acattgtagcaaaatggcgactgtcattcacaaccccctgaaagcgctcggggaccagttctacaaggaagccattgagcactgccggagttacaactcacggctgtgtgcagagcgcagcgtgcgtcttcccttcctggactcacagactggggtggcccagaacaactgctacatctggatggagaagaggcaccgaggcccaggccttgccccgggccagctgtatacataccctgcccgctgctggcgcaagaagagacgattgcacccacctgaagatccaaaactgcggctgctggagataaaacctgaagtggagcttcccctgaagaaggatgggttcacctcagagagcaccacgctggaagccttgctccgtggcgagggggttgagaagaaggtggatgccagggaggaggaaagcatccaggaaatacagagggttttggaaaatgatgaaaatgtagaagaagggaatgaagaagaggatttggaagaggatattcccaagcgaaagaacaggactagaggacgggctcgcggctctgcagggggcaggaggaggcacgacgccgcctctcaggaagaccacgacaaaccttacgtctgtgacatctgtggcaagcgctacaagaaccgaccggggctcagctaccactatgctcacactcacctggccagcgaggagggggatgaagctcaagaccaggagactcggtccccacccaaccacagaaatgagaaccacaggccccagaaaggaccggatggaacagtcattcccaataactactgtgacttctgcttggggggctccaacatgaacaagaagagtgggcggcctgaagagctggtgtcctgcgcagactgtggacgctctgctcatttgggaggagaaggcaggaaggagaaggaggcagcggccgcagcacgtaccacggaggacttattcggttccacgtcagaaagtgacacgtcaactttccacggctttgatgaggacgatttggaagagcctcgctcctgtcgaggacgccgcagtggccggggttcgcccacagcagataaaaagggcagttgctaaacccacggaacagactctctgggcaattagccatccccctctgactttggtcattgtgctggttctgatatatattttttttaatgaaaggcaactttagattttccctctatccttgctttttttcccttcacctcccacgtgtccctccatccctccccccacccctctgttttgggtatgtacaacagaagcacaaactactgaaacaaaacaaaacagcagaatgagcgttcttccgagagatggcatcgtgatgcgctatttattttccatagaaataggaagttagacggattgtctcttttctgaggggagggggtctttttgacaggagcagagttgatgtcctcaattttcatatttattggcaaaaggaagagaagaggaactttgggttggaaacaaagaaccaataacattaaaacattattatttatatattctagctgttattagaatcagactttttttgcgagagagagagagagagagagagaagggaaatcaaagaaatcgaagcaatatcctgtttagaggcaagccgcccggtggggagaatttcctcaatgggagacggttgcactattctgtgccccacggagtttgcggctccccgcggcagacccctccctcattctcctccctgacctttccatcttcctctctgcttgcgagaaaatgtcagtagttccagagaagtcggggtgcctatgcctggcctccctccacacctgggccctgaccagccgcctcctgggctcctcctcctccgtcagtagagctgctgttttgttattgctggtttttcctcactttcctcctggcaaagaacgacttccaaatgcagggatggaatataagcagaacgtcatgggctcagcagtgactccaccacccgaggccgaggccgtgcttctggaagatagaaggagacatcatcgtgtgtttcccctccccttgcccctgttaagaaacgtatcaatacccattggatgatcaaggctaccgtatttcttctatttttttttatagtgcctgccaggcactttgttttatgtttccaatagcacttcctgaaataaaccaaagcaacactgctcaaggcccctggggcgatggagaaggccacccacctcactgacagtcccaagaatgaccggctgcgaggtcctagtcaaaagtcaacattatgacctggggactccagcatccttcaagcaagccatttccgaagaaggtgaaaagaagccaggatgattggcacctcctcctcctcctcctcttcttcctcttcccttgcccagccccctcctgtgcgtgtgtttcagacaacacaggagccagcacaggagtggaaaatcctgcagcgcaactcagctcagcccacagaagccttgggaatggcctcagtttgtgcaataagaagattttttttttctttttaaatcttcattatattttctttgattgtctgtgagaaagtacccaggtccgcctggaattactctacagtagaaataactgaacacaaacaaactgatggaaaaaaagagttaactattttatttatttcaatatttaaaaggaaaaaagtgctgacatggcacagtatttttgtttaaagtacctcctacttcaaaagttaagcgcaattttgtgaagacatgaaatcataagagtacttaatgtaaaataaaagactgcatattaactctaaagaaaaatgccccacattttaaataagaaaataaagatcaactctgctctctcaggctttttaaaaagccattcatgtatgtgctttaggtatttttatttctgcgagttggatgtggtaagtgaggagtgctcagtttttttttcctccttcaaaagtctattgaaagtgttggtgatgttaaatgattgtgtgttaagatttgactgaaataacttagccacaaatcagcagtttcccccaccctcattgccccctcaccccaggcaagccccttttatctgaatgtcagaagcagcctgcctcctagttatcatgtctgatgaggtctagctcaggaaggaattccatctattgatggaatatatcccctcaagttcaatagattcgaacacagagagctttgtttaaaataatgcagcaaaaaaaaaaaaaaaaaaaaagcaaaaataaaagcatcagctgaggtgatattagttcagtcacctaacaactcctagaagagatgaggaaagggaaccttctgctgagctggcttctggggcctgagcttccagagctgtccccaagggctaggaaggccgacctgaaggatgagaacctcaaattcagttgctggtgggagccaaggaagacggcgggtgttctaacatggccctttctggctgagctggcggaagtgggcgttttggccgatgggatgtatctcggcgctgtgtctgtggcccagcaaaggtgcagggctgactggctgagccactgggttctacccgcaggctccccactgcactgggctttcacacagccatgctcttgggtttccctcccttgtaagcagagtcataataacacacgaatagtctaaggctgggtattctggtcagcagaggtccttgagtcacagtgttactgaaatggttctgagcctgagaatctctttggcctctgaaagggcagggcaggtgggcaccgacttcctgccagtcctttcaggtttcctgttcaaagccagtcctgttggtggaggggatcaccgagagtgtctgtatcattttgtagcccttttctctgacgttttctggtagaaaatgtcccttgtcaaaatgctaataattatcataataatctgctttccaaccaactcccacaagtgacaacctgtgtagaactgtgataaaggtttgcataatgtagggtttgtaccaagtgtgtgtaagtttctgttaaataaaaagtctgtttccaatg

SEQ ID NO: 44 - DNA, 2357 bp, Homo sapiens
  source 1..2357: mol_type = genomic DNA
aaacaggagcgaggtgtctccctagctcgctgcctctggcaagtggagtttttaaaaagctccagcagatcatgtcatgacgacttcgctgctcctgcatccacgctggccggagagccttatgtacgtctatgaggacagcgcggcggagagcggcatcggcggcggcggcggaggaggaggcggcggcacgggcggagcggggggtggctgcagcggagcgagccccggcaaagccccgagcatggatggtctgggcagcagctgcccggccagccactgccgcgacctgcttccgcaccccgtgctgggccgcccgccggctcccctgggcgcccctcagggcgccgtctatacggacatcccggccccggaggcggcgcgccagtgtgccccgccgcccgcaccccccacctcgtccagcgccaccctgggctacggctaccccttcgggggcagctactacggctgccgcctgtcgcacaacgtgaacctgcagcagaagccttgcgcctaccacccgggcgataaatacccggagccgtcgggcgccctgcccggtgacgacctgtcctctagggccaaggagttcgccttctaccccagcttcgccagctcctaccaggcgatgcccggctacctggacgtgtcggtggtgcccgggatcagcgggcacccggagccgcgtcacgacgccctcatccccgtcgaaggctaccagcactgggctctctccaatggctgggacagtcaggtgtactgctccaaggagcagtcgcagtccgcccacctctggaagtctcccttcccagacgtggttcccctgcagcccgaggtgagcagctaccggcgcgggcgcaagaaacgcgtgccctacactaaggtgcagctgaaggagctagagaaggaatacgcggctagcaagttcatcaccaaagagaagcgccggcgcatctccgccaccacgaacctctctgagcgccaggtaaccatctggttccagaaccggcgggtcaaagagaagaaggtggtcagcaaatcgaaagcgcctcatctccactccacctgaccacccacccgctgcttgccccatctatttatgtctccgctttgtaccataaccgaacccacggaaagacgctgcgcgggtgcagaagagtatttaatgttaaggaaagagaagaaccgcgccgcccggaggcagagaggctccatggccgtgctgctgggccatccccaactccctatcccatccccagcctccacccccatccagatgggactcacgtggcttcaacagctttggaaatgggtcccgagtgggccgtgcgaggaaggctgtcgacctctactcctccttgcgctcaccttgccagaaagtctggtggcagcgcagagcccaatcattccaaccaaagcagggttggggaatcccgaatggccccaattcttgcctcatcctatgaccaggcttttagaggaccttccctaagggcgcagcttcggagcaggatctgtccagctcatactttccttcgctgtccctcccgcactccttaggcaagatttcccagtaaagattttctgtgcgtattttaaaagtcgtgttaatactcatgataattattagggacctggcagcgtgattggagtatggatgtttccgtaaaagctggaattccgtaaaagcattgacgcagcccctacactccatcccaaccaagaaactgcatttcctggggccaggtgggagctgcctttgccccactgcctcccctgttctgctctctcagtcaacatgtggaaatccaaggaggacaaagactccagccacgctgctaaatagggctcctctctcctctctctctctctaggtggtaaggttggggattagtccaggtacagaagcagaacttttttctaaggataaacatctcttccaaggggatggagagtgggtccctcaacaaagtccctgtccagtcacctttccatcagggcactagcccaggaatgactcctcacactttcacctttactgatttccagaggaaagctagaggatctagttcaagaggcaagaagatctggccctcaattagctagatgtagatgctgcctaacagttccctcctcaaaggccaccttggtgctgtgggggccccttgcctcttcccttcccactggtgcattacaaaacagtgttcttttgaaatgttcatcaggaataggcttttttaaaaaatgttgtgtatctgtatatagtattgtgatgtctgaatgacaatgtactgaatgcaaaaaggaaaaaaacccacaaacatgtttttaaaataaaatatctttttttgccttga

SEQ ID NO: 45 - DNA, 4252 bp, Homo sapiens
  source 1..4252: mol_type = genomic DNA
aggatgtatgggtcattttgaccagtcattccctcgctctggcatcccacaatttttccgcctccctccgaaagcgataataatatttagaggcgttcgctggggctgaatgagaattagccctaggcgcagcctatcattaggacgggacagcagggccactgtgacatttttaagaaagctgagatgatggaaagaataagaaaagagatgattctgatggagagagggctgcacagccctacggcgggcaagagattctccaatttgtccaactcggctggcaatgctgtgctcgaggccctggaaaattcgcagcacccggctcgcctaagcccgcgcctgccgtctgcccccctgcacagcgctctgggagaactccccgccaagggcaaattcgaaatagacactttgttcaacctgcagcacacgggcagcgaaagcaccgtctcctccgaaatctcctccgccgccgagagccgcaagaagccgggccattattcagaggcggccgctgaggccgacatgagcagcgacgtggaggtgggctgctccgcgcttcgctcccccgggggcctcggcgccgctcagcttaaggaaaacaatggcaaagggtacgcagagagcggctcggctgccggcaccacgacgtcggcgtcgggctcaggcctcggaagcctgcatggaggcagcggaggcagcggcgggagcgcggcgctgggtggctccggctctggcgcggatcaagtgcggcgctaccgtacggcgttcacccgcgagcagatcgcgcgcctggagaaggagttctaccgggagaactatgtgtcgcggccccgccggtgcgagctggccgcggcactcaacctgcccgaaaccaccatcaaggtgtggttccagaaccggcgcatgaaggacaagcggcagcgcctggccatgtcctggccgcacccagccgaccccagcttctacacctacatgatgacgcacgcggccgccaccggaagcctgccctaccccttccactcgcacgtgccgctgcactactacccgcacgtgggcgtcacggcggcggcggccgcggctgcagcctcaggcgcggcggccgcggcttcgtcgcccttcgctacttccatccggccactggacaccttccgcgccctctcgcacccctactctcggccggagctgctgtgtagcttccgccaccctggtctctaccaggctcccgcggccgccgcggggctcaacagcgcggcctctgccgcggcagccgcggcagccgcagcggctgcggcctcctcggcggcggcggccggcgcgccccccagcggcggctctgcaccctgctcgtgcctcagttgccacagcagtcagtcggcggcggcagccgcggcagcagctgccgcagccctgggttcccggggtggcggtggcggcggcggtggtggtggtggcggcggcggcgggggcgccggggccgggggaggctcggacttcggctgcagcgcggcggcgccgcgttccgagagcggcttcctgccctactcggccgcggtgcttagtaagacggccgtgagcccgccggaccagagggacgaggctccgctcaccagataactacgtcgccaccgccagggggccgcgcccgcctctctgagtgtgcccgggagtcccctgtgcgccccctgagccctccgcgcgttgcccgctgctgccgctgccgccagggggcgccccggggcgcgggaggccgaagggaacctgctccgaattgggggagaaggagcaggcgcaaaagcctggcgggcatccccctaaattatttctatttttgtatttgccacccagccgtctgcccttctctctcttttctggctcccgggacccactttccacctctgtcctgagcgcttggtttcccaaagaccaacccctcgccccctaagctagtcctggctgtgcactctcaggtccgggttgcttggctgaacggcgtggcccgaagcaacggagaaccacaacaaagacagagggggatgctgtgcgctggtgcttgggggcgggggaggagtcgccccggaggtttctcgctgctgctgtttttcttccttttctttcaacgagggtgggggaggtggcaaacgttctttcagcctgagcttattagggaccctgcccgaagagtgcgggagaacggaggtctgataaggccgggaggacggatggagagcgtgggcagcggtggttgtttgtgagattgtttactacgttcaggagcaaggcgggtagggatgtggccgccttccccgggaaggacaaagccgaggtgctggaaggggtggccgttgcgccgcctccttttcggtccaacacctttctctcttttggtctttggtttcagtgctgatggaatggaagcatcttgcatattttatgcaaaaagtgatggcaaaagaggttgccacggccagggaagggaaaggaagggagttggggtaggggtaagactgatagatcgctgtagtatttggagaggcgttttcagctcagttttccaactaccgggttccactcgatctgggaaatacttgaatctctagatatattaatattatcctaaagcatttccttagacacttttctaagttagaactctctatgtccctcgtccccttctctcctcgctttccttttcctcgggtgaaatggttcttggcatatttctcacgcatgtctattgcgccttcgcctctgcaaagccaccgctgggttgctgagacccgctctgccaacccggctactggaggggtttccttgaacttgaagaaaggtacatcaaaacccactagtgttaactgccacgtcaagtttgatgctaataatgtgcagattatgacgaaaattgccaatcatgctctctgatggacctcctttgaatgtggaattggagaaaagttccccgcacggagacctgctgtcaagtactaacaacccgctaagggaagtcattaggagagacggataagagactcggtacataaaacattgttcttggtgcatagaatgtatgttatttaatgttatctctgttacctgaagtgatttcgcagaagaaagccacgttcttatcatctcaattgccgattttcattttggtaacttggacgccctgggtagacttttctctgttaagttgatattccaccagtgtgaacagcctcattaaatcaagcccagatatttccataatgctccccgcagagccacttggctcctcacataatgagattttctgaagagttgaagccctaaaataacttgctagtgcatgtttagacccagcgaagtgggtatgtgtgtgtacgtgtattaaaatgtaggtaggctacctacatgtgtttgtgtatgtgtatatatatgtatatgtatgtatatgcatatatatatccgtgctcttaaatacatatgtaagagatacacacatattcatgtatacatacacattggcactattttctgtggtttatttcagatttgtgtcctgccatcatattgttattttaaagacactggattttaaaatatcatttcctaaatataggctttcagtaaacttttgaaagaattagttttccaggagaaattatttccatcaaatggaaaatacataaaggggaaacagtatttcgaaattcctggggtctattaaaatattaccataaaatattagagaactttccaatgaaatagcaatgatctaaaacttaagtttttgtttttgtttttaaaaacatgagactacttatgtaaaatattgattataccagctcttattcactgagtttacatttttaaaaatattaatatataatttaaggctaaataaaataaaccaaaatatggtaccctttatcagctttacctatttagattgttaaagtcctttgttatgatcagctaacagatgtatataaggtactttaggcagtaatgtttacatttcccccaaaaaatatatatgattgatgagaacgctggttggtaaaattaacataaattcatcatttgttgggtccattggatctggttattatattgaaataaaattgtaaataaagtcttcgtgctttaaatattgctggatgtatgaactcactgtaagatattttacaaatgtgcaataattataaagccttgtatattacgttatttattgtgtttggtcatgtaaaataaatgttatttaaatcaa

SEQ ID NO: 46 - DNA, 1447 bp, Homo sapiens
  source 1..1447: mol_type = genomic DNA
agtcccaagtgctcaggacagaggctggacgggctccttcctgcacccctgctcttgggacacagtgccctccggctctaataggccaggagttgggggggcaactggctctgctccctggccctggctccccctgctccctggggctgcccgaggtgaaggtgacaggtgggggaggcaggtggagaattgggccagtgagctcatggcaaaggcgcccagctgggcaggggtgggtgctctggcctataaagcccccgaggccctgtggcctgcagaggcggttatggacggcaccatggaggactccgaggcggtgcagagggccacagcgctcatcgagcagcggctggcacaggaggaggagaatgagaaactccgaggagacgcacgccagaagctgcccatggacttgctggtgctggaggatgagaagcaccacggggctcagagtgcagccctgcagaaggtgaagggccaagagcgcgtgcgcaagacgtccctggacctgcggcgggagatcatcgatgtgggcgggatccagaacctcatcgagctgcggaagaaacgcaagcagaagaagcgggacgctctggccgcctcgcatgagccgcccccagagcccgaggagatcactggccctgtggatgaggagaccttcctgaaagctgcggtggaggggaaaatgaaggtcattgagaagttcctggctgacggggggtcagccgacacgtgcgaccagttccgtcggacagcactgcaccgagcttccctggaaggccacatggaaatcctggagaagcttctagataatggggccactgtggacttccaggatcggctggactgcacagccatgcattgggcctgccgcgggggccacttagaggtggtgaaacttctgcaaagccatggagcagacaccaatgtgagggataagctgctgagcaccccgctgcacgtggcagtccggacagggcaggtggagattgtggagcactttctatccctgggcctggaaatcaatgccagagacagggaaggggatactgccctgcatgacgctgtgaggctcaaccgctacaaaatcatcaaactgctgctcctgcatggggctgacatgatgaccaagaacctggcaggaaagaccccgacggacctggtgcagctctggcaggctgatacccggcacgccctggagcatcctgagccgggggctgagcataacgggctggaggggcctaatgatagtgggcgagagacccctcagcctgtgccagcccagtgaatgcgtgccccagcccagccagctacccagcccctctctgtgtgcagccggagggtcctaagaatggctcccggagctaactgagggcccagccttttttctgcatgatccaggagcacataccacaaactaccacaataaaaaagctgtttttgctaa

SEQ ID NO: 47 - DNA, 5501 bp, Homo sapiens
  source 1..5501: mol_type = genomic DNA
gtggctgtggctgcggcggcggcgcaggagcagaaaccggtgcggcgcagaagccccagaggcgggcggggcggagcggagggccagcgggccccggcgtgggtctcccgccaggccgccggatttaacccttagtggcttctgaagaaaatggcagaaacatcgccagagccttctgggcagcttgttgtacactcagacgctcacagtgacactgtgctggccagttttgaggatcagaggaagaaaggcttcctctgtgacattactttaatcgtggagaatgtacatttccgggcccacaaagccttacttgctgccagtagtgaatacttctcaatgatgtttgcagaagagggggaaatcggccaatccatttatatgctggaaggcatggttgcagacacctttggtatcctgctggaatttatctacacaggttatctccatgccagtgagaaaagtacagaacaaatcctggctactgctcagttcttaaaagtctatgacctggtaaaggcttacacagacttccaaaataatcatagctccccaaagccaacaactttgaacactgctggtgccccagtggttgttatctctaataagaaaaacgatcctccaaagcggaaacggggaagaccaaaaaaagtcaatacattgcaggaggagaaatcagaactggctgcagaggaagaaatacagttaagagtgaacaattcagttcagaatagacaaaactttgtggttaaaggagacagtggtgtactgaatgagcaaattgcagcaaaagaaaaggaagaatcggagcctacttgtgagccaagtagagaggaggaaatgccagttgaaaaagatgagaactatgatcccaagaccgaggatggccaggcaagccagagtcgatacagcaagcggaggatttggagatccgtcaaacttaaagattacaaacttgttggggatcaagaggaccatggttcagccaagaggatctgtggaaggagaaagcgccctggaggccctgaggcccgctgtaaagactgtggcaaggtctttaagtacaatcactttttagcaatccaccagaggagccacacaggggagcgacctttcaaatgtaatgagtgtggaaaaggctttgcccagaagcactcgctacaggtccacaccaggatgcacacaggcgagcggccgtacacctgcaccgtgtgcagcaaggctctgaccaccaagcactcactgctggagcacatgagcctgcactcaggacagaagtcttttacctgtgatcaatgcggaaaatatttcagccagaacagacagctaaagagccattaccgagttcatacaggccactcattaccggaatgcaaagactgccatcgcaaattcatggatgtgtctcagctaaagaaacatctgcgaacacacacaggtgagaagccatttacttgtgaaatctgtggcaaatctttcacagcaaagagttctcttcagacccacatcagaatccatcgaggagaaaagccatactcctgtggcatttgtggcaaatccttctctgactccagtgccaaaaggagacactgcattctacacactggcaagaagcctttctcctgccctgagtgtaacttacagtttgctcgcttagacaacttgaaggctcacttgaaaattcatagcaaggagaagcatgcttcagatgccagcagcatttctggcagtagtaatacagaagaggtcaggaatattcttcagctacagccatatcaactctctacctcgggagagcaggaaattcagcttctcgtaaccgattctgtacataacatcaatttcatgcccggtcctagccagggaatcagcattgtgactgcagagagttcccaaaacatgactgcagaccaggctgctaatcttaccctgctcacgcagcagccagagcaactgcagaatttaattctttcagctcaacaggagcaaacagaacacattcagagcctcaatatgattgaaagccagatggggccctcacaaacagagccagtgcacgtgatcactctgtccaaggaaacactggaacatcttcatgcccatcaagagcaaacagaggagctccatttagctacaagtacttcagatccagctcagcacctgcagctgacacaggagcctggtccgccaccacccactcaccacgtgccccagccaacgccacttggccaggagcagagctgacctgtaaacacgtttgactgcttgattgggctcttcttataagccagctctgactggattttgaacgcttgaaatcaggctctccacaccattgacttgcagatgcttctgtgagagctgtgatggctaactgatgtttataatgccctccatccaggtagcaagatgtgtgtatgtcttgtctaaggtactgatttgtattgagcatttcaatattattgaatggttttcattttaatctgcaatgtctcataaagactcgggtagtttttctttgggggcattgatctcagtttttctgtaaggtttaaatgttcacagccataggttatggtcttgccaacctccccagttatgtttttttgttttctaggacaaagggagtcattttggttgaactgggcagaatggtttgttctattttcttatttttcttaggcaacacagttgttaccacaagacaagtcagggctaaatatgacagatcacaatgtatggggaatttgcccttttaataatttccagtcccctaagacaaagtaaccctcaacgttttatctttatgcagtacagtgggcttattgtgttaacacttcagaaagattcaaactcataaactcagtattttaactcaaaattcatgtaacaagatagccaaaagcataggatttcttaaaattctgaattcgtcttttctgagaaacataaatttacatatttttcaacagaaggctatcaaaattttaattaagtttaaaatacaaaattggatcaagacattcaccctaaattctgaatttttgtcagaaaagagatttgattttcatgaaggtaaagctttcttcactattctggaacataaaactttattcagtgtaatttaagtcttatatatactctactgaattctaactgatctcctgaaaatgttgatctacgtgttgtgtctgataactttctggagatggaagcttataattcatttttattgcagactagttaaatcccacaaagtgaatatcttctcctaatctttccttttttagagacagggtctcactgtatcacccaggccagagtgtagtggcacaatcattgctcactgtaacctcgaattcctgggctcaagcaaccctcccaccttagcctccctagtagctgggactacagggatgtaccaccacacccagctaatcttcatctaatcttaaaactttgagcctcagagactttgtagccagcatgtgtctgtaattgtatgatttttaaatcatcatcaaaagattaaattatgtttgggttaggttttcaagctaagcttattgaattcttttaccactttcatgtaagctggtaggggatttttgagaaaagaaggtgggcactagtatatctaaattaacaatgctgatggggagtagggggtaatctactctaaaattaatagagcctttccatcaattgttttgttttagacatccttgatttctccctaaatcaaataagttatttggaggttagagtaatagagaattcttcaggtttgagagaagataaaaacattttggttgcatatttgctaagctatattttattatatgtaattcacaaaacttccaaaatgatatatatcttatatgtatcaaccaaactaatgttaaatcaggcctgactagagcaaagataacaattttttacaaacactgttaagccctaagtattatttcaggttctcgtttaaactgtttaattttattcttaaaacacaaagtttttaccaacttttcaaagcccaccaatccacattttctccaggaaattaaatatttaagtaagtgagtgtttactcactctggatagctaggtgattattcagaaaatacggttacatagctatttataagtcaagtttcactggctctataacagtctacctctacactaagagaactataacatgaacaacataggtcactttaaatgttctagtagcaacttttaaaaacataaaaagaagcaggtgaaaatgttaataaaagtttatttaatccaacatttgccaaatatttcaacatataaaaatatattagtgagacattttacattctctttttcatactaagctttcgaaatctggtgtgtattttacactttctgcacatctccatttggatgctaatgttcattggacatacttgatctgtatttagatttcatacaacttacagttgaaaacagattctcatatcaagtagctaagacaaaagtttttttcaagaattgaaataagtatcagtttaaaaatttatattaaatttattaaaattaaaatccagttccttgttgcatgggacacagtgcaaatgcttgataagcacatgtggctagtggctgctatattggacagcatgggccacctgtttctaacttgagcatcagtcctagctatgcctgaattatttgaccagctgtcaaagagaaacaaggaaatgatagctttgttaatctagcctctttctttgtcccacatatacattctacccctttgaataaaaaagttcaatgacgccgggcgtggaggctcatgcctgtaatcccagcactttgggaggccaaggtgggcggatcacgaggtcaggagttcgagggcagcctgaccaacatagtgaaaccccatctctactaaaaatacaaaaattagctgggcgtggtggtgtacccctgtaatcccagctactcgggaggctgagacaggagaatcacttgaacccaggaggcggaggctgcagtgagctgagatcacgccattgcactcaagcctggcgacagagcgagactccgtctcaaaaaaaaaagttaaatgacttgagtacccaaaagaaatgtcgtgaacatgaaagtacttctcatgtgtccttgagaaatgatgttgctagtgcagtgtttcacaattcattttctactaaatgtgtaccattttttaaattgttttaacagaaatatagagtgtgtttcttaggaaatagaatgttatgctttggggctcctgaaacacctcgttttgaaggtgaatctttggttttctcccagaagagggtctgccttgggatgattggagctcttaccatcaaaatcagttattcaagtgcttgcccagtgcttcttgaaactcacctctactctgtattttctacagttggttcttgtgttataaatgttgtgagcaacagattaaatgatgctgaggaatgaaaaaa

SEQ ID NO: 48 - DNA, 4416 bp, Homo sapiens
  source 1..4416: mol_type = genomic DNA
gcgccgcttttgaggggcgaaccaagatggcggcggttttggctgtgtgaggaagacggaagagacggcggcggagggaaaccgacttccactagtccgggtcgcttgggcggccgggggccctcagagtctcccgggcagtggtagcagttgcagcaggatcaggcgcctgtcggcttctgacgtttaaaacagggggagcggaagggagccactggccgcggtggcagggccaggtataaggaaggaaaatatggcggcggcggcggcggcctgaggaggcggcggcggcgcgggaagctgcttcgcggagatcatggcggaggcgggagcagggcagtgacgggagccccgagttcctagcgctgcggggcgggaggctacgaagcgctgcgcggccccctcggggctgccgggcgccgggctcgccaggcctggacaatagcgccggggagccggaggcgaggaaaggcggcggcccagagctcggtccctggagcgggccatgcaggcggcggcgcggccccgcggcgcccagcggcggcagtgaggccggggagccctccgctcgcgggcgccctcacgcctcgcccctcgcctctccagggcccctttcctgggcgtctactggcggggcccccgccccggttcccgggcggcacgatgaccgacacccggcggcgggtgaaggtgtacacgctcaacgaggaccggcagtgggacgaccggggcaccgggcatgtgtcgtctggctacgtggagcggctgaagggcatgtccctgcttgtcagggctgagagcgacggttctctacttttagagtcgaaaataaatcctaacactgcataccagaaacaacaggacactctgattgtgtggtctgaagcagaaaattatgacttggcccttagctttcaagaaaaagctggatgtgatgaaatttgggagaaaatatgtcaggttcaaggaaaggacccttccgtggacatcactcaggaccttgtggatgaatctgaagaggagcgttttgatgatatgtcatcgccaggcttagaattgccatcttgtgaattaagtcgccttgaagaaattgcagaacttgtggcatcatctttaccttcacctcttcgtcgtgaaaaacttgcactggcactagaaaatgagggttatattaaaaagctcctggagctttttcatgtgtgtgaagatttggaaaatattgaaggactgcaccacttgtatgaaattatcaaaggcatctttctcttgaatcgaactgctctttttgaagttatgttctctgaagaatgtataatggacgtcattggatgtttagaatatgatcctgctttatcacaaccacgaaaacacagggaatttctaacaaaaacagccaagtttaaagaagtgattcccatatcagatcctgagctgaaacaaaaaattcatcagacatacagagttcagtatatacaagatatggttctaccaactccttcggtctttgaagaaaacatgttatcaacacttcactcttttatctttttcaataaggtagagattgttggcatgttgcaggaagatgaaaaatttctgacagatttgtttgcacaactaacagatgaagcaacagatgaggaaaaaagacaggaattggttaactttttaaaagaattttgtgcgttttcccaaacgctacagcctcaaaacagagatgcttttttcaagactttgtcaaacatgggcatattaccagctttagaagtcatccttggcatggatgatacacaggtgcgaagtgctgctactgatatattctcatacttggttgaatataatccatccatggtacgagagtttgtcatgcaggaggcacaacagaatgatgatgtaagtaagaagttaacagagcaaaaaataaccagcaaggatattttgctcatcaacctcattatagaacatatgatttgtgatacagatcctgaacttggaggagcagtccagcttatgggcctgcttcgaactttagttgacccagagaacatgctagccactgccaataaaacagaaaagactgaatttctgggtttcttctacaagcactgtatgcatgttctcactgctcctttactagcaaatacaacagaagacaaacctagtaaagatgattttcagactgcccaactattggcacttgtattggaattgttaacattttgtgtggagcaccatacctaccacataaagaactacattattaataaggatatcctccggagagtgctagttcttatggcctcgaagcatgctttcttggcattatgtgcccttcgttttaaaagaaagattattggattaaaagatgagttttacaaccgctacataatgaaaagttttttgtttgaaccagtagtgaaagcatttctcaacaatggatcccgctacaatctgatgaactctgccataatagagatgtttgaatttattagagtggaagatataaaatcattaactgctcatgtaattgaaaattactggaaagcactggaagatgtagattatgtacagacatttaaaggattaaaactgagatttgaacaacaaagagaaaggcaagataatcccaaacttgacagtatgcgttccattttgaggaatcacagatatcgaagagatgccagaacactagaagatgaagaagagatgtggtttaacacagatgaagatgacatggaagatggagaagctgtagtgtctccatctgacaaaactaaaaatgatgatgatattatggatccaataagtaaattcatggaaaggaagaaattaaaagaaagtgaggaaaaggaagtgcttctgaaaacaaacctttctggacggcagagcccaagtttcaagctttccctgtccagtggaacgaagactaacctcaccagccagtcatctacaacaaatctgcctggttctccgggatcacctggatccccaggatctccaggctctcctggatccgtacctaaaaatacatctcagacggcagctattactacaaagggaggcctcgtgggtctggtagattatcctgatgatgatgaagatgatgatgaggatgaagataaggaagatacgttaccattgtcaaagaaagcaaaatttgattcataataatggcaacggcctaggatcagtacctgttgaaaaaaactggttctccacccctcccccatacaaaatccacaacaaagcgcagtggtctcttgtgaatgactgacacagatcagcctcttacacttgacttctgctcatcaagtgccaattcaatggagcaggaggaggggatatcatatatttaggggaaagacttaagcctttgagctctccagcttggaccacacattgcccttttctcagggaaggaaatggaaacaaaaagccaacagggcaggggttttgtaagtggaactctggattgactggtcagttgctacaatcagaatatgctttcttggaccatgtttgagactcagaagaatgggcctttctgccataattcttcactagtcaagaatgccagcagtttctttgtataaagagacctgcctttaaaatcatacattctgaacattttagtcaagctacaacaggtttggaaaacctctgtgggggaggggcgagtataaagttttcctcttttttaactgttccctttgcccttcaaactgcagatatttttttttttaagtggggacttctccctacttgattaaagattgagtggaattctagatgtggtcatttgtgtcataatttttttgttttattttgtttttgattttttttttcctcccctgagtgtatgcttagttgttgagtatatatatttgggaccattaaaactttttttgatgtaatataacctaacgttgtgctggtacctgttttaccatgtgtaatttttgttctacatcacagttcttaatttgtttagagttttatgaaagatggtatagtttttattgacaaaagcaaagtaatcttacaactatgtgcatacaaaagcaatactattttgtgactaaatattttatattaaaatttacatcagcaactgtcttgagaattcagggaaatagaatggaatttaaaacttcaacagttttgttaaatctagaaacatgaaattagtattccaaagagattctgaaatttcttttcttggggaaatgacggtacattaaatcaaaattgaggatggatgatttaaaaacatttgacttttgaataataaaaagaaaagtgaagagtaagagaaattgtattagttgtatgtttcttgtttttgtttttaactactccctttggaataccagtttgacttgtaagtggtggtttctaaataaatacctggccagagga

SEQ ID NO: 49 - DNA, 2440 bp, Homo sapiens
  source 1..2440: mol_type = genomic DNA
actgtgctccattgctctggagccagcaggggaagtgtgtacactatgtgcactgagggtgacacttctgagtgtgggggccctggctagggagtgggactggcaggcttgggtggggttcccaagctttaatcttcctaccccacagaatgttttctctgcccttcaggcagcctgagccgggcagggcccctgcctctgctacggcagcccccgatcatgcagcccccactggacctcaagcagatcctgcccttcccactcgagccagcccctacccttggcctcttcagcaactacagcaccatggaccctgtgcagaaggctgtgctctcccacacttttgggggacccttgctcaagaccaagcggcccgtcatttcctgtaatatctgtcaaatccgcttcaattctcagagccaggctgaggcgcactacaaaggtaatcgccacgcccgacgagtcaaaggcattgaggctgccaagaccagaggcagggagcctggcgtccgagaacctggagacccagctcccccaggcagcaccccaacaaatggggatggtgtagcaccccgtccagtttccatggagaatggactggggccagccccaggatccccagagaaacagcctggctccccatcccctcccagcattccggagactggtcagggtgtaaccaagggtgaaggggggactccagccccggcttccttgcctgggggtagcaaggaagaggaggagaaagccaagcggctgctctactgtgctctgtgcaaggtggctgtgaactccctgtcccagcttgaggcacataacaaaggtactaagcacaagacaattctggaggcccgaagtgggctcgggcccatcaaagcttaccctcggctggggcctcccaccccgggggaaccagaggctcctgcccaggaccgaactttccactgtgagatctgcaatgtcaaggtcaactcggaggtccaactgaaacagcacatctccagccggcggcaccgagacggcgtggccgggaagcccaacccactactgagccgtcacaagaagtctaggggcgccggggagctggcgggcacgctgactttctccaaggagctgcccaagtccctggcgggcggcctgctccccagccccctggcggtggctgcagtgatggcagcggcagcaggctcgccgctgtccctgcgcccggctccagccgcacctcttctccagggaccgccgatcactcaccctctgcttcacccggcccccggacccatccgaactgcgcacggacccatcctcttctccccgtactgacctcaaccctgaacccctcccattcaactccccacctccagccgggacccaggcgtccgggctcccagcccgcccctcctcccggcactccctaaatgatctctccttccccccccccaccccgagatacggggttccaggaaaggggaggggtagcgggggaggggggcttcagaagggggggaacaccccagatctcagggaaccccgccccctgcctttccctctcccctagaaaagggggggccgtctcacccccgagcccccttggagacaccccccctcccaaaagccatgtccatccagcccttccccccaaacctagcacaaaacggggttcacaagccatggtcggggtccgggggggacagaaatggattttcttggcaataagcggactctgggactccggctccctaccccaaactgaagcgcttccgtgaacacccccgtcctccgtagggggaggggagcaggcgggatcctgggtccctcataagcactttggttttaccgcctgcaacctcactgtgcccgccccgcaccatgccctagccccaggtctagccgggcccattgcagggggcagcacttgggggcatctccggcacttgggtgggaccaaggagatgccaccatagacctttccctcgccttcttcctccctagtccgggttccattcttttcaccagcacccatcgcccaaggggtaccgaggggggcaaggggtgtccagtccaagcccacccccgcctcgccttccgcaaaactgtgagcaaaaagcaatagaagcctcgcccccgcccttccccttcgcaggattcgccgagtctgtagcctccccgatccaagttcctagacctcatggctgtcccctcccaccagtcacctccactgcacaactcgggcgggggtgtgacacctctcccccacccccgactccgtggtttccgtatcgtcaacccttcagccgccgacccgggaggggtctggcctacactggtcttccccttcccatcaactctttctgcttgacaatgtagcaacccaggccccccacccacggtcctcccctttttcctctccctgacaataaagtctgaatttgttctgccctcc

SEQ ID NO: 50 - DNA, 2050 bp, Homo sapiens
  source 1..2050: mol_type = genomic DNA
ctttcctaccctcaacgccgttccggggggccgggcctggcaagtcctcttggaacgcctccctcttgccttccccttttggggacagatcccgaagttcgagcatccctcggataggccgggtgtcaggcctggtctctcaggcccgtccaggcccatcttgacgattccaagaccacccccttgagcaagaatgccatcccctctgggtcccccatgcctgcccgtcatggacccagagaccacccttgaggagcctgagactgcccgcctccgcttccgagggttctgctaccaggaggtggcaggtccccgagaagccctggcccggctgcgtgagctgtgttgccagtggctgcagcctgaggcacactccaaggagcagatgctggagatgctggtgctggagcagttcctgggcacactgcctcccgagatccaggcctgggtgcgcggtcagcggccaggcagtcctgaggaggccgctgccctagtcgaaggactgcagcatgaccctgggcaactgttgggctggatcacagcccatgtcctgaagcaggaggtgctccctgcagcccagaagacagaggaaccacttgggagcccccacccctcagggacagtggagtcccctggggaaggtccccaggacaccagaatagaggggtctgtccagctcagctgcagtgtgaaggaggagcccaatgtcgatggacaggaagtggcaccctccagccctccacttgcagcacagtcccctgaggggaaccatggacaccaagaaccagcctccacatccttccacccacccaggattcaggaggagtgggggctgctggaccggtcacagaaggaactgtactgggatgcgatgctggagaagtacggcacagtggtctccctggggttaccgccccaccagccagaggcacaggcccagtcagagctggggatgctgctcacggggacaggcgtctgcagaagcctgcgctcgggaaatgagagtgagggtccacctggctgcccagaggcccagccgccccagggcccagggccggcagcctgggagggcttgtctggggctgccactcctgcccccactgtgcgcccagggacaccgccagtgcccactcagcccacacctgcagagacgagactggagccggctgccacccccaggaagccctacacgtgcgagcagtgtggccgcggcttcgactggaagtcagtgttcgtcatccaccaccggacacacacgagtgggccaggtgtgcagtccccggggctagccaccggggaaagcacagagaagccaccacaaggggaggtggcctttccgcaccacccccgacgctcactcacaggcccccggagttacccgtgtgaggagtgcgggtgcagcttcagctggaagtcgcagctggtcatccaccgcaagagccacacaggccagcggcgtcacttctgcagtgactgtggccgcgccttcgactggaagtcgcagctggtcatccaccgcaagggccaccggccggaggttccatgagcagccagacagcacagtccctcggggcctcggtgttctcggggcctggatacagcctctggggcaccagcagaagactctggaggcagcaggggatgccagagtgaacaaggggtcccaagccagttccctgcccctggtctggtctcccccaaaagacctgggtgcaaggaaaaggagctgctctctctcttcttgcccctgcctcctagagggaggtctgggttcccttctatggctgaccagtgcctgtggggtgactgccaagcaccaggctccctccctccctgtgacatggcctgggctgacaacactccctctcctgggacctccttgcctcaggtgggtgttcaaaaactgtgccttcccactcgtctgtgcagaggctgggcctgaggtctcagtgtggagagcagcagaagacccaggaaagcacagttggcttccgtttctcctgctccctgtgtgtgttagaattttaacataaattccactttcataata

SEQ ID NO: 51 - DNA, 1183 bp, Homo sapiens
  source 1..1183: mol_type = genomic DNA
gagtcggcgccatgaccccatcgaggcttccctggttgcttagctgggtctcggccacggcgtggagagcggcaagatcaccccttctgtgtcattctctgaggaaaacaagttcttctcaaggaggaaagtctgaacttgtcaaacagtcccttaagaagccgaagttaccagaaggtcgttttgatgcaccagaggattcccatttagagaaagaaccactggaaaaatttccagatgatgttaatccagtgaccaaagaaaaaggtggacccaggggcccagaacctacccgatatggagattgggaacgaaaaggacgctgtattgatttttaagtcgcatattctttaacttcaatattgttttctgaatatgtacatctgaattaacttatttctgattattttctttctttatatcctttatgtcgtgtagtttgtataatgtgtttaaatatatatatatatgatggctttggaagaaaatatgctgctgtaaattaggaaagggagaccagcctgaccaatatggagaaatctcgcctttactaaaaatacaaaattagccaggcgtggtggcatgcacctgtaatcccaactactctggaggctgaggcaggagaatggcttaaacccaggaggtggaggttgcggtgagctgaggtcgcgccattgcactccagcctgggcaacaagagcgaaactccgtctcaaaaaaaaagataggaaagggaaaatattttttcattaacatatattaatttaatgagtatagaaatgattcttgtttttttgagacagagtctctctctctcacccaggctggagtggagtggcactatcccaactcactgcaacttccgccccgccaggttcaagcaattctcctgcctcagcctcctgagtagctgggattacagacacccaccattgcgcctggctaatttttgtatttttagtaaagacaaggtttcaccatgttagccaggctggtctcgaactcctgacctcagttgatccacctgcctcagcctcccaaagtgctgggattgcaggtgtgagccaccacgcccagcctagaaatgattcttagagctgtaggcctttacttcatcatttttcagtttaaattaatcagttgcttataaaatgcatacataaataaaataatttttaaaaaa

SEQ ID NO: 52 - DNA, 575 bp, Homo sapiens
  source 1..575: mol_type = genomic DNA
gagtcggcgccatgaccccatcgaggcttccctggttgcttagctgggtctcggccacggcgtggagagcggcaagatcaccccttctgtgtcattctctgaggaaaacaagttcttctcaaggaggaaagtctgaacttgtcaaacagtcccttaagaagccgaagttaccagaaggtcgttttgatgcaccagaggattcccatttagagaaagaaccactggaaaatctttcatttgcaggccggatgtcgtggctcataactgtaatcccagcattttggaaggccgaggtgggtggatcccttgaagtcaggagtttgagaccagcctggctaatatggtgaaaccctgttctactaaaactacaaaaaaagtagccaggcatgctggcgggcacctgtaaccccaactacttgggaggccgaagcaggagaattgcttgaacccaggaggcagaggttgcagtgagctgaggtggagccactgcactccagcgtgggcaacacagtgagactctgtctgaaaaaaataaaaataaaggaaaaataaaatatcttttatttgtagaaaca

SEQ ID NO: 53 - DNA, 1614 bp, Homo sapiens
  source 1..1614: mol_type = genomic DNA
agtagagttttgctgttaagactgcgcgaggagctagagagagcggagagcgctgagccgggccgcacccgccgagccgtgaaaaaagtacatctcctggaagggatgctttttagctgagctctggtggatgagaggagctagccttgaaaaacttgatactgatggacattgtgtgggccagaggcagggatggttggctatgaccccaaaccagatggcaggaataacaccaagttccaggtggcagtggctgggtctgtgtctggacttgttactcgggcgctgatcagtcccttcgacgtcatcaagatccgtttccagcttcagcatgagcgcctgtctcgcagtgaccccagcgcaaagtaccatggcatcctccaggcctctaggcagattctgcaggaggagggtccgacagctttctggaaaggacacgtcccagctcagattctctccataggctatggagctgtccaattcttgtcatttgaaatgctgacggagctggtccacagaggcagcgtgtatgacgcccgggaattctcagtgcactttgtatgtggtggcctggctgcctgtatggccaccctcactgtgcaccccgtggatgttctgcgcacccgctttgcagctcagggtgagcccaaggtctataatacgctgcgccacgccgtggggaccatgtataggagcgaaggcccccaggttttctacaaaggcttggctcccaccttgatcgccatcttcccctacgccgggctgcagttctcttgctacagctccttgaagcacctgtacaagtgggccataccagccgaaggaaagaaaaatgagaacctccaaaacctgctttgtggcagtggagctggtgtcatcagcaagaccctgacatatccgctggacctcttcaagaagcggctacaggttggagggtttgagcatgccagagctgcctttggccaggtacggagatacaagggcctcatggactgtgccaagcaggtgctgcaaaaggaaggcgccctgggcttcttcaagggcctgtcccccagcttgctgaaggctgccctctccacaggcttcatgttcttctcgtatgaattcttctgtaatgtcttccactgcatgaacaggacagccagccagcgctgagtgcaggaaggaccccaggtcttccctggaggcagcctcctgaaggaaggaagattcagtctccactgagaggtgccgtctggcccttccctgcaggccagctgccccaagcggggtagcagccttgaacccacccagctgggacaccaccagaaggtccagggctctccccatgagagaatcagagggaatgcaggacgtggtctatggtgagccaacgacacagtgagaaggagcaggaagttgctgtttctcctctgaccagcccacactccaaaggaaacagacgccatcctacacctatcagccctgcctgccaggagaacagaacacactcctggtctggatggggctgctgcttgagtgcagagggctgcggtaggccctttgcaggagtcaggtccctacacttggcctgtttttccaacctatttaatagacattaaagctaaacacaca

SEQ ID NO: 54 - DNA, 1308 bp, Homo sapiens
  source 1..1308: mol_type = genomic DNA
acttccgtccagaccggaacccaagatggctgcgctgttgctgagacacgttggtcgtcattgcctccgagcccactttagccctcagctctgtatcagaaatgctgttcctttgggaaccacggccaaagaagagatggagcggttctggaataagaatataggttcaaaccgtcctctgtctccccacattactatctacagttggtctcttcccatggcgatgtccatctgccaccgtggcactggtattgctttgagtgcaggggtctctctttttggcatgtcggccctgttactccctgggaactttgagtcttatttggaacttgtgaagtccctgtgtctggggccagcactgatccacacagctaagtttgcacttgtcttccctctcatgtatcatacctggaatgggatccgacacttgatgtgggacctaggaaaaggcctgaagattccccagctataccagtctggagtggttgtcctggttcttactgtgttgtcctctatggggctggcagccatgtgaagaaaggaggctcccagcatcatcttcctacacattattacattcacccatctttctgtttgtcattcttatctccagcctgggaaaagttctccttatttgtttagatccttttgtattttcagatctccttggagcagtagagtacctggtagaccataatagtggaaaagggtctagttttccccttgtttctaaagatgaggtggctgcaaaaactccccttttttgcccacagcttgcctactctcggcctagaagcagttattctctctccatattgggctttgatttgtgctgagggtcagcttttggctccttcttcctgagacagtggaaacaatgccagctctgtggcttctgccctggggatgggccgggttggggggtgggttggtgaggctttgggtgccactgcctgtgggttgctggcttaaaggacaattctcttcattggtgagagcccaggccattaacacctacacagtgttattgaaagaagagaggtgggggtggaggggaattagtctgtcccagctagagggagataaagagggctagttagttcttggagcagctgcttttgaggagaaaatatatagctttggacacgaggaagatctagaaaattatcattgaacatattaatggttatttctttttcttggatttccagaaaagcctcttaattttatgctttctcatcgaagtaatgtaccctttttttctgaaactgaattaaatactcattttatctttgactctccttgaaa

SEQ ID NO: 55 - DNA, 1605 bp, Homo sapiens
  source 1..1605: mol_type = genomic DNA
gcgttcgcagccgccgtcatcccgcggaggagcgcgcagccccggggaggccggaggacgcgcccatagaatgcccaggggcgacaaactgtcctgagcccctctggtgcagccacctgcctgtcccatacgccccgcccaccatggagtccagagggaagtcagccagcagccccaagcccgacaccaaggtgccccaggtcaccaccgaggccaaggtacccccggcagccgatgggaaagcccccttgaccaagccctcgaagaaggaggccccggccgagaagcagcagccgccagcagcccccaccacggcacctgccaagaagacctcggccaaggccgaccctgcccttctcaacaaccacagcaacctgaagccagcccccacggtccccagcagtcccgatgcaaccccggagcccaagggtcctggggacggggcggaggaagatgaggctgccagtggggggcctgggggccgaggtccctggtcctgtgagaacttcaaccccctgctggtggctgggggtgtggccgtggcagccatagccctgattctcggtgtggccttcctggtccggaaaaaataatacctgggggccaggcggggggcacggagccacttcctgtacagacccgaggaagccagtgcatgcagagttcacccttacctattcgtacacacgcacattcattacacacctacatacgcccccaacacacgcgcacggtgaagaggacgcccgagcccacccctgctgacccaggacttccccaacctccagggcagaaagagcccaggctccggggtccacagcacaggatgtggggcgagggcacagctggggaacggcaagaaaggaatggaccctgtgtgtggccccccccacccctggcggctgggtgatcctgggcccccagggctggtctgagtgcaggtgggggtgccgggatgggttgggcctgggcccggccctcgtggggacattaaagggcgcggtggctccactcgccccatttctgctctttgcgtaccccccgggggtctgcctgggcgaatgcagtgggacggacctggggtgggcactcacctcagcagagcctcaatgccacctccccacaccacccaagcctagctgggtgggaagacggagccctgaagcctgcgtgtggagggggtgctgcccaagggtcgccttccccagcttgtgctccaggggagccccgacgccggcggggaggcagggatgtcgagggcccctcacagctgtgcccgccctgccccgctagactgcccctccagcttgcacccagcaccacctgagtctaaccagcgtataatgcaataacaggtagagtagaactgcttttggcggcgaccgtcacacactgtcctcagcccactgcacctggggaagcccctcgtgcagcccctccttagcacatccagtcccagcgtggcccttggcgcgcagcagcaggcgaactcccagaaggtggtggtgggcactgtgagaacgtggcctgccctcactggccagtggccacaggaaccccgggcctgagtggaggctgagtctgaaataaactctgtcgtctgaa

SEQ ID NO: 56 - DNA, 9031 bp, Homo sapiens
  source 1..9031: mol_type = genomic DNA
ctctcggccgctcttcgggccgcctctgcgtgtggggccgcccgcgccagtgtgagcctgagctgacggcggctccgggaggctcgcagaaggggagggccgggcggcgcgggagctgagcatcgccagggcgggcggcagggcgcggcctctccgccgggtgtaccacctgtcgcggcgcgagacctctggtgaaagaaaagatgttgtcccggttaagagtagtttccaccacttgtactttggcatgtcgacatttgcacataaaagaaaaaggcaagccacttatgctgaacccaagaacaaacaagggaatggcatttactttacaagaacgacaaatgcttggtcttcaaggacttctacctcccaaaatagagacacaagatattcaagccttacgatttcatagaaacttgaagaaaatgactagccctttggaaaaatatatctacataatgggaatacaagaaagaaatgagaaattgttttatagaatactgcaagatgacattgagagtttaatgccaattgtatatacaccgacggttggtcttgcctgctcccagtatggacacatctttagaagacctaagggattatttatttcgatctcagacagaggtcatgttagatcaattgtggataactggccagaaaatcatgttaaggctgttgtagtgactgatggagagagaattctgggtcttggagatctgggtgtctatggaatgggaattccagtaggaaaactttgtttgtatacagcttgtgcaggaatacggcctgatagatgcctgccagtgtgtattgatgtgggaactgataatatcgcactcttaaaagacccattttacatgggcttgtaccagaaacgagatcgcacacaacagtatgatgacctgattgatgagtttatgaaagctattactgacagatatggccggaacacactcattcagttcgaagactttggaaatcataatgcattcaggttcttgagaaagtaccgagaaaaatattgtactttcaatgatgatattcaagggacagctgcagtagctctagcaggtcttcttgcagcacaaaaagttattagtaaaccaatctccgaacacaaaatcttattccttggagcaggagaggctgctcttggaattgcaaatcttatagttatgtctatggtagaaaatggcctgtcagaacaagaggcacaaaagaaaatctggatgtttgacaagtatggtttattagttaagggacggaaagcaaaaatagatagttatcaggaaccatttactcactcagccccagagagcatacctgatacttttgaagatgcagtgaatatactgaagccttcaactataattggagttgcaggtgctggccgtcttttcactcctgatgtaatcagagccatggcctctatcaatgaaaggcctgtaatatttgcattaagtaatcctacagcacaggcagagtgcacggctgaagaagcatatacacttacagagggcaggtgtttgtttgccagtggcagtccatttgggccagtgaaacttacagatgggcgagtctttacaccaggtcaaggaaacaatgtttatatttttccaggtgtggctttagctgttattctctgtaacacccggcatattagtgacagtgttttcctagaagctgcaaaggccctgacaagccaattgacagatgaagagctagcccaagggagactttacccaccgcttgctaatattcaggaagtttctattaacattgctattaaagttacagaatacctatatgctaataaaatggctttccgatacccagaacctgaagacaaggccaaatatgttaaagaaagaacatggcggagtgaatatgattccctgctgccagatgtgtatgaatggccagaatctgcatcaagccctcctgtgataacagaatagaagcactcccctgataaatactttctgtgctccagggaaccccttttttcagacaagaagagataatgtcttcagttttatggtgttttctgtgttttgttctccctgaccactttggttgatgtattttttccatgcgtctccacatctgttggggtagacgtgttgattgattgcattgcccaccagcaccctacagtcagatagttgtgatgctttaattctaacatacagcccgtaccacatccaggagatgtaaaaagtgtgtttgtgaatgtcttcacttgtactctaattcagacttgccaaagtatttgctatttactattatgggtaatactcttctctggcctagttcttacagagctactaaaatagaaatttacttttatggatagaagtacagaattttgagaagaaactaaattttcaccaaattttaaggaaaaattgtcattatctaaaaatgttcttatatatctgcttcatcttaccttcatactctgaaattccctatagcagacagagctagggaaatattaaaaatttaccctatttattttctggaactaaatcaagccttaactataacattatgagagtaatgggaactactgctggctttaagtaaataaaagtcattgttttcaacagtgtataaaaatcatagtgtaacctttttatttaataaatatcttacatttaattgcttcagttatgctatcttattgcccaactagaaatttagatttgcttatgaaaaacacatatttttgttacttctagatgattctaggagggagtataagatacctaatcatatgggaacatcacgtgactttttaaatctaaattagtcatgcttcacatcaaaatgagtcatatttaactggagaaactatgcccttattccagaactgatgctctttattgcttgagataattttggaatgttactatgatactgtttctttgaatatcattagtagtgacaactctttctccattgaaggcagacttaatttttttaaaccaccaacattaatttagagctaaatggggatgaataaggttggtgttcatctgggaaatgccttttttaatcccagtaaaatgggtaataatggctatccagaacagttttcttgtgtggcttgcatattgaagaattctaaaggaaaatatatctgcctaccatgcttgctcttagggaagaaaacttaaatgctcattctaggatagtaacaacaacagataattcatcgataatatgagcatgcacagtatttgatattgctctgtaggaaaaaagagacttcatttttttcatcttctgcctatgaatacaaattgattctttttttttttttttttttttgagacagagtctcactctgttaccaggctggagtgcagtggtgtgatctcagctcactgcaacctctgcctcccaggttcaagcagttctcctgcctcagcctcccaagtagctgggactacaggtacacgccaccacgcccagctaatttttgtatttttagtagagacgggatttcaccatgttggccaggagggtctcgatttcctgacctcgcgatccgcccacctcagccttccaaagtgctgggattacaggcgtgagccaccgcacccagccaattttttttttttttttttttgagacagagtctcgcttttgtcgcccagtctggagtgtagtggcatgatctctgcaacctctgcctcctcggttcaggcgattcttctgccttagcctcccaagtagctgggattacaggcaccggccaccacacccagctaatttttgtatttttagtagagatggggtttcaccatgttggccaggctggtctcaaaatcctgacttcaggtgatccacccactttggcctcccaaagtgctgggattacaggcgtgagccaccacacctggtccaattcatcataattctgaatataatttaataattttaaaatggtcgcaatggcattgccatttggagtatgggaaaatgcagttggttaattcattgaacagattagtaaagtcagaagtctttttcataacatacggtcacaattcctttcttttaattaaagacaaaaggtttatgggttttggtttgtttgttttagatatggggttttgctatgtcacccttgctggagggcggtggctgttcacaggcgtgggtcatagagcactgcagcctggaactcctgggttcaagctctcctcctgcctcagcctcctgagcagcttggactaaaggtgtgcaccaccgcacctggctgagaggcttctgtttttcatgttccctttatcaaaacaaaatcacgcctaaacatactctctgtgtgaaatcttgattttagttcaacctaagaaatataataaatataaacctgtttcttgcttagtgtttattgaaggcaggaacgtatgctgaattaaagtaattttactacgaagtttgcttattaagtatgcctggtacagtcttcatggaagttattaagtattttaaggaaaaaaataggctaaatggcatatagcctgcttaaataaaataattctatttttttaaaaagctcccaattgtcattgttatttaccagtcaatctttcaacaagtatcaattattggatgtaaattaaagtaaaattgcttaatatttaagctaagaatacaggaaggactatgagcattgaggaaatcgggggaggccagaatggcatcaagctggaaaagaaattaaaacattaccttcgatgagactttacctagcaagaaggtaattattttgaataacagcaatagagaatcttttattttgaattgagaagaaattctgatgattagagtgtcagtaagtagtaggaaaaggtaagaatacatgtgtttctaatctcccaccaccacctgttgtatggaacaaattggattactagcaagggtaaggtttattgacaatgagaagttttagtccaagattttagttaaaatattttaaaattttttgattcataaccaagaagttgcctcagatcactgtgaagctttaaaatgcaggtgcctttgctcctcctccctccaggcccccgggggcagagccctggctggtgtctcttcaggaggacccctgacacaccgattctgccctcacaatcactgccttaaagcaggcagcagaatcttttggagaactaacaagattctgatgctgatgcatagggcctggggtggggcctcagattctgcttttttttttttttttttttttttttttaaacagggtctctttctttttcctttctcactcaggctgcagtgagtggcgtgatcttggctcactgcaacctccacttcctgggctcaaacgatcctcccgcctcagcctcctgagtagctgggactataggcgcaagtctatgcaaccacccagctaatttttgtatttttagaagagtgggggttttgccacgttgcctgggctggtctcgaactcctgacctcaagtgatccaaccacctcagcctcccaaagtgctggaattacaggcatgagccactgcacccagccaagaatgtccacttctgacaagctcccaggtgatgcctatgctgctggtacagtgaacaacatgctgagaggcattgctccaggatactgcatgtagtgagcagttctttagcgtttatgtgtcatatacttcaagttacatgaaaaagatcaactgaaattatgttcttacacagaatagttttcatctagttttattatgtttttacaggctgtttacaagaacaaaaatagagaatttttgaacatatttttctgaaaacataaaaactagagtactctttaattctaaattatagatttgtagcatacttagtaaacttagtaaccaattttcatttgtaaaagattagctatcaaatggacatttatttctcttagttttcttatgacatgacttgccttatatcctttgtcctttttctgtaacttgtcagcctctggagaaataggattttacatttttgcaaatgagttgccaatgaattaagatcaaaacgtcattattaaggagtatgaagcattatttactggctctcagataacagaaaacccaaaagggatggtggcggagtgaagaaagccttgccttgggaaaaatacttcaaatgctcccatagttttttttcctcaagcccagtcagtatccttcaaggataactttttcataccttttttttggtttgtttgtttctgctagctttatgacagaggttataatattcttttctcctctcttctttgacgtgttacgttgtctttttttttttttttttaacttcttcatactcttaaaaagctccagaatggccaggcgcagtggctcacgcctataatcccagcactttgggaggccaaggcaggcggatcacgaggtcaggagatcaagaccatcctggctgacacggtgaaaccctgtctctactaaaaatacaaaaacttagccaggcgtggtggcgggcgcctgtagtcccaggtactcgggagtctgaggagtgaacccgggaggtggaggttgcagtgagctgagatcgcaccgctgcactccagcctgggcgacagagtgagcctccatctcaaaaaaaaaaaaaaaaaaaaaaaaaaaaaaaaaaaaatctccagggttctgttgaattcaccaaaatggggtcagctctggtgagcctatgtaatactcgagaacattaatataaacccatagctctttactgggcttccatttaaagggaagtcctaagaatgaaatggcaccagcatcctgtttccatccaagacaagaccacacgtgttcaggtgccatgtattcatatagctctcaactgattggtgatttgcactttgtgcatctaattattaaaatgaaactctctacaggtgagctgaagtaacatacttcatatttaaaaagttcacagcaatttgaccatagaaaactttctcatctgaaatcagggtaagtttttttaatgtaagcagagaaaaaaataaagcagtgttttatttcaaatttcagtaagttcaggtgggcctaattaagacatgactattattacaaatcctattcttgccagaagttacttagatagattagtgattagattttaaaaacattattttcctttcttgaataaggaaaattgtgcatctcaaagagaaaagtaaatatggaaatatgttcatagttcagttgcataaaatagacacattaagtcaagtgatttgaatgcttagtgtcttttggtaaacagcattttgtcaacaaacttttcatatggctatagaatcttgttttttccaaataaaaactttgcaattgggagttattcctccattcagcacactttttaatgttaagactaggggaaacttgtgggtatatattcagtggttttcaggattttcttttatgttctagaaaagtaatttttcctttcctagttttaagtatatattcaagcaaatcgttgattcctatacagccattggattatcaccaacacatttggtatatacagtgggccgataatgacaaactaaagtttgtggttaatacctactctataccagacactgttctaaggatccgacatatttaatcctctgtttaccctctgtttctgtgcctgatttacagacagagacagaggcacagagaggttaaatgacttgtcaaaggtcttatgatgtgtggcagagcctgggtttttagctcaaacccctctcagtgaagaaagccttctgtgtaggcaatttcatctaataccttcatgaattgttacttcagatgagaattctcacttattcttttttttttttttttttcttttctttgagacagccttgctctgtctcccaggctggagtgcagtggcgcaatctcagctcactgcaagctccgcctcccgggttatgccattctcctgcctcagccttccgagtagctcggactacaggcgcccgccacctcgcccagctaattttttgtatttttagtagagacggggtttcaccgtgttagccaggatggtctcgatctcctgacctcatgatccacccgcctcggcctcccaaagtgctgggattacaggcgtgagccaccgcgcccggcctcacttagtcttttaccagttcatctgatttttactttgttcccatttataattcaaatataatataaacgtcttattcatttagtcttcagcattttaagtattcgacacgaacattaaagatttggtgttgaatttaataaattacaccaaaaagtctcagtagccaattcactaacatcttttgatttggtaatcttcacagaatcaacaactgttaacacttctgtgcaaataaatttcagttgtgcttatatattataatattcactttaggaatccacctgtcaatttgcaattcaattataaggtatattttaagtgatatctatgtattgtatacacatattccattgaaaggtgagaataattagaaagtagtaatataaaaatgtgtatgaaataagaactttcttaaaagacctagtttagctaaatatatgacaatataaaatgtctgcccaatgttttattatactttgtctaatgacctttaaatttaactgaaattaaaaccgtacttttgcagttgatatttttgtgttccctttgttaacataccaagcactaattattgacttcaaggaagccacgtattgcaagtcattgcttagcctttcacatctgagtgttgataagatgacggaatgggtagtcttgtagtaattgcttatgggaatgctagtttaagaggaatgccttgtatagaacatattataccagattgacttgttaaaaatggttccaagaatcagtctaaaaacttcagaattatgtgaattattttttctctgttgtgatatgttgtaatgagatgtgtgttcttatattttcttagtaaaattttcttaagcataaaa

SEQ ID NO: 57 - DNA, 2244 bp, Homo sapiens
  source 1..2244: mol_type = genomic DNA
agtgaagaatttggcgccatcttgagaaggtcaaagtgattcttcgattaaatgccttacttatcgcctagtgtgacctggcgcttgggtttactctccccagcaactgcgcctcaaacgcaaagaaaactgcaggtcctcggggaaggatcgatttcaccattcaagagaaaaatccatttcccagattcctcccctttatgtaaacgtctggtacctagtagacgttaaatacacgtgagttttccggttttataagatcacatactgcaaaccaacagtaaattacattaatacgcatttctttagaaattcttaattgctaggcactgacatataaattgagtttgctgttgttttagagagaaaaatagacatattcattttttttcaatcaggacatggtattcgtttccttcttaagcttcccatttttgaggtaggaaagtattggtgtgtgtctctatctctaccttgctccctcattagcaggcttaatgggcaggaattatgtctgttttgctttccgttggatccccgggtctagtagagcgccttgacacatagtaggtactcgacacagatgctggcttcatgatgaagcaattgtcctcgaattaatacaaaagggaagattcagactcagcgagccggtaccattcgtctagggacacacagccaggaaggaaccgaaccaggattctaacccaggattacccaagttcgaattctaattgaacataagtccttagcgaccttggacggacttactaaactgccctgaatctcggcttccttagctgcaaaatagggttaggaaaagtggactcggtgctcctctgcgccgcattcccgcagcgggatttgtgaaagcgaacttgagccccacactgcagaagccctgaggtcctgtctcaaccagaatccagcacccaggacaagccctccagcgacttcagaccttcccgctcccgaccccgcccgcccccgagaggcgtggtcaaggcggggccagaaggcggacgcgtgcacggagcggtaaagcgcaggcgcaaagcttcggtgacgtcagagaggcgcgctcccagctcggagccgactcgcagacgcgccccgcccctcggcgtcgctctggactggcgcaggcgcaagccggcaagatggcggcggctggggctttccgtctgaggcgggcggcatcggctctgctgctgcggagcccccgcctgcccgcccgggagctgtcggccccggcccgactctatcacaagaaggtatctcaaatctgtgaagtattgtagaggagacacaaaaggaattgggggtcacaaatggttctcattgacatgagtgtagacctttctactcaggttgttgatcattatgaaaatcctagaaacgtggggtcccttgacaagacatctaaaaatgttggaactggactggtgggggctccagcatgtggtgacgtaatgaaattacagattcaagtggatgaaaaggggaagattgtggatgctaggtttaaaacatttggctgtggttccgcaattgcctccagctcattagccactgaatgggtgaaaggaaagacggtggaggaagccttgactatcaaaaacacagatatcgccaaggagctctgccttcctcccgtgaaactgcactgctccatgctggctgaagatgcaatcaaggccgccctggctgattacaaattgaaacaagaacccaaaaaaggagaggcagagaagaaatgagccctccctcggcgaagcctccagcaggccacaccagctgtttcccacctgctgtgcagtcaccttagatgttcagaagccgcttcctctccactgaagagctatgagatacgcacaatacttgctgttcacgttatgactctcatgcaagcaaaatacacagtttcattgttctgaatcctgtggtttctttcagcccacttttatcgccttaacctagttaatgtatattttgaattgtgtgtatgacctcagaactgaaattgataatgaagttgcaagttttgatagcccgtgaagtgcataagtatctaattttacctgaattgatttggggggaaattaccagtagaatgccttggtctgaatatttgatagaaccaattgttgtacataaaacagattgcgcatatatatatatgtataaaaaataataaaataatggaagatgatggtgttctctagtaaaaaaaaaaaaaaaaaa

SEQ ID NO: 58 - DNA, 5426 bp, Homo sapiens
  source 1..5426: mol_type = genomic DNA
acttccgtccaggtccagcgggtgctgtgatcttcaagtatggggtctgctggcttgtcgcggctgcatgggcttttcgcggtctataagcccccggggctaaaatggaagcacctgcgggatacagtggagctacaacttctgaagggtctcaatgccaggaagcctcccgctcctaaacagcgtgttcgcttcttgctgggccccatggaaggcagcgaagagaaggagctgaccctcacagccaccagcgtaccctctttcatcaaccatccactggtatgtggaccagcattcgcccatctcaaggttggcgtgggacatcggttggatgcccaggcttctggagtacttgtgctcggcgtgggacatggatgcaggctcctcaccgatatgtacaatgctcatcttaccaaggattacacagtgcgtggcctcctgggcaaagctacagatgacttccgtgaggacgggaggctggtagagaagacaacctatgaccacgtgaccagagagaagctggaccgcattctggccgttatccaaggctcccatcagaaggccctggtgatgtactccaacctcgacctgaagacccaggaggcctatgagatggccgtgagaggcctgatccggcccatgaacaagtccccgatgctgataactggcatccgatgcctctactttgcacctccggaattcctcttagaggtgcagtgcatgcatgagacgcagaaagagctgcggaagttggttcatgaaatcggcctggaactaaagaccactgctgtctgcacccaagtgcggcgcacgcgcgacggcttcttcacgctagacagtgccctcctgaggacccagtgggacctaaccaacatccaggatgctatccgggctgctacccctcaggtagctgcagagctggagaagagcttgagcccggggctggacaccaagcagctccccagtccgggatggtcctgggactcccagggcccgagctctaccttggggctggagaggggtgcggggcagtgaatgcccaggcagctcctggggcagtggatggataaatagaagaactgctatgagctggacctgatagctgtgcagaaagtgagagcaggggcaccttttctacgtaagacacaaaactcaagctgtaacagaaaggtcaatggacttgacccaactgagaaggaaaaacttctgtatggaggaaaagacagtaaacatgacagactgatagaaagtattggcagaccaggcgtggtggcgggcacttgtaatcccagctactcccaaggctgaggcaggagaattgcttgagcccaggagtttaaggttgtagtgcgctagacggcgcctgtgaatagccactgcactctagtctggggaacatatgagacccctgccatctcaaaaaaaaaaaaattctcaataaactatgtgcccacgaaaattaaaaattaaaaaaaattgtaagtatttgcagtgtcattaaccaaggactaatatccatagtatacagagagcccttagaaagcaataagaagacaacgacaaaattaggcaaatgagatgaagtgtgtatcagaagaaatccaaatggctagtaagccagaaatgacactcaacctctttaatactggaagacacagatgaaaatgagatgatttttgttgtttttctgttgttgcccaggctggagtgcagtggcgccatctttgctcactgcagcctccgcctcccaggttcaagtgattctcctgcctcagcctcccaagtagctggggtcacaggcgcccgccaccacacctggctaattttttgtatccgagatgaggtttcaccatgttggcctggctcagcctcccaaagtgctgggattacaggcgtgagccaccgcacctggccgggacctcagtcttttctcttaaaggtcttcagtttattgaatgagctccgcccacaattgtggtggataatcagcgtgactcaaagtctacccattgactgattgaaatgttaatcatttttttgtttttgtttttgtttttgagacttgctttgtggcccaggctggagtgcagtggtgcgatctaggctcactgcaacctccgcttcccaggttcacgccattctcctgcctcagcctcccgagtagctgggactacaggtgcccaccaccacgcccagctaatttttttgtatttttagtagagacggtgttgcaccgtgttagccaggatggtctcaatctcctgacctcgtgatccgcctggcttggcctcccaaagtactgggattacagccgtgagccaccacacccggcctgttttggttttttttttttctgagacggagtctcactccgtcatccaggttggagtgcagtggcacaatctcagctcactgcaacctctgcctcccaggttcaagcgattctcctgcctcagcttctcaagtagctgggattacagatgtgtaccaccatgcctggctaatttttgtatttttagtagagacagggtttcaccatgttggccaggctggtctggaactcctgacctcaggtgatccgcccacctcagcctcccagagcgctgggattatgccaacctctgcctcccaaagcactgggatggcaggcatgagccaccacacccggcctgtttttgttttttagacagggtcttgctctgtcacccaggctggagtgcagtgatgcaaacatgactcactgcagctttgtcctctcaggcttaaccatcttcccacctcagcttccagaatagctgggactgcaggcgtgcacacagtatctggctaatttatttattttattttatttttgagacagggcctcgcactgtcgcccaggctagagtgcagtggcgcggtttcaactcattgcagcctcgaccctcctgggtcaagcgattcttcctgccttagtctccagagttgctgggattaccggtgtgcaccaccaagcctggctaatttttgtatttttagtagagacagggtttcactatgttggccaggctggtctggaactcctgacctgaggtgatccgcctacctcggccttccaaagtactgggaatataggcatgagccactgtttccggcctgttttttttagacagagtcttgctctgtcacccaggctggagtgcaatggtgcaaacatgactcactgcaccgtcatcctcccaggctcaaccatcttcccacctcaccctccagaatagctgggactacaggcgtgcaccacgatgcctggctaaatatttttttccagctaattttattatttttttttgagatggagtcttgctctgtcacccaggctggagtgcaatggcacaatcttggctcactgcaacctccgcctcccagggtcaagtgattctcctgcctcagccacccaagtagctgggattataggcgttcaccaccacgcccagctaacttttgtatttttagtagagacggggtttcaccatgttggacatgctggtctcaaagtcctgacctcaggtgatctgcccgcttcagcctcccaaagtgctgggattacaggcgtgagccaccatacctggcccacctgagatcaggagttcaagaccagcctggccaacatagtgaaaccctgtctctactaaaaatacaaaaattagccagctatggtgacgcacttctgtaatcctagctacttgggaggctgaggcacaagaatcgctgaagcccaggaggtggagattgccattagctgagattgtgcccctgcactccagtctggacgagagagtgagactctgtctcagaaaagaaaaaaaaaaaggctgggcacggtggctcacgcctgtaatcccagcactttgggaggccagggtgggcggatcacgaggtcaggagttcaagaccagcctgaccaacatggtgaaacctgtctctactaaaaatacaaaattagccgggcgtgcttgcacgtgcctgtagtcctggctacccgggaggctgatgcagaataactggaactcgggaggcggagcttgcagtgagtggagattgcgccactgcactccagcctaggtgacagagtgagactctgtctcaaaaaaaaaaaaaaaagagaaaaatcagaacaaaatcagcaatatttagacaagcagaatccaatgaaatggtgaggaaagctgggtgggagtggagggtatcagcaaaactcagttctttggaaaaattaggtaacttgaacgtagggcacatgggctgtcacaccaccccatttagagcccttattggcatagagatgataaaagtggactctgtgatggcctctcatgatctcaggatagcaggataggggaactctgagtcatggccaaaataattggtcaatgtacagcctcagacagctgggaataccaccccaaatcacttggataaggagatgttttccttatcctgtgtctttaactctgagacggatggagttgaacaatcaacgattgttaaaaatgtaccaaatgtgtaagcatcatgaggtgtttaagctgagcttttaaattaaaattctgctgggtgtggcggctcatgcctgtaatcccagcactttgcgatccccacacaggaggattgcttgagcccagaagtttgagaccagcctgggcaacacaaggagaccctgtctctacaaaaaacaaacaaacaaacaaaaaagccgggggtgttggtgtgtgcctgtagtcccagctactagggaggctgagatgggaggatcgcttgaacctagcaagtcaaggcggcagtaaatggagatctgtactccagcctgggcaacagagcgagaccctatgtcagaaaaaaagaaaaagccacccagcaggctgagcgtggtggctcacgcctgtaatccctgcactttgggaagccaaagcaggtgaatcacttgaggtcaggagttcgagaccagcctggccaatatggtgaaaccctgtctctaccaaaaatacaaaaattagccgggcatggtggcgcttgtctgtagtccagctacagctactccagatctgggctgaggtgggagaatcatttgaacccaggaggtggaggttgcagtgagctgaaatcacacgactgtactctagcctgggtgacagggtgagaccctatctcaaaacaaacaaacaaaaaccccaccagcaaccttgctgacaaatccacaggcccccacgtctgccaattgcctgcctgtatgaactgggcacctgggggaggggcgtggcccccaggctactcatcttggctttcacagccaaagggcctgggtggaacctcagcttactgtgtttactgactgggtaatgtggcctctgaagctgtcacctatctataaaatggggattaaaacgaca

SEQ ID NO: 59 - DNA, 2693 bp, Homo sapiens
  source 1..2693: mol_type = genomic DNA
agggactggcgggactgcgcggcggcaacagcagacatgtcgggggtccggggcctgtcgcggctgctgagcgctcggcgcctggcgctggccaaggcgtggccaacagtgttgcaaacaggaacccgaggttttcacttcactgttgatgggaacaagagggcatctgctaaagtttcagattccatttctgctcagtatccagtagtggatcatgaatttgatgcagtggtggtaggcgctggaggggcaggcttgcgagctgcatttggcctttctgaggcagggtttaatacagcatgtgttaccaagctgtttcctaccaggtcacacactgttgcagcacagggaggaatcaatgctgctctggggaacatggaggaggacaactggaggtggcatttctacgacaccgtgaagggctccgactggctgggggaccaggatgccatccactacatgacggagcaggcccccgccgccgtggtcgagctagaaaattatggcatgccgtttagcagaactgaagatgggaagatttatcagcgtgcatttggtggacagagcctcaagtttggaaagggcgggcaggcccatcggtgctgctgtgtggctgatcggactggccactcgctattgcacaccttatatggaaggtctctgcgatatgataccagctattttgtggagtattttgccttggatctcctgatggagaatggggagtgccgtggtgtcatcgcactgtgcatagaggacgggtccatccatcgcataagagcaaagaacactgttgttgccacaggaggctacgggcgcacctacttcagctgcacgtctgcccacaccagcactggcgacggcacggccatgatcaccagggcaggccttccttgccaggacctagagtttgttcagttccaccctacaggcatatatggtgctggttgtctcattacggaaggatgtcgtggagagggaggcattctcattaacagtcaaggcgaaaggtttatggagcgatacgcccctgtcgcgaaggacctggcgtctagagatgtggtgtctcggtccatgactctggagatccgagaaggaagaggctgtggccctgagaaagatcacgtctacctgcagctgcaccacctacctccagagcagctggccacgcgcctgcctggcatttcagagacagccatgatcttcgctggcgtggacgtcacgaaggagccgatccctgtcctccccaccgtgcattataacatgggcggcattcccaccaactacaaggggcaggtcctgaggcacgtgaatggccaggatcagattgtgcccggcctgtacgcctgtggggaggccgcctgtgcctcggtacatggtgccaaccgcctcggggcaaactcgctcttggacctggttgtctttggtcgggcatgtgccctgagcatcgaagagtcatgcaggcctggagataaagtccctccaattaaaccaaacgctggggaagaatctgtcatgaatcttgacaaattgagatttgctgatggaagcataagaacatcggaactgcgactcagcatgcagaagtcaatgcaaaatcatgctgccgtgttccgtgtgggaagcgtgttgcaagaaggttgtgggaaaatcagcaagctctatggagacctaaagcacctgaagacgttcgaccggggaatggtctggaacacggacctggtggagaccctggagctgcagaacctgatgctgtgtgcgctgcagaccatctacggagcagaggcacggaaggagtcacggggcgcgcatgccagggaagactacaaggtgcggattgatgagtacgattactccaagcccatccaggggcaacagaagaagccctttgaggagcactggaggaagcacaccctgtcctatgtggacgttggcactgggaaggtcactctggaatatagacccgtgatcgacaaaactttgaacgaggctgactgtgccaccgtcccgccagccattcgctcctactgatgagacaagatgtggtgatgacagaatcagcttttgtaattatgtataatagctcatgcatgtgtccatgtcataactgtcttcatacgcttctgcactctggggaagaaggagtacattgaagggagattggcacctagtggctgggagcttgccaggaacccagtggccagggagcgtggcacttacctttgtcccttgcttcattcttgtgagatgataaaactgggcacagctcttaaataaaatataaatgaacaaactttcttttatttccaaatccatttgaaatattttactgttgtgactttagtcatatttgttgacctaaaaatcaaatgtaatctttgtattgtgttacatcaaaatccagatattttgtatagtttcttttttctttttcttttcttttttttttttgagacaggatcggtgcagtagtacaatcacagctcactgcagcctcaaactcctgggcagctcaggtgatcttcctgactcagccttctgagtagttggggctacaggtgtgcaccaccatgcccagctcatttattttgtaattgtagggacagggtctcactgtgttgcctaggctggtctcaagtgatcctccctccttggcctcccaaggtgctggaattataggtgtgaacaaacca

SEQ ID NO: 60 - DNA, 6018 bp, Homo sapiens
  source 1..6018: mol_type = genomic DNA
ggaaacaaaacagcggctggtctggaaggaacctgagctacgagccgcggcggcagcggggcggcggggaagcgtatacctaatctgggagcctgcaagtgacaacagcctttgcggtccttagacagcttggcctggaggagaacacatgaaagaaagaacctcaagaggctttgttttctgtgaaacagtatttctatacagttgctccaatgacagagttacctgcaccgttgtcctacttccagaatgcacagatgtctgaggacaaccacctgagcaatactgtacgtagccagaatgacaatagagaacggcaggagcacaacgacagacggagccttggccaccctgagccattatctaatggacgaccccagggtaactcccggcaggtggtggagcaagatgaggaagaagatgaggagctgacattgaaatatggcgccaagcatgtgatcatgctctttgtccctgtgactctctgcatggtggtggtcgtggctaccattaagtcagtcagcttttatacccggaaggatgggcagctaatctataccccattcacagaagataccgagactgtgggccagagagccctgcactcaattctgaatgctgccatcatgatcagtgtcattgttgtcatgactatcctcctggtggttctgtataaatacaggtgctataaggtcatccatgcctggcttattatatcatctctattgttgctgttctttttttcattcatttacttgggggaagtgtttaaaacctataacgttgctgtggactacattactgttgcactcctgatctggaattttggtgtggtgggaatgatttccattcactggaaaggtccacttcgactccagcaggcatatctcattatgattagtgccctcatggccctggtgtttatcaagtacctccctgaatggactgcgtggctcatcttggctgtgatttcagtatatgatttagtggctgttttgtgtccgaaaggtccacttcgtatgctggttgaaacagctcaggagagaaatgaaacgctttttccagctctcatttactcctcaacaatggtgtggttggtgaatatggcagaaggagacccggaagctcaaaggagagtatccaaaaattccaagtataatgcagaaagcacagaaagggagtcacaagacactgttgcagagaatgatgatggcgggttcagtgaggaatgggaagcccagagggacagtcatctagggcctcatcgctctacacctgagtcacgagctgctgtccaggaactttccagcagtatcctcgctggtgaagacccagaggaaaggggagtaaaacttggattgggagatttcattttctacagtgttctggttggtaaagcctcagcaacagccagtggagactggaacacaaccatagcctgtttcgtagccatattaattggtttgtgccttacattattactccttgccattttcaagaaagcattgccagctcttccaatctccatcacctttgggcttgttttctactttgccacagattatcttgtacagccttttatggaccaattagcattccatcaattttatatctagcatatttgcggttagaatcccatggatgtttcttctttgactataacaaaatctggggaggacaaaggtgattttcctgtgtccacatctaacaaagtcaagattcccggctggacttttgcagcttccttccaagtcttcctgaccaccttgcactattggactttggaaggaggtgcctatagaaaacgattttgaacatacttcatcgcagtggactgtgtccctcggtgcagaaactaccagatttgagggacgaggtcaaggagatatgataggcccggaagttgctgtgccccatcagcagcttgacgcgtggtcacaggacgatttcactgacactgcgaactctcaggactaccgttaccaagaggttaggtgaagtggtttaaaccaaacggaactcttcatcttaaactacacgttgaaaatcaacccaataattctgtattaactgaattctgaacttttcaggaggtactgtgaggaagagcaggcaccagcagcagaatggggaatggagaggtgggcaggggttccagcttccctttgattttttgctgcagactcatcctttttaaatgagacttgttttcccctctctttgagtcaagtcaaatatgtagattgcctttggcaattcttcttctcaagcactgacactcattaccgtctgtgattgccatttcttcccaaggccagtctgaacctgaggttgctttatcctaaaagttttaacctcaggttccaaattcagtaaattttggaaacagtacagctatttctcatcaattctctatcatgttgaagtcaaatttggattttccaccaaattctgaatttgtagacatacttgtacgctcacttgccccagatgcctcctctgtcctcattcttctctcccacacaagcagtctttttctacagccagtaaggcagctctgtcgtggtagcagatggtcccattattctagggtcttactctttgtatgatgaaaagaatgtgttatgaatcggtgctgtcagccctgctgtcagaccttcttccacagcaaatgagatgtatgcccaaagacggtagaattaaagaagagtaaaatggctgttgaagcactttctgtcctggtattttgtttttgcttttgccacacagtagctcagaatttgaacaaatagccaaaagctggtggttgatgaattatgaactagttgtatcaacacaaagcaagagttggggaaagccatatttaacttggtgagctgtgggagaacctggtggcagaaggagaaccaactgccaaggggaaagagaaggggcctccagcagcgaaggggatacagtgagctaatgatgtcaaggaggagtttcaggttattctcgtcagctccacaaatgggtgctttgtggtctctgcccgcgttacctttcctctcaatgtacctttgtgtgaactgggcagtggaggtgcctgctgcagttaccatggagttcaggctctgggcagctcagtcaggcaaaacacacaaacagccatcagcctgtgtgggctcagggcacctctggacaaaggcttgtggggcataaccttctttaccacagagagcccttagctatgctgatcagaccgtaagcgtttatgagaaacttagtttcctcctgtggctgaggaggggccagctttttcttcttttgcctgctgttttctctcccaatctatgatatgatatgacctggtttggggctgtctttggtgtttagaatatttgttttctgtcccaggatatttcttataagaacctaacttcaagagtagtgtgcgagtactgatctgaatttaaattaaaattggcttatattaggcagtcacagacaggaaaaataagagctatgcaaagaaagggggatttaaagtagtaggttctatcatctcaattcatttttttccatgaaatcccttcttccaagattcattccctctctcagacatgtgctagcatgggtattatcattgagaaagcacagctacagcaaagccacctgaatagcaatttgtgattggaagcattcttgagggatccctaatctagagtaatttatttgtgtaaggatcccaaatgtgttgcacctttcatgatacatttcttctctgaagagggtacgtggggtgtgtgtatttaaatccatcctatgtattactgattgtcctgtgtagaaagatggcaattattctgtctctttctccaagtttgagccacatctcagccacattgttagacagtgtacagagaacctatctttcctttttttttttttaaaggacaggattttgctgtgttgcccaggctagacttgaactcctgggctcaagtaatccacctcagcctgagtagctgagactacagcccatcttatttctttaaatcattcatctcaggcagagaacttttccctcaaacattctttttagaattagttcagtcattcctaaaacatccaaatgctagtcttccaccatgaaaaatagattgtcactggaaagaacagtagcaatttccataaggatgtgccttcactcacacgggacaggcggtggttatagagtcgggcaaaaccagcagtagagtatgaccagccaagccaatctgcttaataaaaagatggaagacagtaaggaaggaaagtagccactaagagtctgagtctgactgggctacagaataaagggtatttatggacagaatgtcattacatgcctatgggaataccaatcatatttggaagatttgcagattttttttcagagaggaaagactcaccttcctgtttttggttctcagtaggttcgtgtgtgttcctagaatcacagctctgactccaaatgactcaatttctcaattagaaaaagtagaagctttctaagcaacttggaagaaaacagtcataagtaagcaatttgttgattttactacagaagcaacaactgaagaggcagtgtttttactttcagactccgggattcccattctgtagtctctctgcttttaaaaaccctccttttgcaatagatgcccaaacagatgatgtttattacttgttatttacgtggcctcagacagtgtatgtattctcgatataacttgtagagtgtgaaatataagtttaactaccaaataaggtctcccagggttagatgactgcgggaagcctttgatcccaacccccaaggctttgtatatttgatcatttgtgatctaaccctggaagaaaaagagctcagaaaccactatgaaaaaatttgttcagtgttttctgtgttcccgtaggttctggagtctgaggatgcaaagatgaataagataaattctcagaatgtagttataatctcttgttttctggtatatgccatctttctttaacttctctaaaatattgggtatttgtcaaataaccacttttaacagttaccattactgagggcttatacattggtgttataaaagtgacttgattcagaaatcaatccattcagtaaagtactccttctctaaatttgctgttatgtctataaggaacagtttgacctgcccttctcctcacctcctcacctgccttccaacattgaatttggaaggagacgtgaaaattggacatttggttttgcccttgggctggaaactatcatataatcataagtttgagcctagaagtgatccttgtgatcttctcacctctttaaattcccacaacacaagagattaaaaacagaggtttcagctcttcatagtgcgttgtgaaatggctggccagagtgtaccaacaaagctgtcatcgggctcacagctcagagacatctgcatgtgatcatctgcatagtcctctcctctaacgggaaacacctcagatttgcatataaaaaagcaccctggtgctgaaatgaacccctttcttgaacatcaaagctgtctcccacagccttgggcagcagggtgcctcttagtggatgtgctgggtccaccctgagccctgacatgtggtggcagcattgccagttggtctgtgtgtctgtgtagcagggacgatttcccagaaagcaattttccttttgaaatacgtaattgttgagactaggcagtttcaaagtcagctgcatatagtagcaagtacaggactgtcttgtttttggtgtccttggaggtgctggggtgagggtttcagtgggatcatttactctcacatgttgtctgccttctgcttctgtggacactgctttgtacttaattcagacagactgtgaatacaccttttttataaatacctttcaaattcttggtaagatataattttgatagctgattgcagattttctgtatttgtcagattaataaagactgcatgaatcca

SEQ ID NO: 61 - DNA, 1306 bp, Homo sapiens
  source 1..1306: mol_type = genomic DNA
gtcaatggaggaagactggacctactgattcaacttggagatgagcgggtctgtcctcttcacggcgggagagaggtggagatgctttctgaccccgtcgaggtcatccctgtactgggccttacataatttctgctgtcggaaaaaatccactacacctaagaaaattactcccaatgttactttttgtgatgaaaatgcaaaggagcccgaaaatgcacttgacaagctcttctcttcagaacagcaggcttccatcttgcatgtgttgaatacagcatctactaaagaacttgaagctttccgattgcttcgtggaagaaggtccatcaatatcgtagagcacagagaaaactttgggccatttcagaatttagagagtttaatgaatgtgcccttgtttaagtataaaagtacagttcaagtttgtaactccatactttgtccaaagactggacgggaaaaaagaaagtcaccggaaaaccggttcctgagaaagctcctcaaaccagacatagaaagagaaagacttaaggcagttaatagtatcatatctatcgtttttggtactcgaagaattgcctgggctcaccttgatcgtaagttgacagtgctggactggcagcaaagtgaccgttggagtttaatgagaggaatatactcatcatcagtctatttagaagagatttcctcgatcatttcaaagatgcctaaagcagatttctatgttctggaaaaaacaggactttccattcagaactcatctctgtttccaatactgttacattttcatatcatggaagccatgctgtatgccttattaaataaaacttttgcccaggatgggcagcatcaggtgctgagcatgaatcgaaatgcagtggggaagcattttgaactgatgattggtgactcccggactagtggaaaagagctagtgaagcagtttctcttcgattctatactgaaggcggatcctcgggtgttcttcccatcagataaaatagttcactacagacagatgtttttatctactgaactacaaagagtagaagagctttatgattcattattacaagctattgccttctatgaattagcagtgtttgactctcagccttagaattctgaggttaacgtgctaaagtataattattagctctaacgtaacaccaactgttgtgaacatccatgttattggaaaagaacacattttcagtgtattttagatgtttaaattctgacttttggctattaaatggtttacacaataagccaagaccaaatcaataaacattttatgaga

SEQ ID NO: 62 - DNA, 2348 bp, Homo sapiens
  source 1..2348: mol_type = genomic DNA
accccgctccgggcgcgctctcgggaaggcttggaccgacgcggcccagaggccaggaacattccgcgcgtggaccagccgggccagggcgatgctgcgggtgcggtgtctgcgcggcgggagccgcggcgccgaggcggtgcactacatcggatctcggcttggacgaaccttgacaggatgggtgcagcgaactttccagagcacccaggcagctacggcttcctcccggaactcctgtgcagctgacgacaaagccactgagcctctgcccaaggactgccctgtctcttcttacaacgaatgggaccccttagaggaagtgatagtgggcagagcagaaaacgcctgtgttccaccgttcaccatcgaggtgaaggccaacacatatgaaaagtactggccattttaccagaagcaaggagggcattattttcccaaagatcatttgaaaaaggctgttgctgaaattgaagaaatgtgcaatattttaaaaacggaaggagtgacagtaaggaggcctgaccccattgactggtcattgaagtataaaactcctgattttgagtctacgggtttatacagtgcaatgcctcgagacatcctgatagttgtgggcaatgagattatcgaggctcccatggcatggcgttcacgcttctttgagtaccgagcgtacaggtcaattatcaaagactacttccaccgtggcgccaagtggacaacagctcctaagcccacaatggctgatgagctttataaccaggattatcccatccactctgtagaagacagacacaaattggctgctcagggaaaatttgtgacaactgagtttgagccatgctttgatgctgctgacttcattcgagctggaagagatatttttgcacagagaagccaggttacaaactacctaggcattgaatggatgcgtaggcatcttgctccagactacagagtgcatatcatctcctttaaagatcccaatcccatgcatattgatgctaccttcaacatcattggacctggtattgtgctttccaaccctgaccgaccatgtcaccagattgatcttttcaagaaagcaggatggactatcattactcctccaacaccaatcatcccagacgatcatccactctggatgtcatccaaatggctttccatgaatgtcttaatgctagatgaaaaacgtgttatggtggatgccaatgaagttccaattcaaaagatgtttgaaaagctgggtatcactaccattaaagttaacattcgtaatgccaattccctgggaggaggcttccattgctggacctgcgatgtccggcgccgaggcaccttacagtcctacttggactgaacaggcctgatggagcttgtggctggcctcagatacacctaagaagcttaggggcaaggttcattctcctgctttaaaaagtgcatgaactgtagtgctttaaacaatcatctccttaacaggggtcgtaagcctggtttgcttctattacttttctttgacataaagaaaataacttctgctaggtattactctctactcctaaagttatttactatttggcttcaagtataaaattttggtgaatgtgtaccaagaaaaaattagtcacctgagtaacttggccactaataattaaccatctacctctgtttttaattttctttccaaaaggcagcttgaaatgttggtcctaatcttaattttttttcctcttctatagacttgagaatgtttttctctaaatgagagaaagacttagaatgtacacagatccaaaatagaatcagattatctctttttttctaaaggagagaaagacttagaacatacacagatcctaagtagaaccaggtaattgtctctttttctaataaggaatttgggtaatttttaattttttgttttttaaaaaataacctagactatgcaaaacatcaaagtgaattttccatgaatgtttttaatattctcatctcaacattgtgatatatgctactaaaaaccttttcatatacatcttacctcatttcaagtgaattattttaatctttttctctctttccaaaaatttaggaatgtttagtgtaattggatttcgctatcagttcccatccttaagttttgatattcaatatctgatagatacactgcatctttggtcatctaagatttgtttacaaatgtgcaaattatttagagcatagactttataagcattaaaaaaaactaatggaggtaaaacctaaatgcgatgtgaaataattttagtgttgataccgtatgtgtatttttattctaataaacttttgtgttccagattgaa

SEQ ID NO: 63 - DNA, 4404 bp, Homo sapiens
  source 1..4404: mol_type = genomic DNA
agaagcgctgccgccaccgcatcgcgcagctctttgccgtcggagcgcttgtttgctgcctcgtactcctccatttatccgccatgataagtgccagccgagctgcagcagcccgtctcgtgggcgccgcagcctcccggggccctacggccgcccgccaccaggatagctggaatggccttagtcatgaggcttttagacttgtttcaaggcgggattatgcatcagaagcaatcaagggagcagttgttggtattgatttgggtactaccaactcctgcgtggcagttatggaaggtaaacaagcaaaggtgctggagaatgccgaaggtgccagaaccaccccttcagttgtggcctttacagcagatggtgagcgacttgttggaatgccggccaagcgacaggctgtcaccaacccaaacaatacattttatgctaccaagcgtctcattggccggcgatatgatgatcctgaagtacagaaagacattaaaaatgttccctttaaaattgtccgtgcctccaatggtgatgcctgggttgaggctcatgggaaattgtattctccgagtcagattggagcatttgtgttgatgaagatgaaagagactgcagaaaattacttggggcacacagcaaaaaatgctgtgatcacagtcccagcttatttcaatgactcgcagagacaggccactaaagatgctggccagatatctggactgaatgtgcttcgggtgattaatgagcccacagctgctgctcttgcctatggtctagacaaatcagaagacaaagtcattgctgtatatgatttaggtggtggaacttttgatatttctatcctggaaattcagaaaggagtatttgaggtgaaatccacaaatggggataccttcttaggtggggaagactttgaccaggccttgctacggcacattgtgaaggagttcaagagagagacaggggttgatttgactaaagacaacatggcacttcagagggtacgggaagctgctgaaaaggctaaatgtgaactctcctcatctgtgcagactgacatcaatttgccctatcttacaatggattcttctggacccaagcatttgaatatgaagttgacccgtgctcaatttgaagggattgtcactgatctaatcagaaggactatcgctccatgccaaaaagctatgcaagatgcagaagtcagcaagagtgacataggagaagtgattcttgtgggtggcatgactaggatgcccaaggttcagcagactgtacaggatctttttggcagagccccaagtaaagctgtcaatcctgatgaggctgtggccattggagctgccattcagggaggtgtgttggccggcgatgtcacggatgtgctgctccttgatgtcactcccctgtctctgggtattgaaactctaggaggtgtctttaccaaacttattaataggaataccactattccaaccaagaagagccaggtattctctactgccgctgatggtcaaacgcaagtggaaattaaagtgtgtcagggtgaaagagagatggctggagacaacaaactccttggacagtttactttgattggaattccaccagcccctcgtggagttcctcagattgaagttacatttgacattgatgccaatgggatagtacatgtttctgctaaagataaaggcacaggacgtgagcagcagattgtaatccagtcttctggtggattaagcaaagatgatattgaaaatatggttaaaaatgcagagaaatatgctgaagaagaccggcgaaagaaggaacgagttgaagcagttaatatggctgaaggaatcattcacgacacagaaaccaagatggaagaattcaaggaccaattacctgctgatgagtgcaacaagctgaaagaagagatttccaaaatgagggagctcctggctagaaaagacagcgaaacaggagaaaatattagacaggcagcatcctctcttcagcaggcatcactgaagctgttcgaaatggcatacaaaaagatggcatctgagcgagaaggctctggaagttctggcactggggaacaaaaggaagatcaaaaggaggaaaaacagtaataatagcagaaattttgaagccagaaggacaacatatgaagcttaggagtgaagagacttcctgagcagaaatgggcgaacttcagtctttttactgtgtttttgcagtattctatatataatttccttaatttgtaaatttagtgaccattagctagtgatcatttaatggacagtgattctaacagtataaagttcacaatattctatgtccctagcctgtcatttttcagctgcatgtaaaaggaggtaggatgaattgatcattataaagatttaactattttatgctgaagtgaccatattttcaaggggtgaaaccatctcgcacacagcaatgaaggtagtcatccatagacttgaaatgagaccacatatggggatgagatccttctagttagcctagtactgctgtactggcctgtatgtacatggggtccttcaactgaggccttgcaagtcaagctggctgtgccatgtttgtagatggggcagaggaatctagaacaatgggaaacttagctatttatattaggtacagctattaaaacaaggtaggaatgaggctagacctttaacttccctaaggcatacttttctagctaccttctgccctgtgtctggcacctacatccttgatgattgttctcttacccattctggaatttttttttttttaaataaatacagaaagcatcttgatctcttgtttgtgaggggtgatgccctgagatttagcttcaagaatatgccatggctcatgcttcccatatttcccaaagagggaaatacaggatttgctaacactggttaaaaatgcaaattcaagatttggaagggctgttataatgaaataatgagcagtatcagcatgtgcaaatcttgtttgaaggattttattttctccccttagacctttggtacatttagaatcttgaaagtttctagatctctaacatgaaagtttctagatctctaacatgaaagtttttagatctctaacatgaaaaccaaggtggctattttcaggttgctttcagctccaagtagaaataaccagaattggcttacattaaagaaactgcatctagaaataagtcctaagatactatttctatggctcaaaaataaaaggaacccagatttctttccctatttctgcttttctgtcctcagtgtactggcttgtcccaaattctctgatgtagttctaataattatcatgtgctgacaacctgagcagaaaggcaactacatctttattcaatgttaagatttcccaaaagtcttccagcacatttcccttatgtttcatggatgaatgtcatatgccatgttcaaaagagtctgttatcaagatgggcttagaatatcagacacaatggctccttttataatgttttgtaatatgtagaaatgagattactgtatttccaaaaatcagtataagaaatcaatttaatttgcaagtatgaaggaagtgatttagacaacatttttggaagattcagtgcttgttaacacccatgtaaaagattcttgggattcgtgaaaaattctaggctcaggtttccaaccacatgaacactcaatgggaacattgtaggattatgaaggattgttatatggtgtgttggaaaataacttttagcatttttggttggcccttgcccactgcctctactgtctcctaaagataactttaaaagccctccaggatttccaaagtactccagttgagaactactggcttaatgctagtcaagattcatccttgggatcagagagaggagccaaatgcccaagcaaaatatatatttgagttctattaggagggagaagttgcttgggtatgggtggacacagtatttcagtcacctatcagtaaagctaatagcataaatagcaagaaggtatatggagatttcctacagccaataggatatatatctagaataagatttactgtaagaaagaaactggctcacaattatggaaccgaggtcccacccacagtccactgctgggaaagctgttggggtaaaatttcagtgaccaggaacacagtggtgtcctagtccgaggttagaagaatatggatgtcttagctctggcagttctatataggcccttaactgattagatgatgtccactcacatcaaagcagagcaatctccaattcaatactaatctcttctggagagagacaaatgtttaacccgttaaatggcatcaatggctaaactgacacataaaattaactatcacaaaaggcaaaaa

SEQ ID NO: 64 - DNA, 3348 bp, Homo sapiens
  source 1..3348: mol_type = genomic DNA
agaagtccttcttgtcctggtcgttgttcccgtctgagtaccagctccccactgccctgagggcgggccggcctgcggcggagggaaaaaggaagaggagaaggaaattgtcccgaatccctgcagtctttctgtaggttgcggcacaacgccaggcaaaagaagaggaaggaatttaatcctaatcggtggaggtcgatttgagggtctgctgtagcaggtggctccgcttgaagcgagggaggaagtttcctccgatcagtagagattggaaagattgttgggagtggcacaccactagggaaaagaagaaggggcgaactgcttgtcttgaggaggtcaacccccagaatcagctcttgtggccttgaagtggctgaagacgatcaccctccacaggcttgagcccagtcccacagccttcctcccccagcctgagtgactactctattccttggtccctgctattgtcggggacgattgcatgggctacgccaggaaagtaggctgggtgaccgcaggcctggtgattggggctggcgcctgctattgcatttatagactgactaggggaagaaaacagaacaaggaaaaaatggctgagggtggatctggggatgtggatgatgctggggactgttctggggccaggtataatgactggtctgatgatgatgatgacagcaatgagagcaagagtatagtatggtacccaccttgggctcggattgggactgaagctggaaccagagctagggccagggcaagggccagggctacccgggcacgtcgggctgtccagaaacgggcttcccccaattcagatgataccgttttgtcccctcaagagctacaaaaggttctttgcttggttgagatgtctgaaaagccttatattcttgaagcagctttaattgctctgggtaacaatgctgcttatgcatttaacagagatattattcgtgatctgggtggtctcccaattgtcgcaaagattctcaatactcgggatcccatagttaaggaaaaggctttaattgtcctgaataacttgagtgtgaatgctgaaaatcagcgcaggcttaaagtatacatgaatcaagtgtgtgatgacacaatcacttctcgcttgaactcatctgtgcagcttgctggactgagattgcttacaaatatgactgttactaatgagtatcagcacatgcttgctaattccatttctgacttttttcgtttattttcagcgggaaatgaagaaaccaaacttcaggttctgaaactccttttgaatttggctgaaaatccagccatgactagggaactgctcagggcccaagtaccatcttcactgggctccctctttaataagaaggagaacaaagaagttattcttaaacttctggtcatatttgagaacataaatgataatttcaaatgggaagaaaatgaacctactcagaatcaattcggtgaaggttcactttttttctttttaaaagaatttcaagtgtgtgctgataaggttctgggaatagaaagtcaccatgattttttggtgaaagtaaaagttggaaaattcatggccaaacttgctgaacatatgttcccaaagagccaggaataacaccttgattttgtaatttagaagcaacacacattgtaaactattcattttctccaccttgtttatatggtaaaggaatcctttcagctgccagttttgaataatgaatatcatattgtatcatcaatgctgatatttaactgagttggtctttaggtttaagatggataaatgaatatcactacttgttctgaaaacatgtttgttgctttttatctcgctgcctagattgaaatattttgctatttcttctgcataagtgacagtgaaccaattcatcatgagtaagctcccttctgtcattttcattgatttaatttgtgtatcatcaataaaattgtatgttaatgctggaaagaaaaaaagaagaaagaaagaaaccatccctgtccttcagtttataatctagttggagagataagaaacgtacaaaccaaaagataacagaatatctgaagcatgtactcattgtcagatgttccctctgagagcacagaggaggcaaaagcttctgtgggatgtgctagtcggctaaagcttcacagaggaggtggcaattgaaaatgagtcctgaatggggtagggtggttagggaattccatgagacaagacaaggggggcatggtgtgagaaaggcatggaagtaggaaccctcttcctatgacaggagatcattctgcttagagtggagagtgtggagagtgggagtagataattttggaaagctgggtgaagccagttgtggagaattgtttgaatattatcccattgaatacccagagccactaaatctttttttactagaaaataattggggtccatatgaaagtctctattactgagtagtgtcaatgagggtgtggcaaaatggagcctttcacatcctagtggtggccatttggtaatacagatataagccttaaactatgtaaacccttgtcctaaggaagtaattgaataattgcccaaagattgtatgtatgaggctgttcatcccagcactgtctaagctagtaaaaattggaaacaatttaagtatctagcacattggattggttataaagcaaggaatgttcacacagtaggatattataagtatgctgatggaaatctatattgccaggaaaagctattcattatgcgttgtgaagtcagaaagtaaaaaagggtagatagaagtattcgaagtatagttccattttttgagactaataaaacatatgtttaaaaggacactaaaaactggagttatagatatccagatagaaacagtagttatctttgggtagaagaataatgagtgatctttacttttttactttttattcatctttgtgtttttatttatctaaaatgggtattgatttttaggacggttttgaaaaagaaaagtgttgggaatgaagcaagtgattgattggaaaacatactgaatggaagaaatatttagattaaaaatgaggtaggttgaagtttcttctctgaaatgatagataaatggtgaagataaggcttattgtgaggattcagtgaggtaatatatgcaaagtacttacaatgttctggcacatagtaattaattaagaaaatcgagcacccttaattacctagaatgcagggttgttagttttttggttgacttttgttttgctggggcattctgccatgttttagtgtcatttaataaataatagtaacaataaaggttaacatttattaagtga

SEQ ID NO: 65 - DNA, 1694 bp, Homo sapiens
  source 1..1694: mol_type = genomic DNA
gcactccagtccgggctggcggacagagggctggaaacaagacgctccagaatcaggagcttcccctcaggaaatagcatcctgtgtccccgcactgcagttgtctggtctctccagcagtttggtacttccggctgctgcaatgcgtgtggtggtgattggagcaggagtcatcgggctgtccaccgccctctgcatccatgagcgctaccactcagtcctgcagccactggacataaaggtctacgcggaccgcttcaccccactcaccaccaccgacgtggctgccggcctctggcagccctacctttctgaccccaacaacccacaggaggcggactggagccaacagacctttgactatctcctgagccatgtccattctcccaacgctgaaaacctgggcctgttcctaatctcgggctacaacctcttccatgaagccattccggacccttcctggaaggacacagttctgggatttcggaagctgacccccagagagctggatatgttcccagattacggctatggctggttccacacaagcctaattctggagggaaagaactatctacagtggctgactgaaaggttaactgagaggggagtgaagttcttccagcggaaagtggagtcttttgaggaggtggcaagagaaggcgcagacgtgattgtcaactgcactggggtatgggctggggcgctacaacgagaccccctgctgcagccaggccgggggcagatcatgaaggtggacgccccttggatgaagcacttcattctcacccatgacccagagagaggcatctacaattccccgtacatcatcccagggacccagacagttactcttggaggcatcttccagttgggaaactggagtgaactaaacaatatccaggaccacaacaccatttgggaaggctgctgcagactggagcccacactgaagaatgcaagaattattggtgaacgaactggcttccggccagtacgcccccagattcggctagaaagagaacagcttcgcactggaccttcaaacacagaggtcatccacaactatggccatggaggctacgggctcaccatccactggggatgtgccctggaggcagccaagctctttgggagaatcctggaagaaaagaaattgtccagaatgccaccatcccacctctgaagactccagtgactgctgcctccccccacaagaactcccttctcccctcagccaatgaatcaatgtgctccttcataagccattgcttctccctcacttctttcctcaaagaagcatgaggtgagagaaagccacaaagtcagtgcctggagaagggttcagcccaacatggggcccctctcatcactgaaatccctctaccttctctgggtctggcattataaagaacagctgaggctgtcattccatgagtcttcagaagaaaggacagctcagaaaatcaaagaggccaactgcccagagccacagaaaatggaggataattgaggctaagtaacctgattacaagttgtactaacatattaaaggttctgaaaagtcctgcagcaaagacaactatctgatgttgtttaacccagtgcttgctaaacctatctggctatggaactcttttgcccagagcacccatgaatgccatgacacaaatctgagaaaatgctggaacaga

SEQ ID NO: 66 - DNA, 3927 bp, Homo sapiens
  source 1..3927: mol_type = genomic DNA
taccgcgccggagccttccgcagctgccgcttcagtccgaaggaggaagggaaccaacccactttctcggcgccgcggctcttttctaaaagtaatgtgaaaacctttgcatcttctgatagtctagccaaggtccaagaagtagcaagctggcttttggaaatgaatcaggaactgctctctgtgggcagcaaaagacgacgaactggaggctctctgagaggtaacccttcctcaagccaggtagatgaagaacagatgaatcgtgtggtagaggaggaacagcaacagcaactcagacaacaagaggaggagcacactgcaaggaatggtgaagttgttggagtagaacctagacctggaggccaaaatgattcccagcaaggacagttggaagaaaacaataatagatttatttcggtagatgaggactcctcaggaaaccaagaagaacaagaggaagatgaagaacatgctggtgaacaagatgaggaggatgaggaggaggaggagatggaccaggagagtgacgattttgatcagtctgatgatagtagcagagaagatgaacatacacatactaacagtgtcacgaactccagtagtattgtggacctgcccgttcaccaactctcctccccattctatacaaaaacaacaaaaatgaaaagaaagttggaccatggttctgaggtccgctctttttctttgggaaagaaaccatgcaaagtctcagaatatacaagtaccactgggcttgtaccatgttcagcaacaccaacaacttttggggacctcagagcagccaatggccaagggcaacaacgacgccgaattacatctgtccagccacctacaggcctccaggaatggctaaaaatgtttcagagctggagtggaccagagaaattgcttgctttagatgaactcattgatagttgtgaaccaacacaagtaaaacatatgatgcaagtgatagaaccccagtttcaacgagacttcatttcattgctccctaaagagttggcactctatgtgctttcattcctggaacccaaagacctgctacaagcagctcagacatgtcgctactggagaattttggctgaagacaaccttctctggagagagaaatgcaaagaagaggggattgatgaaccattgcacatcaagagaagaaaagtaataaaaccaggtttcatacacagtccatggaaaagtgcatacatcagacagcacagaattgatactaactggaggcgaggagaactcaaatctcctaaggtgctgaaaggacatgatgatcatgtgatcacatgcttacagttttgtggtaaccgaatagttagtggttctgatgacaacactttaaaagtttggtcagcagtcacaggcaaatgtctgagaacattagtgggacatacaggtggagtatggtcatcacaaatgagagacaacatcatcattagtggatctacagatcggacactcaaagtgtggaatgcagagactggagaatgtatacacaccttatatgggcatacttccactgtgcgttgtatgcatcttcatgaaaaaagagttgttagcggttctcgagatgccactcttagggtttgggatattgagacaggccagtgtttacatgttttgatgggtcatgttgcagcagtccgctgtgttcaatatgatggcaggagggttgttagtggagcatatgattttatggtaaaggtgtgggatccagagactgaaacctgtctacacacgttgcaggggcatactaatagagtctattcattacagtttgatggtatccatgtggtgagtggatctcttgatacatcaatccgtgtttgggatgtggagacagggaattgcattcacacgttaacagggcaccagtcgttaacaagtggaatggaactcaaagacaatattcttgtctctgggaatgcagattctacagttaaaatctgggatatcaaaacaggacagtgtttacaaacattgcaaggtcccaacaagcatcagagtgctgtgacctgtttacagttcaacaagaactttgtaattaccagctcagatgatggaactgtaaaactatgggacttgaaaacgggtgaatttattcgaaacctagtcacattggagagtggggggagtgggggagttgtgtggcggatcagagcctcaaacacaaagctggtgtgtgcagttgggagtcggaatgggactgaagaaaccaagctgctggtgctggactttgatgtggacatgaagtgaagagcagaaaagatgaatttgtccaattgtgtagacgatatactccctgcccttccccctgcaaaaagaaaaaaagaaaagaaaaagaaaaaaatcccttgttctcagtggtgcaggatgttggcttggggcaacagattgaaaagacctacagactaagaaggaaaagaagaagagatgacaaaccataactgacaagagaggcgtctgctgtctcatcacataaaaggcttcacttttgactgagggcagctttgcaaaatgagactttctaaatcaaaccaggtgcaattatttctttattttcttctccagtggtcattgggcagtgttaatgctgaaacatcattacagattctgctagcctgttcttttaccactgacagctagacacctagaaaggaactgcaataatatcaaaacaagtactggttgactttctaattagagagcatctgcaacaaaaagtcatttttctggagtggaaaagcttaaaaaaattactgtgaattgtttttgtacagttatcatgaaaagcttttttttttttttttttgccaaccattgccaatgtcaatcaatcacagtattagcctctgttaatctatttactgttgcttccatatacattcttcaatgcatatgttgctcaaaggtggcaagttgtcctgggttctgtgagtcctgagatggatttaattcttgatgctggtgctagaagtaggtcttcaaatatgggattgttgtcccaaccctgtactgtactcccagtggccaaacttatttatgctgctaaatgaaagaaagaaaaaagcaaattatttttttttattttttttctgctgtgacgttttagtcccagactgaattccaaatttgctctagtttggttatggaaaaaagactttttgccactgaaacttgagccatctgtgcctctaagaggctgagaatggaagagtttcagataataaagagtgaagtttgcctgcaagtaaagaattgagagtgtgtgcaaagcttattttcttttatctgggcaaaaattaaaacacattccttggaacagagctattacttgcctgttctgtggagaaacttttctttttgagggctgtggtgaatggatgaacgtacatcgtaaaactgacaaaatattttaaaaatatataaaacacaaaattaaaataaagttgctggtcagtcttagtgttttacagtatttgggaaaacaactgttacagttttattgctctgagtaactgacaaagcagaaactattcagtttttgtagtaaaggcgtcacatgcaaacaaacaaaatgaatgaaacagtcaaatggtttgcctcattctccaagagccacaactcaagctgaactgtgaaagtggtttaacactgtatcctaggcgatcttttttcctccttctgtttatttttttgtttgttttatttatagtctgatttaaaacaatcagattcaagttggttaattttagttatgtaacaacctgacatgatggaggaaaacaacctttaaagggattgtgtctatggtttgattcacttagaaattttattttcttataacttaagtgcaataaaatgtgttttttcatgttaaaaaaaaaaaaaaaaaaa

SEQ ID NO: 67 - DNA, 870 bp, Homo sapiens
  source 1..870: mol_type = genomic DNA
gttctccgcccctgccactgggccatggagactgtggcacagtagactgtagtgtgaggctcgcgggggcagtggccatggaggccgtgctgaacgagctggtgtctgtggaggacctgctgaagtttgaaaagaaatttcagtctgagaaggcagcaggctcggtgtccaagagcacgcagtttgagtacgcctggtgcctggtgcggagcaagtacaatgatgacatccgtaaaggcatcgtgctgctcgaggagctgctgcccaaagggagcaaggaggaacagcgggattacgtcttctacctggccgtggggaactaccggctcaaggaatacgagaaggccttaaagtacgtccgcgggttgctgcagacagagccccagaacaaccaggccaaggaactggagcggctcattgacaaggccatgaagaaagatggactcgtgggcatggccatcgtgggaggcatggccctgggtgtggcgggactggccggactcatcggacttgctgtgtccaagtccaaatcctgaaggagacgcgggagcccacggagaacgctccaggagggcctgtccatcctcgctgtcctttccctgttctccccctgccccccgtctctatcctctgtggccttcagctaatttctgctcccctgagattcgtccttcagccccatcatgtgctttgggatgagtgtaaataaaacggggctgtggcttgggaacccccgtgtctgcgttgaggggaggggtgggttctgaacgctgggcacagggggcggagacgtcactcctggtcctctcctgcccctgccctccaggttggcacggggttctttgtccaccccgcagcccagagcccaca

SEQ ID NO: 68 - DNA, 1669 bp, Homo sapiens
  source 1..1669: mol_type = genomic DNA
gaggtgagaagtacgtccactcttctgtccagcttttaacaatctaactaatgccctctccagggtcaccctagaatcagatctgctccccagcatcttctgtttcctggtgagtgattcctgctactttggatggccatgacgggctggagctgccttgtgacaggagcaggagggtttctgggacagaggatcatccgcctcttggtgaaggagaaggagctgaaggagatcagggtcttggacaaggccttcggaccagaattgagagaggaattttctaaactccagaacaagaccaagctgacagtgctggaaggagacattctggatgagccattcctgaagagagcctgccaggacgtctcggtcatcatccacaccgcctgtatcattgatgtcttcggtgtcactcacagagagtctatcatgaatgtcaatgtgaaaggtacccagctcctgttagaggcctgtgtccaagctagtgtgccagtcttcatctacaccagtagcatagaggtagccgggcccaactcctacaaggaaatcatccagaatggccatgaagaagagcctctggaaaacacatggcccgctccatacccacacagcaaaaagcttgctgagaaggctgtactggcggctaacgggtggaatctgaaaaacggcggcaccctgtacacttgtgccttacgacccatgtatatctatggggaaggaagccgattcctttctgctagtataaacgaggccctgaacaacaatgggatcctgtcaagtgttggaaagttctccactgttaacccagtctatgttggcaatgtggcctgggcccacattctggccttgagggccctgcaggaccccaagaaggccccaagcatccgaggacagttctactatatctcagatgacacgcctcaccaaagctatgataaccttaattacaccctgagcaaagagttcggcctccgccttgattccagatggagctttcctttatccctgatgtattggattggcttcctgctggaaatagtgagcttcctactcaggccaatttacacctatcgaccgcccttcaaccgccacatagtcacattgtcaaatagcgtattcaccttctcttataagaaggctcagcgagatctggcgtataagccactctacagctgggaggaagccaagcagaaaacggtggagtgggttggttcccttgtggaccggcacaaggagaccctgaagtccaagactcagtgatttaaggatgacagagatgtgcatgtgggtattgttaggagatgtcatcaagctccaccctcctggcctcatacagaaagtgacaagggcacaagctcaggtcctgctgcctccctttcatacaatggccaacttattgtattcctcatgtcatcaaaacctgcgcagtcattggcccaacaagaaggtttctgtcctaatcatataccagaggaaagaccatgtggtttgctgttaccaaatctcagtagctgattctgaacaatttagggactcttttaacttgagggtcgttttgactactagagctccatttctactcttaaatgagaaaggatttcctttctttttaatcttccattccttcacatagtttgataaaaagatcaataaatgtttgaatgtttaa

SEQ ID NO: 69 - DNA, 2173 bp, Homo sapiens
  source 1..2173: mol_type = genomic DNA
agttctgccgagatcgcagaatacacacaagctacctttgggcaccagagcagacagaaccgcggagcttcagggtggaagattcgtggaaactttgccaaggccaggacctcgtgtgttcccgtccgcccctctgggacggcgccagcccggcaggccgccgaccgtcctggggctcccgcgcagcgcgatgccggcctcgtccaccgtccacgtgctgcagctgctgcgggagctgctcgccttcgtgctcctcagctacacggtgctcatcggggcgctgctgctggccggctggaccacttacttcctggtgctgaagtgacagcgccgtcgccgcgcccggccccgcctcccgcccggccccgcctcccgcccggccccgcctccctaactcaccaggaaattcccttcaagccctggcccgaactgagtccccgcccacccgccagcgtcacggcgcccgactcagctccgcgccggacccacctccgcgccctcaggccctgcatatgccccgccccgcgcggaagttccggcggttggttgccttgcgcggccgttacagcctttgccctaagcctcgccccctttccccctgcctgcccaatcccgactgcttccttgggtgggggcgtggctatggggcgaggcgctctcaggtggaggccgtgccccgctccgcgctcacgaagctgcgtcacttccggcgtgtgcgtctggcgtccgcgcgctgcacaatggcggctctgaagagttggctgtcgcgcagcgtaacttcattcttcaggtacagacagtgtttgtgtgttcctgttgtggctaactttaagaagcggtgtttctcagaattgataagaccatggcacaaaactgtgacgattggctttggagtaaccctgtgtgcggttcctattgcacagaaatcagagcctcattcccttagtagtgaagcattgatgaggagagcagtgtctttggtaacagatagcacctctacctttctctctcagaccacatatgcgttgattgaagctattactgaatatactaaggctgtttataccttaacttctctttaccgacaatatacaagtttacttgggaaaatgaattcagaggaggaagatgaagtgtggcaggtgatcataggagccagagctgagatgacttcaaaacaccaagagtacttgaagctggaaaccacttggatgactgcagttggtctttcagagatggcagcagaagctgcatatcaaactggcgcagatcaggcctctataaccgccaggaatcacattcagctggtgaaactgcaggtggaagaggtgcaccagctctcccggaaagcagaaaccaagctggcagaagcacagatagaagagctccgtcagaaaacacaggaggaaggggaggagcgggctgagtcggagcaggaggcctacctgcgtgaggattgagggcctgagcacactgccctgtctccccactcagtggggaaagcaggggcagatgccaccctgcccagggttggcatgactgtctgtgcaccgagaagaggcggcagatcctgccctggccaatcaggcgagacgcctttgtgagctgtgagtgcctcctgtggtctcaggcttgcgctggacctggttcttagcccttgggcactgcaccctgtttaacatttcaccccactctgtacagctgctcttacccattttttttacctcacacccaaagcattttgcctacctgggtcagagagaggagtcctttttgtcatgcccttaagttcagcaactgtttaacctgttttcagtcttatttacgtcgtcaaaaatgatttagtacttgttccctctgttgggatgccagttgtggcagggggaggggaacctgtccagtttgtacgatttctttgtatgtatttctgatgtgttctctgatctgcccccactgtcctgtgaggacagctgaggccaaggagtgaaaaacctattactactaagagaaggggtgcagagtgtttacctggtgctctcaacaggacttaacatcaacaggacttaacacaggcctcttgttccttcctttctttccgtttctctattgtatccaaaggagaagagtgtaagattttgtttgcatctgaaagagaaaatgcgtctctcctggg

SEQ ID NO: 70 - DNA, 5626 bp, Homo sapiens
  source 1..5626: mol_type = other DNA
acattgttgcatgaaactccggcgcaggagtcccgggctgccgctggcaacatcgtgtcacccagctaagaaaatccgcgggcccgagccacgcgcctgtgaatcggagaggtcccactgcccgagtggagccgggctgagattcttctcaagttgagcctcagtgatcctgtggccgaagttagcgccttgacgtgggacaaccggacacgtcgccaggagagaactgaggcgccttctagcagttgtgacgccaaaatcacgtctccggagacccgcgccctccgccagccgggcgcaccctcgccggtagccttctttgtgcgccgtccggactcccagctcccggcccggcagccgagccccagcacaaagcagtcggaccgcgccgcccgcctcccctctcgcgtctccgcctcggtttcccaactctgcgccgtcgggccgcggcaggatgattgcctcgcatctgcttgcctacttcttcacggagctcaaccatgaccaagtgcagaaggttgaccagtatctctaccacatgcgcctctctgatgagaccctcttggagatctctaagcggttccgcaaggagatggagaaagggcttggagccaccactcaccctactgcagcagtgaagatgctgcccacctttgtgaggtccactccagatgggacagaacacggagagttcctggctctggatcttggagggaccaacttccgtgtgctttgggtgaaagtaacggacaatgggctccagaaggtggagatggagaatcagatctatgccatccctgaggacatcatgcgaggcagtggcacccagctgtttgaccacattgccgaatgcctggctaacttcatggataagctacaaatcaaagacaagaagctcccactgggttttaccttctcgttcccctgccaccagactaaactagacgagagtttcctggtctcatggaccaagggattcaagtccagtggagtggaaggcagagacgttgtggctctgatccggaaggccatccagaggagaggggactttgatatcgacattgtggctgtggtgaatgacacagttgggaccatgatgacctgtggttatgatgaccacaactgtgagattggtctcattgtgggcacgggcagcaacgcctgctacatggaagagatgcgccacatcgacatggtggaaggcgatgaggggcggatgtgtatcaatatggagtggggggccttcggggacgatggctcgctcaacgacattcgcactgagtttgaccaggagattgacatgggctcactgaacccgggaaagcaactgtttgagaagatgatcagtgggatgtacatgggggagctggtgaggcttatcctggtgaagatggccaaggaggagctgctctttggggggaagctcagcccagagcttctcaacaccggtcgctttgagaccaaagacatctcagacattgaaggggagaaggatggcatccggaaggcccgtgaggtcctgatgcggttgggcctggacccgactcaggaggactgcgtggccactcaccggatctgccagatcgtgtccacacgctccgccagcctgtgcgcagccaccctggccgccgtgctgcagcgcatcaaggagaacaaaggcgaggagcggctgcgctctactattggggtcgacggttccgtctacaagaaacacccccattttgccaagcgtctacataagaccgtgcggcggctggtgcccggctgcgatgtccgcttcctccgctccgaggatggcagtggcaaaggtgcagccatggtgacagcagtggcttaccggctggccgatcaacaccgtgcccgccagaagacattagagcatctgcagctgagccatgaccagctgctggaggtcaagaggaggatgaaggtagaaatggagcgaggtctgagcaaggagactcatgccagtgcccccgtcaagatgctgcccacctacgtgtgtgctaccccggacggcacagagaaaggggacttcttggccttggaccttggaggaacaaatttccgggtcctgctggtccgtgttcggaatgggaagtggggtggagtggagatgcacaacaagatctacgccatcccgcaggaggtcatgcacggcaccggggacgagctctttgaccacattgtccagtgcatcgcggacttcctcgagtacatgggcatgaagggcgtgtccctgcctctgggttttaccttctccttcccctgccagcagaacagcctggacgagagcatcctcctcaagtggacaaaaggcttcaaggcatctggctgcgagggcgaggacgtggtgaccctgctgaaggaagcgatccaccggcgagaggagtttgacctggatgtggttgctgtggtgaacgacacagtcggaactatgatgacctgtggctttgaagaccctcactgtgaagttggcctcattgttggcacgggcagcaatgcctgctacatggaggagatgcgcaacgtggaactggtggaaggagaagaggggcggatgtgtgtgaacatggaatggggggccttcggggacaatggatgcctagatgacttccgcacagaatttgatgtggctgtggatgagctttcactcaaccccggcaagcagaggttcgagaaaatgatcagtggaatgtacctgggtgagattgtccgtaacattctcatcgatttcaccaagcgtggactactcttccgaggccgcatctcagagcggctcaagacaaggggcatctttgaaaccaagttcttgtctcagattgagagtgactgcctggccctgctgcaagtccgagccatcctgcaacacttagggcttgagagcacctgtgacgacagcatcattgttaaggaggtgtgcactgtggtggcccggcgggcagcccagctctgtggcgcaggcatggccgctgtggtggacaggatacgagaaaaccgtgggctggacgctctcaaagtgacagtgggtgtggatgggaccctctacaagctacatcctcactttgccaaagtcatgcatgagacagtgaaggacctggctccgaaatgtgatgtgtctttcctgcagtcagaggatggcagcgggaagggggcggcgctcatcactgctgtggcctgccgcatccgtgaggctggacagcgatagaacccctgaaatcggaagggacttcctctttctctccttcttccctgttttaaattataagatgtcatccccttgtgtcagagacagaccccttggcttttgcttggcagagaggaccccactggactgggttttgtctctgcatctcattgtagagcttggtggctgagcttggccctattaagataaatagagttccaaataaggatttgttcacatgcatcataaccattcccattggttctcctaaaacatgaaaattatctcccttagtaatcccccttgccaaattccatgtccctgtataattctacaggatggggacactaatgaagatacggttgcttcaccttggagcctgaacatgacatttctaagtggggtgcatcccccagcactgatgttgttactgattctcctgtcagagatctgggaggtctccactgaggatgtgagcctgattatcctataggcagacgtggggagggtggaggggtgacagtggaggaaaatccatggatatccacgcagcagcccctctttaacctcatctacaagcatttgccctgtggattccagcatttgccattcctggaatcaaggaatcctgagtctgggcaatgaaaccaaagccaggagttgacgcatcctgcagttgggccagctgtcgcatctcagcggggcgcacatgttatccacaagcaatggacctttggggaagggggagtttttagtttgttttacaaatttttcctgcaaaagtggaatcactgtattttcattttaatttatatttgaaattttatttagttcttgagtagatctgcttcttcatcttgacatgtaatgaatggtcagttgtacgtaatgtatttatatgttaatttgttatgtatatagatgtgcaagtcttgtcagaattggcctcagtgtagttaaagggcagaaggggaagatactgactagtcatagaaatacctcattcgcctgtgggaagagaagggaagcctcttcagggtgagtgaatggcaaagcggttgcttctggctcctccttcccctgtggtcttggaagtgtgtggaaggcagggacagagatggaggccgagccaatagactgaagagaccacagcaattggctcctccatctagagattttcttggcagtattccatgggatgttaagcaaaggaaaccaaaggaatcgtttcaaatggactcatggcttagaaatctttattcttagggcagtcagtagtattctaaagctttctgacaagataaaggaagtcaccaaaatttctttttttaaattgtatctaatcctcaacaacaaaccaaaacagaacaattaaacagccaaataaaacctcagggacaacatttttggtgtatttgagccctcccagcaagtttcaccttgggtttgtattttaaatgttttacaagaattgtccatgtgcttccctaggctgagctggcattggtctgctgacctgtttttgtgtttttcttttttttatacacaacatttatttcaaactattgggagggatgagagtggcttaaaaacttccatccctacttttcaagagtgcagttgattctgaatctgaaagcccgcctctgtcctaaaatacaaacaagcacagacattaaacctggatactatatgataaagagggatgtaactattgaattggatacaaggatcagaatggaaagaaactcacgatgaaattgaacctggtttttgtatatttatcaaacttgtgctgagaatagtgtctgattatacgacttttaagcaaagttgggtgtaattaggtgaaaacagcccaggtcctcccgggagcacagaggggctaggggctggtccttctcgtttgctctagtcttgctttgctgtctggtgtagctcctctgctgctcccatctgcactaattgacccaaaacgtgggtatttcctgctacacaaaagccaaaaggtttcatgtagattttagttcactaaagggtgcccacaaaatagagattaattttaacttaaattttaagcttgaagattaggtactatctgtgaagttacacttttttttttttttttaaaggtagagatgtgtgtgtgtgtaggtattaaagatgtgttgttggtttccaaaaaggaacactggaaaataaattttgaatgtttatgttctcagaatcaggttgacagtcccttgctgacatggctttgctttgtgtaaatacagtggatctcaatcttcggggtgtgatgaatagcgaatcatctcaaatccttgagcactcagtctagtgaagatgttgtcattatgtacaatacataactagtttaattaactatgtgatgttaactattattaataaattttaacattttccaaaataatcaaa

SEQ ID NO: 71 - DNA, 2943 bp, Homo sapiens
  source 1..2943: mol_type = genomic DNA
agaggcgctctccactgctgtcctcttcagctcaagatggtggcctgccgggcgattggcatcctcagccgcttttctgccttcaggatcctccgctcccgaggttatatatgccgcaattttacagggtcttctgctttgctgaccagaacccatattaactatggagtcaaaggggatgtggcagttgttcgaattaactctcccaattcaaaggtaaatacactgagtaaagagctacattcagagttctcagaagttatgaatgaaatctgggctagtgatcaaatcagaagtgccgtccttatctcatcaaagccaggctgctttattgcaggtgctgatatcaacatgttagccgcttgcaagacccttcaagaagtaacacagctatcacaagaagcacagagaatagttgagaaacttgaaaagtccacaaagcctattgtggctgccatcaatggatcctgcctgggaggaggacttgaggttgccatttcatgccaatacagaatagcaacaaaagacagaaaaacagtattaggtacccctgaagttttgctgggggccttaccaggagcaggaggcacacaaaggctgcccaaaatggtgggtgtgcctgctgctttggacatgatgctgactggtagaagcattcgtgcagacagggcaaagaaaatgggactggttgaccaactggtggaacccctgggaccaggactaaaacctccagaggaacggacaatagaatacctagaagaagttgcaattacttttgccaaaggactagctgataagaagatctctccaaagagagacaagggattggtggaaaaattgacagcgtatgccatgactattccatttgtcaggcaacaggtttacaaaaaagtggaagaaaaagtgcgaaagcagactaaaggcctttatcctgcacctctgaaaataattgatgtggtaaagactggaattgagcaagggagtgatgccggttatctctgtgaatctcagaaatttggagagcttgtaatgaccaaagaatcaaaggccttgatgggactctaccatggtcaggtcctgtgcaagaagaataaatttggagctccacagaaggatgttaagcatctggctattcttggtgcagggctgatgggagcaggcatcgcccaagtctccgtggataaggggctaaagactatacttaaagatgccaccctcactgcgctagaccgaggacagcaacaagtgttcaaaggattgaatgacaaagtgaagaagaaagctctaacatcatttgaaagggattccatcttcagcaacttgactgggcagcttgattaccaaggttttgaaaaggccgacatggtgattgaagctgtgtttgaggaccttagtcttaagcacagagtgctaaaggaagtagaagcggtgattccagatcactgtatctttgccagtaacacatctgctctcccaatcagtgaaatcgctgctgtcagcaaaagacctgagaaggtgattggcatgcactacttctctcccgtggacaagatgcagctgctggagattatcacgaccgagaaaacttccaaagacaccagtgcttcagctgtagcagttggtctcaagcaggggaaggtcatcattgtggttaaggatggacctggcttctatactaccaggtgtcttgcgcccatgatgtctgaagtcatccgaatcctccaggaaggagttgacccgaagaagctggattccctgaccacaagctttggctttcctgtgggtgccgccacactggtggatgaagttggtgtggatgtagcgaaacatgtggcggaagatctgggcaaagtctttggggagcggtttggaggtggaaacccagaactgctgacacagatggtgtccaagggcttcctaggtcgtaaatctgggaagggcttttacatctatcaggagggtgtgaagaggaaggatttgaattctgacatggatagtattttagcgagtctgaagctgcctcctaagtctgaagtctcatcagacgaagacatccagttccgcctggtgacaagatttgtgaatgaggcagtcatgtgcctgcaagaggggatcttggccacacctgcagagggagacatcggagccgtctttgggcttggcttcccgccttgtctgggagggcctttccgctttgtggatctgtatggcgcccagaagatagtggaccggctcaagaaatatgaagctgcctatggaaaacagttcaccccatgccagctgctagctgaccatgctaacagccctaacaagaagttctaccagtgagcaggcctcatgcctcgctcagtcagtgcactaaccccagctgccggcagtgctggttctccaacagagtggtgtctagatttatcagagtaacgagaagacaaactccggcactgggtttgctccctgattaaagtgccttcagccaagaccatctctccctcctggtgaagtgtgacttcgaattagtttgcacttcctgttggaaggtagagcccactgctcattgtataagccccgaggcctagagtggcagccaagagccatctgaagccacctctctgcctgttcctcccaagaggccagggtggccaggggtggtgagggcagttctgcacccagccaaacacataacaataaaaaccaaactctgtgtcagcatctttgcccttctggtttaaacgcctccttcaaaaagcaatctggaagaaagccctgtgctttgggggagtaagaatgtgtgtgcagaattctaggcagcaccttagggagggactgggatgagagaaagtgggacctggtgggctcaaccacacacacctgtctgtgcagatgctttgcccaggcttctcaccacggtgtaccgggatattaaacctctttccccagcctgga

SEQ ID NO: 72 - DNA, 1186 bp, Homo sapiens
  source 1..1186: mol_type = genomic DNA
ggtttccggtgcaggtggggaaaatggcggtgtctacagtgttctcgacttcgtcgctgatgcttgctctgtcaaggcacagcctattgtctcctttgctcagtgtgacatcattcagacgcttctacagaggtgacagcccaacagattcccaaaaggacatgattgaaatccctttgcctccatggcaggagagaactgatgaatccatagaaaccaaaagagcccgcctgctctatgagagcagaaagaggggaatgttggaaaactgcattcttcttagtctttttgctaaagaacatctgcagcacatgacagaaaagcagctgaacctctatgaccgcctgattaacgagcctagtaatgactgggatatttactactgggccacagaagctaaaccagccccagaaatatttgaaaatgaagtcatggccctgctgagagactttgctaaaaacaaaaacaaagagcagagactgcgtgccccagatcttgagtacctctttgaaaagccacgttgagctgtgctccacggcctggcatgggggttcagtctgtggatggtaactacttatgatggacgttagccttgcttccggcttcttagatgcccagctgccctaccccagaccactggtcctgcctcaagtgatggacataaccctctcctaggacatacatgtaaatgcacaatgtgactcattctcatacttttttgttcagctctgagcctcaaaagtgatttgtcagcagcgctggcatgcaggctttgcctggctgctatctctagaggcaagtctgccacgagagggcactgcaggcgaagcagttgtgcttgctcattgcctcagcccaagtgtactcaaagaaaagaggcagccagctgtgcgtgctgtatggaaagcctcccgccctccctgcagctccccgccctcagtggcccagggctttgttgaagtggaactccccacttccaacctggtatggctccttctgcgaagggaagctgatcccagcctcctgagctgaatccttcaaaggcacagcaggcagaatgagggccacaggcaggagtggtgtgggcacactgcttaggagtcaacatcttcatcattgggctttctttaaaacgtgcactttgtaatttgaaagagaattattaaagcatactgaaaaaaggaaa

SEQ ID NO: 73 - DNA, 2646 bp, Homo sapiens
  source 1..2646: mol_type = genomic DNA
acatgcgcaggcaggtgatggcggcgctggtcgtatccggggcagcggagcagggcggccgagacggccctggcagaggtcgggcccctcggggccgcgtggccaatcagatcccccctgagatcctgaagaaccctcagctgcaggcagcaatccgggtcctgccttccaactacaactttgagatccccaagaccatctggaggatccaacaagcccaggccaagaaggtggccttgcaaatgccggaaggcctcctcctctttgcctgtaccattgtggatatcttggaaaggttcacggaggccgaagtgatggtgatgggtgacgtgacctacggggcttgctgtgtggatgacttcacagcgagggccctgggagctgacttcttggtgcactacggccacagttgcctgattcccatggacacctcggcccaagacttccgggtgctgtacgtctttgtggacatccggatagacactacacacctcctggactctctccgcctcacctttcccccagccactgcccttgccctggtcagcaccattcagtttgtgtcgaccttgcaggcagccgcccaggagctgaaagccgagtatcgtgtgagtgtcccacagtgcaagcccctgtcccctggagagatcctgggctgcacatccccccgactgtccaaagaggtggaggccgttgtgtatcttggagatggccgcttccatctggagtctgtcatgattgccaaccccaatgtccccgcttaccggtatgacccatatagcaaagtcctatccagagaacactatgaccaccagcgcatgcaggctgctcgccaagaagccatagccactgcccgctcagctaagtcctggggccttattctgggcactttgggccgccagggcagtcctaagatcctggagcacctggaatctcgactccgagccttgggcctttcctttgtgaggctgctgctctctgagatcttccccagcaagcttagcctacttcctgaggtggatgtgtgggtgcaggtggcatgtccacgtctctccattgactggggcacagccttccccaagccgctgctgacaccctatgaggcggccgtggctctgagggacatttcctggcagcagccctacccgatggacttctacgctggcagctccttggggccctggacggtgaaccacggccaggaccgccgtccccacgccccgggccggcccgcgcgggggaaggtgcaggaggggtccgcgcgtcccccttcggccgtggcttgcgaggactgcagctgcagggacgagaaggtggcgccgctggctccttgacgcgctcccgggcctcagggtcctgccctccggaggagcagcctcgaggctggtggttttcagagcaggaggccgacgttttctccgcattggaagagcccgccgtctgcaggggcctggaggaatcactggggatggtggcacaggcactgaacaggctggggccttttgacggccttcttggtttcagccaaggggctgcgctagcagcccttgtgtgtgccctgggccaggcaggcgatccccgcttccccttgccacggtttatcctcttggtgtctggtttctgtccccggggcattgggttcaaggaatccatcctgcaaaggcccttgtcattgccttcgctccatgtttttggggacactgacaaagtcatcccctctcaggagagtgtgcaactggccagccaatttcccggagccatcaccctcacccactctggtggccacttcattccagcagctgcaccccagcgtcaggcctacctcaagttcttggaccagtttgcagagtgaaagatcaagaaatgtctctgctcctacatccagctcctctaggggcagcctccgtcatccatgccctcccaggaccctccactcactgctgtgagtgcgcctcaccagaaccagttaagagacaactatcaattcttgagacccaaattataagggccctgccctgtactgaagaaaaggggagcacaaggccttaatggacattgacttgtgaaaacgcaaacatgaatatggttggagagccctggattaggagggtgacatggggaaggcagaggctggcaccatggtgactgccacataataaagtggtgatttggattttgagcatctttttcctggtacacacagaaaaacattttataatggaagtcggtttcttggccatctacatagttttctgggcagcgccaagcagggaggtgtctccagttgtgagtcctcggacaggctgctgcatgggtgcacatactcacgttattggtggaagttttaagtcccaaactgaagggaaggaggcctaggtggcacaatcagggaggaatatacatctgagaagttttgggaagacatcacctggcaaggctgcctgaaccacagtaatttagtcttcctctatccagatcactgagagttgtgtgctggtcttctctcaaaccctggatgtgttacctgcttctccctaagtgctctaacctcgctgaactatgagcctgaagggtgggtcgatgccagaccttaggtggaggccaaagacagtacagaataaacagcttcttcctaaattgacccca

SEQ ID NO: 74 - DNA, 2466 bp, Homo sapiens
  source 1..2466: mol_type = genomic DNA
agtagttaggatggctgaaggggatactcaccggctgaaggccgactgtgattccccctacccccacaaggcgattttgaccccctgagggctgctctagaggactcaggccccgaagctgtcccagggaggtccccgctgcatcccaccacccaagctgtgcctcatggagtcgatgtttagcagccctgccgaggcggcgctgcagcgagagaccggggtgccaggactgcttactcctcttccggacctggacggagtgtacgagctggagcgagtcgctggatttgtccgcgacctggggtgtgaacgagttgccttgcagttccctgaccagctattgggagatgctgtggctgtggctgcacgactggaggagacgacagggtcaaagatgttcattctgggtgacacagcctacggcagctgctgcgtggatgtgctgggtgctgagcaagctggagctcaggctctcatacattttggccctgcctgcttaagccctccagcccgcccactgcccgttgccttcgtgcttcgtcaacgttctgtggccttggagctctgtgtcaaggcctttgaggcccagaacccagaccccaaagcgcctgtggtgctgctgagtgagccggcctgtgcccatgccctggaggctttggctactctcctgcgcccacggtacctggacctgctagtctccagcccagcttttccccaaccagtgggttccctgagtccagagcctatgcccctagagcgttttgggcgccgcttcccccttgccccagggaggcgtctagaagagtatggtgccttctatgtagggggctctaaggccagccctgacccagaccttgacccagacctgagtcggctgctcttggggtgggcaccaggtcaacccttctcctcctgctgtccagatacagggaagactcaggatgagggtgcccgggctggacggctaagggcacgaagacgatatctggtagagagggccagagatgcccgcgtggtagggctgctggcaggcacactgggtgtagcccaacaccgtgaggcactggcccacttgcggaacctgactcaggctgctggcaagcgtagctatgtgttggccctggggcggcccacccctgccaagcttgccaacttccctgaggtggatgtctttgtgctattagcctgtcctctgggtgctctagccccccagctttctggtagcttcttccagcctatactggcaccatgtgagctggaagctgcctgcaaccctgcctggccacctccaggcctggctccccacctcacacattatgcggacttattgcctggctctcccttccacgtggctctcccaccacctgagtcagagctgtgggaaaccccagacgtgtcactcattactggagatctccgacccccacctgcctggaagtcatcaaatgatcatggaagcttggctctgaccccacggccccagctggagctggctgagagcagtcctgcagcctcattccttagttcccggagctggcaagggctggagccccgcctgggtcagacgccagtgacagaagctgtgagtggaagacgagggattgccatcgcctatgaggatgagggaagcggctgataccatgtggggctggagacatagatggacttatgaatggctgctaggacctttagtgctccctgcaccaacctcccatccccctgccaagatccttgaaggaccctggaaggagggagagcaggcagcccttcacaggataggatccgtctctgtcctgtcctggcactggcacaagctcagcacatgcccagtaatgcgtgttgtttggctgatggaataaagggcttagggacttccctgaggcctctggacccatctgtcttcctgagggcagcccaggacctttggccaatcccagttcccaggctgcagttgagggtctgtccttgtcaaaaggcaggtgctagacagtctagaccagggtttctcaaactcgtacttgacatttggggccagataattctttgttgtggggctgtctggtgtatggtagggtgctcagcagcatccctggcctctgcccactagacatcagaagcactcccccagttgtgacaaccaaaaatatctccagaccttggcaaatgttatctgtgggggaaaattgccctcaattgagaaccactggtctagctagacctgcactgtccagtacagtagccactaaatacatgtggctaaacttaaatttaagttaattaagattaaaagctcagtttctcagtcacattagtcattcaagtgttcagacagccacatgaggggacagtgcagctacaggatatgccatcatggcagaaagttctattggttggacagcgttggtctatactgactcttatttctcagggagatcacagcaacctaaataaaccagataccttttc

SEQ ID NO: 75 - DNA, 2434 bp, Homo sapiens
  source 1..2434: mol_type = genomic DNA
agaactcgggtgcagccaatcgagggcaacgctgctacttatcagagcagaatgggctgtagtttagtgaaataggaaagctgcaaaacactgtggagtgctcccgtgtaaataaaaagaggaaaaaagtttctcaagtcgccgctgcacgacgtctggccggcgctggagcgggggtctgcgctctcccgagcggccgcgcgctggactttattgtgccgcaaccagccccagttcccattgtttgtgtttttttcaaaatatggcaaaggttcaggtgaacaatgtagtggtgctggataacccttctcctttctacaacccgttccagttcgagatcaccttcgagtgcatcgaggacctgtctgaagacttggaatggaaaattatctatgtgggctctgcagaaagtgaagaatacgatcaagttttagactctgttttagtgggtcctgttcccgcaggaaggcatatgtttgtatttcaggctgatgcacctaatccaggactcattccagatgcagatgcagtaggcgtaactgttgtgctaattacttgtacctatcgaggacaagaatttattagagttggctattatgtaaataatgaatatactgagacagaattaagggaaaatccaccagtaaaaccagacttttctaagcttcaaaggaatattttggcatctaatcccagggtcacaagattccacattaattgggaagataacacagaaaaactggaagatgcagagagcagtaatccaaatctacagtcacttctttcaacagatgcattaccttcagcatcaaagggatggtccacatcagaaaactcactaaatgtcatgttagaatcccacatggactgcatgtgaccacctaccatccctttagtacaaattaagctattaaaaatacacagaactatttccctgaaattccgtaagtacatagtcaaaacacaatgtgaagaatttgtttaaaaacatcctgtagaaagtttataagaaaaccagtatttgaacaaattgtggaatataaatacaactatttttaagtaatttttttctctaatgtgttattttatttgttctgaaactaatctgattaaagcatatatattattttcttctcctttatatgtaatgaaagcacttataaagaaacaggaatcattagaccaggttgtaaagatgtcttggcctcccagacagtctttggaccactattttactggcctttgaaagaacaaagtttacttcaactaaaatgtttcttcctgtgaagacatatggtatcattttaatttaaggggcagatttccattcttttttggcatgtccttaaaaagaggatttgaaatcaactatatgctaccaagaactttaggatccaattttccaagccaccgtgaagcctcttatggctactattatagttcatgagatgttggacagcatttggtttgtttggtaagagaagaacaaatatggctaattgttaataaggtcccctggctatggtttttgttcttataacagagtttagaatatcagagcatttcttgaatcatacatcattattgtccagtgaattcaagaccaaatacaatatcgggagaaaatacaaactccctgtgtttaagaataggggattacaatggctcaaattggttcatttgatttctaaaaataccatgacctttaaaaattcttttaatagatcatgttattggcagtatttatataaaaaccatggatttagaaaggtatatttagctttattataatagatacgttactattttggaaatatatatattttcacacctgtagtactcttccccatttcctctgacactcatgcagaatgagatcaggcatatttgtggttgacatactctagatagcttttccaacaaatctttgaaaagcaatcttggaaaggaattcattaactaaatggggtaaaaggtttctttttctttgagagttaagtccttcctcaaaaaattttcttatgctccataaaattgaggtagaatattttcattattcttgcagctaagcaagacagccatatgatgcaggttatgcagtgccttcatatctaaaacttttatactgcactttttaaagcttttatgttgaggaaaggaaaagggcatttgtctaaacatggattctgagttgtatatatttcctatcattctaaaaaagtgaatttgtgaagcaaagttttgccagatgttaaactttgaatttaatataccagattattaaaatattgtccattaactagttcatagattttaaaagtaaaatacttctgactgttaaaactatataaagaaaatctcatttgtctaattgcaattaaaagaaaaatgttaaaatattaaaatgaaaataaaagcattactttccaacaaa

SEQ ID NO: 76 - DNA, 2286 bp, Homo sapiens
  source 1..2286: mol_type = genomic DNA
ggcaggggcgcggggcttctggaggcggcggcggtggccagcgcggcggcgccggccgtattctccgggctgcggagggtaaagagcgggctcgggccgaggctggagggctgggtggggccagagcggcgcttcgggggcccgcggaggacgagggagggagagaatctgaggagctgggttgccattaggggactcctgaggtcctatctccaggctgcggtgactgcactttccctggagtggaagctgctggaaggcggaccggccgccatgtccacgttcaggcaggaggacgtggaggaccattatgagatgggggaggagctgggcagcggccagtttgcgatcgtgcggaagtgccggcagaagggcacgggcaaggagtacgcagccaagttcatcaagaagcgccgcctgtcatccagccggcgtggggtgagccgggaggagatcgagcgggaggtgaacatcctgcgggagatccggcaccccaacatcatcaccctgcacgacatcttcgagaacaagacggacgtggtcctcatcctggagctggtctctggcggggagctctttgacttcctggcggagaaggagtcgctgacggaggacgaggccacccagttcctcaagcagatcctggacggcgttcactacctgcactctaagcgcatcgcacactttgacctgaagccggaaaacatcatgctgctggacaagaacgtgcccaacccacgaatcaagctcatcgacttcggcatcgcgcacaagatcgaggcggggaacgagttcaagaacatcttcggcaccccggagtttgtggccccagagattgtgaactatgagccgctgggcctggaggcggacatgtggagcatcggtgtcatcacctatatcctcctgagcggtgcatccccgttcctgggcgagaccaagcaggagacgctcaccaacatctcagccgtgaactacgacttcgacgaggagtacttcagcaacaccagcgagctggccaaggacttcattcgccggctgctcgtcaaagatcccaagcggagaatgaccattgcccagagcctggaacattcctggattaaggcgatccggcggcggaacgtgcgtggtgaggacagcggccgcaagcccgagcggcggcgcctgaagaccacgcgtctgaaggagtacaccatcaagtcgcactccagcttgccgcccaacaacagctacgccgacttcgagcgcttctccaaggtgctggaggaggcggcggccgccgaggagggcctgcgcgagctgcagcgcagccggcggctctgccacgaggacgtggaggcgctggccgccatctacgaggagaaggaggcctggtaccgcgaggagagcgacagcctgggccaggacctgcggaggctacggcaggagctgctcaagaccgaggcgctcaagcggcaggcgcaggaggaggccaagggcgcgctgctggggaccagcggcctcaagcgccgcttcagccgcctggagaaccgctacgaggcgctggccaagcaagtagcctccgagatgcgcttcgtgcaggacctcgtgcgcgccctggagcaggagaagctgcagggcgtggagtgcgggctgcgctaggcgcagtggggtgggccaggccccaggacagccggagctcggcctgcggtgggggcgcttcctgtggacgctgcgcctcccatcgcccgggtgcctgtccttgcccagcgccaccaggctggaggcggagtgggaggagctggagccaggcccgtaagttcgcaggcaggggtgggtgtgggacggggctgcttctctacacagcctccacgctggccttcaccttcacccctgcatcgtcggtgaccctgggaccctccaggcagcgtggcctgtggcaccgtgagggttgggacccaccgaggcgcagaggcggcccgaatgcagccctggttcaggcccggaggagggtttgcgggtagttgcacggacaattcggcggggtgctgcctgttgctgccattagcccaggaggaggtcgtgggacggggagggtgggatggacggcggacaggcagtccccacgctgctgggtggcgccgggcttggtggggtcttccactgtgtgcccttctcgccgaggccggtcccccgggtgtggggtgccctgctgcggactcctccgcgagccccatcgtcgcgcctgtggacgcctaggcaagagcggccctctgcagccaagagaaataaaatactggcttccagata

SEQ ID NO: 77 - DNA, 2333 bp, Homo sapiens
  source 1..2333: mol_type = genomic DNA
gcggacgcaacggtgggcacgatgcggcaacccgccgttcggttggtgcgcctgggtcgggtgccgtacgccgagctactggggctgcaggaccgctggctgcggcggctgcaggccgagccaggcattgaggccccgtcggggactgaggcgggcgcgctcctgctctgcgagcccgcggggcccgtgtatacggccgggctgcgcggcggcctgacgcccgaggaaactgcgcggctacgggccttgggcgccgaggtgcgcgtcacaggccgcggtggcctggccaccttccacggcccgggccagctgctttgccacccggtactcgacctgcggcgtctcggcctgcgcttgcgcatgcacgtagcgtcgctggaggcgtgcgccgtgcgcctgtgcgagctccagggcctgcaggacgcccgcgcgcggcccccgccctacactggcgtctggctagacgatcgcaagatctgcgcgatcggagtccgctgtggaaggcacatcacatcccacggcctggctctcaactgctctaccgacctcacgtggtttgagcacatcgtgccctgtggactggttgggacaggcgtcacttccttgagtaaggagctccagaggcacgtcaccgtggaagaagtaatgccacctttccttgtggcctttaaggagatctacaagtgcacactgatctcagaggacagccccaactgaagagtactcataacagccaggatgctcctgccttgggaagcatgaagtctgacttgagtcacctactgaaacccagattttagacctggcctgtcattcactgatcatgaaactaaggacataccatgagctctgagccactattttcatatgtatcctgttttatttatatcttccccacctacctcagagatactagatgtgacaatactgtgaaaagcaacacatgctatataaaacattagcactgctattgacagcagtgtctcagagaaattcagatgtcatagattgttatttctcgattgttgatgtcatagattgtcatttctcaattgttgcttcctcaatactgtgtctaactgagcaattctgggctctagttcctttgattttgtgggaatcaaatctctgaatggcccaggtgaaggtgatgtgatgtagtgctttttcattctcaagagacctgggttctttctgcaataaagggaaaagggaagaaccttctaagaataaaatgggatcttaccttcctgggtttgattccccaagtttgataatttagcagtttttagttgggttaccctgagctaggccaaagactggaggaaggataagaataatttctcacactgtacaaatgccaaagatagaagtgagtttttcttatatgagacctctaaatttgtaaggctgcgtattatctgttgctgtgtaacaaatcatcaaaacttagtgacttaacagtaaacattaattatctcgtttgtgggtcaggaatttgggggaagcttagctgggtgtttctaggttagggtctcacacgaacttgcagttaagatgtcagctggaactgcacttattcaaaggcttggctgggctggaggatcagcgctccatatggtgcattcacttgcgtggttggttatggttgctgacaagaggcctcagttccttaccgtgtggacctccctatagggttgcttaattgttctcatgacagggcaactggcttcccctagagcaagtcatgccaaagagagacaagctggaagccacaatgttttttatgacccagcctcagaagtcacacaggcatttctgtagtcttctaacagatgagccctgttcagtgtggaagggactgcactagggcaacagaaggtgaggattattaggggccatcttgaagaatggccaccacagcctattaagagtggaaagagcctcgaatgagaagtcaggggaccctggttccagttctggctctgtggaaaagtcatttactctctggtcttcagtttctccttaaaccagtgctgttcaatagaaatacaatgcaagccacattgtaatttgacattttctaggatccatgttaaaaaggtaaaagaaatgggtgatgttatttttattttattttatttttattttttttaagaaatgaggctttgctatgttgcccaagctggtctcagactcctgggctcaagggatccttctgcctcagcctcacaaagtgctggaattacaggtgtgagccaccatgcctggtctatgatgttaattttaatattttaattaacccaatatataaaaatatcaatataaagcaa

(19) United States
(12) Patent Application Publication
(10) Pub. No.: US 2025/0066769 A1
(43) Pub. Date: Feb. 27, 2025

(54) METHODS OF SCREENING AND TREATING FRAGILE X SYNDROME

(71) Applicant: YISSUM RESEARCH DEVELOPMENT COMPANY OF THE HEBREW UNIVERSITY OF JERUSALEM LTD., Jerusalem (IL)

(72) Inventor: Nissim BENVENISTY, Jerusalem (IL)

(73) Assignee: YISSUM RESEARCH DEVELOPMENT COMPANY OF THE HEBREW UNIVERSITY OF JERUSALEM LTD., Jerusalem (IL)

(21) Appl. No.: 18/724,244

(22) PCT Filed: Dec. 26, 2022
(86) PCT No.: PCT/IL2022/051390

§ 371 Date: Jun. 26, 2024

Related U.S. Application Data
(60) Provisional application No. 63/293,792, filed on Dec. 26, 2021.

(51) Int. Cl. C12N 15/10

(52) U.S. Cl. CPC C12N 15/1072; C12N 15/1082

(57) ABSTRACT

Methods of treating fragile X syndrome by administering agents that reduce expression or function of various proteins are provided. Methods of determining suitability to be treated by the methods of the invention are also provided.

### CROSS-REFERENCE TO RELATED APPLICATIONS

This application claims the benefit of priority of U.S. Provisional Patent Application No. 63/293,792 filed Dec. 26, 2021, the contents of which are incorporated herein by reference in their entirety.

### REFERENCE TO AN ELECTRONIC SEQUENCE LISTING

The contents of the electronic sequence listing (HUJI-P-083-PCT_SQL.xml; Size: 273,980 bytes; and Date of Creation: Dec. 26, 2022) is herein incorporated by reference in its entirety.

### FIELD OF INVENTION

The present invention is in the field of fragile X syndrome treatment.

### BACKGROUND OF THE INVENTION

Since the identification of the trinucleotide CGG repeat expansion in the fragile X mental retardation 1 (FMR1) gene as the causative mutation in Fragile X syndrome (FXS), many efforts have been invested in deciphering the epigenetic processes that disrupt FMR1 expression in patients' cells. Although several studies have characterized the heterochromatic configuration of full mutation alleles (>200 CGG repeats), the factors involved in causing and maintaining FMR1 heterochromatinization remain elusive. It is assumed that the recruitment of repressive DNA-binding factors by the expanded CGG repeats mediates the DNA hypermethylation and inactivation of the FMR1 locus, similar to other disease-associated repeat expansions that are characterized by the acquisition of abnormal DNA hypermethylation.

As the FXS-causing mutation is located in the non-coding region of FMR1, understanding and targeting the mechanisms responsible for FMR1 inactivation would have a therapeutic value. Rare existence of individuals with apparent normal intelligence who carry an unmethylated CGG expansion indicates that the expression of full mutation alleles produces functional FMRP (Fragile X mental retardation protein, the protein product of FMR1) and can prevent the neurocognitive manifestations of FXS.

hPSC-based models of FXS allow one to study the mechanisms responsible for FMR1 silencing and to explore novel treatments capable of reactivating FMR1 expression. Compound screening using FXS induced pluripotent stem cells (iPSCs) that harbor a completely silenced FMR1 allele have highlighted the importance of DNA methylation in the maintenance of FMR1 silencing and identified several candidate compounds that are able to target FMR1 heterochromatinization (Vershkov et al., 2019, “FMR1 reactivating treatments in fragile X iPSC-derived neural progenitors in vitro and in vivo”, Cell Rep. 26, 2531-2539). However, other therapeutic targets beyond just DNA methylation pathway genes are greatly needed in order to uncover other therapeutic modalities.

### SUMMARY OF THE INVENTION

The present invention provides methods of treating fragile X syndrome by administering agents that reduce expression or function of at least one of SMARCD1, QRICH1, CDK4, TAF8, ZFP90, ADNP, SPOP, ZNF217, CTBP2, TBX5, NHLH2, SOHLH1, UBR5, TP53BP1, EME1, PHF5A, ZBTB14, ZNF200, SATB2, USP47, DPF3, HOXC13, EVX2, ANKRD2, ZBTB24, SMEK1, ZNF385A, ZNF446, C6orf57, SLC25A19, SDHC, CEND1, ME2, ISCU, TRUB2, SDHA, PSEN1, TEFM, GATM, HSPA9, ARMCX3, DAO, FBXW7, FIS1, HSD3B1, DIABLO, HK2, HADHA, SDHAF2, DPH1, DPH2, ASF1A, DAPK3, and LIPT2. Methods of determining suitability to be treated by the methods of the invention are also provided.

According to a first aspect, there is provided a method of treating Fragile X syndrome (FXS) in a subject in need thereof, the method comprising administering to the subject an agent that inhibits expression or function of a protein selected from the group consisting of: SMARCD1, QRICH1, CDK4, TAF8, ZFP90, ADNP, SPOP, ZNF217, CTBP2, TBX5, NHLH2, SOHLH1, UBR5, TP53BP1, EME1, PHF5A, ZBTB14, ZNF200, SATB2, USP47, DPF3, HOXC13, EVX2, ANKRD2, ZBTB24, SMEK1, ZNF385A, ZNF446, C6orf57, SLC25A19, SDHC, CEND1, ME2, ISCU, TRUB2, SDHA, PSEN1, TEFM, GATM, HSPA9, ARMCX3, DAO, FBXW7, FIS1, HSD3B1, DIABLO, HK2, HADHA, SDHAF2, DPH1, DPH2, ASF1A, DAPK3, and LIPT2, thereby treating FXS.

According to some embodiments, the group consists of: QRICH1, CDK4, TAF8, SMARCD1, ZFP90, ADNP, SPOP, ZNF217, CTBP2, TBX5, NHLH2, SOHLH1, UBR5, EME1, PHF5A, ZBTB14, ZNF200, SATB2, USP47, DPF3, EVX2, ZBTB24, SMEK1, ZNF446, C6orf57, SLC25A19, SDHC, CEND1, ME2, ISCU, TRUB2, SDHA, PSEN1, TEFM, GATM, HSPA9, ARMCX3, DAO, FBXW7, FIS1, HSD3B1, HK2, HADHA, SDHAF2, DPH1, DPH2, ASF1A, DAPK3, and LIPT2.

According to some embodiments, the group consists of QRICH1, TAF8, SMARCD1, ZFP90, ADNP, ZNF217, CTBP2, ZBTB14, SATB2, and C6orf57.

According to some embodiments, the group consists of QRICH1, TAF8, SMARCD1, ZFP90, ZNF217, CTBP2, SATB2, and C6orf57.

According to some embodiments, the group consists of SMARCD1, ZNF217 and C6orf57.

According to some embodiments, the agent inhibits ZNF217 and is an LSD1 inhibitor, a CoREST inhibitor or both.

According to some embodiments, the LSD1 inhibitor, CoREST inhibitor or both are selected from GSK2879552, bizine, corin, pargyline, and Rodin-A.

According to some embodiments, the agent inhibits ZNF217 and is an HDAC inhibitor.

According to some embodiments, the HDAC inhibitor is selected from MS-275, SAHA, and LBH589.

According to some embodiments, the agent inhibits SMARCD1 and is I-BRD9.

According to some embodiments, the agent inhibits C6orf57 and is a succinate dehydrogenase (SDH) inhibitor.

According to some embodiments, the SDH inhibitor is selected from Oxaloacetic acid, Malonate, Harzianopyridone, and 2-thenoyltrifluoroacetone.

According to some embodiments, the agent is selected from an inhibitory RNA, a blocking antibody and a small molecule inhibitor.

According to some embodiments, the treating comprises demethylation of a CGG repeat region of a 5′ UTR of a FMR1 gene in the subject.

According to some embodiments, the treating comprises inducing expression of FMR1 in the subject.

According to some embodiments, the method further comprises inhibiting expression or function of at least one other protein selected from the group provided in Table 1.

According to another aspect, there is provided a method of determining suitability of a subject in need thereof to be treated by a method of the invention, the method comprising measuring in a sample from the subject expression or function of a protein selected from SMARCD1, QRICH1, CDK4, TAF8, ZFP90, ADNP, SPOP, ZNF217, CTBP2, TBX5, NHLH2, SOHLH1, UBR5, TP53BP1, EME1, PHF5A, ZBTB14, ZNF200, SATB2, USP47, DPF3, HOXC13, EVX2, ANKRD2, ZBTB24, SMEK1, ZNF385A, ZNF446, C6orf57, SLC25A19, SDHC, CEND1, ME2, ISCU, TRUB2, SDHA, PSEN1, TEFM, GATM, HSPA9, ARMCX3, DAO, FBXW7, FIS1, HSD3B1, DIABLO, HK2, HADHA, SDHAF2, DPH1, DPH2, ASF1A, DAPK3, and LIPT2, wherein expression or function above a predetermined threshold indicates the subject is suitable to be treated by a method of the invention, thereby determining suitability of a subject.

According to some embodiments, the group consists of: QRICH1, CDK4, TAF8, SMARCD1, ZFP90, ADNP, SPOP, ZNF217, CTBP2, TBX5, NHLH2, SOHLH1, UBR5, EME1, PHF5A, ZBTB14, ZNF200, SATB2, USP47, DPF3, EVX2, ZBTB24, SMEK1, ZNF446, C6orf57, SLC25A19, SDHC, CEND1, ME2, ISCU, TRUB2, SDHA, PSEN1, TEFM, GATM, HSPA9, ARMCX3, DAO, FBXW7, FIS1, HSD3B1, HK2, HADHA, SDHAF2, DPH1, DPH2, ASF1A, DAPK3, and LIPT2.

According to some embodiments, the group consists of QRICH1, TAF8, SMARCD1, ZFP90, ADNP, ZNF217, CTBP2, ZBTB14, SATB2, and C6orf57.

According to some embodiments, the group consists of QRICH1, TAF8, SMARCD1, ZFP90, ZNF217, CTBP2, SATB2, and C6orf57.

According to some embodiments, the group consists of SMARCD1, ZNF217 and C6orf57.

According to some embodiments, a cell from the sample comprises an expansion of a repetitive CGG sequence in a 5′ UTR of a FMR1 gene.

According to some embodiments, the expansion comprises at least 55 CGG repeats.

According to some embodiments, the subject suffers from FXS or is at risk of suffering from FXS.

According to another aspect, there is provided a method for producing an agent, the method comprising:
a. providing an agent that inhibits expression or function of a protein selected from the group consisting of: SMARCD1, QRICH1, CDK4, TAF8, ZFP90, ADNP, SPOP, ZNF217, CTBP2, TBX5, NHLH2, SOHLH1, UBR5, TP53BP1, EME1, PHF5A, ZBTB14, ZNF200, SATB2, USP47, DPF3, HOXC13, EVX2, ANKRD2, ZBTB24, SMEK1, ZNF385A, ZNF446, C6orf57, SLC25A19, SDHC, CEND1, ME2, ISCU, TRUB2, SDHA, PSEN1, TEFM, GATM, HSPA9, ARMCX3, DAO, FBXW7, FIS1, HSD3B1, DIABLO, HK2, HADHA, SDHAF2, DPH1, DPH2, ASF1A, DAPK3, and LIPT2;
b. contacting the provided agent with a cell comprising an FMR1 gene comprising a methylated CGG repeat region; and
c. selecting an agent that increases expression of FMR1 in the cell;
thereby producing an agent.

According to some embodiments, the agent is an agent that treats FXS.

According to some embodiments, the providing comprises contacting a plurality of agents with a cell expressing the protein and selecting at least one agent of the plurality that inhibits expression or function of the protein in the cell.

According to some embodiments, the group consists of SMARCD1, C6orf57, ZNF217, QRICH1, TAF8, ZFP90, CTBP2, and SATB2.

According to some embodiments, the group consists of SMARCD1, ZNF217 and C6orf57.

According to another aspect, there is provided an agent produced by a method of any the invention.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-1I: Analysis of DNMT1 disruption in FXS-iPSCs. (1A) Changes in FMR1 expression in FXS-iPSCs 7 and 12 days after the delivery of sgRNA and Cas9, as detected by RT-PCR, as compared to treatment with 5-azadC (5 μM, 4 days). (1B) Volcano plot showing the median log fold expression change (x axis) and −log (FDR) (y axis) for each gene in FXS-iPSCs following DNMT1 perturbation. (1C) Percentage of upregulated genes (FDR<0.05, log (Fold change)>1) per chromosome following DNMT1 disruption in FXS-iPSCs. ***hypergeometric P-values<0.001. (1D) Positional enrichment analysis in GSEA for upregulated genes upon DNMT1 disruption. (1E) Heatmap of expression levels (Z score TPM) across tissues (data from the GTEx study) of the significantly upregulated Xq27-28 genes (FDR<0.05) in FXS-iPSCs following DNMT1 disruption. (1F) Enriched GO and Human Phenotype ontology terms (analyzed using GSEA, FDR q values<0.05) among the upregulated genes in FXS-iPSCs following DNMT1 disruption. (1G) Activation of testis-specific marker genes involved in transcriptional regulation upon DNMT1 disruption. (1H) Heatmap of expression levels across tissues (data from the GTEx study) of genes located in the Fragile X adjacent region (140 MB-148 MB). (1I) Heatmap of expression levels across tissues (data from the GTEx study) of all the upregulated genes (FDR<0.05) in FXS-iPSCs following DNMT1 disruption.

FIGS. 2A-2F: Establishment of a screening protocol for the identification of genes involved in FMR1 silencing. (2A) Schematic illustration depicting the loss-of-function genetic screening experimental setup. Haploid hESCs transduced with the lentiviral CRISPR/Cas9 sgRNA library targeting 18,166 genes with ˜10 sgRNAs per gene were transfected with methylated pFMR1-(240) CGG-EGFP construct in four independent replicates. The least (˜30%) and the most (3-4%) GFP-fluorescent cells were sorted 48 hours following transfection, and DNA sequencing of the sgRNA segment was used to analyze the distribution of sgRNAs within the GFP positive and GFP negative populations, and to identify enriched genes within the GFP fluorescent fraction. (2B) GFP fluorescence shown for the transient transfection of haploid human ESCs with unmethylated pFMR1-(240) CGG-EGFP construct, 48 hours post transfection. (2C) Analysis of methylation of the pFMR1-(240) CGG-EGFP construct using the methylation sensitive restriction enzyme McrBC, which cleaves DNA containing methylcytosine on one or both strands. The control plasmid contains one McrBC site. From left to right: 1—ladder; 2—control plasmid; 3—unmethylated construct; 4—methylated construct. (2D-2E) Transient transfection of haploid human ESCs with either unmethylated (2E, middle panel) or methylated (2E, right panel) pFMR1-(240) CGG-EGFP construct, followed by (2E) flow cytometry analysis 48 hours post transfection. (2D) Analysis of GFP fluorescence revealed more than 10-times fold enrichment in GFP positive fraction between the cultures. (2F) Heatmap of pyrosequencing analysis of DNA methylation of the FMR1 promoter sequence (in 11 CpG positions) in the methylated pFMR1-CGG (240)-EGFP plasmid, and the corresponding genomic CpG positions in FXS- and WT-iPSCs (positions (−456) to (−409) from the start site of FMR1 translation).

FIGS. 3A-3G: Screening for genes involved in FMR1 inactivation using CRISPR/Cas9 library in haploid hESCs. (3A) Volcano plot showing the median log 2 fold change (x axis) and −log (FDR) (y axis) of the mutants included in the library, calculated based on the distribution of the normalized sgRNA read counts between the GFP (+) and GFP (−) populations. Genes in the upper-right quadrant are defined as enriched (log 2 fold change>0.5 and FDR<0.05). Representative genes of the main groups among the enriched genes are indicated. Metabolic factors appear in boxes, growth restricting tumor suppressor genes appear in ovals and the remaining genes are in the ‘Epifactors’ database. (3B) Flowchart demonstrating the main steps of the analysis for defining the candidate list of genes predicted to be involved in FMR1 inactivation. (3C) Enriched gene ontology (GO) terms (analyzed by GSEA, FDR q values<0.05) for the top significant genes with over-represented gRNAs in the GFP positive population (average log 2 fold change of gRNA abundance>0.5 and FDR<0.05, in genes expressed in haploid hESCs (TPM>0.1)). Two groups of enriched terms: terms associated with chromatin regulation (top) and terms associated with mitochondrial function. (3D) Bar graph of the percent of genes included in the Epifactors database. (3E) log 2 fold change of enriched epigenetic factors in the mFMR1-(240) CGG-GFP screen, compared to the their log 2 fold change values, also known as CRISPR score, previously observed in an essentiality screen in hESCs. (3F) log 2 fold change of enriched mitochondrial factors in the mFMR1-(240) CGG-GFP screen, compared to the their log 2 fold change values, also known as CRISPR score, in a previous hESC essentialome screen. (3G) Representatives genes for different functional groups within the candidate gene list. Black lines connecting the gene names indicate protein-protein interactions identified by STRING analysis.

FIGS. 4A-4F. Verification of candidate hit genes predicted to be involved in FMR1 inactivation. (4A) Experimental workflow. Mutant haploid hESCs were transfected with either methylated or unmethylated pFMR1-(240) CGG-GFP construct and analyzed by flow cytometry. (4B) Analysis of the mutants of candidate genes reveals higher relative GFP fluorescence following transfection with methylated pFMR1-(240) CGG-EGFP, relative to samples infected with Cas9 without sgRNA. Bars indicate the normalized ratio between GFP fluorescence in the cell culture transfected with methylated pFMR1-(240) CGG-EGFP and the GFP fluorescence in the cell culture transfected with the unmethylated construct. 5-azadc was used as a positive control. (4C) Analysis of heterogenous mutant populations of FXS-iPSCs for candidate genes SMARCD1, C6orf57, ZNF217, ZFP90, CTBP2, SATB2 and TAF8. Statistical tests were performed from three experiments. Error bars represent SEM. (4D) Bar plot showing the number of the significantly upregulated (blue) and downregulated (red) genes for the DNMT1, SMARCD1 and ZNF217 samples (p value<0.001, |log FC|>1). (4E) Enriched gene sets among the genes upregulated following either SMARCD1 or ZNF217 disruption. (4F) Schematic representation summarizing a proposed model of FMR1 epigenetic regulation following CGG repeat expansion.

### DETAILED DESCRIPTION OF THE INVENTION

The present invention, in some embodiments, provides methods of reactivating transcription of a fragile X mental retardation 1 (FMR1) gene in a cell and treating an FMR1-associated disease in a subject by administering an agent that reduces expression or function of at least one of SMARCD1, QRICH1, CDK4, TAF8, ZFP90, ADNP, SPOP, ZNF217, CTBP2, TBX5, NHLH2, SOHLH1, UBR5, TP53BP1, EME1, PHF5A, ZBTB14, ZNF200, SATB2, USP47, DPF3, HOXC13, EVX2, ANKRD2, ZBTB24, SMEK1, ZNF385A, ZNF446, C6orf57, SLC25A19, SDHC, CEND1, ME2, ISCU, TRUB2, SDHA, PSEN1, TEFM, GATM, HSPA9, ARMCX3, DAO, FBXW7, FIS1, HSD3B1, DIABLO, HK2, HADHA, SDHAF2, DPH1, DPH2, ASF1A, DAPK3, and LIPT2. Methods of determining suitability to be treated by the methods of the invention are also provided.

The invention is based on the surprising finding of pathways regulating FMR1 silencing, and identification of novel targets for FMR1 reactivation. While establishing complete loss-of-function phenotypes is somewhat challenging in diploid cells that require the establishment of homozygous mutations, the use of haploid cells, that harbor a single set of chromosomes, increases the chances of conducting a comprehensive functional genetic screen. Therefore, a tractable model system was generated to study FMR1 inactivation in haploid human embryonic stem cells (ESCs) and it was used to screen for genes involved in FMR1 inactivation. These results are also provided in Vershkov et al., “Genome-wide screening for genes involved in the epigenetic basis of fragile X syndrome”, Stem Cell Reports, 2022 May 10; 17 (5): 1048-1058, herein incorporated by reference in its entirety.

As DNMT1 disruption resulted in only partial reactivation of FMR1 expression, to levels comparable with 5-azadC treatment, a search was performed to find additional mechanisms that contribute to FMR1 inactivation. To this aim, a genetic screening platform was developed that provides a traceable readout of the transcriptional output of an exogenous methylated FMR1 promoter followed by an expanded CGG repeat tract. This platform allowed for the utilization of haploid hESCs for the study of FMR1 silencing, although these cells harbor a normal range of endogenous CGG repeats and actively express FMR1.

While transfection with a plasmid containing full mutation length CGG repeats was not sufficient to repress EGFP expression, in vitro methylation of the construct efficiently silenced the reporter gene, establishing a clear and traceable phenotype suitable for large scale screening. Using the screen, a list of 155 candidate genes potentially important for FMR1 regulation was established. Those genes are provided in Table 1.

TABLE 1
155 Candidate genes predicted to be involved in FMR1 silencing

| # | gene_name | logFC | FDR |
|---|---|---|---|
| 1 | QRICH1 | 1.797 | 0.0271 |
| 2 | RABGGTA | 1.699 | 0.0185 |
| 3 | LIPT2 | 1.644 | 0.0003 |
| 4 | EMC7 | 1.416 | 0.0467 |
| 5 | SART1 | 1.389 | 0.0049 |
| 6 | FIP1L1 | 1.339 | 0.0341 |
| 7 | CDK4 | 1.335 | 0.0102 |
| 8 | C6orf57 | 1.320 | 0.0005 |
| 9 | SLC25A19 | 1.316 | 0.0009 |
| 10 | TACR3 | 1.284 | 0.0387 |
| 11 | MAP1A | 1.274 | 0.0017 |
| 12 | KCNQ5 | 1.256 | 0.0018 |
| 13 | MAN1C1 | 1.255 | 0.0259 |
| 14 | SDHC | 1.203 | 0.0001 |
| 15 | TAF8 | 1.188 | 0.0316 |
| 16 | MACF1 | 1.141 | 0.0027 |
| 17 | IFT74 | 1.134 | 0.0066 |
| 18 | CEND1 | 1.130 | 0.0066 |
| 19 | PPFIA4 | 1.128 | 0.0066 |
| 20 | SMARCD1 | 1.126 | 0.0094 |
| 21 | RNF220 | 1.116 | 0.0485 |
| 22 | ME2 | 1.115 | 0.0025 |
| 23 | FIGN | 1.086 | 0.0140 |
| 24 | NUP50 | 1.065 | 0.0271 |
| 25 | HSPB8 | 1.037 | 0.0237 |
| 26 | CASC5 | 1.028 | 0.0285 |
| 27 | ZFP90 | 1.023 | 0.0103 |
| 28 | STRADA | 1.013 | 0.0004 |
| 29 | ADNP | 1.000 | 0.0262 |
| 30 | SPOP | 0.998 | 0.0223 |
| 31 | ZNF217 | 0.997 | 0.0316 |
| 32 | CTBP2 | 0.978 | 0.0086 |
| 33 | LRRC32 | 0.978 | 0.0040 |
| 34 | TBX5 | 0.978 | 0.0262 |
| 35 | C5orf30 | 0.960 | 0.0056 |
| 36 | ITPA | 0.952 | 0.0059 |
| 37 | C11orf1 | 0.939 | 0.0052 |
| 38 | ISCU | 0.938 | 0.0413 |
| 39 | TRUB2 | 0.929 | 0.0348 |
| 40 | NHLH2 | 0.927 | 0.0041 |
| 41 | RASEF | 0.925 | 0.0399 |
| 42 | SDHA | 0.918 | 0.0485 |
| 43 | SOHLH1 | 0.917 | 0.0329 |
| 44 | UBR5 | 0.909 | 0.0126 |
| 45 | DEFB127 | 0.908 | 0.0103 |
| 46 | EIF4E2 | 0.900 | 0.0003 |
| 47 | TP53BP1 | 0.895 | 0.0002 |
| 48 | WNT8B | 0.886 | 0.0403 |
| 49 | C1orf204 | 0.885 | 0.0162 |
| 50 | EME1 | 0.882 | 0.0123 |
| 51 | WNT3 | 0.863 | 0.0385 |
| 52 | DCAF11 | 0.845 | 0.0285 |
| 53 | FLCN | 0.829 | 0.0100 |
| 54 | PSEN1 | 0.821 | 0.0481 |
| 55 | SMIM15 | 0.805 | 0.0136 |
| 56 | TEFM | 0.802 | 0.0235 |
| 57 | KIF18B | 0.800 | 0.0072 |
| 58 | GATM | 0.798 | 0.0341 |
| 59 | THEMIS | 0.796 | 0.0285 |
| 60 | DEF8 | 0.795 | 0.0281 |
| 61 | KLHL41 | 0.790 | 0.0274 |
| 62 | GDF5 | 0.789 | 0.0395 |
| 63 | SIAH1 | 0.787 | 0.0059 |
| 64 | MAP7D1 | 0.785 | 0.0454 |
| 65 | CATSPER2 | 0.782 | 0.0156 |
| 66 | DPH1 | 0.777 | 0.0382 |
| 67 | PHF5A | 0.777 | 0.0065 |
| 68 | DPH2 | 0.777 | 0.0474 |
| 69 | SLC30A3 | 0.767 | 0.0368 |
| 70 | TPTE2 | 0.760 | 0.0131 |
| 71 | ALG9 | 0.759 | 0.0119 |
| 72 | HSPA9 | 0.757 | 0.0347 |
| 73 | DUOXA1 | 0.757 | 0.0464 |
| 74 | KRTAP5-8 | 0.752 | 0.0446 |
| 75 | ZBTB14 | 0.748 | 0.0395 |
| 76 | ZNF200 | 0.748 | 0.0041 |
| 77 | NBEAL1 | 0.745 | 0.0368 |
| 78 | SATB2 | 0.738 | 0.0407 |
| 79 | RIOK1 | 0.736 | 0.0284 |
| 80 | USP47 | 0.735 | 0.0052 |
| 81 | ANGPTL3 | 0.735 | 0.0139 |
| 82 | CLK1 | 0.732 | 0.0384 |
| 83 | PCDHAC2 | 0.731 | 0.0327 |
| 84 | PRKG2 | 0.731 | 0.0481 |
| 85 | TMEM43 | 0.730 | 0.0048 |
| 86 | AXIN2 | 0.730 | 0.0468 |
| 87 | ARMCX3 | 0.727 | 0.0246 |
| 88 | SNX27 | 0.727 | 0.0333 |
| 89 | DAO | 0.726 | 0.0220 |
| 90 | HSPA2 | 0.722 | 0.0178 |
| 91 | EPG5 | 0.712 | 0.0467 |
| 92 | VPS37B | 0.712 | 0.0395 |
| 93 | FAM46D | 0.711 | 0.0061 |
| 94 | ACSBG1 | 0.710 | 0.0359 |
| 95 | FBXW7 | 0.710 | 0.0431 |
| 96 | DPM1 | 0.704 | 0.0395 |
| 97 | KIAA1377 | 0.704 | 0.0057 |
| 98 | DPF3 | 0.703 | 0.0383 |
| 99 | KIAA0100 | 0.700 | 0.0485 |
| 100 | PTPLAD2 | 0.697 | 0.0463 |
| 101 | GSTM2 | 0.695 | 0.0083 |
| 102 | ABCA1 | 0.694 | 0.0444 |
| 103 | CACNG1 | 0.693 | 0.0317 |
| 104 | RBM41 | 0.692 | 0.0360 |
| 105 | HOXC13 | 0.691 | 0.0387 |
| 106 | SEPW1 | 0.683 | 0.0446 |
| 107 | C20orf112 | 0.680 | 0.0347 |
| 108 | FIS1 | 0.676 | 0.0131 |
| 109 | GRP | 0.675 | 0.0379 |
| 110 | CCDC71 | 0.674 | 0.0393 |
| 111 | HSD3B1 | 0.672 | 0.0041 |
| 112 | SGK2 | 0.669 | 0.0332 |
| 113 | RXFP2 | 0.666 | 0.0329 |
| 114 | TSPAN32 | 0.666 | 0.0329 |
| 115 | VWA3A | 0.665 | 0.0395 |
| 116 | LAMTOR5 | 0.661 | 0.0494 |
| 117 | SPATA33 | 0.658 | 0.0459 |
| 118 | BMP7 | 0.658 | 0.0335 |
| 119 | FBLN1 | 0.655 | 0.0413 |
| 120 | EVX2 | 0.654 | 0.0351 |
| 121 | KCNK2 | 0.653 | 0.0451 |
| 122 | EDN1 | 0.652 | 0.0326 |
| 123 | TNFRSF14 | 0.649 | 0.0364 |
| 124 | MSH5 | 0.649 | 0.0052 |
| 125 | NACA2 | 0.647 | 0.0149 |
| 126 | CCBL1 | 0.645 | 0.0386 |
| 127 | DIABLO | 0.644 | 0.0340 |
| 128 | ASAP1 | 0.642 | 0.0382 |
| 129 | GULP1 | 0.636 | 0.0318 |
| 130 | ANKRD2 | 0.634 | 0.0150 |
| 131 | ZBTB24 | 0.627 | 0.0379 |
| 132 | STPG2 | 0.621 | 0.0429 |
| 133 | BAG6 | 0.619 | 0.0289 |
| 134 | FAM49B | 0.616 | 0.0109 |
| 135 | ANAPC13 | 0.613 | 0.0352 |
| 136 | HK2 | 0.612 | 0.0379 |
| 137 | CDK5R1 | 0.610 | 0.0493 |
| 138 | SMEK1 | 0.609 | 0.0285 |
| 139 | ZNF385A | 0.600 | 0.0160 |
| 140 | NPY4R | 0.592 | 0.0041 |
| 141 | PEX11G | 0.588 | 0.0286 |
| 142 | MYL7 | 0.579 | 0.0482 |
| 143 | SWAP70 | 0.578 | 0.0114 |
| 144 | RAC2 | 0.576 | 0.0060 |
| 145 | DHX40 | 0.562 | 0.0288 |
| 146 | RPRML | 0.558 | 0.0269 |
| 147 | DNAL1 | 0.557 | 0.0124 |
| 148 | HADHA | 0.554 | 0.0372 |
| 149 | SLAMF8 | 0.553 | 0.0417 |
| 150 | SBF2 | 0.549 | 0.0482 |
| 151 | ZNF446 | 0.532 | 0.0488 |
| 152 | XKR8 | 0.519 | 0.0488 |
| 153 | ANAPC15 | 0.512 | 0.0223 |
| 154 | ZNF99 | 0.505 | 0.0353 |
| 155 | SDHAF2 | 0.500 | 0.0351 |

By a first aspect, there is provided a method of treating a fragile X mental retardation 1 (FMR1)-associated disease or disorder in a subject in need thereof, the method comprising administering to said subject an agent that inhibits expression or function of a protein provided in Table 1, thereby treating the FRM1-associated disease or disorder.

By another aspect, there is provided a method of reactivating FMR1 expression in a cell, the method comprising contacting the cell with an agent that inhibits expression or function of a protein provided in Table 1, thereby reactivating FMR1 expression in the cell.

By another aspect, there is provided a composition comprising an agent that inhibits expression or function of a protein provided in Table 1.

In some embodiments, the FMR1 is silenced. In some embodiments, the FMR1 is silenced in the cell. In some embodiments, the disease or disorder is characterized by FMR1 silencing. As used herein, the term “silenced” refers a region of the genome that can be transcribed, but in which transcription has been shut off. Thus, a silenced FMR1 is not merely lacking binding of a transcription factor, polymerase or other activator to start transcription, but rather is actively shut off. In some embodiments, a silenced FMR1 comprises heterochromatin. In some embodiments, a silenced FMR1 comprises a heterochromatic promoter. In some embodiments, a silenced FMR1 comprises silencing DNA methylation. In some embodiments, a silenced FMR1 comprises silencing DNA methylation in the promoter of FMR1. In some embodiments, a silenced FMR1 comprises silencing histone modification. In some embodiments, the silencing histone modification are in the promotor of FMR1. Examples of silencing histone modification include but are not limited to Histone H3 lysine 9 (H3K9) di- and tri-methylation, H3K27 trimethylation and H4K20 trimethylation. In some embodiments, the silenced locus comprises H3K27 trimethylation. In some embodiments, the silenced FMR1 comprises DNA and histone methylation. In some embodiments, the silenced FMR1 comprises DNA methylation and H3K27 trimethylation. In some embodiments, the silenced FMR1 comprises a methylated CGG repeat region. In some embodiments, the silenced FMR1 comprises H3K27me3 at the CGG repeat region. In some embodiments the CGG repeat region is consists of the CGG repeats.

In some embodiments, reactivating is reactivating transcription. In some embodiments, reactivating transcription comprises production of FMR1 RNA. In some embodiments, reactivating transcription comprises production of FMR1 RNA from the FMR1 gene, when no previous RNA was produced. In some embodiments, the reactivation is reactivation of transcription of a silenced FMR1. In some embodiments, the reactivating comprises an increased expression of the silenced transcript as compared to reactivation with a known reactivating agent. In some embodiments, the reactivating agent is 5′-azacytidine (5′-aza). In some embodiments, the reactivating agent is 5-aza-2′deoxycytidine (5-azaDC or decitabine). In some embodiments, the reactivating agent is 3-Deazaneoplanocin A (DZNep). In some embodiments, the reactivating agent is a combination of 5′-aza and a histone acyltransferase (HAT). In some embodiments, the reactivating agent is a combination of 5′-azaDC and a HAT. In some embodiments, the HAT is DZNep. Example of HATs include, but are not limited to DZNep, GSK126 and UNC1999. In some embodiments, the method further comprises administering to the subject or contacting the cell with a known reactivation agent.

In some embodiments, the reactivating begins immediately after the contacting or administering. In some embodiments, the reactivating begins within 1 hour, 2 hours, 4 hours, 6 hours, 8 hours, 12 hours, 18 hours, 24 hours, 48 hours or 72 hours after the contacting or administering. Each possibility represents a separate embodiment of the invention. In some embodiments, the reactivating occurs within 3 days of the contacting or administering. In some embodiments, the increased expression as compared to a different treatment begins immediately after the contacting or administering. In some embodiments, the increased expression as compared to a different treatment begins within 1 hour, 2 hours, 4 hours, 6 hours, 8 hours, 12 hours, 18 hours, 24 hours, 48 hours or 72 hours after the contacting or administering. Each possibility represents a separate embodiment of the invention. In some embodiments, the increased expression as compared to a different treatment occurs within 3 days of the contacting or administering.

In some embodiments, the increased expression as compared to a different treatment comprises at least a 10%, 20%, 30%, 40%, 50%, 60%, 70%, 80%, 90%, or 100% increase in expression of the silenced transcript over the expression of the transcript after reactivation with the other treatment. Each possibility represents a separate embodiment of the invention. In some embodiments, the increased expression as compared to a different treatment is at least 30%. In some embodiments, the increased expression is at least 30% and occurs within 4 days after the contacting or administering. In some embodiments, the increased expression is at least 30% and occurs within 3 days after the contacting or administering.

In some embodiments, the reactivating persists for at least 10, 20, 25, 30, 35, 40, 45, or 50 days. Each possibility represents a separate embodiment of the invention. In some embodiments, the reactivating persists for at least 30 days. In some embodiments, the reactivating persists for longer than 10, 20, 25, 30, 35, 40, 45, or 50 days. Each possibility represents a separate embodiment of the invention. In some embodiments, the reactivating persists for longer than 30 days. In some embodiments, expression of the previously silenced transcript persists for at least or longer than 10, 20, 25, 30, 35, 40, 45, or 50 days. Each possibility represents a separate embodiment of the invention. In some embodiments, expression of the previously silenced transcript persists for at least or longer than 30 days.

In some embodiments, the cell is in vivo. In some embodiments, the cell is in a subject. In some embodiments, cell is in vitro. In some embodiments, cell is ex vivo and is administered to a subject after the contacting. In some embodiments, the cell is a eukaryotic cell. In some embodiments, the cell is a mammalian cell. In some embodiments, the cell is a human cell. In some embodiments the cell is from a female subject. In some embodiments, the cell is from a male subject. In some embodiments, the cell is from a subject with only one X chromosome. In some embodiments, the subject is heterozygous for CGG repeat expansion. In some embodiments, the subject is homozygous for CGG repeat expansion. In some embodiments, the cell is a diseased cell. In some embodiments, the cell is a neuronal cell. In some embodiments, the cell is not a cancerous cell. In some embodiments, the cell is not a hematological cell. In some embodiments, the neuronal cell is from a region of the brain effected by Fragile X Syndrome (FXS). In some embodiments, the neuronal cell is from a region of the brain selected from the hippocampus, temporal cortices, visual cortex, cerebral cortex, amygdala, caudate nucleus, and temporal gyrus. In some embodiments, the cell is from the hippocampus. In some embodiments, the cell is a hippocampal cell.

In some embodiments, the cell comprises a silenced FMR1. In some embodiments, the FMR1 is abnormally silenced. In some embodiments, the silencing of FMR1 is pathological. As used herein, the term “abnormal” refers to a state that is different that the state in a comparable cell that does not suffer from a pathology. A skilled artisan will appreciate that a cell of a given type (neuron, cardiomyocyte, T cell, etc.) can be compared to another cell of the same type, or to many cells of the same type and if a particular gene's expression is grossly different, its expression can be considered abnormal. In some embodiments, abnormal silencing comprises at least a 50%, 60%, 70%, 80%, 90%, 95%, 97%, 99% or 100% reduction in transcription as compared to a healthy cell of the same cell type. Each possibility represents a separate embodiment of the invention.

In some embodiments, the cell comprises abnormal imprinting. In some embodiments, the cell comprises an abnormally silenced X chromosome. In some embodiments, the cell does not comprise canonical X chromosome inactivation (XCI). In some embodiments, abnormal silencing is any X chromosome silencing that is not XCI. In some embodiments, the cell comprises silencing of the FMR1 gene. In some embodiments, the cell comprises abnormal silencing of the FMR1 gene. In some embodiments, the cell comprises an expansion of a repetitive CGG sequence. In some embodiments, the CGG sequence is in a 5′ UTR. In some embodiments, the 5′ UTR is the 5′ UTR of the FMR1 gene. In some embodiments, the expansion comprises at least 55 CGG repeats. In some embodiments, the expansion comprises at least 50 CGG repeats. In some embodiments, the expansion comprises at least 200 repeats. In some embodiments, a cell with at least 50 or 55 repeats is a cell with a FMR1 permutation. In some embodiments, the CGG repeat is methylated. In some embodiments, the CGG repeat region is methylated. In some embodiments, a cell comprising at least 50, 55 or 200 repeats is a cell with silenced FMR1. Each possibility represents a separate embodiment of the invention. In some embodiments, a subject comprising cells with at least 50 or 55 repeats suffers from FMR1 permutation condition. In some embodiments, a cell with at least 200 repeats is a cell with FXS. In some embodiments, a cell with between 50 repeats and 200 repeats is a cell with a FMR1 permutation. In some embodiments, a cell with between 55 repeats and 200 repeats is a cell with a FMR1 permutation.

In some embodiments, the subject is a mammal. In some embodiments, the subject is a human. In some embodiments, the subject is in need of a method of the invention. In some embodiments, the subject is in need of treatment of a disease or disorder. In some embodiments, the subject is a fetus. In some embodiments, the subject is a child. In some embodiments, the subject is an adult. In some embodiments the subject is female. In some embodiments, the subject is male. In some embodiments, the subject has only 1 X chromosome per cell. In some embodiments, the subject is less than 1, 2, 3, 4, 5, 6, 7, 8, 9 or 10 years old. Each possibility represents a separate embodiment of the invention. In some embodiments, the administration occurs post-partum. In some embodiments, the administration occurs from 1 month, 3 months, 6 months, 9 months, 1 year, 2 years, or 3 years and on. In some embodiments, the subject is elderly. In some embodiments, the subject is older than 10, 15, 20, 30, 40, 50, 60, 70 or 80 years old. Each possibility represents a separate embodiment of the invention. In some embodiments, the administration is continued indefinitely. In some embodiments, the administration is continued for as long as symptoms of the condition persists. In some embodiments, the administration is for a set time, but is restarted if re-silencing of the genetic locus occurs.

In some embodiments, the subject suffers from the disease or disorder. In some embodiments, the subject is at risk of developing the disease or disorder. In some embodiments, the disease of disorder is a FMR1-associated disease or disorder. In some embodiments, the disease of disorder is a FMR1 silencing-associated disease or disorder.

In some embodiments, the disease is characterized by silenced transcription of FMR1. In some embodiments, the disease is not cancer. In some embodiments, the disease is characterized by an expansion of a repetitive CGG sequence in a 5′ UTR of a FMR1 gene. In some embodiments, the disease is a fragile X mental retardation 1 (FMR1) gene-associated disease. In some embodiments, the disease is a monoallelic disease. In some embodiments, the disease is a single-gene disorder or disease. In some embodiments, the disease is a mendelian disorder. In some embodiments, the disease is a neurological disease. In some embodiments, the disease is an autoimmune disease. In some embodiments, the disease is selected from Fragile X Syndrome (FXS), fragile X-associated tremor/ataxia syndrome (FXTAS), and FMR1-related primary ovarian insufficiency (POI). In some embodiments, the disease is FXS. In some embodiments, the disease is selected from FXS and FXTAS. In some embodiments, the disease is selected from FXS and POI. In some embodiments, the disease is FXTAS. In some embodiments, the disease is POI. In some embodiments, the subject suffers from FXS.

In some embodiments, the treating comprises reactivation of a silenced FMR1. Although the treatment may reactivate other genetic loci, these may be considered side effects and not required for treating the disease. In some embodiments, the treating and or reactivating does not cause cell death. In some embodiments, the treating and or reactivating does not induce apoptosis. In some embodiments, the treating and or reactivating improves the health of the cell comprising the reactivating. In some embodiments, the treating and or reactivating does not kill the cell comprising the reactivating.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life. In some embodiments, the method of the invention prevents the disease, disorder or condition.

As used herein, the terms “administering,” “administration,” and like terms refer to any method which, in sound medical practice, delivers a composition containing an active agent to a subject in such a manner as to provide a therapeutic effect. One aspect of the present subject matter provides for intravenous administration of a therapeutically effective amount of an agent to a patient in need thereof. Other suitable routes of administration can include intracranial, intrathecal, parenteral, subcutaneous, intravenous, intramuscular, or intraperitoneal. In some embodiments, the administration comprises intracranial administration. In some embodiments, the administration comprises intrathecal administration. In some embodiments, the administration comprises intravenous administration. In some embodiments, the administration comprises intramuscular administration.

In some embodiments, a therapeutically effective dose of an agent is administered. In some embodiments, a therapeutically effective dose of the composition of the invention is administered. The term “therapeutically effective amount” refers to an amount of a drug effective to treat a disease or disorder in a mammal. The term “a therapeutically effective amount” refers to an amount effective, at dosages and for periods of time necessary, to achieve the desired therapeutic or prophylactic result. The exact dosage form and regimen would be determined by the physician according to the patient's condition.

The dosage administered will be dependent upon the age, health, and weight of the recipient, kind of concurrent treatment, if any, frequency of treatment, and the nature of the effect desired.

In some embodiments, the composition is a pharmaceutical composition. In some embodiments, the pharmaceutical composition comprises a pharmaceutically acceptable carrier, adjuvant or excipient. In some embodiments, the pharmaceutical composition comprises a therapeutically effective amount of an agent. In some embodiments, the pharmaceutical composition is configured for systemic administration. In some embodiments, the pharmaceutical composition is configured for administration to a subject.

As used herein, the term “carrier,” “adjuvant” or “excipient” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelies, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

In some embodiments, the pharmaceutical composition of the invention is for use in reactivating transcription of a silenced genetic locus in a cell. In some embodiments, the pharmaceutical composition of the invention is for use in treating a disease or condition characterized by silenced transcription of at least one genetic locus in a subject in need thereof. In some embodiments, the pharmaceutical composition of the invention is for use in reactivating transcription of a fragile X mental retardation 1 (FMR1) gene in a cell. In some embodiments, the pharmaceutical composition of the invention is for use in treating a FMR1-associated disease in a subject in need thereof.

In some embodiments, inhibiting is decreasing. In some embodiments, decreasing is decreasing expression, function or both. In some embodiments, inhibiting is inhibiting function. In some embodiments, inhibiting is inhibiting expression. In some embodiments, expression is protein expression. In some embodiments, expression is mRNA expression. In some embodiments, inhibiting is inhibiting expression and function. In some embodiments, the agent is a small molecule. In some embodiments, the agent is an inhibitor. In some embodiments, the agent is a nucleic acid molecule. In some embodiments, the nucleic acid molecule is an RNA. In some embodiments, the RNA is a regulatory RNA. In some embodiments, the RNA is an inhibitory RNA. In some embodiments, the agent is an antibody. In some embodiments, the antibody is a blocking antibody. In some embodiments, the agent is specific to its target protein. In some embodiments, the nucleic acid molecule is specific to its target protein. In some embodiments, the antibody is specific to its target protein. As used herein, the term “specific” refers to binding to or directly modulating only the target protein. A specific nucleic acid molecule will bind only to the genetic locus or mRNA that encodes the protein and not significantly bind to another target. In some embodiments, specific is binding with at least 100% homology. In some embodiments, specific is binding with at least 95% homology. In some embodiments, specific is binding with at least 90% homology. In some embodiments, specific is binding without a mismatch. In some embodiments, specific is not decreasing expression or function of protein other than the target protein.

In some embodiments, the agent inhibits translation. In some embodiments, the agent inhibits transcription. In some embodiments, the agent is a nucleic acid molecule that induces mRNA degradation. In some embodiments, the agent is a nucleic acid molecule that alters the genetic locus encoding the target protein. In some embodiments, the agent is a nucleic acid molecule that modifies the genetic locus encoding the target protein. In some embodiments, altering is deleting a portion of the locus. In some embodiments, the altering is knocking out the locus. In some embodiments, altering is removing a functional locus.

In some embodiments, the nucleic acid molecule is an siRNA. In some embodiments, the nucleic acid molecule is an anti-sense oligonucleotide (ASO). In some embodiments, the nucleic acid molecule is a GAPmer. In some embodiments, the nucleic acid molecule is peptide nucleic acid (PNA). In some embodiments, the nucleic acid molecule is PMO. In some embodiments, the nucleic acid molecule is LNA. In some embodiments, the nucleic acid molecule is a guide RNA (gRNA). In some embodiments, the nucleic acid molecule is a sgRNA. In some embodiments, the nucleic acid molecule is complementary to an mRNA encoding the protein. In some embodiments, the nucleic acid molecule is complementary to a genomic locus that transcribes to a mRNA encoding the protein. In some embodiments, complementary is reverse complementary. In some embodiments, the nucleic acid molecule is an antisense oligonucleotide (ASO). In some embodiments, the pharmaceutical composition further comprises a genome editing enzyme. Genome editing is well known in the art and any such system may be used. In some embodiments, the genome editing enzyme comprises CRISPR/Cas9. In some embodiments, the genome editing enzyme comprises CRISPR/Cas9 or a derivative thereof.

Examples of nucleic acid molecules that can be used as agents are found in Table 2. Table 2 provides DNA sequences comprising “T”s, but it will be understood that an RNA molecule with uracils instead can also be used. Further, provided herein are the sequences of the genes that encodes the various recited proteins. Skilled artisan can generate nucleic acid molecules that are complementary/reverse complementary to these genes or their mRNA products. Further, there are numerous programs and websites known in the art for generating ASOs against target sequences or gRNAs that bind to target sequences. A few non-limiting examples include, nadcro.com, synbio-tech.com, the pfred.github.io, idtdna.com, snapgene.com and many more. Any such method, website or program may be used for designing nucleic acid agents for use in the methods of the invention.

In some embodiments, the agent is an inhibitor of the target protein. As used herein, the term “inhibitor” refers to any agent which may be a small molecule, an amino acid based molecule, or nucleic acid based molecule, that inhibits or decreases the function of the target protein within a cell. In some embodiments, the inhibitor is a small molecule. In some embodiments, the inhibitor is an inhibitory compound. In some embodiments, the inhibitor is an antibody. In some embodiments, the inhibitor is not a nucleic acid molecule that specifically decreases transcription, translation or both of the target. In some embodiments, the inhibitor is not a CRISPR/CAS9 or other genome editing composition for excision or editing of the genetic locus encoding the target protein. In some embodiments, the inhibitor is not an antibody.

As used herein, the term “antibody” refers to a polypeptide or group of polypeptides that include at least one binding domain that is formed from the folding of polypeptide chains having three-dimensional binding spaces with internal surface shapes and charge distributions complementary to the features of an antigenic determinant of an antigen. An antibody typically has a tetrameric form, comprising two identical pairs of polypeptide chains, each pair having one “light” and one “heavy” chain. The variable regions of each light/heavy chain pair form an antibody binding site. An antibody may be oligoclonal, polyclonal, monoclonal, chimeric, camelised, CDR-grafted, multi-specific, bi-specific, catalytic, humanized, fully human, anti-idiotypic and antibodies that can be labeled in soluble or bound form as well as fragments, including epitope-binding fragments, variants or derivatives thereof, either alone or in combination with other amino acid sequences. An antibody may be from any species. The term antibody also includes binding fragments, including, but not limited to Fv, Fab, Fab′, F(ab′)2 single stranded antibody (svFC), dimeric variable region (Diabody) and disulphide-linked variable region (dsFv). In particular, antibodies include immunoglobulin molecules and immunologically active fragments of immunoglobulin molecules, i.e., molecules that contain an antigen binding site. Antibody fragments may or may not be fused to another immunoglobulin domain including but not limited to, an Fc region or fragment thereof. The skilled artisan will further appreciate that other fusion products may be generated including but not limited to, scFv-Fc fusions, variable region (e.g., VL and VH)˜Fc fusions and scFv-scFv-Fc fusions.

Immunoglobulin molecules can be of any type (e.g., IgG, IgE, IgM, IgD, IgA and IgY), class (e.g., IgG1, IgG2, IgG3, IgG4, IgA1 and IgA2) or subclass.

Methods of generating antibodies are well known in the art and an antibody against the target protein may be generated by any such means and its blocking ability may further be tested by any assay known in the art. In some embodiments, the amino acid sequence of the target protein or a fragment thereof is used to generate an antibody. In some embodiments, the protein or a fragment thereof is administered to an animal (i.e., a mouse, rat, camel, shark, pig, rabbit, etc.) and the produced antibodies are extracted. Antibodies against the proteins recited herein are well known in the art as are blocking antibodies of those proteins. Any such antibody can be used as an agent of the invention.

In some embodiments, the protein is a target protein. In some embodiments, the protein is a protein provided in Table 1. In some embodiments, the genetic locus encodes a protein provided in Table 1. In some embodiments, the protein is selected from the group consisting of the proteins provided in Table 1. In some embodiments, the protein is selected from the group consisting of the proteins provided in Table 2.

In some embodiments, the protein is selected from the group consisting of: SMARCD1, QRICH1, CDK4, TAF8, ZFP90, ADNP, SPOP, ZNF217, CTBP2, TBX5, NHLH2, SOHLH1, UBR5, TP53BP1, EME1, PHF5A, ZBTB14, ZNF200, SATB2, USP47, DPF3, HOXC13, EVX2, ANKRD2, ZBTB24, SMEK1, ZNF385A, ZNF446, C6orf57, SLC25A19, SDHC, CEND1, ME2, ISCU, TRUB2, SDHA, PSEN1, TEFM, GATM, HSPA9, ARMCX3, DAO, FBXW7, FIS1, HSD3B1, DIABLO, HK2, HADHA, SDHAF2, DPH1, DPH2, ASF1A, DAPK3, and LIPT2. In some embodiments, the protein is selected from the group consisting of: QRICH1, CDK4, TAF8, SMARCD1, ZFP90, ADNP, SPOP, ZNF217, CTBP2, TBX5, NHLH2, SOHLH1, UBR5, EME1, PHF5A, ZBTB14, ZNF200, SATB2, USP47, DPF3, EVX2, ZBTB24, SMEK1, ZNF446, C6orf57, SLC25A19, SDHC, CEND1, ME2, ISCU, TRUB2, SDHA, PSEN1, TEFM, GATM, HSPA9, ARMCX3, DAO, FBXW7, FIS1, HSD3B1, HK2, HADHA, SDHAF2, DPH1, DPH2, ASF1A, DAPK3, and LIPT2. In some embodiments, the protein is selected from the group consisting of: QRICH1, TAF8, SMARCD1, ZFP90, ADNP, ZNF217, CTBP2, ZBTB14, SATB2, and C6orf57. In some embodiments, the protein is selected from the group consisting of: QRICH1, TAF8, SMARCD1, ZFP90, ZNF217, CTBP2, SATB2, and C6orf57. In some embodiments, the protein is selected from the group consisting of: SMARCD1, ZNF217 and C6orf57.

In some embodiments, the protein is SWI/SNF-related matrix-associated actin-dependent regulator of chromatin subfamily D member 1 (SMARCD1). The Entrez gene ID for human SMARCD1 is 6602 and the Uniprot ID is Q96GM5. In some embodiments, SMARCD1 is human SMARCD1. Agents that bind to and/or inhibit SMARCD1 are well known in the art and any such agent may be used for the method of the invention. In some embodiments, the agent is a nucleic acid molecule that binds to the SMARCD1 genetic locus. In some embodiments, the human SMARCD1 mRNA is provided in accession number NM_003076.5, or NM_139071.3. In some embodiments, the human SMARCD1 mRNA comprises or consists of SEQ ID NO: 21. In some embodiments, the human SMARCD1 mRNA comprises or consists of SEQ ID NO: 22. In some embodiments, the agent is an antisense nucleic acid molecule that hybridizes to SEQ ID NO: 21 or 22.

In some embodiments, the protein is Glutamine-rich protein 1 (QRICH1). The Entrez gene ID for human QRICH1 is 54870 and the Uniprot ID is Q2TAL8. In some embodiments, QRICH1 is human QRICH1. Agents that bind to and/or inhibit QRICH1 are well known in the art and any such agent may be used for the method of the invention. In some embodiments, the agent is a nucleic acid molecule that binds to the QRICH1 genetic locus. In some embodiments, the human QRICH1 mRNA is provided in accession number NM 198880.3. In some embodiments, the human QRICH1 mRNA comprises or consists of SEQ ID NO: 23. In some embodiments, the agent is an antisense nucleic acid molecule that hybridizes to SEQ ID NO: 23.

In some embodiments, the protein is Cyclin-dependent kinase 4 (CDK4). The Entrez gene ID for human CDK4 is 1019 and the Uniprot ID is P11802. In some embodiments, CDK4 is human CDK4. Agents that bind to and/or inhibit CDK4 are well known in the art and any such agent may be used for the method of the invention. In some embodiments, the agent is a nucleic acid molecule that binds to the CDK4 genetic locus. In some embodiments, the human CDK4 mRNA is provided in accession number NM_000075.4. In some embodiments, the human CDK4 mRNA comprises or consists of SEQ ID NO: 24. In some embodiments, the agent is an antisense nucleic acid molecule that hybridizes to SEQ ID NO: 24.

In some embodiments, the protein is Transcription initiation factor TFIID subunit 8 (TAF8). The Entrez gene ID for human TAF8 is 129685 and the Uniprot ID is Q727C8. In some embodiments, TAF8 is human TAF8. Agents that bind to and/or inhibit TAF8 are well known in the art and any such agent may be used for the method of the invention. In some embodiments, the agent is a nucleic acid molecule that binds to the TAF8 genetic locus. In some embodiments, the human TAF8 mRNA is provided in accession number NM 138572.3. In some embodiments, the human TAF8 mRNA comprises or consists of SEQ ID NO: 25. In some embodiments, the agent is an antisense nucleic acid molecule that hybridizes to SEQ ID NO: 25.

In some embodiments, the protein is Zinc Finger Protein 90 (ZFP90). The Entrez gene ID for human ZFP90 is 146198 and the Uniprot ID is Q8TF47. In some embodiments, ZFP90 is human ZFP90. Agents that bind to and/or inhibit ZFP90 are well known in the art and any such agent may be used for the method of the invention. In some embodiments, the agent is a nucleic acid molecule that binds to the ZFP90 genetic locus. In some embodiments, the human ZFP90 mRNA is provided in accession number NM_133458.4. In some embodiments, the human ZFP90 mRNA comprises or consists of SEQ ID NO: 26. In some embodiments, the agent is an antisense nucleic acid molecule that hybridizes to SEQ ID NO: 26.

In some embodiments, the protein is Activity-dependent neuroprotector homeobox (ADNP). The Entrez gene ID for human ADNP is 23394 and the Uniprot ID is Q9H2P0. In some embodiments, ADNP is human ADNP. Agents that bind to and/or inhibit ADNP are well known in the art and any such agent may be used for the method of the invention. In some embodiments, the agent is a nucleic acid molecule that binds to the ADNP genetic locus. In some embodiments, the human ADNP mRNA is provided in accession number NM 015339.5. In some embodiments, the human ADNP mRNA comprises or consists of SEQ ID NO: 27. In some embodiments, the agent is an antisense nucleic acid molecule that hybridizes to SEQ ID NO: 27.

In some embodiments, the protein is Speckle-type POZ protein (SPOP). The Entrez gene ID for human SPOP is 8405 and the Uniprot ID is 043791. In some embodiments, SPOP is human SPOP. Agents that bind to and/or inhibit SPOP are well known in the art and any such agent may be used for the method of the invention. In some embodiments, the agent is a nucleic acid molecule that binds to the SPOP genetic locus. In some embodiments, the human SPOP mRNA is provided in accession number NM_001007226.1. In some embodiments, the human SPOP mRNA comprises or consists of SEQ ID NO: 28. In some embodiments, the agent is an antisense nucleic acid molecule that hybridizes to SEQ ID NO: 28.

In some embodiments, the protein is Zinc finger protein 217 (ZNF217). The Entrez gene ID for human ZNF217 is 7764 and the Uniprot ID is 075362. In some embodiments, ZNF217 is human ZNF217. Agents that bind to and/or inhibit ZNF217 are well known in the art and any such agent may be used for the method of the invention. In some embodiments, the agent is a nucleic acid molecule that binds to the ZNF217genetic locus. In some embodiments, the human ZNF217mRNA is provided in accession number NM_001385034.1, or NM_006526.3. In some embodiments, the human ZNF217mRNA comprises or consists of SEQ ID NO: 29. In some embodiments, the human ZNF217mRNA comprises or consists of SEQ ID NO: 30. In some embodiments, the agent is an antisense nucleic acid molecule that hybridizes to SEQ ID NO: 29 or 30.

In some embodiments, the protein is C-terminal-binding protein 2 (CTBP2). The Entrez gene ID for human CTBP2 is 1488 and the Uniprot ID is P56545. In some embodiments, CTBP2 is human CTBP2. Agents that bind to and/or inhibit CTBP2 are well known in the art and any such agent may be used for the method of the invention. In some embodiments, the agent is a nucleic acid molecule that binds to the CTBP2 genetic locus. In some embodiments, the human CTBP2 mRNA is provided in accession number NM 001329.4. In some embodiments, the human CTBP2 mRNA comprises or consists of SEQ ID NO: 31. In some embodiments, the agent is an antisense nucleic acid molecule that hybridizes to SEQ ID NO: 31.

In some embodiments, the protein is T-box transcription factor TBX5 (TBX5). The Entrez gene ID for human TBX5 is 6910 and the Uniprot ID is Q99593. In some embodiments, TBX5 is human TBX5. Agents that bind to and/or inhibit TBX5 are well known in the art and any such agent may be used for the method of the invention. In some embodiments, the agent is a nucleic acid molecule that binds to the TBX5 genetic locus. In some embodiments, the human TBX5 mRNA is provided in accession number NM 181486.4. In some embodiments, the human TBX5 mRNA comprises or consists of SEQ ID NO: 32. In some embodiments, the agent is an antisense nucleic acid molecule that hybridizes to SEQ ID NO: 32.

In some embodiments, the protein is Nescient Helix-Loop-Helix 2 (NHLH2). The Entrez gene ID for human NHLH2 is 7818 and the Uniprot ID is Q02577. In some embodiments, NHLH2 is human NHLH2. Agents that bind to and/or inhibit NHLH2 are well known in the art and any such agent may be used for the method of the invention. In some embodiments, the agent is a nucleic acid molecule that binds to the NHLH2 genetic locus. In some embodiments, the human NHLH2 mRNA is provided in accession number NM 005599.3. In some embodiments, the human NHLH2 mRNA comprises or consists of SEQ ID NO: 33. In some embodiments, the agent is an antisense nucleic acid molecule that hybridizes to SEQ ID NO: 33.

In some embodiments, the protein is Spermatogenesis and Oogenesis Specific Basic Helix-Loop-Helix 1 (SOHLH1). The Entrez gene ID for human SOHLH1 is 27845 and the Uniprot ID is Q5JUK2. In some embodiments, SOHLH1 is human SOHLH1. Agents that bind to and/or inhibit SOHLH1 are well known in the art and any such agent may be used for the method of the invention. In some embodiments, the agent is a nucleic acid molecule that binds to the SOHLH1 genetic locus. In some embodiments, the human SOHLH1 mRNA is provided in accession number NM_001101677.2. In some embodiments, the human SOHLH1 mRNA comprises or consists of SEQ ID NO: 34. In some embodiments, the agent is an antisense nucleic acid molecule that hybridizes to SEQ ID NO: 34.

In some embodiments, the protein is E3 ubiquitin-protein ligase (UBR5). The Entrez gene ID for human UBR5 is 51366 and the Uniprot ID is 095071. In some embodiments, UBR5 is human UBR5. Agents that bind to and/or inhibit UBR5 are well known in the art and any such agent may be used for the method of the invention. In some embodiments, the agent is a nucleic acid molecule that binds to the UBR5 genetic locus. In some embodiments, the human UBR5 mRNA is provided in accession number NM_015902.6. In some embodiments, the human UBR5 mRNA comprises or consists of SEQ ID NO: 35. In some embodiments, the agent is an antisense nucleic acid molecule that hybridizes to SEQ ID NO: 35.

In some embodiments, the protein is Tumor suppressor p53-binding protein 1 (TP53BP1). The Entrez gene ID for human TP53BP1 is 7158 and the Uniprot ID is Q12888. In some embodiments, TP53BP1 is human TP53BP1. Agents that bind to and/or inhibit TP53BP1 are well known in the art and any such agent may be used for the method of the invention. In some embodiments, the agent is a nucleic acid molecule that binds to the TP53BP1 genetic locus. In some embodiments, the human TP53BP1 mRNA is provided in accession number NM_005657.4. In some embodiments, the human TP53BP1 mRNA comprises or consists of SEQ ID NO: 36. In some embodiments, the agent is an antisense nucleic acid molecule that hybridizes to SEQ ID NO: 36.

In some embodiments, the protein is Crossover junction endonuclease EME1 (EME1). The Entrez gene ID for human EME1 is 146956 and the Uniprot ID is Q96AY2. In some embodiments, EME1 is human EME1. Agents that bind to and/or inhibit EME1 are well known in the art and any such agent may be used for the method of the invention. In some embodiments, the agent is a nucleic acid molecule that binds to the EME1 genetic locus. In some embodiments, the human EME1 mRNA is provided in accession number NM 152463.4. In some embodiments, the human EME1 mRNA comprises or consists of SEQ ID NO: 37. In some embodiments, the agent is an antisense nucleic acid molecule that hybridizes to SEQ ID NO: 37.

In some embodiments, the protein is PHD Finger Protein 5A (PHF5A). The Entrez gene ID for human PHF5A is 84844 and the Uniprot ID is Q7RTV0. In some embodiments, PHF5A is human PHF5A. Agents that bind to and/or inhibit PHF5A are well known in the art and any such agent may be used for the method of the invention. In some embodiments, the agent is a nucleic acid molecule that binds to the PHF5A genetic locus. In some embodiments, the human PHF5A mRNA is provided in accession number NM_032758.4. In some embodiments, the human PHF5A mRNA comprises or consists of SEQ ID NO: 38. In some embodiments, the agent is an antisense nucleic acid molecule that hybridizes to SEQ ID NO: 38.

In some embodiments, the protein is Zinc Finger and BTB Domain Containing 14 (ZBTB14). The Entrez gene ID for human ZBTB14 is 7541 and the Uniprot ID is 043829. In some embodiments, ZBTB14 is human ZBTB14. Agents that bind to and/or inhibit ZBTB14 are well known in the art and any such agent may be used for the method of the invention. In some embodiments, the agent is a nucleic acid molecule that binds to the ZBTB14 genetic locus. In some embodiments, the human ZBTB14 mRNA is provided in accession number NM_003409.5. In some embodiments, the human ZBTB14 mRNA comprises or consists of SEQ ID NO: 39. In some embodiments, the agent is an antisense nucleic acid molecule that hybridizes to SEQ ID NO: 39.

In some embodiments, the protein is Zinc Finger Protein 200 (ZNF200). The Entrez gene ID for human ZNF200 is 7752 and the Uniprot ID is P98182. In some embodiments, ZNF200 is human ZNF200. Agents that bind to and/or inhibit ZNF200 are well known in the art and any such agent may be used for the method of the invention. In some embodiments, the agent is a nucleic acid molecule that binds to the ZNF200 genetic locus. In some embodiments, the human ZNF200 mRNA is provided in accession number NM 198087.3. In some embodiments, the human ZNF200 mRNA comprises or consists of SEQ ID NO: 40. In some embodiments, the agent is an antisense nucleic acid molecule that hybridizes to SEQ ID NO: 40.

In some embodiments, the protein is SATB Homeobox 2 (SATB2). The Entrez gene ID for human SATB2 is 23314 and the Uniprot ID is Q9UPW6. In some embodiments, SATB2 is human SATB2. Agents that bind to and/or inhibit SATB2 are well known in the art and any such agent may be used for the method of the invention. In some embodiments, the agent is a nucleic acid molecule that binds to the SATB2 genetic locus. In some embodiments, the human SATB2 mRNA is provided in accession number NM_015265.4. In some embodiments, the human SATB2 mRNA comprises or consists of SEQ ID NO: 41. In some embodiments, the agent is an antisense nucleic acid molecule that hybridizes to SEQ ID NO: 41.

In some embodiments, the protein is Ubiquitin Specific Peptidase 47 (USP47). The Entrez gene ID for human USP47 is 55031 and the Uniprot ID is Q96K76. In some embodiments, USP47 is human USP47. Agents that bind to and/or inhibit USP47 are well known in the art and any such agent may be used for the method of the invention. In some embodiments, the agent is a nucleic acid molecule that binds to the USP47 genetic locus. In some embodiments, the human USP47 mRNA is provided in accession number NM 017944.4. In some embodiments, the human USP47 mRNA comprises or consists of SEQ ID NO: 42. In some embodiments, the agent is an antisense nucleic acid molecule that hybridizes to SEQ ID NO: 42.

In some embodiments, the protein is Double PHD Fingers 3 (DPF3). The Entrez gene ID for human DPF3 is 8110 and the Uniprot ID is Q92784. In some embodiments, DPF3 is human DPF3. Agents that bind to and/or inhibit DPF3 are well known in the art and any such agent may be used for the method of the invention. In some embodiments, the agent is a nucleic acid molecule that binds to the DPF3 genetic locus. In some embodiments, the human DPF3 mRNA is provided in accession number NM_012074.5. In some embodiments, the human DPF3 mRNA comprises or consists of SEQ ID NO: 43. In some embodiments, the agent is an antisense nucleic acid molecule that hybridizes to SEQ ID NO: 43.

In some embodiments, the protein is Homeobox C13 (HOXC13). The Entrez gene ID for human HOXC13 is 3229 and the Uniprot ID is P31276. In some embodiments, HOXC13 is human HOXC13. Agents that bind to and/or inhibit HOXC13 are well known in the art and any such agent may be used for the method of the invention. In some embodiments, the agent is a nucleic acid molecule that binds to the HOXC13 genetic locus. In some embodiments, the human HOXC13 mRNA is provided in accession number NM 017410.3. In some embodiments, the human HOXC13 mRNA comprises or consists of SEQ ID NO: 44. In some embodiments, the agent is an antisense nucleic acid molecule that hybridizes to SEQ ID NO: 44.

In some embodiments, the protein is Even-Skipped Homeobox 2 (EVX2). The Entrez gene ID for human EVX2 is 344191 and the Uniprot ID is Q03828. In some embodiments, EVX2 is human EVX2. Agents that bind to and/or inhibit EVX2 are well known in the art and any such agent may be used for the method of the invention. In some embodiments, the agent is a nucleic acid molecule that binds to the EVX2 genetic locus. In some embodiments, the human EVX2 mRNA is provided in accession number NM_001080458.2. In some embodiments, the human EVX2 mRNA comprises or consists of SEQ ID NO: 45. In some embodiments, the agent is an antisense nucleic acid molecule that hybridizes to SEQ ID NO:

In some embodiments, the protein is Ankyrin Repeat Domain 2 (ANKRD2). The Entrez gene ID for human ANKRD2 is 26287 and the Uniprot ID is Q9GZV1. In some embodiments, ANKRD2 is human ANKRD2. Agents that bind to and/or inhibit ANKRD2 are well known in the art and any such agent may be used for the method of the invention. In some embodiments, the agent is a nucleic acid molecule that binds to the ANKRD2 genetic locus. In some embodiments, the human ANKRD2 mRNA is provided in accession number NM_020349.4. In some embodiments, the human ANKRD2 mRNA comprises or consists of SEQ ID NO: 46. In some embodiments, the agent is an antisense nucleic acid molecule that hybridizes to SEQ ID NO: 46.

In some embodiments, the protein is ZBTB24. The Entrez gene ID for human ZBTB24 is 9841 and the Uniprot ID is 043167. In some embodiments, ZBTB24 is human ZBTB24. Agents that bind to and/or inhibit ZBTB24 are well known in the art and any such agent may be used for the method of the invention. In some embodiments, the agent is a nucleic acid molecule that binds to the ZBTB24 genetic locus. In some embodiments, the human ZBTB24 mRNA is provided in accession number NM_014797.3. In some embodiments, the human ZBTB24 mRNA comprises or consists of SEQ ID NO: 47. In some embodiments, the agent is an antisense nucleic acid molecule that hybridizes to SEQ ID NO: 47.

In some embodiments, the protein is Protein Phosphatase 4 Regulatory Subunit 3A also known as SMEK1 protein (SMEK1). The Entrez gene ID for human SMEK1 is 55671 and the Uniprot ID is Q8N6W1. In some embodiments, SMEK1 is human SMEK1. Agents that bind to and/or inhibit SMEK1 are well known in the art and any such agent may be used for the method of the invention. In some embodiments, the agent is a nucleic acid molecule that binds to the SMEK1 genetic locus. In some embodiments, the human SMEK1 mRNA is provided in accession number NM_001366432.2. In some embodiments, the human SMEK1 mRNA comprises or consists of SEQ ID NO: 48. In some embodiments, the agent is an antisense nucleic acid molecule that hybridizes to SEQ ID NO: 48.

In some embodiments, the protein is Zinc Finger Protein 385A (ZNF385A). The Entrez gene ID for human ZNF385A is 25946 and the Uniprot ID is Q96PM9. In some embodiments, ZNF385A is human ZNF385A. Agents that bind to and/or inhibit ZNF385A are well known in the art and any such agent may be used for the method of the invention. In some embodiments, the agent is a nucleic acid molecule that binds to the ZNF385A genetic locus. In some embodiments, the human ZNF385A mRNA is provided in accession number NM_015481.3. In some embodiments, the human ZNF385A mRNA comprises or consists of SEQ ID NO: 49. In some embodiments, the agent is an antisense nucleic acid molecule that hybridizes to SEQ ID NO: 49.

In some embodiments, the protein is Zinc Finger Protein 446 (ZNF446). The Entrez gene ID for human ZNF446 is 55663 and the Uniprot ID is Q9NWS9. In some embodiments, ZNF446 is human ZNF446. Agents that bind to and/or inhibit ZNF446 are well known in the art and any such agent may be used for the method of the invention. In some embodiments, the agent is a nucleic acid molecule that binds to the ZNF446 genetic locus. In some embodiments, the human ZNF446 mRNA is provided in accession number NM 017908. In some embodiments, the human ZNF446 mRNA comprises or consists of SEQ ID NO: 50. In some embodiments, the agent is an antisense nucleic acid molecule that hybridizes to SEQ ID NO: 50.

In some embodiments, the protein is Succinate Dehydrogenase Complex Assembly Factor 4 (C6orf57 or SDHAF4). The Entrez gene ID for human C6orf57 is 135154 and the Uniprot ID is Q5VUM1. In some embodiments, C6orf57 is human C6orf57. Agents that bind to and/or inhibit C6orf57 are well known in the art and any such agent may be used for the method of the invention. In some embodiments, the agent is a nucleic acid molecule that binds to the C6orf57 genetic locus. In some embodiments, the human C6orf57 mRNA is provided in accession number NM 145267.3 or XM 047418210.1. In some embodiments, the human C6orf57 mRNA comprises or consists of SEQ ID NO: 51. In some embodiments, the human C6orf57 mRNA comprises or consists of SEQ ID NO: 52. In some embodiments, the agent is an antisense nucleic acid molecule that hybridizes to SEQ ID NO: 51 or 52.

In some embodiments, the protein is Solute Carrier Family 25 Member 19 (SLC25A19). The Entrez gene ID for human SLC25A19 is 60386 and the Uniprot ID is Q9HC21. In some embodiments, SLC25A19 is human SLC25A19. Agents that bind to and/or inhibit SLC25A19 are well known in the art and any such agent may be used for the method of the invention. In some embodiments, the agent is a nucleic acid molecule that binds to the SLC25A19 genetic locus. In some embodiments, the human SLC25A19 mRNA is provided in accession number NM_021734.5. In some embodiments, the human SLC25A19 mRNA comprises or consists of SEQ ID NO: 53. In some embodiments, the agent is an antisense nucleic acid molecule that hybridizes to SEQ ID NO: 53.

In some embodiments, the protein is Succinate Dehydrogenase Complex Subunit C (SDHC). The Entrez gene ID for human SDHC is 6391 and the Uniprot ID is Q99643. In some embodiments, SDHC is human SDHC. Agents that bind to and/or inhibit SDHC are well known in the art and any such agent may be used for the method of the invention. In some embodiments, the agent is a nucleic acid molecule that binds to the SDHC genetic locus. In some embodiments, the human SDHC mRNA is provided in accession number NM_003001.5. In some embodiments, the human SDHC mRNA comprises or consists of SEQ ID NO: 54. In some embodiments, the agent is an antisense nucleic acid molecule that hybridizes to SEQ ID NO: 54.

In some embodiments, the protein is Cell Cycle Exit and Neuronal Differentiation 1 (CEND1). The Entrez gene ID for human CEND1 is 51286 and the Uniprot ID is Q8N111. In some embodiments, CEND1 is human CEND1. Agents that bind to and/or inhibit CEND1 are well known in the art and any such agent may be used for the method of the invention. In some embodiments, the agent is a nucleic acid molecule that binds to the CEND1 genetic locus. In some embodiments, the human CEND1mRNA is provided in accession number NM 016564.4. In some embodiments, the human CEND1mRNA comprises or consists of SEQ ID NO: 55. In some embodiments, the agent is an antisense nucleic acid molecule that hybridizes to SEQ ID NO: 55.

In some embodiments, the protein is Malic Enzyme 2 (ME2). The Entrez gene ID for human ME2 is 4200 and the Uniprot ID is P23368. In some embodiments, ME2 is human ME2. Agents that bind to and/or inhibit ME2 are well known in the art and any such agent may be used for the method of the invention. In some embodiments, the agent is a nucleic acid molecule that binds to the ME2 genetic locus. In some embodiments, the human ME2 mRNA is provided in accession number NM_002396.5. In some embodiments, the human ME2 mRNA comprises or consists of SEQ ID NO: 56. In some embodiments, the agent is an antisense nucleic acid molecule that hybridizes to SEQ ID NO: 56.

In some embodiments, the protein is Iron-Sulfur Cluster Assembly Enzyme (ISCU). The Entrez gene ID for human ISCU is 23479 and the Uniprot ID is Q9H1K1. In some embodiments, ISCU is human ISCU. Agents that bind to and/or inhibit ISCU are well known in the art and any such agent may be used for the method of the invention. In some embodiments, the agent is a nucleic acid molecule that binds to the ISCU genetic locus. In some embodiments, the human ISCU mRNA is provided in accession number NM_014301.4. In some embodiments, the human ISCU mRNA comprises or consists of SEQ ID NO: 57. In some embodiments, the agent is an antisense nucleic acid molecule that hybridizes to SEQ ID NO: 57.

In some embodiments, the protein is TruB Pseudouridine Synthase Family Member 2 (TRUB2). The Entrez gene ID for human TRUB2 is 26995 and the Uniprot ID is 095900. In some embodiments, TRUB2 is human TRUB2. Agents that bind to and/or inhibit TRUB2 are well known in the art and any such agent may be used for the method of the invention. In some embodiments, the agent is a nucleic acid molecule that binds to the TRUB2 genetic locus. In some embodiments, the human TRUB2 mRNA is provided in accession number NM_015679.3. In some embodiments, the human TRUB2 mRNA comprises or consists of SEQ ID NO: 58. In some embodiments, the agent is an antisense nucleic acid molecule that hybridizes to SEQ ID NO: 58.

In some embodiments, the protein is Succinate Dehydrogenase Complex Flavoprotein Subunit A (SDHA). The Entrez gene ID for human SDHA is 6389 and the Uniprot ID is P31040. In some embodiments, SDHA is human SDHA. Agents that bind to and/or inhibit SDHA are well known in the art and any such agent may be used for the method of the invention. In some embodiments, the agent is a nucleic acid molecule that binds to the SDHA genetic locus. In some embodiments, the human SDHA mRNA is provided in accession number NM_004168.4. In some embodiments, the human SDHA mRNA comprises or consists of SEQ ID NO: 59. In some embodiments, the agent is an antisense nucleic acid molecule that hybridizes to SEQ ID NO: 59.

In some embodiments, the protein is Presenilin 1 (PSEN1). The Entrez gene ID for human PSEN1 is 5663 and the Uniprot ID is P49768. In some embodiments, PSEN1 is human PSEN1. Agents that bind to and/or inhibit PSEN1 are well known in the art and any such agent may be used for the method of the invention. In some embodiments, the agent is a nucleic acid molecule that binds to the PSEN1 genetic locus. In some embodiments, the human PSEN1 mRNA is provided in accession number NM_000021.4. In some embodiments, the human PSEN1 mRNA comprises or consists of SEQ ID NO: 60. In some embodiments, the agent is an antisense nucleic acid molecule that hybridizes to SEQ ID NO: 60.

In some embodiments, the protein is Transcription Elongation Factor, Mitochondrial (TEFM). The Entrez gene ID for human TEFM is 79736 and the Uniprot ID is Q96QE5. In some embodiments, TEFM is human TEFM. Agents that bind to and/or inhibit TEFM are well known in the art and any such agent may be used for the method of the invention. In some embodiments, the agent is a nucleic acid molecule that binds to the TEFM genetic locus. In some embodiments, the human TEFM mRNA is provided in accession number NM 024683.4. In some embodiments, the human TEFM mRNA comprises or consists of SEQ ID NO: 61. In some embodiments, the agent is an antisense nucleic acid molecule that hybridizes to SEQ ID NO: 61.

In some embodiments, the protein is Glycine Amidinotransferase (GATM). The Entrez gene ID for human GATM is 2628 and the Uniprot ID is P50440. In some embodiments, GATM is human GATM. Agents that bind to and/or inhibit GATM are well known in the art and any such agent may be used for the method of the invention. In some embodiments, the agent is a nucleic acid molecule that binds to the GATM genetic locus. In some embodiments, the human GATM mRNA is provided in accession number NM 001482.3. In some embodiments, the human GATM mRNA comprises or consists of SEQ ID NO: 62. In some embodiments, the agent is an antisense nucleic acid molecule that hybridizes to SEQ ID NO: 62.

In some embodiments, the protein is Heat Shock Protein Family A (Hsp70) Member 9 (HSPA9). The Entrez gene ID for human HSPA9 is 3313 and the Uniprot ID is P38646. In some embodiments, HSPA9 is human HSPA9. Agents that bind to and/or inhibit HSPA9 are well known in the art and any such agent may be used for the method of the invention. In some embodiments, the agent is a nucleic acid molecule that binds to the HSPA9 genetic locus. In some embodiments, the human HSPA9 mRNA is provided in accession number NM 004134.7. In some embodiments, the human HSPA9 mRNA comprises or consists of SEQ ID NO: 63. In some embodiments, the agent is an antisense nucleic acid molecule that hybridizes to SEQ ID NO: 63.

In some embodiments, the protein is Armadillo Repeat Containing X-Linked 3 (ARMCX3). The Entrez gene ID for human ARMCX3 is 51566 and the Uniprot ID is Q9UH62. In some embodiments, ARMCX3 is human ARMCX3. Agents that bind to and/or inhibit ARMCX3 are well known in the art and any such agent may be used for the method of the invention. In some embodiments, the agent is a nucleic acid molecule that binds to the ARMCX3 genetic locus. In some embodiments, the human ARMCX3 mRNA is provided in accession number NM_177947.3. In some embodiments, the human ARMCX3 mRNA comprises or consists of SEQ ID NO: 64. In some embodiments, the agent is an antisense nucleic acid molecule that hybridizes to SEQ ID NO: 64.

In some embodiments, the protein is D-Amino Acid Oxidase (DAO). The Entrez gene ID for human DAO is 1610 and the Uniprot ID is P14920. In some embodiments, DAO is human DAO. Agents that bind to and/or inhibit DAO are well known in the art and any such agent may be used for the method of the invention. In some embodiments, the agent is a nucleic acid molecule that binds to the DAO genetic locus. In some embodiments, the human DAO mRNA is provided in accession number NM_001917.5. In some embodiments, the human DAO mRNA comprises or consists of SEQ ID NO: 65. In some embodiments, the agent is an antisense nucleic acid molecule that hybridizes to SEQ ID NO: 65.

In some embodiments, the protein is F-Box and WD Repeat Domain Containing 7 (FBXW7). The Entrez gene ID for human FBXW7 is 55294 and the Uniprot ID is Q969H0. In some embodiments, FBXW7 is human FBXW7. Agents that bind to and/or inhibit FBXW7 are well known in the art and any such agent may be used for the method of the invention. In some embodiments, the agent is a nucleic acid molecule that binds to the FBXW7 genetic locus. In some embodiments, the human FBXW7 mRNA is provided in accession number NM_033632.3. In some embodiments, the human FBXW7 mRNA comprises or consists of SEQ ID NO: 66. In some embodiments, the agent is an antisense nucleic acid molecule that hybridizes to SEQ ID NO: 66.

In some embodiments, the protein is Fission, Mitochondrial 1 (FIS1). The Entrez gene ID for human FIS1 is 51024 and the Uniprot ID is Q9Y3D6. In some embodiments, FIS1 is human FIS1. Agents that bind to and/or inhibit FIS1 are well known in the art and any such agent may be used for the method of the invention. In some embodiments, the agent is a nucleic acid molecule that binds to the FIS1 genetic locus. In some embodiments, the human FIS1 mRNA is provided in accession number NM_016068.3. In some embodiments, the human FIS1 mRNA comprises or consists of SEQ ID NO: 67. In some embodiments, the agent is an antisense nucleic acid molecule that hybridizes to SEQ ID NO: 67.

In some embodiments, the protein is Hydroxy-Delta-5-Steroid Dehydrogenase, 3 Beta- And Steroid Delta-Isomerase 1 (HSD3B1). The Entrez gene ID for human HSD3B1 is 3283 and the Uniprot ID is P14060. In some embodiments, HSD3B1 is human HSD3B1. Agents that bind to and/or inhibit HSD3B1 are well known in the art and any such agent may be used for the method of the invention. In some embodiments, the agent is a nucleic acid molecule that binds to the HSD3B1 genetic locus. In some embodiments, the human HSD3B1 mRNA is provided in accession number NM_000862.3. In some embodiments, the human HSD3B1 mRNA comprises or consists of SEQ ID NO: 68. In some embodiments, the agent is an antisense nucleic acid molecule that hybridizes to SEQ ID NO: 68.

In some embodiments, the protein is Diablo IAP-Binding Mitochondrial Protein (DIABLO). The Entrez gene ID for human DIABLO is 56616 and the Uniprot ID is Q9NR28. In some embodiments, DIABLO is human DIABLO. Agents that bind to and/or inhibit DIABLO are well known in the art and any such agent may be used for the method of the invention. In some embodiments, the agent is a nucleic acid molecule that binds to the DIABLO genetic locus. In some embodiments, the human DIABLO mRNA is provided in accession number NM_019887.6. In some embodiments, the human DIABLO mRNA comprises or consists of SEQ ID NO: 69. In some embodiments, the agent is an antisense nucleic acid molecule that hybridizes to SEQ ID NO: 69.

In some embodiments, the protein is Hexokinase 2 (HK2). The Entrez gene ID for human HK2 is 3099 and the Uniprot ID is P52789. In some embodiments, HK2 is human HK2. Agents that bind to and/or inhibit HK2 are well known in the art and any such agent may be used for the method of the invention. In some embodiments, the agent is a nucleic acid molecule that binds to the HK2 genetic locus. In some embodiments, the human HK2 mRNA is provided in accession number NM_000189.5. In some embodiments, the human HK2 mRNA comprises or consists of SEQ ID NO: 70. In some embodiments, the agent is an antisense nucleic acid molecule that hybridizes to SEQ ID NO: 70.

In some embodiments, the protein is Hydroxyacyl-CoA Dehydrogenase Trifunctional Multienzyme Complex Subunit Alpha (HADHA). The Entrez gene ID for human HADHA is 3030 and the Uniprot ID is P40939. In some embodiments, HADHA is human HADHA. Agents that bind to and/or inhibit HADHA are well known in the art and any such agent may be used for the method of the invention. In some embodiments, the agent is a nucleic acid molecule that binds to the HADHA genetic locus. In some embodiments, the human HADHA mRNA is provided in accession number NM_000182.5. In some embodiments, the human HADHA mRNA comprises or consists of SEQ ID NO: 71. In some embodiments, the agent is an antisense nucleic acid molecule that hybridizes to SEQ ID NO: 71.

In some embodiments, the protein is Succinate Dehydrogenase Complex Assembly Factor 2 (SDHAF2). The Entrez gene ID for human SDHAF2 is 54949 and the Uniprot ID is Q9NX18. In some embodiments, SDHAF2 is human SDHAF2. Agents that bind to and/or inhibit SDHAF2 are well known in the art and any such agent may be used for the method of the invention. In some embodiments, the agent is a nucleic acid molecule that binds to the SDHAF2 genetic locus. In some embodiments, the human SDHAF2 mRNA is provided in accession number NM_017841.4. In some embodiments, the human SDHAF2 mRNA comprises or consists of SEQ ID NO: 72. In some embodiments, the agent is an antisense nucleic acid molecule that hybridizes to SEQ ID NO: 72.

In some embodiments, the protein is Diphthamide Biosynthesis 1 (DPH1). The Entrez gene ID for human DPH1 is 1801 and the Uniprot ID is Q9BZG8. In some embodiments, DPH1 is human DPH1. Agents that bind to and/or inhibit DPH1 are well known in the art and any such agent may be used for the method of the invention. In some embodiments, the agent is a nucleic acid molecule that binds to the DPH1 genetic locus. In some embodiments, the human DPH1 mRNA is provided in accession number NM_001383.6. In some embodiments, the human DPH1 mRNA comprises or consists of SEQ ID NO: 73. In some embodiments, the agent is an antisense nucleic acid molecule that hybridizes to SEQ ID NO: 73.

In some embodiments, the protein is Diphthamide Biosynthesis 2 (DPH2). The Entrez gene ID for human DPH2 is 1802 and the Uniprot ID is Q9BQC3. In some embodiments, DPH2 is human DPH2. Agents that bind to and/or inhibit DPH2 are well known in the art and any such agent may be used for the method of the invention. In some embodiments, the agent is a nucleic acid molecule that binds to the DPH2 genetic locus. In some embodiments, the human DPH2 mRNA is provided in accession number NM 001384.5. In some embodiments, the human DPH2 mRNA comprises or consists of SEQ ID NO: 74. In some embodiments, the agent is an antisense nucleic acid molecule that hybridizes to SEQ ID NO: 74.

In some embodiments, the protein is Anti-Silencing Function 1A Histone Chaperone (ASF1A). The Entrez gene ID for human ASF1A is 25842 and the Uniprot ID is Q9Y294. In some embodiments, ASF1A is human ASF1A. Agents that bind to and/or inhibit ASF1A are well known in the art and any such agent may be used for the method of the invention. In some embodiments, the agent is a nucleic acid molecule that binds to the ASF1A genetic locus. In some embodiments, the human ASF1A mRNA is provided in accession number NM_014034.3. In some embodiments, the human ASF1A mRNA comprises or consists of SEQ ID NO: 75. In some embodiments, the agent is an antisense nucleic acid molecule that hybridizes to SEQ ID NO: 75.

In some embodiments, the protein is Death Associated Protein Kinase 3 (DAPK3). The Entrez gene ID for human DAPK3 is 1613 and the Uniprot ID is Q43293. In some embodiments, DAPK3 is human DAPK3. Agents that bind to and/or inhibit DAPK3 are well known in the art and any such agent may be used for the method of the invention. In some embodiments, the agent is a nucleic acid molecule that binds to the DAPK3 genetic locus. In some embodiments, the human DAPK3 mRNA is provided in accession number NM_001348.3. In some embodiments, the human DAPK3 mRNA comprises or consists of SEQ ID NO: 76. In some embodiments, the agent is an antisense nucleic acid molecule that hybridizes to SEQ ID NO: 76.

In some embodiments, the protein is Lipoyl (Octanoyl) Transferase 2 (LIPT2). The Entrez gene ID for human LIPT2 is 387787 and the Uniprot ID is A6NK58. In some embodiments, LIPT2 is human LIPT2. Agents that bind to and/or inhibit LIPT2 are well known in the art and any such agent may be used for the method of the invention. In some embodiments, the agent is a nucleic acid molecule that binds to the LIPT2 genetic locus. In some embodiments, the human LIPT2 mRNA is provided in accession number NM 001144869.3. In some embodiments, the human LIPT2 mRNA comprises or consists of SEQ ID NO: 77. In some embodiments, the agent is an antisense nucleic acid molecule that hybridizes to SEQ ID NO: 77.

In some embodiments, an agent that inhibits a plurality of proteins is administered or contacted. In some embodiments, a plurality of agents is administered or contacted. In some embodiments, the plurality of agents inhibits at least 1, 2, 3, 4, 5, 6, 7, 8, 9, 10, 11, 12, 13, 14, 15, 16, 17, 18, 19 or 20 proteins. Each possibility represents a separate embodiment of the invention. In some embodiments, the plurality of agents inhibits at least 2 proteins. In some embodiments, each agent of the plurality is specific to a protein. In some embodiments, each agent of the plurality inhibits only one protein. In some embodiments, the method comprises inhibiting at least a first protein from Table 1. In some embodiments, the method comprises inhibiting at least a second protein from Table 1.

In some embodiments, the protein is a chromatin regulator. In some embodiments, the protein is a metabolic factor. In some embodiments, the protein produces the greatest reactivation of FMR1. In some embodiments, the plurality of proteins is selected based on their inhibition's effect on reactivation of FMR1.

In some embodiments, the agent inhibits ZNF217 and is a Lysine specific demethylase 1 (LSD1) inhibitor. In some embodiments, the agent inhibits ZNF217 and is a REST corepressor 1 (CoREST) inhibitor. In some embodiments, a CoREST inhibitor inhibits a CoREST complex. In some embodiments, the agent inhibits ZNF217 and is a LSD1 and CoREST inhibitor. LSD1 and CoREST inhibitors are well known in the art and any such inhibitor may be used. Examples of LSD1 and CoREST inhibitors include but are not limited to GSK2879552, bizine, corin, pargyline, and Rodin-A. In some embodiments, the agent is Rodin-A. In some embodiments, the agent is pargyline. In some embodiments, the agent is corin. In some embodiments, the agent is bizine. In some embodiments, the agent is GSK2879552.

In some embodiments, the LSD1 inhibitor is a histone deacetylase (HDAC) inhibitor. In some embodiments, the CoREST inhibitor is a histone deacetylase (HDAC) inhibitor. In some embodiments, the agent that inhibits ZNF217 is a histone deacetylase (HDAC) inhibitor. HDAC inhibitors are well known in the art and any such inhibitor may be employed. Example of HDAC inhibitors include, but are not limited to MS-275, SAHA, and LBH589. Further, HDAC inhibitors can be found for example in Suraweera et al., “Combination therapy with deacetylase inhibitors (HDACi) for the treatment of cancer: achieving the full therapeutic potential of HDACi”, 2018, Front. Oncol., Mar 29; 8:92, herein incorporated by reference in its entirety.

In some embodiments, the agent inhibits SMARCD1 and is a chromatin remodeling complex inhibitor. In some embodiments, the chromatin remodeling complex is a SWI/SNF complex. In some embodiments, the SWI/SNF complex is a SWI/SNF BAF complex. In some embodiments, the agent inhibits SMARCD1 and is bromodomain inhibitor. In some embodiments, a bromodomain inhibitor is a bromodomain containing protein inhibitor. In some embodiments, the bromodomain containing protein is a tandem bromodomain containing protein. In some embodiments, the bromodomain containing protein is a bromodomain and extra-terminal motif (BET) containing protein. In some embodiments, the agent is a BET inhibitor. In some embodiments, the agent inhibits SMARCD1 and is I-BRD9. In some embodiments, the bromodomain inhibitor is I-BRD9. In some embodiments, the bromodomain containing protein is BRD9. Chromatin remodeling complex inhibitors in general and bromodomain inhibitors in specific are well known in the art and any such inhibitors may be employed.

In some embodiments, the agent inhibits C6orf57 and is a succinate dehydrogenase (SDH) inhibitor. SDH inhibitors are well known in the art and any such inhibitor may be employed. Examples of SDH inhibitors include, but are not limited to Oxaloacetic acid, Malonate, Harzianopyridone, and 2-thenoyltrifluoroacetone. In some embodiments, the SDH inhibitor is Oxaloacetic acid. In some embodiments, the SDH inhibitor is malonate. In some embodiments, the SDH inhibitor is Harzianopyridone. In some embodiments, the SDH inhibitor is 2-thenoyltrifluoroacetone.

By another aspect, there is provided a method of determining suitability of a subject to be treated by a method of the invention, the method comprising measuring in a sample from the subject expression or function of a protein selected from those provided in Table 1, wherein elevated expression or function indicates the subject is suitable to be treated by a method of the invention.

In some embodiments, the sample is a tissue sample. In some embodiments, the sample is a bodily fluid. In some embodiments, the sample comprises cells. In some embodiments, the bodily fluid is selected from at least one of: blood, serum, plasma, gastric fluid, intestinal fluid, saliva, bile, tumor fluid, breast milk, urine, interstitial fluid, cerebral spinal fluid and stool. In some embodiments, the bodily fluid is blood.

In some embodiments, the method further comprises receiving the sample. In some embodiments, the method further comprises obtaining the sample from the subject. In some embodiments, the receiving comprises isolating cells from the sample. In some embodiments, the receiving comprises isolating proteins from the sample. In some embodiments, the receiving comprises isolating RNA from the sample.

In some embodiments, measuring comprises quantifying. In some embodiments, measuring is measuring protein expression. In some embodiments, measuring is measuring RNA expression. In some embodiments, expression is expression in cells. In some embodiments, expression is cell surface expression. In some embodiments, expression is secretion. In some embodiments, measuring is measuring protein function. In some embodiments, measuring is measuring cellular function. Methods of measuring protein expression are well known in the art and include, for example, western blotting, immunohistochemistry, immunocytochemistry, immunofluorescent quantification, ELISA, FACS and protein arrays. Any such method may be employed. Methods of measuring mRNA/gene expression are also well known in the art and include, for example, northern blotting, RT-PCR, qPCR, microarray hybridization and sequencing (e.g., whole transcriptome sequencing). Any such method may be employed. Assays of cellular function and protein function are well known in the art. A skilled artisan will appreciate that the particular assay will be selected based on the protein/cellular function to be measured. A skilled artisan will be able to select the proper assay for measuring a given proteins function.

In some embodiments, elevated is above a predetermined threshold. In some embodiments, the predetermined threshold is the expression in a control. In some embodiments, the predetermined threshold is the function level in a control. In some embodiments, the control is a healthy control. In some embodiments, a healthy control is a subject that does not suffer from the disease. In some embodiments, a healthy control is a subject that is not at risk of developing the disease. In some embodiments, in a control is in a sample from a control. In some embodiments, the control sample is the same type of sample as taken from the subject.

By another aspect, there is provide a method for producing an agent, the method comprising providing an agent that inhibits expression or function of a protein provided in Table 1, contacting said agent with a cell comprising a silenced FMR1 gene and selecting an agent that increases expression of FMR1, thereby selecting an agent.

By another aspect, there is provide a method for producing an agent, the method comprising providing an agent that inhibits expression or function of a protein selected from the group consisting of: SMARCD1, QRICH1, CDK4, TAF8, ZFP90, ADNP, SPOP, ZNF217, CTBP2, TBX5, NHLH2, SOHLH1, UBR5, TP53BP1, EME1, PHF5A, ZBTB14, ZNF200, SATB2, USP47, DPF3, HOXC13, EVX2, ANKRD2, ZBTB24, SMEK1, ZNF385A, ZNF446, C6orf57, SLC25A19, SDHC, CEND1, ME2, ISCU, TRUB2, SDHA, PSEN1, TEFM, GATM, HSPA9, ARMCX3, DAO, FBXW7, FIS1, HSD3B1, DIABLO, HK2, HADHA, SDHAF2, DPH1, DPH2, ASF1A, DAPK3, and LIPT2, contacting said agent with a cell comprising a silenced FMR1 gene and selecting an agent that increases expression of FMR1, thereby selecting an agent.

In some embodiments, the method is an ex vivo method. In some embodiments, the method is an in vitro method. In some embodiments, producing is selecting. In some embodiments, the agent is for use in a method of the invention. In some embodiments, the agent reactivates FMR1. In some embodiments, the agent increases expression of FMR1. In some embodiments, the agent is an agent of the invention. In some embodiments, the agent is an agent that treats a disease or disorder. In some embodiments, the disease or disorder is FXS. In some embodiments, the agent inhibits expression of the protein. In some embodiments, the agent inhibits the function of the protein. In some embodiments, the agent is a small molecule inhibitor. In some embodiments, the agent is a nucleic acid molecule. In some embodiments, the method further comprises testing the ability of the agent to inhibit expression or function of the protein. In some embodiments, the method comprises providing an agent and contacting said agent with a cell expressing the protein and selecting an agent that inhibits expression or function of the protein.

In some embodiments, the cell is an FXS model cell. In some embodiments, the cells are FXS-iPSCs. In some embodiments, the cell comprises a transgene of FMR1. In some embodiments, the transgene is on a plasmid. In some embodiments, the transgene is an exogenous nucleic acid molecule. In some embodiments, the transgene is integrated into the genome of the cell. In some embodiments, the silencing is heterochromatin silencing. In some embodiments, the silencing comprises methylation of the gene. In some embodiments, the FMR1 gene in the silenced cell comprises an expanded CGG repeat. In some embodiments, expanded comprises at least 55 repeats. In some embodiments, expanded comprises at least 200 repeats. In some embodiments, the CGG repeat region is methylated. In some embodiments, an expanded repeat is a methylated repeat. In some embodiments, the silenced FMR1 comprises a methylated CGG repeat region. In some embodiments, the transgene comprises a reporter moiety. In some embodiments, the reporter moiety is a fluorescent moiety. In some embodiments, an increase in the reporter moiety indicates an increase in expression of FMR1. In some embodiments, an increase in expression of FMR1 comprises FMR1 activation or reactivation. In some embodiments, the cell is a haploid cell.

In some embodiments, increasing expression is increasing expression in the cell. In some embodiments, the increased expression is from the silenced FMR1 gene. In some embodiments, an agent that produces the greatest increase is selected. In some embodiments, an agent that produces a statistically significant increase is selected. In some embodiments, an agent that produces an increase by at least a predetermined threshold is selected. In some embodiments, an agent that produces an increase of at least 5, 10, 15, 20, 25, 30, 35, 40, 45, 50, 55, 60, 65, 70, 75, 80, 85, 90, 92, 95, 97, 99 or 100% increase is selected. Each possibility represents a separate embodiment of the invention. In some embodiments, an agent that produces an increase of at least a 25% increase is selected. In some embodiments, an agent that produces an increase to close to unsilenced levels of expression is selected. In some embodiments, close to unsilenced levels is at least 25, 30, 35, 40, 45, 50, 55, 60, 65, 70, 75, 80, 85, 90, 92, 95, 97, 99 or 100% of unsilenced levels. Each possibility represents a separate embodiment of the invention. In some embodiments, close to unsilenced levels is at least 70% of unsilenced levels. In some embodiments, unsilenced levels are levels in a WT cell. In some embodiments, unsilenced levels are levels in a control cell. In some embodiments, unsilenced levels are levels are levels in a euchromatic FMR1 gene. In some embodiments, unsilenced levels are levels are levels in an FMR1 gene without an expanded CGG repeat. In some embodiments, unsilenced levels are levels are levels in an FMR1 gene with an unmethylated CGG repeat. In some embodiments, unsilenced levels are levels are levels in an FMR1 gene with fewer than 55 CGG repeats.

By another aspect, there is provided an agent produced by a method of the invention. In some embodiments, the agent is for use in a method of the invention.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization-A Laboratory Course Manual” CSHL Press (1996); all of which are incorporated by reference. Other general references are provided throughout this document.

### Materials and Methods

Cell lines: Throughout the study the following cell lines were used: The FXS-iPSCs male cell line A52. Haploid hpESCs-hPES10. CSES7 and CSES9 hESCs and their derivatives were used as a reference for wild type expression. 293T cells were used for the construction of lentiviral constructs.

Cell culture conditions: hESCs were cultured at 37° C. and 5% CO2 on growth-arrested mouse embryonic fibroblasts (MEF) in KnockOut Dulbecco's modified Eagle's medium (Gibco-Invitrogen, CA) supplemented with 15% KnockOut Serum Replacement (Gibco-Invitrogen, CA), 1 mM glutamine, 0.1 mM β-mercaptoethanol (Sigma-Aldrich, MO), 1% nonessential amino acids stock (Gibco-Invitrogen, CA), penicillin (50 units/ml), streptomycin (50 μg/ml), and 8 ng/ml FGF2 (Gibco-Invitrogen, CA). The medium was supplemented with 10 μM ROCK inhibitor (Y27632, Stemgent) in the first 24 h after thawing and passaging. Cells were passaged using short treatment with Trypsin or Trypsin-EDTA (Biological Industries, Beit Haemek, Israel). 293T cells and feeder layer MEFs were cultured in DMEM supplemented with 10% fetal bovine serum (Invitrogen), 50 U/ml penicillin (Biological Industries), and 50 μg/ml streptomycin (Biological Industries).

RNA isolation and reverse transcription: Total RNA was isolated using NucleoSpin RNA Plus kit (Macherey-Nagel) and reverse transcribed using the qScript® cDNA Synthesis Kit. Quantitative real time PCR (qRT-PCR) was performed using SYBR Green qPCR Supermix (Applied Biosystems) and analyzed with the 7300 real-time PCR system (Applied biosystems). FMR1 expression was assessed using specific primers, which amplified a 147 bp product spanning exons 5-6 of FMR1. GAPDH was used for normalization.

RNA sequencing analysis: RNA sequencing libraries were created using TruSeq RNA Library Prep Kit (Illumina) and sequenced using Illumina NextSeq 500 with 75 bp single-end reads. Raw RNA sequencing samples were aligned to the human reference genome (GRCh38/hg38) using STAR 2.4.0.1 software. Next, sequences originating from the feeder layer of mouse embryonic fibroblasts were filtered using the XenofilteR package. Following the filtering of mouse sequences, normalization of the read counts and identification of statistically significant differentially expressed genes were performed using edgeR.

DNMT1 disruption assay: As human pluripotent stem cells require DNA methylation for their viability, heterogenous DNMT1-mutant populations of FXS-iPSCs were generated by infecting the cells with a lentiCRISPR v2 lentiviral vector containing an sgRNA that targets the open reading frame of DNMT1. The sgRNA sequence was cloned into the lentiCRISPR v2 plasmid and transformed into Stb13 chemically competent bacteria. Following plasmid isolation, 293T cells were transfected with sgRNA-containing lentiCRISPR V2, psPAX2, and pCMV-VSV-G plasmids at a ratio of 13.3:10:6.6, respectively, in the presence of polyethylenimine ‘Max’ (Polysciences). Lentiviral particle-containing supernatant was collected ˜65 hours after transfection and filtered using 0.45 μM filters. For transduction, FXS-iPSCs were trypsinized, centrifuged, and suspended in embryonic stem cell media supplemented with 10 μM ROCK inhibitor (Y27632) and 8 μg/ml polybrene (Sigma). The viruses were added to the cell suspension, and the cells were plated on MEFs overnight. After ˜36 hours, the cells' medium was replaced with puromycin-containing medium (0.3 mg/ml, Sigma). After 2-14 days selection with puromycin, cells were harvested for RNA extraction. To confirm DNMT1 mutagenesis in the transduced cells, qRT-PCR primers that were designed to have their 3′ ends around the Cas9 cut-site of DNMT1 were used to analyze the abundance of wild-type copies to DNMT1 transcripts.

In vitro DNA methylation: pFMR1-n (CGG)-eGFP construct was methylated using an M.SssI CpG methyltransferase (NEB) according to manufacturer's recommendations. SAM concentration was adjusted to 640 μM. DNA was extracted using ethanol precipitation.

Methylation analysis using McrBC digestion: 500 ng of plasmid DNA was digested in a 30 μl reaction using McrBC restriction enzyme (NEB) for 3 hours at 37° C. and heat inactivated at 65C for 20 minutes.

CRISPR/Cas9 loss-of-function library: For the genome-wide screen, a previously established CRISPR/Cas9 based genome-wide loss-of-function library of haploid hESCs (Yilmaz et al., 2018, “Haploid human embryonic stem cells: half the genome, double the value”, Cell Stem Cell, 19, 569-572, herein incorporated by reference in its entirety) was used. Briefly, haploid-enriched ESC cultures of hpES10 cell line were infected with a lentivirus CRISPR/Cas9 genome-wide library at a multiplicity of infection of 0.3. Infected cells were then selected with puromycin (Sigma) and cultured for about fifteen doublings before harvesting and freezing. The library was then thawed and cultured at 37° C. with 5% CO2 in feeder-free conditions using matrigel-coated plates (Corning) and mTeSR1 medium (STEMCELL Technologies) supplemented with 10 μM ROCK inhibitor (Y27632, Stemgent) for one day after thawing or splitting.

Library transfection: 10 μM ROCK inhibitor (Y27632) was added 1 hour prior to trypsinization and transfection. Library cells were harvested using TrypLE™ Select Enzyme solution (Thermo Fisher Scientific, Cat #12563029) in a single cell state. Following the first centrifugation, cells were re-suspended twice with DMEM/F12 and centrifuged again before incubation with the plasmid DNA. Transfection of the methylated pFMR1-CGG (240)-EGFP construct was performed using the Xtreme Gene9 reagent (Roche), using standard conditions. 48 hours following transfection, cells were washed with PBS, harvested using TrypLE™ Select, resuspended in PBS supplemented with 15% fetal bovine serum (FBS) and filtered through a 70-μm cell strainer (Corning) and sorted using BD FACDAria III. Transfection efficiency was assessed using the simultaneous transfection of control haploid hESCs with an unmethylated pFMR1-CGG (240)-EGFP construct.

DNA sequencing and sgRNA distribution analysis: DNA was extracted using the gSYNC DNA extraction kit (Geneaid). The sgRNA integration-containing region was amplified, and the DNA library was established as previously described (Yilmaz et al., 2018). Briefly, sgRNA sequences were aligned to the DNA sequencing reads using the Bowtie 2 aligner. To exclude sgRNAs with low coverage in the library, sgRNAs which were represented in each of the GFP-negative control replicates were included in the statistical analysis. EdgeR package was used to normalize and compare sgRNA sequence counts. To compare between GFP-positive and GFP-negative populations that were derived from the same frozen library aliquot, each GFP-positive replicate was matched to its control, (GFP-positive replicates 3 and 4 were both matched to GFP-negative replicate 3, as they were derived from the same frozen library aliquot). Enrichment scores were calculated as the average of the log 2 ratio of sgRNA abundance for each gene between the GFP-positive and GFP-negative populations. Statistical significance was determined by the Kolmogorov-Smirnov test function in R. Genes that passed the following filters were considered enriched: (1) FDR-corrected P-value<0.05; (2) Log2FC>0.5 (3) TPM>1 (as calculated from RNA-seq of the CRISPR/Cas9 genome-wide haploid ESC library (Yilmaz et al., 2018); (3) Growth restricting genes as defined by Yilmaz et al. were discarded.

Generation of gene knockouts: Ten candidate genes were targeted using a lentiviral construct containing Cas9 and a specific sgRNA (Table 2). Two sgRNAs were used to target each candidate gene. Each sgRNA sequence was inserted into a separate lentiCRISPR v2 vector (Addgene cat. No. 52961). As a control, a lentiCRISPR v2 vector without any sgRNA (empty vector) was used. Lentiviruses were produced using 293T cells cultured in 10 cm plates using transfection with the sgRNA-containing-lentiCRISPR v2 construct, psPAX2 (Addgene cat. No. 12260) and pCMV-VSV-G (Addgene cat. No. 8454) at a ratio of 5.32:4:2.64 ratio, in the presence of polyethylenimine “Max” (PEI-Max) (Polysciences) at a 1:2 ratio of DNA to PEI-Max. After 24 hours, the medium was exchanged with standard hESC medium, which was harvested 60-65 hours following transfection, centrifuged, and filtered through 0.45 μm cellulose acetate filters (Millipore). The filtered supernatant was frozen in aliquots at −70° C. For transduction, FXS-iPSCs or haploid human ESCs were trypsinized with TrypLE™ Select, centrifuged, and resuspended in hESC medium supplemented with 8 μg ml-1 polybrene (Sigma) and 10 μM ROCK inhibitor (Y27632). Thawed or fresh viruses were added to the cell suspension, followed by plating the cells on feeder layer MEFs. 24 hours following transduction, the medium was replaced with hESC medium containing puromycin (0.3 μg/ml, Sigma). Cells were kept under antibiotic selection for the entire culturing periods.

TABLE 2
sgRNAs

| | | SEQ ID |
|---|---|---|
| Gene | gRNA_sequence | NO: |
| ADNP | GGAGTCAAATCTGTAGGCCA | 1 |
| ADNP | GTAAGCAACAGATTCAGCTG | 2 |
| C6orf57 | GAGACCCAGCTAAGCAACCA | 3 |
| C6orf57 | TCCCAATCTCCATATCGGGT | 4 |
| CTBP2 | TGCAGCCGTGGAAGAGACAG | 5 |
| CTBP2 | GGCACGTACCAAAGCCAATG | 6 |
| DNMT1 | GAGGCCAGAAGGAGGAACCG | 7 |
| QRICH1 | GAGACGGGGTCTGAATGGAG | 8 |
| SATB2 | GGGGAAGGTTCAGGAAGCGA | 9 |
| SATB2 | GATGCTGGTGAGCCAGGAGC | 10 |
| SMARCD1 | GGAGCCGGGCCCATTCGCAC | 11 |
| SMARCD1 | GCCACTACCCAGGAGACCGA | 12 |
| TAF8 | GAACAAGATGGCCGACGCGG | 13 |
| TAF8 | GAGTGCCGAGAAAGCATCCG | 14 |
| ZBTB14 | GTGCAGGAGCCACACACAAA | 15 |
| ZBTB14 | TGATGATGTAGAGGAAATCG | 16 |
| ZFP90 | TACCTGGATAGAAAGGTCGT | 17 |
| ZFP90 | CCTTACCAAGAGAAACCAGG | 18 |
| ZNF217 | GTGGTGAACGGATCGAGCTG | 19 |
| ZNF217 | TCTGGATGAAAATGGAGCCG | 20 |

Quantification and statistics: All analyses were performed using the python-based library pandas (pandas.pydata.org/). For the statistical analysis, SciPy (scipy.org/) and Statmodels (statsmodels.sourceforge.net/) libraries were used. Data was judged to be statistically significant when p<0.05 by two-tailed Student's T-Test.

Pyrosequencing analysis of the pFMR1-CGG-EGFP construct: Pyrosequencing of the FMR1 promoter both in FXS-iPSCs and in the methylated pFMR1-CGG-EGFP plasmid was performed by EpigenDx according to standard procedures (assay ADS1451-FS2). 11 CpG positions that showed high DNA methylation in the methylation control of the assay and across FXS-iPSC samples were included in the analysis.

### Example 1: CRISPR/Cas9-Based Disruption of DNMT1 in FXS-IPSCs

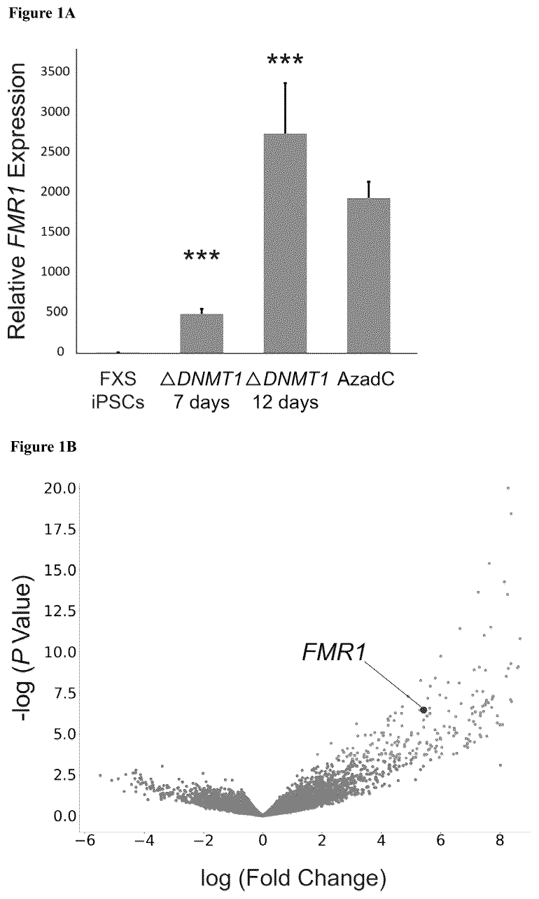

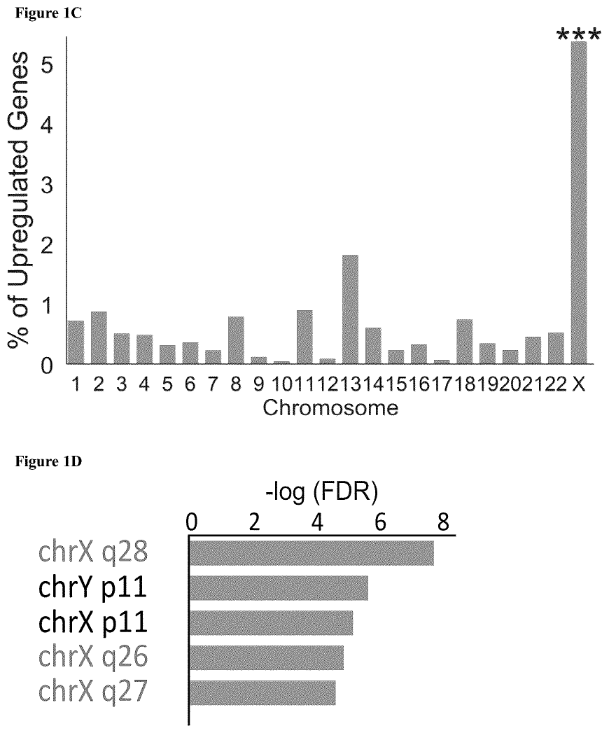

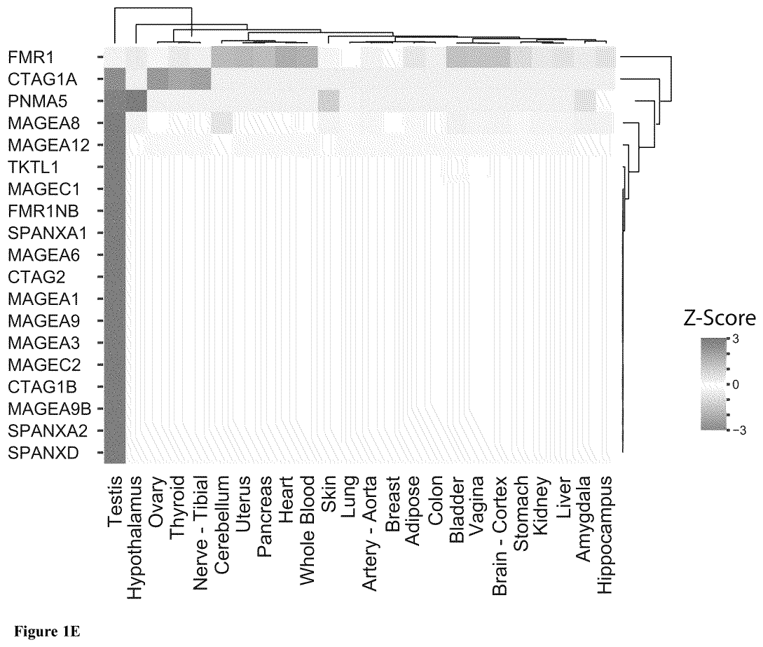

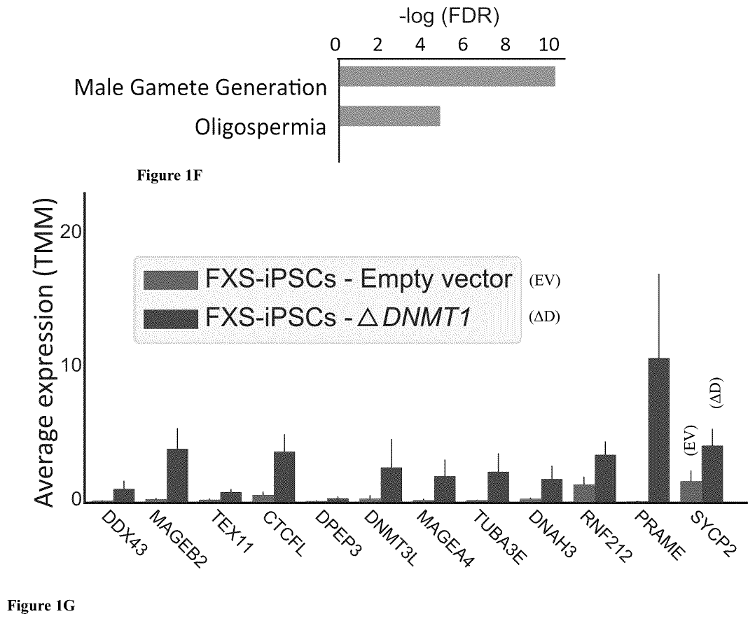

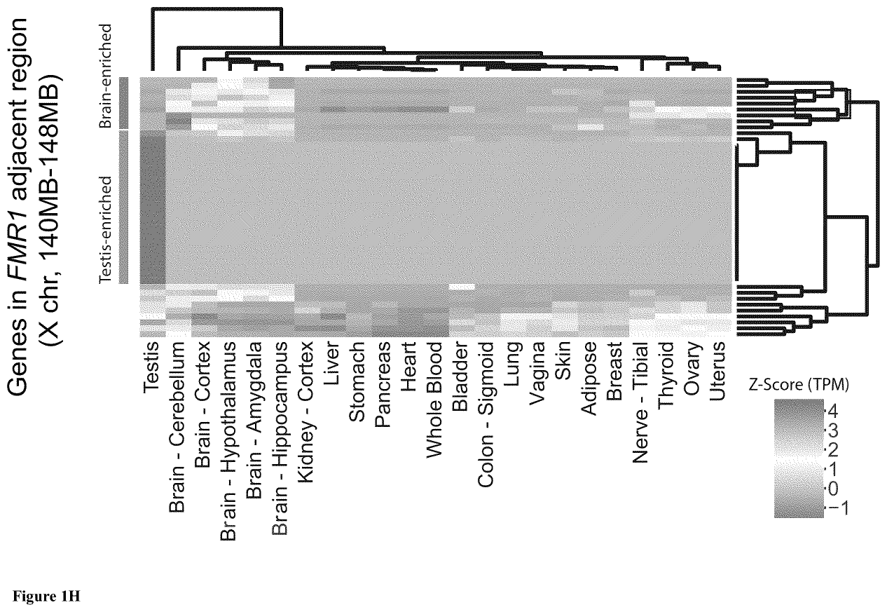

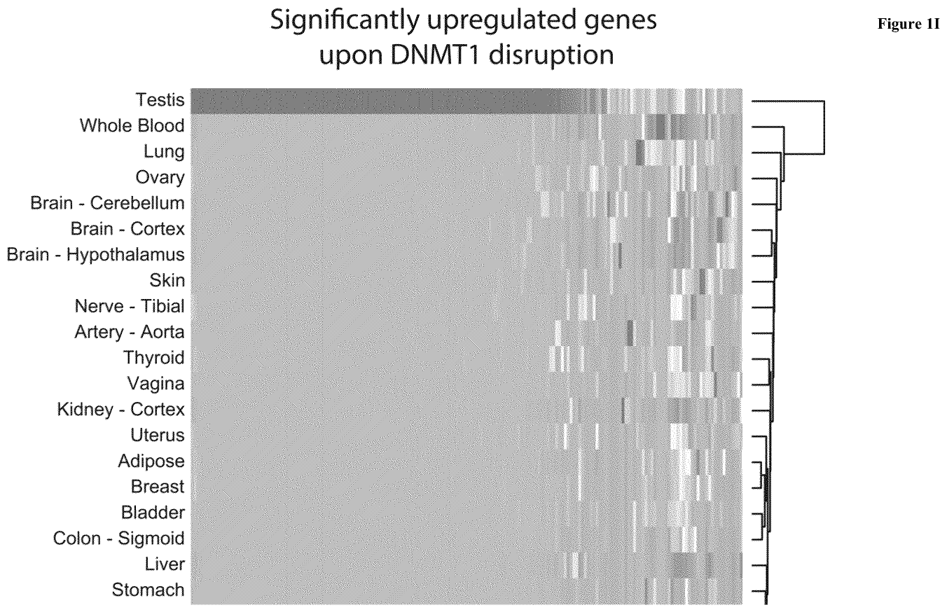

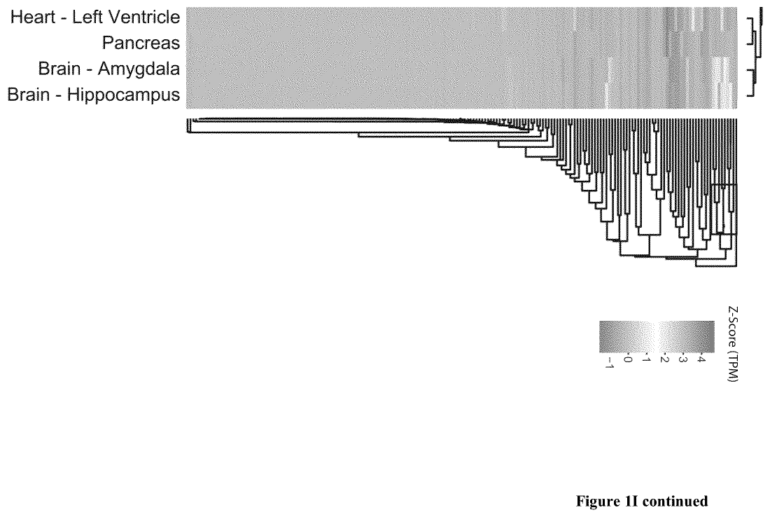

Previous work has demonstrated that chemical disruption of DNA methylation induces FMR1 expression in FXS-iPSCs. To further explore the maintenance of DNA methylation in the FMR1 locus, and to test the possibility of FMR1 reactivation using gene targeting of a single epigenetic factor, the consequences of targeted DNA methyltransferase 1 (DNMT1) perturbation in FXS-iPSCs was analyzed. To overcome the sensitivity of human PSCs to the loss of DNMT1, FXS-iPSCs were infected with a lentiviral vector containing sgRNA targeting DNMT1 and the culture was collected 7-14 days following the initiation of selection. This way, despite the apparent cell death following lentiviral transduction, viable cultures could be collected for gene expression analysis. RT-PCR analysis of the mutated samples compared to control FXS-iPSCs samples demonstrated the reactivation of FMR1 expression following DNMT1 disruption to levels comparable with 5′-aza-2′-deoxycytidine (5-azadC) treatment (FIG. 1A).

Next, a global transcriptional analysis of the DNMT1 mutants and control FXS-iPSC samples was performed, which identified FMR1 as one of the top significantly upregulated genes following DNMT1 mutagenesis (FIG. 1B, FDR<0.0001). Analysis of the global transcriptional response following DNMT1 depletion revealed a striking enrichment of genes located in the regions adjacent to the FMR1 locus, around the distal end of the long (q) arm of the X chromosome (Xq27-28) (FIG. 1C-1D). Tissue expression analysis of the top upregulated Xq27-28 genes (FIG. 1E-1F, FDR<0.05), as well as of all the genes in the Fragile X adjacent region (FIG. 1H, X chromosome 140 MB-148 MB) revealed a cluster of testis-specific expressed genes, that are mostly silenced in normal human PSCs. Analysis of the genome-wide transcriptional response following DNMT1 disruption also revealed a significant enrichment of testis-specific expressed genes, that were associated with GO terms such as gonadal development, reproduction, male gamete generation, oligospermia (FIG. 1F, 1I), including testis-specific marker genes involved in transcriptional regulation (FIG. 1G). The activation of germ cell genes upon DNA demethylation of human PSCs is in line with the global erasure of DNA methylation during normal development of primordial germ cells, reflecting the role of global demethylation in the activation of germline-specific expression. However, the association between Xq27-28 testis-specific gene expression and FMR1 activation might have specific implications on FMR1 regulation, which was previously linked with testicular differentiation.

### Example 2: Establishment of a Screening Protocol for the Identification of Genes Involved in FMR1 Silencing

To search for novel regulators involved in maintaining FMR1 inactivation, a loss-of-function genetic screen using a genome-wide CRISPR/Cas9 library established in haploid hESCs was conducted. Since haploid human ESCs harbor a standard range of CGG repeats and actively express FMR1, a tractable model system to analyze FMR1 regulation in hPSCs without an endogenous CGG expansion was established.

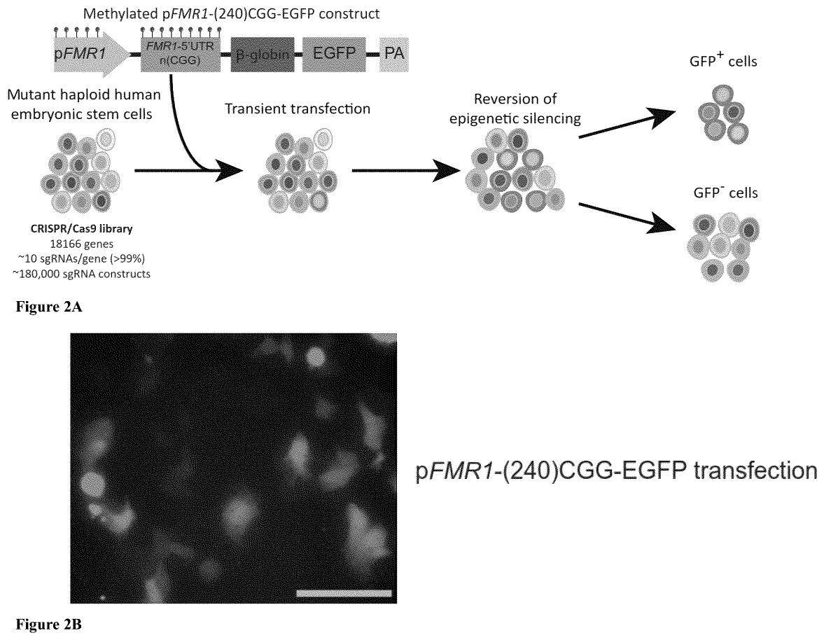

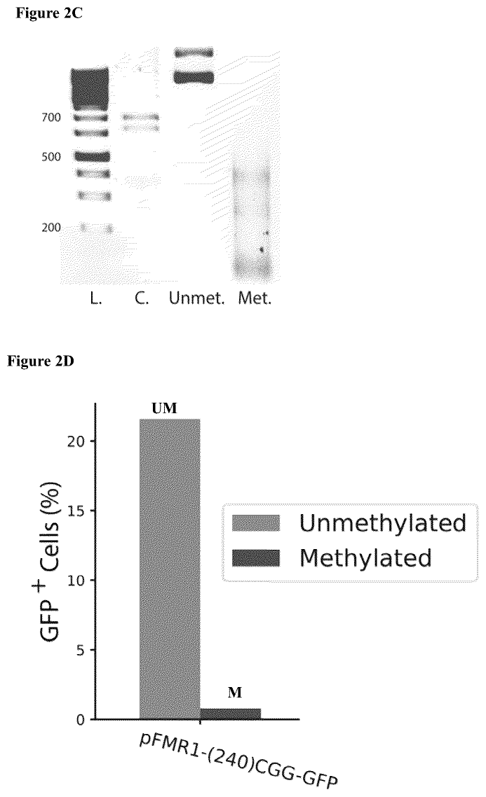

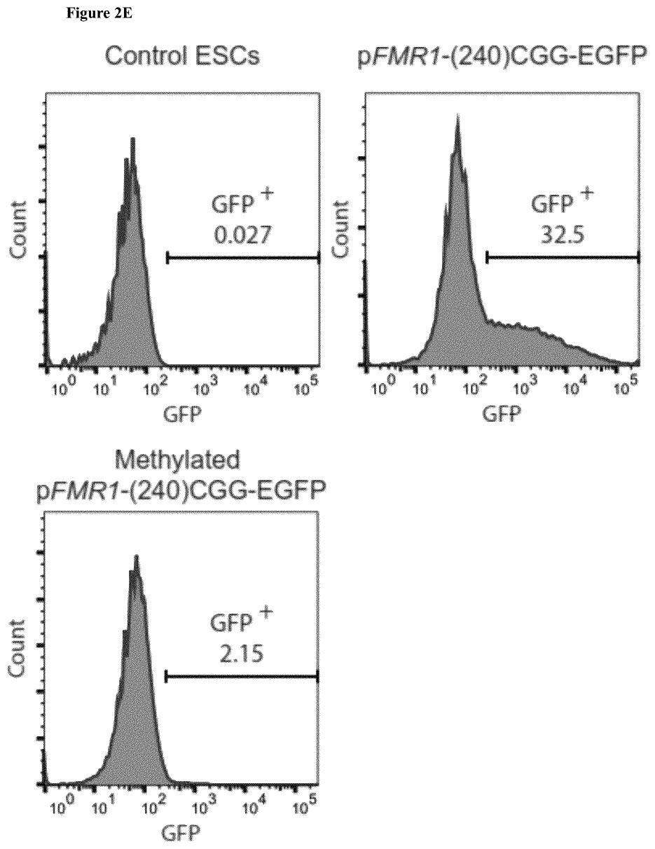

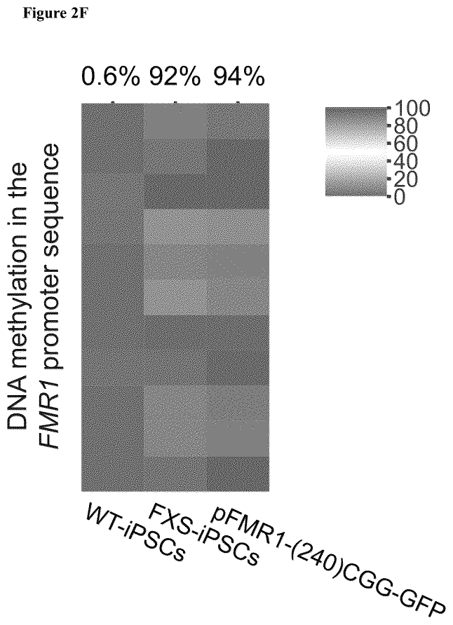

To this aim, a reporter plasmid in which the enhanced green fluorescent protein (EGFP) was placed under the control of a 696 base-pair human FMR1 minimal promoter (pFMR1), continued by the FMR1 5′-UTR sequence, starting from 112 bp upstream to the CGG repeats to 68 bp downstream the repetitive sequence was used. The continuous FMR1 promoter and 5′-UTR fragment with 240 CGG repeats were positioned upstream to the rabbit β-globin intron II followed by the EGFP reporter, in a similar position concerning the mutant CGG repetitive tract as the coding sequence of the endogenous FMR1 gene in FXS patients (FIG. 2A).

To avoid the effect of transgene integration site variability, the expression of the pFMR1-CGG-EGFP construct was tested using transient transfection in haploid hESCs (FIG. 2A). As expected, the presence of the full mutation length 240 CGG repeat tract was not sufficient for the transcriptional inactivation of EGFP expression (FIG. 2B). Therefore, to induce the epigenetic repression of the pFMR1-(240) CGG-EGFP construct, in vitro methylation produced the recombinant CpG methyltransferase M.SssI was used. DNA methylation of the construct was validated by its digestion with the methylation-sensitive McrBC restriction enzyme, the activity of which depends on the presence of methylated CpG sites (FIG. 2C). Bisulfite-pyrosequencing analysis also used for confirmation (FIG. 2F). In vitro methylation using M.SssI efficiently silenced pFMR1-(240) CGG-EGFP expression following transient transfection, with >10 fold enrichment of the GFP-positive cell fraction between the cultures transfected with unmethylated and methylated constructs (FIG. 2D-2E).

### Example 3: Using a Loss-of-Function Genome-Wide Library to Screen for Genes Involved in FMR1 Inactivation

Next, the assay was applied to the CRISPR/Cas9 haploid hESCs library, which contains 178,896 different gRNA constructs, targeting 18,166 genes. 48 hours following transfection with methylated pFMR1-(240) CGG-EGFP construct, library cells were harvested and sorted to GFP-positive and GFP-negative populations (FIG. 2A). The abundance of different gRNAs represented in both populations was assessed by the amplification of the sgRNA-containing genomic DNA segment and high throughput sequencing. Following the mapping of the reads to the sgRNA sequences, an enrichment score was assigned to each gene by calculating the log 2 fold change of its sgRNA counts between GFP-positive populations (n=4) and the GFP-negative populations (n=3) using the EdgeR software.

This scoring system allowed for analysis of the changes in abundance of mutants between the GFP-positive and the GFP-negative populations to determine candidate genes predicted to be involved in repression of the methylated pFMR1-(240) CGG-EGFP expression.

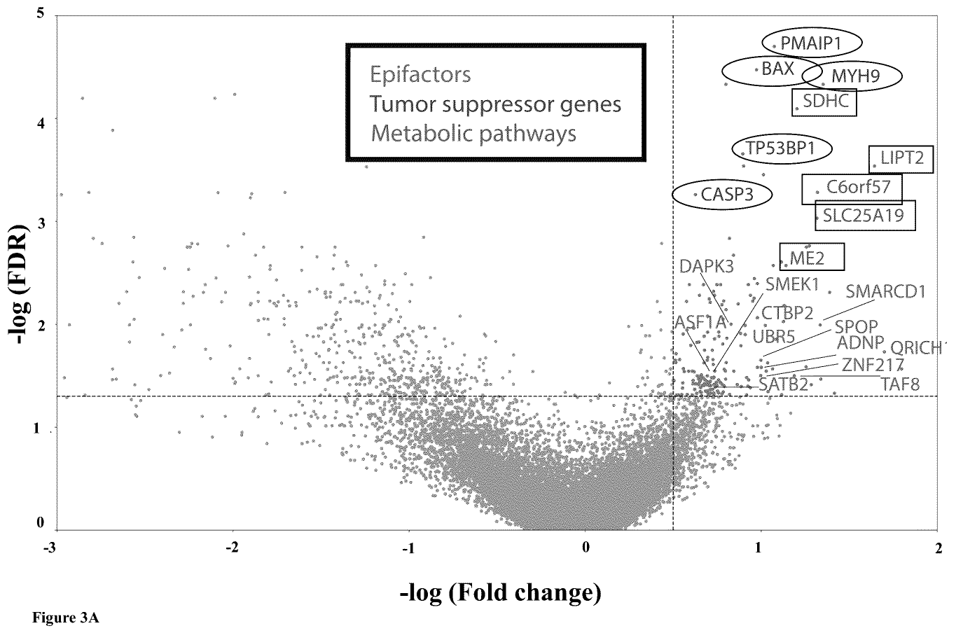

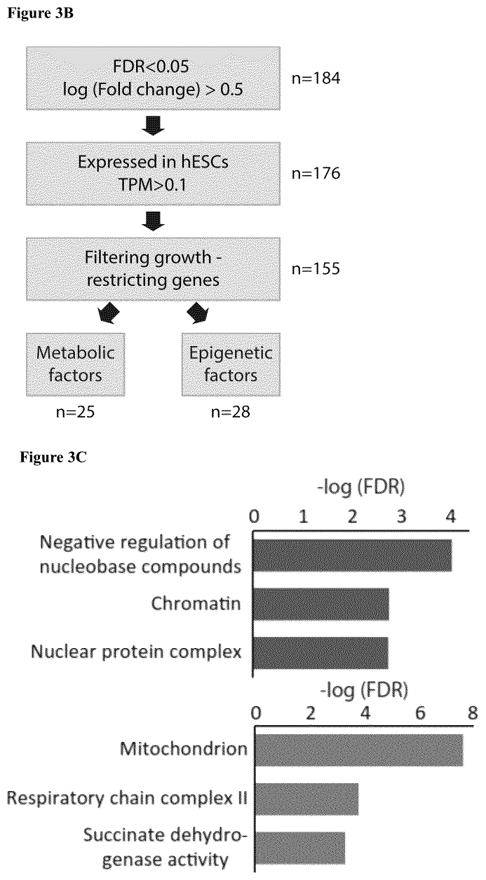

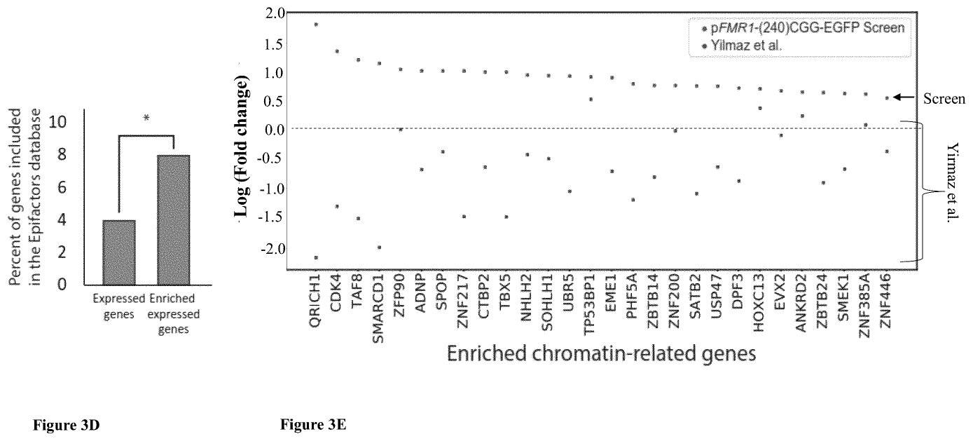

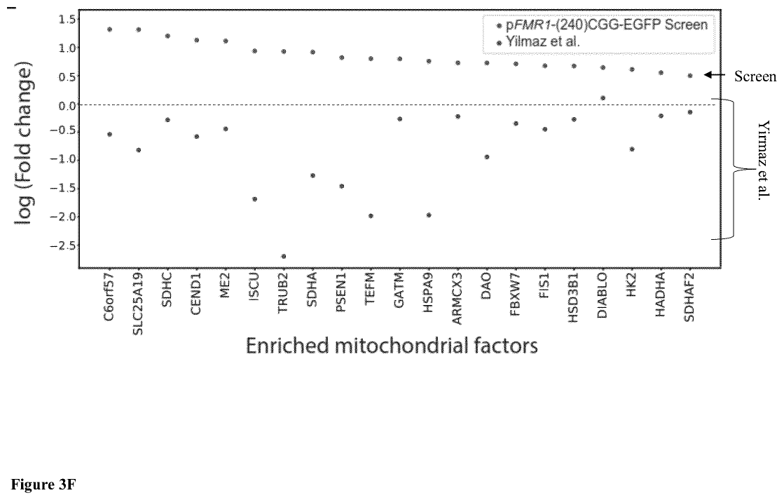

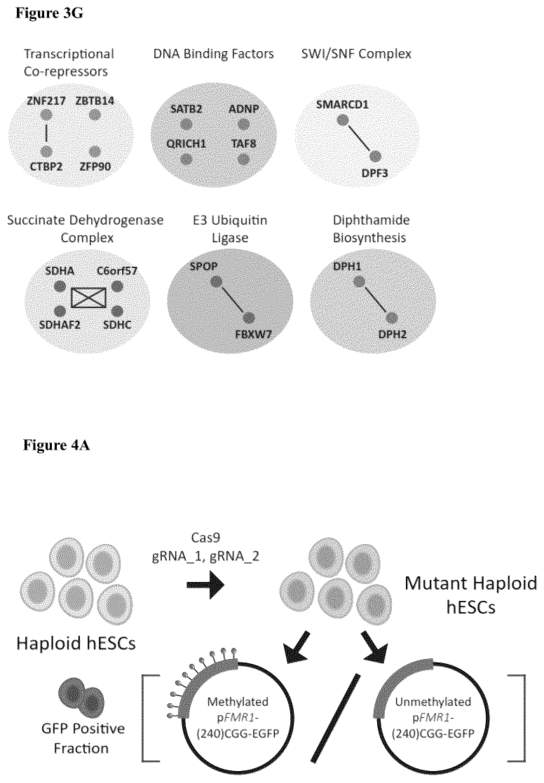

Analysis of the significantly enriched genes in the GFP-positive population revealed several functionally related gene groups (FIG. 3A-3C): First, a subset of the candidate genes was related to chromatin regulation and transcriptional repression, identified either by the Epifactors database, as being listed in databases of transcription factors or as associated with chromatin-related Gene Ontology (GO) annotations. Functional annotation analysis by GSEA revealed a significant association of the top enriched genes with GO terms related to chromatin regulation (FIG. 3C), and a canonical pathway analysis demonstrated a significant association with the reactome of RNA polymerase II, the central enzyme that catalyzes the expression of protein-coding genes (FDR<0.0001). Specifically, genes included in the Epifactor database were significantly enriched in the candidate list compared to their representation in the library (7.6% vs. 3.8%, P-value=0.02 using Fisher's exact test, FIG. 3D). Interestingly, another subset of the enriched genes was related to several metabolic pathways, specifically the mitochondrial respiration pathway, including four different subunits that assemble the succinate dehydrogenase complex (SDHA, SDHAF2, SDHC, C6orf57) (FIG. 3E-3F). This enrichment can be explained by the well-known influence of metabolic enzymes on the epigenome, mainly by catalyzing the production and degradation of metabolites that function as substrates, cofactors, or inhibitors of chromatin-modifying enzymes. Finally, some enriched genes were categorized as tumor suppressor genes previously found to be growth restricting in human PSCs (FIG. 3A). Although cell-cycle-related genes may influence the epigenetic landscape, these genes might be overrepresented in the screen because their disruption confers growth advantage under selection pressure. To filter out genes that were overrepresented due to selection pressure, genes identified as growth-restricting in human ESCs were excluded (FDR<0.05, CRISPR score>1). This led to the establishment of a candidate list of 155 genes predicted to be involved in maintaining gene repression, 28 of which were previously shown to have a role in chromatin regulation (FIG. 3E). Among these genes were transcriptional co-repressors (e.g., ZNF217, ZFP90, CTBP2), chromatin remodeling factors (e.g., SMARCD1), and RNA polymerase II transcription initiation factors (e.g., TAF8) (FIG. 3G). The enrichment of both epigenetic and mitochondrial factors among the GFP-positive population was not correlated with their association with growth restriction in a previous essentiality screen in haploid hPSCs (FIG. 3E-3F) (see, Yilmaz et al., 2018).

### Example 4: Characterization of Genes Predicted to be Involved in FMR1 Epigenetic Silencing

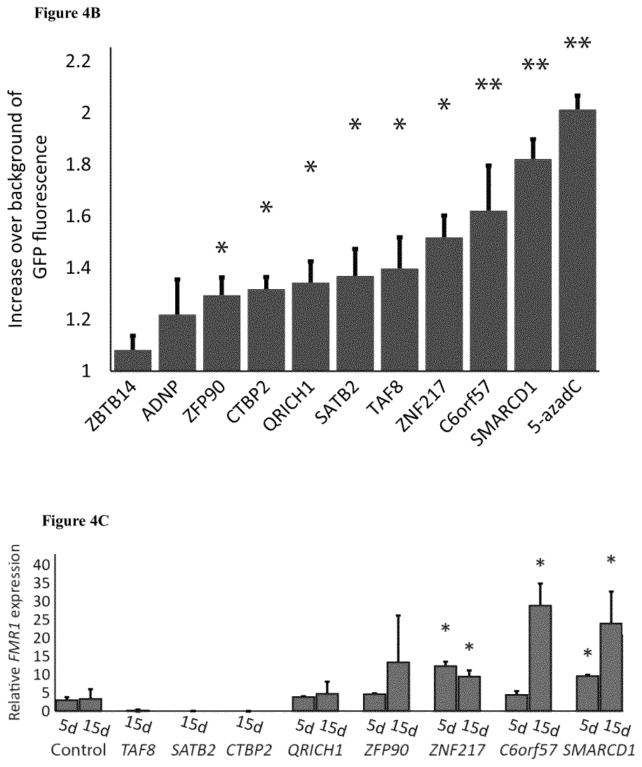

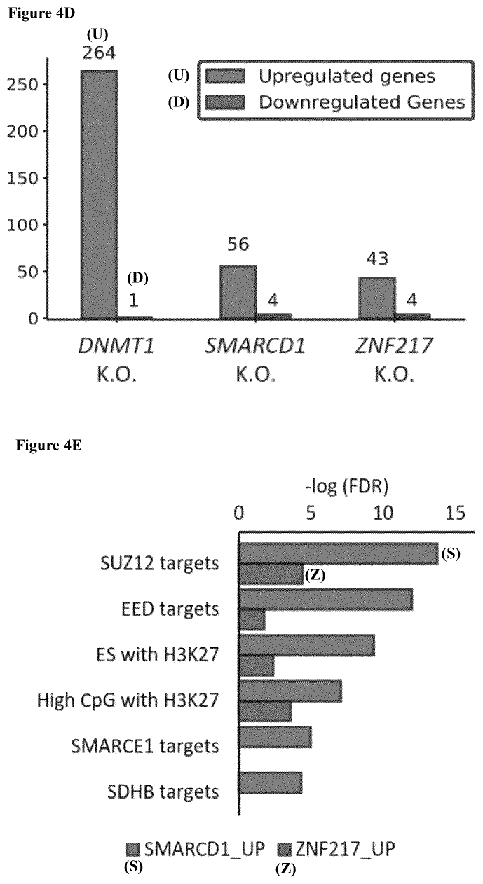

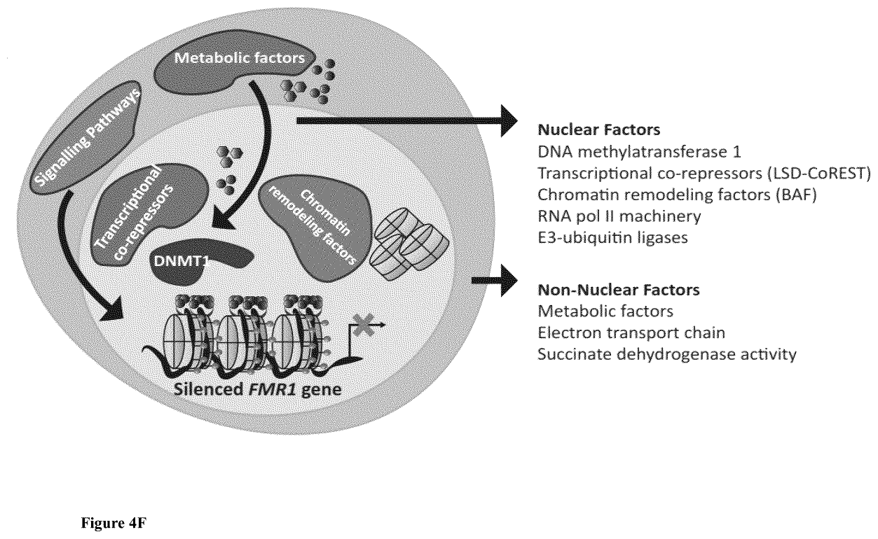

Next, the effect of disrupting selected candidate genes on the expression of the methylated pFMR1-(240) CGG-EGFP construct was validated. Using transduction with CRISPR/Cas9 and two sgRNAs per gene, the selected genes were mutated in haploid human ESCs, followed by transient pFMR1-(240) CGG-EGFP transfection. Mutant cultures were transfected with both unmethylated and methylated pFMR1-(240) CGG-EGFP construct, and the GFP fluorescent population was compared between the two cultures (FIG. 4A). This way, the epigenetic effect of candidate gene disruption could be isolated and confounding factors such as transfection efficiency or translational control could be excluded. Haploid hESCs infected with lentiviral vectors targeting the transcriptional regulators SMARCD1 and ZNF217, as well as the metabolic factor C6orf57, showed the highest levels of methylated construct expression compared to haploid hESCs infected with Cas9 without sgRNA. The mutant cultures demonstrated a range of 1.5-2 fold average increase in relative GFP fluorescence upon methylated construct transfection, reflecting the reversal of DNA methylation-mediated silencing (FIG. 4B).

Finally, as transiently transfected promoters might not be subjected to the same regulatory mechanisms which operate on the endogenous FMR1 promoter, the effect of candidate gene disruption in FXS-iPSCs was evaluated. FXS-iPSCs were infected with lentiviral constructs containing Cas9 and two different sgRNAs targeting each candidate gene. RT-PCR analysis of the mutated samples revealed some increase above basal FMR1 transcription levels following the disruption of SMARCD1, ZNF217 and C6orf57 (FIG. 4C). Although none of the mutated samples reached the FMR1 expression levels associated with DNMT1 perturbation, the relative increase in FMR1 mRNA in the mutant samples suggests an interaction of these candidate genes with the endogenous silenced FMR1 locus. To further characterize the regulatory effect of the identified genes, a global transcriptional analysis of the ZNF217- and SMARCD1-mutated samples was performed. Upon comparison of upregulated and downregulated genes (p-values<0.001 and |FC|>1), the mutated samples showed a bias towards a positive effect on gene expression, supporting the repressive function of these genes (FIG. 4D). Analysis of the enriched gene sets among the upregulated genes following SMARCD1 or ZNF217 disruption identified overlapping regulators of both gene groups, pointing to common regulatory pathways for these target genes (FIG. 3E). In the case of SMARCD1, there was also an enrichment of the targets of SMARCE1, another component of SWI/SNF chromatin remodeling complex, and of the targets of SDHB, a component of the succinate dehydrogenase complex (FIG. 4E).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A method of treating Fragile X syndrome (FXS) in a subject in need thereof, the method comprising administering to said subject an agent that inhibits expression or function of a protein selected from the group consisting of: SMARCD1, C6orf57, ZNF217, QRICH1, CDK4, TAF8, ZFP90, ADNP, SPOP, CTBP2, TBX5, NHLH2, SOHLH1, UBR5, TP53BP1, EME1, PHF5A, ZBTB14, ZNF200, SATB2, USP47, DPF3, HOXC13, EVX2, ANKRD2, ZBTB24, SMEK1, ZNF385A, ZNF446, SLC25A19, SDHC, CEND1, ME2, ISCU, TRUB2, SDHA, PSEN1, TEFM, GATM, HSPA9, ARMCX3, DAO, FBXW7, FIS1, HSD3B1, DIABLO, HK2, HADHA, SDHAF2, DPH1, DPH2, ASF1A, DAPK3, and LIPT2, thereby treating FXS.

2. (canceled)

3. (canceled)

4. (canceled)

5. (canceled)

6. The method of claim 1, wherein said agent inhibits ZNF217 and is an LSD1 inhibitor, a CoREST inhibitor or both.

7. The method of claim 6, wherein said LSD1 inhibitor, CoREST inhibitor or both are selected from GSK2879552, bizine, corin, pargyline, and Rodin-A.

8. The method of claim 1, wherein said agent inhibits ZNF217 and is an HDAC inhibitor.

9. The method of claim 8, wherein said HDAC inhibitor is selected from MS-275, SAHA, and LBH589.

10. The method of claim 1, wherein said agent inhibits SMARCD1 and is I-BRD9.

11. The method of claim 1, wherein said agent inhibits C6orf57 and is a succinate dehydrogenase (SDH) inhibitor.

12. The method of claim 11, wherein said SDH inhibitor is selected from Oxaloacetic acid, Malonate, Harzianopyridone, and 2-thenoyltrifluoroacetone.

13. The method of claim 1, wherein said agent is selected from an inhibitory RNA, a blocking antibody and a small molecule inhibitor.

14. The method of claim 1, wherein said treating comprises demethylation of a CGG repeat region of a 5′ UTR of a FMR1 gene in said subject.

15. The method of claim 1, wherein said treating comprises inducing expression of FMR1 in said subject.

16. The method of claim 1, further comprising inhibiting expression or function of at least one other protein selected from the group provided in Table 1.

17. A method of determining suitability of a subject in need thereof to be treated by a method of claim 1, the method comprising measuring in a sample from said subject expression or function of a protein selected from SMARCD1, C6orf57, ZNF217, QRICH1, CDK4, TAF8, ZFP90, ADNP, SPOP, CTBP2, TBX5, NHLH2, SOHLH1, UBR5, TP53BP1, EME1, PHF5A, ZBTB14, ZNF200, SATB2, USP47, DPF3, HOXC13, EVX2, ANKRD2, ZBTB24, SMEK1, ZNF385A, ZNF446, SLC25A19, SDHC, CEND1, ME2, ISCU, TRUB2, SDHA, PSEN1, TEFM, GATM, HSPA9, ARMCX3, DAO, FBXW7, FIS1, HSD3B1, DIABLO, HK2, HADHA, SDHAF2, DPH1, DPH2, ASF1A, DAPK3, and LIPT2, wherein expression or function above a predetermined threshold indicates said subject is suitable to be treated by a method of claim 1, thereby determining suitability of a subject.

18. (canceled)

19. (canceled)

20. (canceled)

21. (canceled)

22. The method of claim 17, wherein a cell from said sample comprises an expansion of a repetitive CGG sequence in a 5′ UTR of a FMR1 gene.

23. The method of claim 22, wherein said expansion comprises at least 55 CGG repeats.

24. The method of claim 17, wherein said subject suffers from FXS or is at risk of suffering from FXS.

25. A method for producing an agent, the method comprising:
a. providing an agent that inhibits expression or function of a protein selected from SMARCD1, C6orf57, ZNF217, QRICH1, CDK4, TAF8, ZFP90, ADNP, SPOP, CTBP2, TBX5, NHLH2, SOHLH1, UBR5, TP53BP1, EME1, PHF5A, ZBTB14, ZNF200, SATB2, USP47, DPF3, HOXC13, EVX2, ANKRD2, ZBTB24, SMEK1, ZNF385A, ZNF446, SLC25A19, SDHC, CEND1, ME2, ISCU, TRUB2, SDHA, PSEN1, TEFM, GATM, HSPA9, ARMCX3, DAO, FBXW7, FIS1, HSD3B1, DIABLO, HK2, HADHA, SDHAF2, DPH1, DPH2, ASF1A, DAPK3, and LIPT2;
b. contacting said provided agent with a cell comprising an FMR1 gene comprising a methylated CGG repeat region; and
c. selecting an agent that increases expression of FMR1 in said cell;
thereby producing an agent.

26. The method of claim 25, wherein the agent is an agent that treats FXS.

27. The method of claim 25, wherein said providing comprises contacting a plurality of agents with a cell expressing said protein and selecting at least one agent of said plurality that inhibits expression or function of said protein in said cell.

28. (canceled)

29. (canceled)

30. An agent produced by the method of claim 25.
